(19) United States
(12) Patent Application Publication
(10) Pub. No.: US 2023/0340446 A1
(43) Pub. Date: Oct. 26, 2023

(54) BIOSYNTHESIS OF CANNABINOIDS AND CANNABINOID PRECURSORS

(71) Applicant: Ginkgo Bioworks, Inc., Boston, MA (US)

(72) Inventors: Kim Cecelia Anderson, Somerville, MA (US); Brian Carvalho, Somerville, MA (US); Nicholas Flores, Boston, MA (US); Seth Ritter, Boston, MA (US); Gabriel Rodriguez, Central, SC (US); Michelle Spencer, Boston, MA (US); Emily E. Wrenbeck, Boston, MA (US)

(73) Assignee: Ginkgo Bioworks, Inc., Boston, MA (US)

(21) Appl. No.: 18/010,666

(22) PCT Filed: Jun. 17, 2021
(86) PCT No.: PCT/US2021/037954

§ 371 Date: Dec. 15, 2022

Related U.S. Application Data
(60) Provisional application No. 63/040,458, filed on Jun. 17, 2020.
(60) Provisional application No. 63/192,476, filed on May 24, 2021.

(51) Int. Cl. C12N 9/88; C12P 7/42

(52) U.S. Cl. CPC C12N 9/88; C12P 7/42; C12Y 404/01026

(57) ABSTRACT

Aspects of the disclosure relate to biosynthesis of cannabinoids and cannabinoid precursors in recombinant cells and in vitro. In particular, the disclosure relates to variants of olivetolic acid cyclase.

### CROSS REFERENCE TO RELATED APPLICATIONS

This application claims the benefit under 35 U.S.C. § 119(e) of U.S. Provisional Application No. 63/040,458, filed Jun. 17, 2020, entitled “BIOSYNTHESIS OF CANNABINOIDS AND CANNABINOID PRECURSORS” and U.S. Provisional Application No. 63/192,476, filed May 24, 2021, entitled “BIOSYNTHESIS OF CANNABINOIDS AND CANNABINOID PRECURSORS” the entire disclosure of each of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Jun. 17, 2021, is named G091970052W000-SEQ-FL.TXT and is 1,058,904 bytes in size.

### FIELD OF INVENTION

The present disclosure relates to the biosynthesis of cannabinoids and cannabinoid precursors, such as in recombinant cells.

### BACKGROUND

Cannabinoids are chemical compounds that may act as ligands for endocannabinoid receptors and have multiple medical applications. Traditionally, cannabinoids have been isolated from plants of the genus Cannabis. The use of plants for producing cannabinoids is inefficient, however, with isolated products often limited to the two most prevalent endogenous cannabinoids, THC and CBD, as minor cannabinoids are typically produced in very low concentrations in Cannabis plants. Further, the cultivation of Cannabis plants is restricted in many jurisdictions. In addition, in order to obtain consistent results, Cannabis plants are often grown in a controlled environment, such as indoor grow rooms without windows, to provide flexibility in modulating growing conditions such as lighting, temperature, humidity, airflow, etc. Growing Cannabis plants in such controlled environments can result in high energy usage per gram of cannabinoid produced, especially for minor cannabinoids that the plants produce only in small amounts. For example, lighting in such grow rooms is provided by artificial sources, such as high-powered sodium lights. As many species of Cannabis have a vegetative cycle that requires 18 or more hours of light per day, powering such lights can result in significant energy expenditures. It has been estimated that between 0.88-1.34 kWh of energy is required to produce one gram of THC in dried Cannabis flower form (e.g., before any extraction or purification). Additionally, concern has been raised over agricultural practices in certain jurisdictions, such as California, where the growing season coincides with the dry season such that the water usage may impact connected surface water in streams (Dillis, Christopher, Connor McIntee, Van Butsic, Lance Le, Kason Grady, and Theodore Grantham. “Water storage and irrigation practices for cannabis drive seasonal patterns of water extraction and use in Northern California.” Journal of Environmental Management 272 (2020): 110955).

Cannabinoids can also be produced through chemical synthesis (see, e.g., U.S. Pat. No. 7,323,576 to Souza et al). However, such methods suffer from low yields and high cost.

Production of cannabinoids, cannabinoid analogs, and cannabinoid precursors using engineered organisms may provide an advantageous approach to meet the increasing demand for these compounds.

### SUMMARY

Aspects of the present disclosure provide methods for production of cannabinoids and cannabinoid precursors from fatty acid substrates using genetically modified host cells.

Aspects of the disclosure relate to host cells that comprise a heterologous polynucleotide encoding a polyketide cyclase (PKC), wherein the PKC comprises an amino acid deletion or substitution at one or more residues corresponding to position 2, 3, 13, 17, 18, 19, 20, 21, 22, 24, 25, 26, 28, 29, 31, 33, 37, 38, 39, 41, 45, 46, 48, 49, 50, 52, 53, 55, 56, 57, 59, 60, 61, 62, 64, 65, 66, 67, 68, 69, 70, 71, 73, 74, 80, 81, 82, 83, 84, 85, 86, 87, 88, 89, 94, 95, 97, 98, 100, and/or 101 in SEQ ID NO: 1.

In some embodiments, the PKC comprises: the amino acid G at a residue corresponding to position 2 in SEQ ID NO: 1 or a deletion of a residue corresponding to position 2 in SEQ ID NO: 1; the amino acid M or Y at a residue corresponding to position 3 in SEQ ID NO: 1; the amino acid R or K at a residue corresponding to position 13 in SEQ ID NO: 1; the amino acid N at a residue corresponding to position 17 in SEQ ID NO: 1; the amino acid D or R at a residue corresponding to position 18 in SEQ ID NO: 1; the amino acid E or D at a residue corresponding to position 19 in SEQ ID NO: 1; the amino acid A at a residue corresponding to position 20 in SEQ ID NO: 1; the amino acid K at a residue corresponding to position 21 in SEQ ID NO: 1; the amino acid T or K at a residue corresponding to position 22 in SEQ ID NO: 1; the amino acid M at a residue corresponding to position 24 in SEQ ID NO: 1; the amino acid A or N at a residue corresponding to position 25 in SEQ ID NO: 1; the amino acid H, R, A, or E at a residue corresponding to position 26 in SEQ ID NO: 1; the amino acid A, S, E, or H at a residue corresponding to position 28 in SEQ ID NO: 1; the amino acid D, S, E, or T at a residue corresponding to position 29 in SEQ ID NO: 1; the amino acid L or A at a residue corresponding to position 31 in SEQ ID NO: 1; the amino acid H, R, D, E, K, Q, or N at a residue corresponding to position 33 in SEQ ID NO: 1; the amino acid L at a residue corresponding to position 37 in SEQ ID NO: 1; the amino acid L at a residue corresponding to position 38 in SEQ ID NO: 1; the amino acid G, S, or Q at a residue corresponding to position 39 in SEQ ID NO: 1; the amino acid K or R at a residue corresponding to position 41 in SEQ ID NO: 1; the amino acid I at a residue corresponding to position 45 in SEQ ID NO: 1; the amino acid F at a residue corresponding to position 46 in SEQ ID NO: 1; the amino acid A at a residue corresponding to position 48 in SEQ ID NO: 1; the amino acid Q at a residue corresponding to position 49 in SEQ ID NO: 1; the amino acid K, F, or W at a residue corresponding to position 50 in SEQ ID NO: 1; the amino acid H at a residue corresponding to position 52 in SEQ ID NO: 1; the amino acid H at a residue corresponding to position 53 in SEQ ID NO: 1; the amino acid F at a residue corresponding to position 55 in SEQ ID NO: 1; the amino acid Y at a residue corresponding to position 56 in SEQ ID NO: 1; the amino acid Y at a residue corresponding to position 57 in SEQ ID NO: 1; the amino acid I or F at a residue corresponding to position 59 in SEQ ID NO: 1; the amino acid I or L at a residue corresponding to position 60 in SEQ ID NO: 1; the amino acid T, D, or S at a residue corresponding to position 61 in SEQ ID NO: 1; the amino acid M at a residue corresponding to position 62 in SEQ ID NO: 1; the amino acid Q or D at a residue corresponding to position 64 in SEQ ID NO: 1; the amino acid N at a residue corresponding to position 65 in SEQ ID NO: 1; the amino acid L, M, or Y at a residue corresponding to position 66 in SEQ ID NO: 1; the amino acid K at a residue corresponding to position 67 in SEQ ID NO: 1; the amino acid E, G, or H at a residue corresponding to position 68 in SEQ ID NO: 1; the amino acid L or Y at a residue corresponding to position 69 in SEQ ID NO: 1; the amino acid A or L at a residue corresponding to position 70 in SEQ ID NO: 1; the amino acid Q, S, or E at a residue corresponding to position 71 in SEQ ID NO: 1; the amino acid L at a residue corresponding to position 73 in SEQ ID NO: 1; the amino acid D, E, H, Q, S, G, or L at a residue corresponding to position 74 in SEQ ID NO: 1; the amino acid V, A, E, S, D, or N at a residue corresponding to position 80 in SEQ ID NO: 1; the amino acid Y at a residue corresponding to position 81 in SEQ ID NO: 1; the amino acid K, V, or R at a residue corresponding to position 82 in SEQ ID NO: 1; the amino acid A, E, G, K, N, Q, R, or S at a residue corresponding to position 83 in SEQ ID NO: 1; the amino acid F, K, M, or Q at a residue corresponding to position 84 in SEQ ID NO: 1; the amino acid A or F at a residue corresponding to position 85 in SEQ ID NO: 1; the amino acid N, G, or S at a residue corresponding to position 86 in SEQ ID NO: 1; the amino acid C, D, or K at a residue corresponding to position 87 in SEQ ID NO: 1; the amino acid Y, N, or S at a residue corresponding to position 88 in SEQ ID NO: 1; the amino acid V or M at a residue corresponding to position 89 in SEQ ID NO: 1; the amino acid V at a residue corresponding to position 94 in SEQ ID NO: 1; the amino acid M or I at a residue corresponding to position 95 in SEQ ID NO: 1; the amino acid F at a residue corresponding to position 97 in SEQ ID NO: 1; the amino acid E, K, or Y at a residue corresponding to position 98 in SEQ ID NO: 1; the amino acid A, G, K, N, Q, or S at a residue corresponding to position 100 in SEQ ID NO: 1; and/or the amino acid A or E at a residue corresponding to position 101 in SEQ ID NO: 1.

In some embodiments, the PKC comprises an amino acid deletion or substitution at one or more residues corresponding to position 2, 3, 13, 17, 18, 19, 20, 21, 22, 24, 25, 26, 28, 29, 31, 33, 37, 38, 39, 45, 46, 48, 49, 50, 52, 53, 55, 56, 57, 59, 60, 61, 62, 64, 65, 66, 67, 68, 70, 71, 74, 80, 82, 83, 84, 85, 86, 87, 88, 89, 94, 95, 97, 98, 100, and 101 in SEQ ID NO: 1.

In some embodiments, the PKC comprises: the amino acid G at a residue corresponding to position 2 in SEQ ID NO: 1 or a deletion of a residue corresponding to position 2 in SEQ ID NO: 1; the amino acid Y at a residue corresponding to position 3 in SEQ ID NO: 1; the amino acid R or K at a residue corresponding to position 13 in SEQ ID NO: 1; the amino acid N at a residue corresponding to position 17 in SEQ ID NO: 1; the amino acid D or R at a residue corresponding to position 18 in SEQ ID NO: 1; the amino acid D at a residue corresponding to position 19 in SEQ ID NO: 1; the amino acid A at a residue corresponding to position 20 in SEQ ID NO: 1; the amino acid K at a residue corresponding to position 21 in SEQ ID NO: 1; the amino acid K at a residue corresponding to position 22 in SEQ ID NO: 1; the amino acid M at a residue corresponding to position 24 in SEQ ID NO: 1; the amino acid A or N at a residue corresponding to position 25 in SEQ ID NO: 1; the amino acid H, R, or A at a residue corresponding to position 26 in SEQ ID NO: 1; the amino acid E or H at a residue corresponding to position 28 in SEQ ID NO: 1; the amino acid S, E, or T at a residue corresponding to position 29 in SEQ ID NO: 1; the amino acid L at a residue corresponding to position 31 in SEQ ID NO: 1; the amino acid H, R, N at a residue corresponding to position 33 in SEQ ID NO: 1; the amino acid L at a residue corresponding to position 37 in SEQ ID NO: 1; the amino acid L at a residue corresponding to position 38 in SEQ ID NO: 1; the amino acid G or Q at a residue corresponding to position 39 in SEQ ID NO: 1; the amino acid I at a residue corresponding to position 45 in SEQ ID NO: 1; the amino acid F at a residue corresponding to position 46 in SEQ ID NO: 1; the amino acid A at a residue corresponding to position 48 in SEQ ID NO: 1; the amino acid Q at a residue corresponding to position 49 in SEQ ID NO: 1; the amino acid F, W, or K at a residue corresponding to position 50 in SEQ ID NO: 1; the amino acid H at a residue corresponding to position 52 in SEQ ID NO: 1; the amino acid H at a residue corresponding to position 53 in SEQ ID NO: 1; the amino acid F at a residue corresponding to position 55 in SEQ ID NO: 1; the amino acid Y at a residue corresponding to position 56 in SEQ ID NO: 1; the amino acid Y at a residue corresponding to position 57 in SEQ ID NO: 1; the amino acid F at a residue corresponding to position 59 in SEQ ID NO: 1; the amino acid I or L at a residue corresponding to position 60 in SEQ ID NO: 1; the amino acid D, S, or T at a residue corresponding to position 61 in SEQ ID NO: 1; the amino acid M at a residue corresponding to position 62 in SEQ ID NO: 1; the amino acid D at a residue corresponding to position 64 in SEQ ID NO: 1; the amino acid N at a residue corresponding to position 65 in SEQ ID NO: 1; the amino acid Y at a residue corresponding to position 66 in SEQ ID NO: 1; the amino acid K at a residue corresponding to position 67 in SEQ ID NO: 1; the amino acid G or H at a residue corresponding to position 68 in SEQ ID NO: 1; the amino acid A or L at a residue corresponding to position 70 in SEQ ID NO: 1; the amino acid Q, S, or E at a residue corresponding to position 71 in SEQ ID NO: 1; the amino acid G at a residue corresponding to position 74 in SEQ ID NO: 1; the amino acid V, A, D, or N at a residue corresponding to position 80 in SEQ ID NO: 1; the amino acid K or R at a residue corresponding to position 82 in SEQ ID NO: 1; the amino acid A, E, G, K, N, Q, R, or S at a residue corresponding to position 83 in SEQ ID NO: 1; the amino acid F or K at a residue corresponding to position 84 in SEQ ID NO: 1; the amino acid A or F at a residue corresponding to position 85 in SEQ ID NO: 1; the amino acid S at a residue corresponding to position 86 in SEQ ID NO: 1; the amino acid C or K at a residue corresponding to position 87 in SEQ ID NO: 1; the amino acid Y, N, or S at a residue corresponding to position 88 in SEQ ID NO: 1; the amino acid M at a residue corresponding to position 89 in SEQ ID NO: 1; the amino acid V at a residue corresponding to position 94 in SEQ ID NO: 1; the amino acid M or I at a residue corresponding to position 95 in SEQ ID NO: 1; the amino acid F at a residue corresponding to position 97 in SEQ ID NO: 1; the amino acid E, K, or Y at a residue corresponding to position 98 in SEQ ID NO: 1; the amino acid A, G, K, N, Q, or S at a residue corresponding to position 100 in SEQ ID NO: 1; and/or the amino acid A or E at a residue corresponding to position 101 in SEQ ID NO: 1.

In some embodiments, the PKC comprises an amino acid substitution at a residue corresponding to position 2, 3, 13, 17, 18, 19, 20, 21, 22, 25, 26, 28, 29, 31, 33, 37, 38, 39, 41, 45, 50, 52, 53, 55, 56, 57, 60, 62, 64, 65, 67, 68, 70, 71, 74, 83, 84, 85, 86, 87, 88, 95, 97, 98, 100, and/or 101 in SEQ ID NO: 1 wherein the PKC is capable of producing olivetolic acid from 3,5,7-trioxododecanoyl-CoA, and wherein, if present: the amino acid substitution at the residue corresponding to position 13 is D13R or D13K; the amino acid substitution at the residue corresponding to position 21 is E21K; the amino acid substitution at the residue corresponding to position 39 is D39G, D39S, or D39Q; the amino acid substitution at the residue corresponding to position 50 is N50K; the amino acid substitution at the residue corresponding to position 52 is E52H; the amino acid substitution at the residue corresponding to position 67 is E67K; the amino acid substitution at the residue corresponding to position 70 is Q70A or Q70L; the amino acid substitution at the residue corresponding to position 71 is D71Q or D71S; the amino acid substitution at the residue corresponding to position 74 is I74D, 174E, I74H, or I74Q; the amino acid substitution at the residue corresponding to position 83 is D83A or D83G; the amino acid substitution at the residue corresponding to position 86 is R86G; the amino acid substitution at the residue corresponding to position 87 is S87D or S87K; and the amino acid substitution at the residue corresponding to position 100 is R100A, R100G, R100K, R100N, R100Q, or R100S.

In some embodiments, the PKC comprises an amino acid substitution at a residue corresponding to position 2, 3, 13, 17, 18, 19, 20, 21, 22, 25, 26, 28, 29, 31, 33, 37, 38, 39, 45, 50, 52, 53, 55, 56, 57, 60, 62, 64, 65, 67, 68, 70, 71, 83, 84, 85, 87, 88, 95, 97, 98, 100, and/or 101 in SEQ ID NO: 1, and wherein, if present: the amino acid substitution at the residue corresponding to position 13 is D13R or D13K; the amino acid substitution at the residue corresponding to position 21 is E21K; the amino acid substitution at the residue corresponding to position 39 is D39G or D39Q; the amino acid substitution at the residue corresponding to position 50 is N50K; the amino acid substitution at the residue corresponding to position 52 is E52H; the amino acid substitution at the residue corresponding to position 67 is E67K; the amino acid substitution at the residue corresponding to position 70 is Q70A or Q70L; the amino acid substitution at the residue corresponding to position 71 is D71Q or D71S; the amino acid substitution at the residue corresponding to position 83 is D83A or D83G; the amino acid substitution at the residue corresponding to position 87 is S87K; and the amino acid substitution at the residue corresponding to position 100 is R100S.

In some embodiments, the PKC comprises one or more amino acid substitutions or deletions located in a domain corresponding to: beta sheet 1 (positions 1-11 in SEQ ID NO: 1); loop 1 (positions 12-16 in SEQ ID NO: 1); helix 1 (positions 17-29 in SEQ ID NO: 1); 310 helix 1 (positions 30-32 in SEQ ID NO: 1); loop 2 (positions 33-38 in SEQ ID NO: 1); beta sheet 2 (positions 39-44 in SEQ ID NO: 1); loop 3 (positions 45-56 in SEQ ID NO: 1); beta sheet 3 (positions 57-62 in SEQ ID NO: 1); loop 4 (positions 63-65 in SEQ ID NO: 1); helix 2 (positions 66-73 in SEQ ID NO: 1); turn 1 (positions 74-75 in SEQ ID NO: 1); helix 3 (positions 76-85 in SEQ ID NO: 1); 310 helix 2 (positions 86-88 in SEQ ID NO: 1); beta sheet 4 (positions 89-97 in SEQ ID NO: 1); and/or loop 5 (positions 98-101 in SEQ ID NO: 1).

In some embodiments, one or more amino acid substitutions are located in a domain corresponding to: helix 1 (positions 17-29 in SEQ ID NO: 1); loop 3 (positions 45-56 in SEQ ID NO: 1); helix 2 (positions 66-73 in SEQ ID NO: 1); and/or helix 3 (positions 76-85 in SEQ ID NO: 1).

In some embodiments, one or more amino acid substitutions increase the stability of a kinked helix defined by the domains corresponding to helix 2 (positions 66-73 in SEQ ID NO: 1), turn 1 (positions 74-75 in SEQ ID NO: 1), and helix 3 (positions 76-85 in SEQ ID NO: 1) and/or improve conformation of a binding pocket of the PKC to 3,5,7-trioxododecanoyl-CoA and/or olivetolic acid.

In some embodiments, one or more amino acid substitutions are located in a domain corresponding to: loop 3 (positions 45-56 in SEQ ID NO: 1); helix 2 (positions 66-73 in SEQ ID NO: 1); and helix 3 (positions 76-85 in SEQ ID NO: 1).

In some embodiments, the PKC comprises a deletion or amino acid substitution at a residue corresponding to position 2, 18, 22, 24, 25, 26, 28, 29, 33, 52, 53, 57, 60, 62, 70, 82, 83, 84, 87, and/or 88 in SEQ ID NO: 1.

In some embodiments, the PKC comprises an amino acid substitution at a residue corresponding to position 18, 29, 53, 70, 82 and/or 83 in SEQ ID NO:1.

In some embodiments, the PKC comprises: a deletion at a residue corresponding to position 2 in SEQ ID NO: 1; the amino acid D or R at a residue corresponding to position 18 in SEQ ID NO: 1; the amino acid K at a residue corresponding to position 22 in SEQ ID NO: 1; the amino acid M at a residue corresponding to position 24 in SEQ ID NO: 1; the amino acid N at a residue corresponding to position 25 in SEQ ID NO: 1; the amino acid A or H at a residue corresponding to position 26 in SEQ ID NO: 1; the amino acid E at a residue corresponding to position 28 in SEQ ID NO: 1; the amino acid E or S at a residue corresponding to position 29 in SEQ ID NO: 1; the amino acid H or R at a residue corresponding to position 33 in SEQ ID NO: 1; the amino acid H at a residue corresponding to position 52 in SEQ ID NO: 1; the amino acid H at a residue corresponding to position 53 in SEQ ID NO: 1; the amino acid Y at a residue corresponding to position 57 in SEQ ID NO: 1; the amino acid I at a residue corresponding to position 60 in SEQ ID NO: 1; the amino acid M at a residue corresponding to position 62 in SEQ ID NO: 1; the amino acid L at a residue corresponding to position 70 in SEQ ID NO: 1; the amino acid K at a residue corresponding to position 82 in SEQ ID NO: 1; the amino acid A, K, or R at a residue corresponding to position 83 in SEQ ID NO: 1; the amino acid F or K at a residue corresponding to position 84 in SEQ ID NO: 1; the amino acid C or K at a residue corresponding to position 87 in SEQ ID NO: 1; and/or the amino acid Y at a residue corresponding to position 88 in SEQ ID NO: 1.

In some embodiments, relative to SEQ ID NO: 1, the PKC comprises: E53H, G82K, and Q70L.

In some embodiments, relative to SEQ ID NO: 1, the PKC comprises: A18D, T26A, V28E, N29S, E53H, E601, Q70L, G82K, D83A, and S87K; A18D, E22K, N29S, E53H, Q70L, G82K, D83R, S87K, and F88Y; A18D, E22K, N29S, E53H, Q70L, G82K, D83K, S87K, and F88Y; A18D, E22K, N29S, I33R, E53H, Q70L, G82K, D83A, S87K, and F88Y; A18D, E22K, K25N, T26A, N29S, E53H, Q70L, G82K, D83A, and S87K; A18D, E22K, N29S, E53H, Q70L, G82K, D83A, V84K, S87K, and F88Y; A18D, E22K, T26A, N29S, E53H, Q70L, G82K, D83A, S87K, and F88Y; A18D, E22K, T26H, N29S, E53H, Q70L, G82K, D83A, S87K, and F88Y; A18D, E22K, N29S, I33H, E53H, Q70L, G82K, D83A, S87K, and F88Y; A18D, E22K, N29S, E53H, Q70L, G82K, D83A, V84F, S87K, and F88Y; A18D, T26A, N29S, E53H, Q70L, G82K, D83A, and F88Y; A18D, E22K, T26A, N29S, E53H, Q70L, G82K, D83A, and S87K; A18D, T26A, N29S, E53H, Q70L, G82K, D83A, S87K, and F88Y; A18D, T26A, N29S, E53H, Q70L, G82K, and D83A; A18D, E22K, T26A, N29S, E53H, Q70L, G82K, D83A, and F88Y; A18D, E22K, N29S, E53H, Q70L, G82K, D83A, S87K, and F88Y; A18D, T26A, V28E, N29S, E53H, Q70L, G82K, D83A, and S87K; A18R, E22K, N29S, E53H, Q70L, G82K, D83A, S87K, and F88Y; deletion of a residue corresponding to position 2 in SEQ ID NO: 1, A18D, E22K, N29S, E53H, Q70L, G82K, D83A, S87K, and F88Y; A18D, E22K, N29S, E53H, T62M, Q70L, G82K, D83A, S87K, and F88Y; A18D, E22K, T26A, N29E, E53H, Q70L, G82K, D83A, and S87K; A18D, E22K, N29S, E52H, E53H, Q70L, G82K, D83A, S87K, and F88Y; A18D, E22K, N29S, E53H, Q70L, G82K, D83A, S87C, and F88Y; A18D, E22K, F24M, N29S, E53H, Q70L, G82K, D83A, S87K, and F88Y; A18D, N29S, E53H, Q70L, G82K, D83A, and F88Y; A18D, N29S, E53H, Q70L, G82K, D83A, S87K, and F88Y; A18D, E22K, T26A, N29S, E53H, H57Y, Q70L, G82K, D83A, and S87K; I33R, E52H, Y55F, Q70A, G80A, D83K, and V84K; D13R, A18D, N29S, Q70L, D71Q, G80A, and D83K; I33R, E52H, Y55F, Q70A, I74G, and D83K; T26R, I33H, E52H, Y55F, Q70A, and D83K; A18D, T26R, E52H, Y55F, Q70A, G80A, D83N, V84K, and Y85A; T26R, I33H, Y55F, Q70A, G80A, D83K, and Y85A; E22K, T26H, N29S, D39G, K49Q, E52H, D83N, and V84K; E17N, A18D, V31L, D71S, D83A, and V84F; or A18D, V31L, D39Q, D71S, D83A, and V84F.

In some embodiments, the PKC comprises an amino acid substitution at a residue corresponding to position 17, 18, 26, 29, 31, 33, 37, 39, 71, 80, 83, 84, and/or 95 in SEQ ID NO: 1.

In some embodiments, the PKC comprises: the amino acid N at a residue corresponding to position 17 in SEQ ID NO: 1; the amino acid D at a residue corresponding to position 18 in SEQ ID NO: 1; the amino acid A or H at a residue corresponding to position 26 in SEQ ID NO: 1; the amino acid T at a residue corresponding to position 29 in SEQ ID NO: 1; the amino acid L at a residue corresponding to position 31 in SEQ ID NO: 1; the amino acid R, H, or N at a residue corresponding to position 33 in SEQ ID NO: 1; the amino acid L at a residue corresponding to position 37 in SEQ ID NO: 1; the amino acid Q or G at a residue corresponding to position 39 in SEQ ID NO: 1; the amino acid S at a residue corresponding to position 71 in SEQ ID NO: 1; the amino acid A at a residue corresponding to position 80 in SEQ ID NO: 1; the amino acid Q, N, or A at a residue corresponding to position 83 in SEQ ID NO: 1; the amino acid F at a residue corresponding to position 84 in SEQ ID NO: 1; and/or the amino acid M at a residue corresponding to position 95 in SEQ ID NO: 1.

In some embodiments, relative to SEQ ID NO: 1, the PKC comprises: E17N, A18D, and D39G; D39Q, and F95M; I33N and F95M; E17N, I33R, and F95M; T26H, D83Q, and F95M; I33H, D39Q, and D71S; N29T, I33N, and D71S; I33N, D39Q, and F95M; D71S, D83N, and F95M; D39Q and D83Q; A18D and M37L; E17N, A18D, and I33N; D39G, D71S, and V84F; N29T and D39G; A18D, D71S, and G80A; N29T, V31L, and D39Q; A18D, V31L, and D39G; T26H, N29T, and D39Q; T26A, N29T, and I33H; D39Q, D71S, and G80A; A18D, D39Q, and V84F; E17N, I33R, and D39Q; E17N, D71S, and F95M; E17N, 133N, and F95M; T26A, N29T, and V31L; E17N, T26A, and D39G; A18D, D39Q, and D71S; V31L, D39G, and V84F; N29T, I33R, and D39Q; D39G, D71S, and D83N; V31L, D71S, and D83Q; I33R, D39Q, and V84F; D39G and D71S; E17N, I33H, and D71S; T26H, D39G, and V84F; A18D, I33R, and D39G; T26A, D83N, and F95M; V31L, I33H, and D71S; V31L, D39G, and D71S; A18D and D39Q; I33R, D39G, and D71S; E17N, G80A, and D83N; A18D, N29T, and V31L; D39Q, D71S, and D83A; A18D, T26H, and D39Q; T26A and F95M; I33H, D39G, and D83Q; T26H, I33N, and G80A; I33R, D39G, and F95M; V31L, I33R, and D83N; N29T, D39G, and V84F; D71S and D83Q; I33H, D39Q, and G80A; E17N, D39G, and D83A; E17N, 133N, and V84F; N29T, I33N, and D83Q; I33N, D71S, and G80A; I33R, D39G, and D83N; T26A and N29T; I33H, D83Q, and F95M; T26A, V31L, and D39Q; E17N, V31L, and I33N; E17N, T26A, and M37L; or N29T and I33N.

In some embodiments, the PKC comprises an amino acid substitution at a residue corresponding to position 13, 18, 22, 26, 29, 33, 39, 49, 52, 55, 61, 64, 65, 66, 68, 70, 71, 74, 80, 83, 84, 85, and/or 98 in SEQ ID NO: 1.

In some embodiments, the PKC comprises: the amino acid R at a residue corresponding to position 13 in SEQ ID NO: 1; the amino acid D at a residue corresponding to position 18 in SEQ ID NO: 1; the amino acid K at a residue corresponding to position 22 in SEQ ID NO: 1; the amino acid R or H at a residue corresponding to position 26 in SEQ ID NO: 1; the amino acid S at a residue corresponding to position 29 in SEQ ID NO: 1; the amino acid R or H at a residue corresponding to position 33 in SEQ ID NO: 1; the amino acid G at a residue corresponding to position 39 in SEQ ID NO: 1; the amino acid Q at a residue corresponding to position 49 in SEQ ID NO: 1; the amino acid H at a residue corresponding to position 52 in SEQ ID NO: 1; the amino acid F at a residue corresponding to position 55 in SEQ ID NO: 1; the amino acid D at a residue corresponding to position 61 in SEQ ID NO: 1; the amino acid D at a residue corresponding to position 64 in SEQ ID NO: 1; the amino acid N at a residue corresponding to position 65 in SEQ ID NO: 1; the amino acid Y at a residue corresponding to position 66 in SEQ ID NO: 1; the amino acid G at a residue corresponding to position 68 in SEQ ID NO: 1; the amino acid A or L at a residue corresponding to position 70 in SEQ ID NO: 1; the amino acid E or Q at a residue corresponding to position 71 in SEQ ID NO: 1; the amino acid G at a residue corresponding to position 74 in SEQ ID NO: 1; the amino acid N or A at a residue corresponding to position 80 in SEQ ID NO: 1; the amino acid K or N at a residue corresponding to position 83 in SEQ ID NO: 1; the amino acid K at a residue corresponding to position 84 in SEQ ID NO: 1; the amino acid F or A at a residue corresponding to position 85 in SEQ ID NO: 1; and/or the amino acid E or K at a residue corresponding to position 98 in SEQ ID NO: 1.

In some embodiments, relative to SEQ ID NO: 1, the PKC comprises: Y85F; E64D, D71E, and T98K; S65N; V66Y; G80N; T68G; D71E; E64D and D71E; D71E and Y85F; I33R, E52H, Y55F, Q70A, G80A, D83K, and V84K; D13R, A18D, N29S, Q70L, D71Q, G80A, and D83K; I33R, E52H, Y55F, Q70A, I74G, and D83K; Y55F, G80A, and D83K; T26R, I33H, E52H, Y55F, Q70A, and D83K; A18D, T26R, E52H, Y55F, Q70A, G80A, D83N, V84K, and Y85A; T98E; T26R, I33H, Y55F, Q70A, G80A, D83K, and Y85A; V61D; or E22K, T26H, N29S, D39G, K49Q, E52H, D83N, and V84K.

In some embodiments, the PKC comprises an amino acid substitution at a residue corresponding to position 13, 17, 18, 22, 26, 29, 31, 33, 39, 52, 55, 61, 64, 65, 66, 70, 71, 74, 80, 82, 83, 84, 85, 88, 95, and/or 98 in SEQ ID NO: 1.

In some embodiments, the PKC comprises: the amino acid R at a residue corresponding to position 13 in SEQ ID NO: 1; the amino acid N at a residue corresponding to position 17 in SEQ ID NO: 1; the amino acid D at a residue corresponding to position 18 in SEQ ID NO: 1; the amino acid K at a residue corresponding to position 22 in SEQ ID NO: 1; the amino acid R at a residue corresponding to position 26 in SEQ ID NO: 1; the amino acid S at a residue corresponding to position 29 in SEQ ID NO: 1; the amino acid L at a residue corresponding to position 31 in SEQ ID NO: 1; the amino acid H, N, or R at a residue corresponding to position 33 in SEQ ID NO: 1; the amino acid G or Q at a residue corresponding to position 39 in SEQ ID NO: 1; the amino acid H at a residue corresponding to position 52 in SEQ ID NO: 1; the amino acid F at a residue corresponding to position 55 in SEQ ID NO: 1; the amino acid S at a residue corresponding to position 61 in SEQ ID NO: 1; the amino acid D at a residue corresponding to position 64 in SEQ ID NO: 1; the amino acid N at a residue corresponding to position 65 in SEQ ID NO: 1; the amino acid Y at a residue corresponding to position 66 in SEQ ID NO: 1; the amino acid A or L at a residue corresponding to position 70 in SEQ ID NO: 1; the amino acid E, Q, or S at a residue corresponding to position 71 in SEQ ID NO: 1; the amino acid G at a residue corresponding to position 74 in SEQ ID NO: 1; the amino acid A at a residue corresponding to position 80 in SEQ ID NO: 1; the amino acid R at a residue corresponding to position 82 in SEQ ID NO: 1; the amino acid A, E, K, or N at a residue corresponding to position 83 in SEQ ID NO: 1; the amino acid F or K at a residue corresponding to position 84 in SEQ ID NO: 1; the amino acid A or F at a residue corresponding to position 85 in SEQ ID NO: 1; the amino acid N at a residue corresponding to position 88 in SEQ ID NO: 1; the amino acid M at a residue corresponding to position 95 in SEQ ID NO: 1; and/or the amino acid K or Y at a residue corresponding to position 98 in SEQ ID NO: 1.

In some embodiments, the PKC comprises an amino acid substitution at a residue corresponding to position 13, 17, 18, 22, 26, 31, 33, 39, 52, 55, 64, 65, 66, 70, 71, 74, 80, 82, 83, 84, 85, 88, and/or 98 in SEQ ID NO: 1.

In some embodiments, the PKC comprises: the amino acid R at a residue corresponding to position 13 in SEQ ID NO:1; the amino acid N at a residue corresponding to position 17 in SEQ ID NO:1; the amino acid D at a residue corresponding to position 18 in SEQ ID NO:1; the amino acid K at a residue corresponding to position 22 in SEQ ID NO:1; the amino acid R at a residue corresponding to position 26 in SEQ ID NO:1; the amino acid L at a residue corresponding to position 31 in SEQ ID NO:1; the amino acid R, H or N at a residue corresponding to position 33 in SEQ ID NO:1; the amino acid G at a residue corresponding to position 39 in SEQ ID NO:1; the amino acid H at a residue corresponding to position 52 in SEQ ID NO:1; the amino acid F at a residue corresponding to position 55 in SEQ ID NO:1; the amino acid D at a residue corresponding to position 64 in SEQ ID NO:1; the amino acid N at a residue corresponding to position 65 in SEQ ID NO:1; the amino acid Y at a residue corresponding to position 66 in SEQ ID NO:1; the amino acid A at a residue corresponding to position 70 in SEQ ID NO:1; the amino acid S or E at a residue corresponding to position 71 in SEQ ID NO:1; the amino acid R at a residue corresponding to position 82 in SEQ ID NO: 1; the amino acid G at a residue corresponding to position 74 in SEQ ID NO:1; the amino acid A at a residue corresponding to position 80 in SEQ ID NO:1; the amino acid E, A, K, or N at a residue corresponding to position 83 in SEQ ID NO:1; the amino acid K or F at a residue corresponding to position 84 in SEQ ID NO:1; the amino acid F at a residue corresponding to position 85 in SEQ ID NO:1; the amino acid N at a residue corresponding to position 88 in SEQ ID NO: 1; and/or the amino acid K or Y at a residue corresponding to position 98 in SEQ ID NO:1.

In some embodiments, relative to SEQ ID NO: 1, the PKC comprises: A18D, T26R, I33R, D39G, E52H, Y55F, E64D, Q70A, G80A, D83N, and V84K; E17N, T26R, V31L, D71S, D83E, and V84F; E17N, V31L, I33N, E64D, D71S, and V84F; E17N, A18D, V31L, D71S, D83A, and V84F; E17N, E22K, V31L, I33N, D71E, and V84F; E17N, A18D, V31L, D71E, D83A, and V84F; E17N, V31L, D71S, D83E, V84F, and T98Y; V84K, T26R, D39G, D83E, T98K, and S65N; E17N, A18D, V31L, I33N, D71S, D83E, and V84K; E17N, A18D, V31L, D71S, I74G, D83E, and V84F; E17N, V31L, I33N, D71S, V84K, and Y85F; A18D, T26R, I33R, E52H, Y55F, V66Y, Q70A, G80A, D83N, V84K, and Y85F; E17N, A18D, V31L, I33N, D71S, D83K, and V84K; E17N, A18D, T26R, V31L, I33N, D71S, and V84K; V84K, T26R, D39G, D13R, T98K, and S65N; E17N, V31L, I33N, S65N, D71S, and V84K; E17N, A18D, E22K, V31L, I33N, D71E, and V84F; D13R, E17N, A18D, V31L, D71S, D83A, and V84K; E17N, V31L, D39G, D71S, D83A, and V84K; D13R, A18D, T26R, E52H, Y55F, Q70A, G80A, D83N, V84K, and Y85F; E17N, A18D, T26R, V31L, D71S, D83A, and V84K; E17N, V31L, I33N, D71S, G82R, and V84F; E17N, V31L, I33N, D71S, V84K, and T98K; V84K, T26R, D39G, D83E, D13R, and T98K; V84K, T26R, D83E, D13R, T98K, and S65N; E17N, V31L, I33N, V66Y, D71E, and V84F; E17N, A18D, V31L, 133N, D71E, and V84F; E17N, A18D, V31L, I33N, D71S, G80A, and V84F; A18D, T26R, E52H, Y55F, Q70A, G80A, D83N, V84K, Y85F, and F88N; E17N, A18D, V31L, I33N, D71S, V84K, and T98K; E17N, A18D, V31L, I33N, S65N, D71S, and V84K; T26R, I33H, Y55F, Q70A, G80A, D83K, V84K, and F88N; A18D, E22K, T26R, E52H, Y55F, Q70A, D71E, G80A, D83N, and V84K; or E17N, V31L, 133N, D71E, G82R, and V84F.

In some embodiments, PKC comprises a sequence that is at least 90% identical to any one of SEQ ID NOs: 19-106, 196-388, 582-669, 671-742, and 745-948.

In some embodiments, the PKC comprises a sequence that is at least 90% identical to any one of SEQ ID NOs: 19, 21, 23, 24, 26, 27, 29, 32, 33, 34, 35, 38, 40, 42, 43, 45, 48, 49, 50, 51, 53, 58, 60, 62, 65, 66, 67, 68, 78, 87, 91, 92, 94, 99, 103, 105, 219, 220, 231, 234, 240, 254, 255, 258 283, 289, 294, 344, 345, 349, 365, 582-603, 605, 606, 607, 608, 610, 611, 612, 614, 616, 617, 618, 622, 625, 627, 629, 631, 632, 633, 634, 637, 638, 639, 640, 641, 642, 643, 644, 646, 648, 651, 653, 654, 655, 656, 657, 658, 659, 660, 661, 662, 663, 666, 667, 668, 672, 673, 674, 675, 676, 679, 680, 683, 684, 685, 686, 691, 693, 694, 695, 696, 697, 699, 700, 703, 707,711, 713, 714, 715, 724, 732, 734, 735, 736, 737, 738, 739, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

In some embodiments, the PKC comprises a sequence selected from any one of SEQ ID NOs: 19-106, 196-388, 582-669, 671-742, and 745-948.

In some embodiments, the PKC comprises a sequence selected from any one of SEQ ID NOs: 19, 21, 23, 24, 26, 27, 29, 32, 33, 34, 35, 38, 40, 42, 43, 45, 48, 49, 50, 51, 53, 58, 60, 62, 65, 66, 67, 68, 78, 87, 91, 92, 94, 99, 103, 105, 219, 220, 231, 234, 240, 254, 255, 258 283, 289, 294, 344, 345, 349, 365, 582-603, 605, 606, 607, 608, 610, 611, 612, 614, 616, 617, 618, 622, 625, 627, 629, 631, 632, 633, 634, 637, 638, 639, 640, 641, 642, 643, 644, 646, 648, 651, 653, 654, 655, 656, 657, 658, 659, 660, 661, 662, 663, 666, 667, 668, 672, 673, 674, 675, 676, 679, 680, 683, 684, 685, 686, 691, 693, 694, 695, 696, 697, 699, 700, 703, 707, 711, 713, 714, 715, 724, 732, 734, 735, 736, 737, 738, 739, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

In some embodiments, the heterologous polynucleotide comprises a sequence that is at least 90% identical to any one of SEQ ID NOs: 107-194, 389-581, 949-1036, 1038-1109 and 1112-1315.

In some embodiments, the heterologous polynucleotide comprises a sequence selected from any one of SEQ ID NOs: 107-194, 389-581, 949-1036, 1038-1109 and 1112-1315.

In some embodiments, the heterologous polynucleotide comprises a sequence that is at least 90% identical to any one of SEQ ID NOs: 107, 109, 111, 112, 114, 115, 117, 120, 121, 122, 123, 126, 128, 130, 131, 133, 136, 137, 138, 139, 141, 146, 148, 150, 153, 154, 155, 156, 166, 175, 179, 180, 182, 187, 191, 193, 412, 413, 424, 427, 433, 447, 448, 451, 476, 482, 487, 537, 538, 542, 558, 949-970, 1074, 972, 973, 974, 975, 977, 978, 979, 981, 983, 984, 985, 989, 992, 994, 996, 998, 999, 1000, 1001, 1004, 1005, 1006, 1007, 1008, 1009, 1010, 1011, 1013, 1015, 1018, 1020, 1021, 1022, 1023, 1024, 1025, 1026, 1027, 1028, 1029, 1030, 1033, 1034, 1035, 1039, 1040, 1041, 1042, 1043, 1046, 1047, 1050, 1051, 1052, 1053, 1058, 1060, 1061, 1062, 1063, 1064, 1066, 1067, 1070, 1078, 1080, 1081, 1082, 1091, 1099, 1101, 1102, 1103, 1104, 1105, 1106, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

In some embodiments, the heterologous polynucleotide comprises a sequence selected from any one of SEQ ID NOs: 107, 109, 111, 112, 114, 115, 117, 120, 121, 122, 123, 126, 128, 130, 131, 133, 136, 137, 138, 139, 141, 146, 148, 150, 153, 154, 155, 156, 166, 175, 179, 180, 182, 187, 191, 193, 412, 413, 424, 427, 433, 447, 448, 451, 476, 482, 487, 537, 538, 542, 558, 949-970, 1074, 972, 973, 974, 975, 977, 978, 979, 981, 983, 984, 985, 989, 992, 994, 996, 998, 999, 1000, 1001, 1004, 1005, 1006, 1007, 1008, 1009, 1010, 1011, 1013, 1015, 1018, 1020, 1021, 1022, 1023, 1024, 1025, 1026, 1027, 1028, 1029, 1030, 1033, 1034, 1035, 1039, 1040, 1041, 1042, 1043, 1046, 1047, 1050, 1051, 1052, 1053, 1058, 1060, 1061, 1062, 1063, 1064, 1066, 1067, 1070, 1078, 1080, 1081, 1082, 1091, 1099, 1101, 1102, 1103, 1104, 1105, 1106, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

In some embodiments, the PKC comprises at least 6, 7, 8, 9, or 10 amino acid substitutions or deletions relative to SEQ ID NO: 1.

In some embodiments, the PKC is capable of producing at least 10%, 20%, 30%, 40%, 50%, 60%, 70%, 80%, 90%, 100%, or more olivetolic acid relative to a control.

In some embodiments, the control is a PKC that does not comprise one or more of the amino acid substitutions or deletions.

Further aspects of the disclosure relate to host cells that comprise a heterologous polynucleotide encoding a polyketide cyclase (PKC), wherein the PKC comprises an amino acid substitution at a residue corresponding to position 13, 17, 18, 22, 26, 31, 33, 39, 52, 55, 64, 65, 66, 70, 71, 74, 80, 82, 83, 84, 85, 88, and/or 98 in SEQ ID NO: 1.

In some embodiments, the PKC comprises: the amino acid R at a residue corresponding to position 13 in SEQ ID NO:1; the amino acid N at a residue corresponding to position 17 in SEQ ID NO:1; the amino acid D at a residue corresponding to position 18 in SEQ ID NO:1; the amino acid K at a residue corresponding to position 22 in SEQ ID NO:1; the amino acid R at a residue corresponding to position 26 in SEQ ID NO:1; the amino acid L at a residue corresponding to position 31 in SEQ ID NO:1; the amino acid R, H or N at a residue corresponding to position 33 in SEQ ID NO:1; the amino acid G at a residue corresponding to position 39 in SEQ ID NO:1; the amino acid H at a residue corresponding to position 52 in SEQ ID NO:1; the amino acid F at a residue corresponding to position 55 in SEQ ID NO:1; the amino acid D at a residue corresponding to position 64 in SEQ ID NO:1; the amino acid N at a residue corresponding to position 65 in SEQ ID NO:1; the amino acid Y at a residue corresponding to position 66 in SEQ ID NO:1; the amino acid A at a residue corresponding to position 70 in SEQ ID NO:1; the amino acid S or E at a residue corresponding to position 71 in SEQ ID NO:1; the amino acid R at a residue corresponding to position 82 in SEQ ID NO: 1; the amino acid G at a residue corresponding to position 74 in SEQ ID NO:1; the amino acid A at a residue corresponding to position 80 in SEQ ID NO:1; the amino acid E, A, K, or N at a residue corresponding to position 83 in SEQ ID NO:1; the amino acid K or F at a residue corresponding to position 84 in SEQ ID NO:1; the amino acid F at a residue corresponding to position 85 in SEQ ID NO:1; the amino acid N at a residue corresponding to position 88 in SEQ ID NO: 1; and/or the amino acid K or Y at a residue corresponding to position 98 in SEQ ID NO:1.

In some embodiments, relative to SEQ ID NO: 1, the PKC comprises: A18D, T26R, I33R, D39G, E52H, Y55F, E64D, Q70A, G80A, D83N, and V84K; E17N, T26R, V31L, D71S, D83E, and V84F; E17N, V31L, I33N, E64D, D71S, and V84F; E17N, A18D, V31L, D71S, D83A, and V84F; E17N, E22K, V31L, I33N, D71E, and V84F; E17N, A18D, V31L, D71E, D83A, and V84F; E17N, V31L, D71S, D83E, V84F, and T98Y; V84K, T26R, D39G, D83E, T98K, and S65N; E17N, A18D, V31L, I33N, D71S, D83E, and V84K; E17N, A18D, V31L, D71S, I74G, D83E, and V84F; E17N, V31L, I33N, D71S, V84K, and Y85F; A18D, T26R, I33R, E52H, Y55F, V66Y, Q70A, G80A, D83N, V84K, and Y85F; E17N, A18D, V31L, I33N, D71S, D83K, and V84K; E17N, A18D, T26R, V31L, I33N, D71S, and V84K; V84K, T26R, D39G, D13R, T98K, and S65N; E17N, V31L, I33N, S65N, D71S, and V84K; E17N, A18D, E22K, V31L, I33N, D71E, and V84F; D13R, E17N, A18D, V31L, D71S, D83A, and V84K; E17N, V31L, D39G, D71S, D83A, and V84K; D13R, A18D, T26R, E52H, Y55F, Q70A, G80A, D83N, V84K, and Y85F; E17N, A18D, T26R, V31L, D71S, D83A, and V84K; E17N, V31L, I33N, D71S, G82R, and V84F; E17N, V31L, I33N, D71S, V84K, and T98K; V84K, T26R, D39G, D83E, D13R, and T98K; V84K, T26R, D83E, D13R, T98K, and S65N; E17N, V31L, I33N, V66Y, D71E, and V84F; E17N, A18D, V31L, 133N, D71E, and V84F; E17N, A18D, V31L, I33N, D71S, G80A, and V84F; A18D, T26R, E52H, Y55F, Q70A, G80A, D83N, V84K, Y85F, and F88N; E17N, A18D, V31L, I33N, D71S, V84K, and T98K; E17N, A18D, V31L, I33N, S65N, D71S, and V84K; T26R, I33H, Y55F, Q70A, G80A, D83K, V84K, and F88N; A18D, E22K, T26R, E52H, Y55F, Q70A, D71E, G80A, D83N, and V84K; or E17N, V31L, 133N, D71E, G82R, and V84F.

In some embodiments, the PKC comprises a sequence that is at least 90% identical to any one of SEQ ID NOs: 255, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

In some embodiments, the PKC comprises any one of SEQ ID NOs: 255, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

In some embodiments, the heterologous polynucleotide comprises a sequence that is at least 90% identical to any one of SEQ ID NOs: 448, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

In some embodiments, the heterologous polynucleotide comprises any one of SEQ ID NOs: 448, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

In some embodiments, relative to SEQ ID NO: 1, the PKC comprises A18D, T26R, I33R, D39G, E52H, Y55F, E64D, Q70A, G80A, D83N, and V84K. In some embodiments, relative to SEQ ID NO: 1, the PKC comprises E17N, T26R, V31L, D71S, D83E, and V84F. In some embodiments, relative to SEQ ID NO: 1, the PKC comprises E17N, V31L, I33N, E64D, D71S, and V84F. In some embodiments, relative to SEQ ID NO: 1, the PKC comprises E17N, A18D, V31L, D71S, D83A, and V84F. In some embodiments, relative to SEQ ID NO: 1, the PKC comprises E17N, E22K, V31L, I33N, D71E, and V84F. In some embodiments, relative to SEQ ID NO: 1, the PKC comprises E17N, A18D, V31L, D71E, D83A, and V84F. In some embodiments, relative to SEQ ID NO: 1, the PKC comprises E17N, V31L, D71S, D83E, V84F, and T98Y. In some embodiments, relative to SEQ ID NO: 1, the PKC comprises V84K, T26R, D39G, D83E, T98K, and S65N. In some embodiments, relative to SEQ ID NO: 1, the PKC comprises E17N, A18D, V31L, I33N, D71S, D83E, and V84K. In some embodiments, relative to SEQ ID NO: 1, the PKC comprises E17N, A18D, V31L, D71S, I74G, D83E, and V84F. In some embodiments, relative to SEQ ID NO: 1, the PKC comprises E17N, V31L, I33N, D71S, V84K, and Y85F. In some embodiments, relative to SEQ ID NO: 1, the PKC comprises A18D, T26R, I33R, E52H, Y55F, V66Y, Q70A, G80A, D83N, V84K, and Y85F. In some embodiments, relative to SEQ ID NO: 1, the PKC comprises E17N, A18D, V31L, I33N, D71S, D83K, and V84K. In some embodiments, relative to SEQ ID NO: 1, the PKC comprises E17N, A18D, T26R, V31L, I33N, D71S, and V84K. In some embodiments, relative to SEQ ID NO: 1, the PKC comprises V84K, T26R, D39G, D13R, T98K, and S65N. In some embodiments, relative to SEQ ID NO: 1, the PKC comprises E17N, V31L, I33N, S65N, D71S, and V84K. In some embodiments, relative to SEQ ID NO: 1, the PKC comprises E17N, A18D, E22K, V31L, I33N, D71E, and V84F. In some embodiments, relative to SEQ ID NO: 1, the PKC comprises D13R, E17N, A18D, V31L, D71S, D83A, and V84K. In some embodiments, relative to SEQ ID NO: 1, the PKC comprises E17N, V31L, D39G, D71S, D83A, and V84K. In some embodiments, relative to SEQ ID NO: 1, the PKC comprises D13R, A18D, T26R, E52H, Y55F, Q70A, G80A, D83N, V84K, and Y85F. In some embodiments, relative to SEQ ID NO: 1, the PKC comprises E17N, A18D, T26R, V31L, D71S, D83A, and V84K. In some embodiments, relative to SEQ ID NO: 1, the PKC comprises E17N, V31L, I33N, D71S, G82R, and V84F. In some embodiments, relative to SEQ ID NO: 1, the PKC comprises E17N, V31L, I33N, D71S, V84K, and T98K. In some embodiments, relative to SEQ ID NO: 1, the PKC comprises V84K, T26R, D39G, D83E, D13R, and T98K. In some embodiments, relative to SEQ ID NO: 1, the PKC comprises V84K, T26R, D83E, D13R, T98K, and S65N. In some embodiments, relative to SEQ ID NO: 1, the PKC comprises E17N, V31L, I33N, V66Y, D71E, and V84F. In some embodiments, relative to SEQ ID NO: 1, the PKC comprises E17N, A18D, V31L, I33N, D71E, and V84F. In some embodiments, relative to SEQ ID NO: 1, the PKC comprises E17N, A18D, V31L, I33N, D71S, G80A, and V84F. In some embodiments, relative to SEQ ID NO: 1, the PKC comprises A18D, T26R, E52H, Y55F, Q70A, G80A, D83N, V84K, Y85F, and F88N. In some embodiments, relative to SEQ ID NO: 1, the PKC comprises E17N, A18D, V31L, I33N, D71S, V84K, and T98K. In some embodiments, relative to SEQ ID NO: 1, the PKC comprises E17N, A18D, V31L, I33N, S65N, D71S, and V84K. In some embodiments, relative to SEQ ID NO: 1, the PKC comprises T26R, I33H, Y55F, Q70A, G80A, D83K, V84K, and F88N. In some embodiments, relative to SEQ ID NO: 1, the PKC comprises A18D, E22K, T26R, E52H, Y55F, Q70A, D71E, G80A, D83N, and V84K. In some embodiments, relative to SEQ ID NO: 1, the PKC comprises E17N, V31L, I33N, D71E, G82R, and V84F.

Further aspects of the disclosure relate to host cells that comprise a heterologous polynucleotide encoding a polyketide cyclase (PKC), wherein the PKC comprises at least 6 amino acid substitutions or deletions relative to SEQ ID NO: 1, wherein the PKC comprises at least two amino acid substitutions in a domain corresponding to helix 3 (positions 76-85 in SEQ ID NO: 1), and wherein the PKC is capable of producing olivetolic acid from 3,5,7-trioxododecanoyl-CoA.

In some embodiments, the at least two amino acid includes an amino acid substitution at position 84 relative to SEQ ID NO: 1.

In some embodiments, at least one of the at least two amino acid substitutions increases the stability of a kinked helix defined by the domains corresponding to (positions 66-73 in SEQ ID NO: 1), turn 1 (positions 74-75 in SEQ ID NO: 1), and helix 3 (positions 76-85 in SEQ ID NO: 1) and/or improves conformation of a binding pocket of the PKC to 3,5,7-trioxododecanoyl-CoA and/or olivetolic acid.

In some embodiments, the at least two amino acid substitutions in the domain corresponding to helix 3 (positions 76-85 in SEQ ID NO: 1) are at residues corresponding to positions 80 and 82 in SEQ ID NO: 1.

In some embodiments, the PKC comprises: the amino acid E, A, or S at the residue corresponding to position 80 in SEQ ID NO: 1; and/or the amino acid V or K at the residue corresponding to position 82 in SEQ ID NO: 1.

In some embodiments, the PKC further comprises an amino acid substitution at a residue corresponding to position 73 or 74 in SEQ ID NO: 1.

In some embodiments, the PKC comprises the amino acid L at the residue corresponding to position 73 in SEQ ID NO: 1; and/or the PKC comprises the amino acid S or L at the residue corresponding to position 74 in SEQ ID NO: 1.

In some embodiments, the PKC comprises an amino acid substitution at a residue corresponding to any of positions position 84 and/or 86-89 in SEQ ID NO: 1.

In some embodiments, the PKC comprises: the amino acid M at the residue corresponding to position 84 in SEQ ID NO: 1; the amino acid G at the residue corresponding to position 86 in SEQ ID NO: 1; the amino acid D at the residue corresponding to position 87 in SEQ ID NO: 1; the amino acid Y at the residue corresponding to position 88 in SEQ ID NO: 1; and/or the amino acid V at the residue corresponding to position 89 in SEQ ID NO: 1.

In some embodiments, the host cell produces at least 10%, 20%, 30%, 40%, 50%, 60%, 70%, 80%, 90%, 100%, or more of the PKC relative to a control host cell.

In some embodiments, the control host cell does not comprise the PKC.

In some embodiments, the PKC is capable of producing at least 10%, 20%, 30%, 40%, 50%, 60%, 70%, 80%, 90%, 100%, or more of olivetolic acid relative to a control.

In some embodiments, the control is a PKC that does not comprise one or more of the at least two amino acid substitutions in the domain corresponding to helix 3 (positions 76-85 in SEQ ID NO: 1).

In some embodiments, the PKC comprises one or more of the following: the amino acid G at a residue corresponding to position 2 in SEQ ID NO: 1; the amino acid M or Y at a residue corresponding to position 3 in SEQ ID NO: 1; the amino acid R or K at a residue corresponding to position 13 in SEQ ID NO: 1; the amino acid N at a residue corresponding to position 17 in SEQ ID NO: 1; the amino acid D or R at a residue corresponding to position 18 in SEQ ID NO: 1; the amino acid E or D at a residue corresponding to position 19 in SEQ ID NO: 1; the amino acid A at a residue corresponding to position 20 in SEQ ID NO: 1; the amino acid K at a residue corresponding to position 21 in SEQ ID NO: 1; the amino acid T or K at a residue corresponding to position 22 in SEQ ID NO: 1; the amino acid A or N at a residue corresponding to position 25 in SEQ ID NO: 1; the amino acid H, R, A, or E at a residue corresponding to position 26 in SEQ ID NO: 1; the amino acid A, S, E, or H at a residue corresponding to position 28 in SEQ ID NO: 1; the amino acid D, S, E, or T at a residue corresponding to position 29 in SEQ ID NO: 1; the amino acid L or A at a residue corresponding to position 31 in SEQ ID NO: 1; the amino acid H, R, D, E, K, Q, or N at a residue corresponding to position 33 in SEQ ID NO: 1; the amino acid L at a residue corresponding to position 37 in SEQ ID NO: 1; the amino acid L at a residue corresponding to position 38 in SEQ ID NO: 1; the amino acid G, S, or Q at a residue corresponding to position 39 in SEQ ID NO: 1; the amino acid K or R at a residue corresponding to position 41 in SEQ ID NO: 1; the amino acid I at a residue corresponding to position 45 in SEQ ID NO: 1; the amino acid K at a residue corresponding to position 50 in SEQ ID NO: 1; the amino acid H at a residue corresponding to position 52 in SEQ ID NO: 1; the amino acid H at a residue corresponding to position 53 in SEQ ID NO: 1; the amino acid F at a residue corresponding to position 55 in SEQ ID NO: 1; the amino acid Y at a residue corresponding to position 56 in SEQ ID NO: 1; the amino acid Y at a residue corresponding to position 57 in SEQ ID NO: 1; the amino acid I or L at a residue corresponding to position 60 in SEQ ID NO: 1; the amino acid M at a residue corresponding to position 62 in SEQ ID NO: 1; the amino acid Q or D at a residue corresponding to position 64 in SEQ ID NO: 1; the amino acid N at a residue corresponding to position 65 in SEQ ID NO: 1; the amino acid K at a residue corresponding to position 67 in SEQ ID NO: 1; the amino acid E, G, or H at a residue corresponding to position 68 in SEQ ID NO: 1; the amino acid A or L at a residue corresponding to position 70 in SEQ ID NO: 1; the amino acid Q or S, at a residue corresponding to position 71 in SEQ ID NO: 1; the amino acid D, E, H, or Q at a residue corresponding to position 74 in SEQ ID NO: 1; the amino acid A or G at a residue corresponding to position 83 in SEQ ID NO: 1; the amino acid F, K, M, or Q at a residue corresponding to position 84 in SEQ ID NO: 1; the amino acid A or F at a residue corresponding to position 85 in SEQ ID NO: 1; the amino acid G at a residue corresponding to position 86 in SEQ ID NO: 1; the amino acid D or K at a residue corresponding to position 87 in SEQ ID NO: 1; the amino acid Y, N, or S at a residue corresponding to position 88 in SEQ ID NO: 1; the amino acid M or I at a residue corresponding to position 95 in SEQ ID NO: 1; the amino acid F at a residue corresponding to position 97 in SEQ ID NO: 1; the amino acid E, K, or Y at a residue corresponding to position 98 in SEQ ID NO: 1; the amino acid A, G, K, N, Q, or S at a residue corresponding to position 100 in SEQ ID NO: 1; and/or the amino acid A or E at a residue corresponding to position 101 in SEQ ID NO: 1.

In some embodiments, the PKC further comprises one or more of the following: the amino acid M at a residue corresponding to position 24 in SEQ ID NO: 1; the amino acid F at a residue corresponding to position 46 in SEQ ID NO: 1; the amino acid A at a residue corresponding to position 48 in SEQ ID NO: 1; the amino acid Q at a residue corresponding to position 49 in SEQ ID NO: 1; the amino acid I or F at a residue corresponding to position 59 in SEQ ID NO: 1; the amino acid T, D, or S at a residue corresponding to position 61 in SEQ ID NO: 1; the amino acid L, M, or Y at a residue corresponding to position 66 in SEQ ID NO: 1; the amino acid L or Y at a residue corresponding to position 69 in SEQ ID NO: 1; the amino acid L at a residue corresponding to position 73 in SEQ ID NO: 1; the amino acid S at a residue corresponding to position 74 in SEQ ID NO: 1; the amino acid V, A, E, S, D, or N at a residue corresponding to position 80 in SEQ ID NO: 1; the amino acid Y at a residue corresponding to position 81 in SEQ ID NO: 1; the amino acid K, V, or R at a residue corresponding to position 82 in SEQ ID NO: 1; the amino acid V or M at a residue corresponding to position 89 in SEQ ID NO: 1; and/or the amino acid V at a residue corresponding to position 94 in SEQ ID NO: 1.

In some embodiments, the PKC comprises at least 6, 7, 8, 9, or 10 amino acid substitutions relative to SEQ ID NO: 1.

In some embodiments, the PKC is capable of producing at least 10%, 20%, 30%, 40%, 50%, 60%, 70%, 80%, 90%, 100%, or more of olivetolic acid relative to a control.

In some embodiments, the control is a PKC that does not comprise an amino acid substitution at a residue corresponding to position 2, 3, 13, 17, 18, 19, 20, 21, 22, 25, 26, 28, 29, 31, 33, 37, 38, 39, 41, 45, 50, 52, 53, 55, 56, 57, 60, 62, 64, 65, 67, 68, 70, 71, 74, 83, 84, 85, 86, 87, 88, 95, 97, 98, 100, and/or 101 in SEQ ID NO: 1.

Further aspects of the disclosure provide host cells that comprise a heterologous polynucleotide encoding a polyketide cyclase (PKC), wherein the PKC comprises one or more of the following amino acid substitutions 17, 18, 29, 31, 33, 39, 71, 83, and/or 84 relative to SEQ ID NO: 1, and wherein the PKC is capable of producing olivetolic acid from 3,5,7-trioxododecanoyl-CoA.

In some embodiments, the PKC comprises one or more of the following amino acid substitutions E17N, A18D, N29T, V31L, I33N, D39Q, D71S, D83A, and/or V84F relative to SEQ ID NO: 1.

In some embodiments, the PKC comprises at least 6, 7, 8, 9, or 10 amino acid substitutions or deletions relative to SEQ ID NO: 1.

In some embodiments, the PKC comprises: E17N, A18D, V31L, D39Q, D71S, and D83A relative to SEQ ID NO: 1; E17N, A18D, V31L, D71S, D83A, and V84F relative to SEQ ID NO: 1; or A18D, V31L, D39Q, D71S, D83A, and V84F relative to SEQ ID NO: 1.

In some embodiments, the PKC comprises a sequence that is at least 90% identical to SEQ ID NO: 255, 220, or 707. In some embodiments, the PKC comprises SEQ ID NO: 255, 220, 707.

In some embodiments, the PKC produces more olivetolic acid as compared to a control PKC comprising SEQ ID NO: 1. In some embodiments, the PKC produces more than 41,000 μg/L olivetolic acid.

In some embodiments, relative to a control PKC comprising SEQ ID NO: 1, the PKC produces a greater ratio of olivetolic acid to olivetol. In some embodiments, the PKC produces a ratio of olivetolic acid to olivetol that is higher than 3.1.

Further aspects of the disclosure provide host cells that comprise a heterologous polynucleotide encoding a polyketide cyclase (PKC), wherein the PKC comprises one or more amino acid substitutions relative to SEQ ID NO: 1 selected from the group consisting of: D13R; A18D; E22K; T26R or T26H; N29S; I33R or I33H; D39G; K49Q; E52H; Y55F; Q70A or Q70L; D71Q; I74G; G80A; D83K or D83N; V84K; and Y85A, wherein the PKC is capable of producing olivetolic acid from 3,5,7-trioxododecanoyl-CoA (4a).

In some embodiments, the PKC comprises at least 6, at least 7, at least 8, at least 9, or at least 10 amino acid substitutions or deletions relative to SEQ ID NO: 1.

In some embodiments, relative to SEQ ID NO: 1, the PKC comprises: I33R, E52H, Y55F, Q70A, G80A, D83K, and V84K; D13R, A18D, N29S, Q70L, D71Q, G80A, and D83K; I33R, E52H, Y55F, Q70A, I74G, and D83K; T26R, I33H, E52H, Y55F, Q70A, and D83K; A18D, T26R, E52H, Y55F, Q70A, G80A, D83N, V84K, and Y85A; T26R, I33H, Y55F, Q70A, G80A, D83K, and Y85A; or E22K, T26H, N29S, D39G, K49Q, E52H, D83N, and V84K.

In some embodiments, the PKC comprises a sequence that is at least 90% identical to SEQ ID NO: 283, 289, 294, 344, 345, 349, or 365. In some embodiments, the PKC comprises SEQ ID NO: 283, 289, 294, 344, 345, 349, or 365.

In some embodiments, the PKC produces more olivetolic acid as compared to a control PKC comprising SEQ ID NO: 1.

In some embodiments, the PKC produces more than 94,000 ug/L olivetolic acid.

In some embodiments, wherein the host cell is a plant cell, an algal cell, a yeast cell, a bacterial cell, or an animal cell. In some embodiments, the host cell is a yeast cell. In some embodiments, the yeast cell is a Saccharomyces cell, a Yarrowia cell, or a Komagataella cell. In some embodiments, the Saccharomyces cell is a Saccharomyces cerevisiae cell. In some embodiments, the yeast cell is a Yarrowia cell. In some embodiments, the host cell is a bacterial cell. In some embodiments, the bacterial cell is an E. coli cell. In some embodiments, the host cell further comprises one or more heterologous polynucleotides comprising one or more of: an acyl activating enzyme (AAE), a polyketide synthase (PKS), a prenyltransferase (PT), and/or a terminal synthase (TS). In some embodiments, the polyketide synthase is an olivetol synthase (OLS). In some embodiments, the terminal synthase is a cannabidiolic acid synthase (CBDAS). In some embodiments, the terminal synthase is a tetrahydrocannabinolic acid synthase (THCAS). In some embodiments, the terminal synthase is a cannabichromenic acid synthase (CBCAS).

Further aspects of the disclosure relate to methods of culturing any of the host cells of the disclosure.

Further aspects of the disclosure relate to methods for producing 2,4-dihydroxybenzoic acid optionally substituted at position 6 comprising contacting: a tetraketide with a polyketide cyclase (PKC), wherein the PKC comprises an amino acid substitution at one or more residues corresponding to one more or more positions selected from the group consisting of position 17, 18, 29, 31, 33, 39, 71, 83 and 84 in SEQ ID NO: 1.

In some embodiments, the PKC comprises at least 6, 7, 8, 9, or 10 amino acid substitutions or deletions relative to SEQ ID NO: 1. In some embodiments, the tetraketide is 3,5,7-trioxododecanoyl-CoA. In some embodiments, the 2,4-dihydroxybenzoic acid optionally substituted at position 6 is olivetolic acid. In some embodiments, contacting the tetraketide with the PKC occurs in vitro. In some embodiments, contacting the tetraketide with the PKC occurs in vivo.

In some embodiments, the PKC comprises at least six amino acid substitutions selected from the group consisting of E17N, A18D, N29T, V31L, I33N, D39Q, D71S, D83A, and V84F relative to SEQ ID NO: 1.

Further aspects of the disclosure relate to non-naturally occurring nucleic acid encoding a polyketide cyclase (PKC), wherein the non-naturally occurring nucleic acid encodes a PKC comprising a sequence with at least one amino acid substitution relative to SEQ ID NO: 1 and with at least 90% identity to SEQ ID NO: 62, 87, 220, 234, 219, 258, 231, 240, 255, 707, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, or 777.

Further aspects of the disclosure relate to non-naturally occurring nucleic acid encoding a polyketide cyclase (PKC) wherein relative to the sequence of SEQ ID NO: 1 the wherein the PKC comprises an amino acid substitution at one or more residues corresponding to one or more positions selected from the group consisting of position 17, 18, 29, 31, 33, 39, 71, 83 and 84 in SEQ ID NO: 1 and wherein the PKC is capable of producing olivetolic acid from 3,5,7-trioxododecanoyl-CoA.

In some embodiments, the PKC comprises at least 6 amino acid substitutions or deletions relative to SEQ ID NO: 1.

Further aspects of the disclosure relate to non-naturally occurring nucleic acid encoding a polyketide cyclase (PKC) wherein relative to the sequence of SEQ ID NO: 1, the PKC comprises an amino acid substitution at one or more residues corresponding to one or more positions selected from the group consisting of position 13, 17, 18, 22, 26, 31, 33, 39, 52, 55, 64, 65, 66, 70, 71, 74, 80, 82, 83, 84, 85, 88, and/or 98 in SEQ ID NO: 1 and wherein the PKC is capable of producing olivetolic acid from 3,5,7-trioxododecanoyl-CoA. Further aspects of the disclosure relate to vectors comprising non-naturally occurring nucleic acids associated with the disclosure. Further aspects of the disclosure relate to exoression cassettes comprising non-naturally occurring nucleic acids associated with the disclosure.

Further aspects of the disclosure relate to non-naturally occurring polyketide cyclase (PKC) wherein the PKC comprises an amino acid substitution at one or more residues corresponding to one or more positions selected from the group consisting of position 17, 18, 29, 31, 33, 39, 71, 83 and 84 in SEQ ID NO: 1, and wherein the PKC is capable of producing olivetolic acid from 3,5,7-trioxododecanoyl-CoA.

Further aspects of the disclosure relate to non-naturally occurring polyketide cyclase (PKC) wherein the PKC comprises an amino acid substitution at one or more residues corresponding to one or more positions selected from the group consisting of position 13, 17, 18, 22, 26, 31, 33, 39, 52, 55, 64, 65, 66, 70, 71, 74, 80, 82, 83, 84, 85, 88, and/or 98 in SEQ ID NO: 1, and wherein the PKC is capable of producing olivetolic acid from 3,5,7-trioxododecanoyl-CoA.

In some embodiments, the PKC comprises at least 6, 7, 8, 9, or 10 amino acid substitutions or deletions relative to SEQ ID NO: 1.

Further aspects of the disclosure relate to host cells transformed with non-naturally occurring nucleic acids, vectors, or expression cassettes associated with the disclosure.

Further aspects of the disclosure relate to host cells that comprise a heterologous polynucleotide encoding a polyketide cyclase (PKC), wherein the PKC comprises one or more amino acid substitutions relative to SEQ ID NO: 1 selected from the group consisting of: A2G; V3M or V3Y; D13R or D13K; E17N; A18D or A18R; Q19E or Q19D; K20A; E21K; E22T or E22K; K25A or K25N; T26H, T26R, T26A, or T26E; V28A, V28S, V28E, or V28H; N29D, N29S, N29E, or N29T; V31L or V31A; 133H, 133R, 133D, 133E, 133K, 133Q, or 133N; M37L; K38L; D39G, D39S, or D39Q; Y41K or Y41R; D45I; N50K; E52H; E53H; Y55F; T56Y; H57Y; E601 or E60L; T62M; E64Q or E64D; S65N; E67K; T68E, T68G, or T68H; Q70A or Q70L; D71Q or D71S; I74D, 174E, I74H, or I74Q; D83A or D83G; V84F, V84K, V84M, or V84Q; Y85A or Y85F; R86G; S87C, S87D, or S87K; F88Y, F88N, or F88S; F95M or F95I; Y97F; T98E, T98K, or T98Y; R100A, R100G, R100K, R100N, R100Q, or R100S; and K101A or K101E.

In some embodiments, the PKC further comprises one or more of the following amino acid substitutions relative to SEQ ID NO: 1: F24M; V46F; Q48A; K49Q; N50F or N50W; V59I or V59F; V61T, V61D, or V61S; V66L, V66M, or V66Y; 169L or 169Y; D71E; I73L; I74S, I74G, or I74L; G80V, G80A, G80E, G80S, G80D, or G80N; F81Y; G82K, G82V, or G82R; D83E, D83K, D83N, D83Q, D83R, or D83S; R86N or R86S; W89V or W89M; and/or I94V. In some embodiments, the PKC comprises at least 6, 7, 8, 9, or 10 amino acid substitutions or deletions relative to SEQ ID NO: 1.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic depicting the native Cannabis biosynthetic pathway for production of cannabinoid compounds, including five enzymatic steps mediated by: (R1a) acyl activating enzymes (AAE); (R2a) olivetol synthase enzymes (OLS); (R3a) olivetolic acid cyclase enzymes (OAC); (R4a) cannabigerolic acid synthase enzymes (CBGAS); and (R5a) terminal synthase enzymes (TS). Formulae 1a-11a correspond to hexanoic acid (1a), hexanoyl-CoA (2a), malonyl-CoA (3a), 3,5,7-trioxododecanoyl-CoA (4a), olivetol (5a), olivetolic acid (6a), geranyl pyrophosphate (7a), cannabigerolic acid (8a), cannabidiolic acid (9a), tetrahydrocannabinolic acid (10a), and cannabichromenic acid (11a). Hexanoic acid is an exemplary carboxylic acid substrate; other carboxylic acids may also be used (e.g., butyric acid, isovaleric acid, octanoic acid, decanoic acid, etc.; see e.g., FIG. 3 below). The enzymes that catalyze the synthesis of 3,5,7-trioxododecanoyl-CoA and olivetolic acid are shown in R2a and R3a, respectively, and can include multi-functional enzymes that catalyze the synthesis of 3,5,7-trioxododecanoyl-CoA and olivetolic acid. The enzymes cannabidiolic acid synthase (CBDAS), tetrahydrocannabinolic acid synthase (THCAS), and cannabichromenic acid synthase (CBCAS) that catalyze the synthesis of cannabidiolic acid, tetrahydrocannabinolic acid, and cannabichromenic acid, respectively, are shown in step R5a. FIG. 1 is adapted from Carvalho et al. “Designing Microorganisms for Heterologous Biosynthesis of Cannabinoids” (2017) FEMS Yeast Research Jun. 1;17(4), which is incorporated by reference in its entirety.

FIG. 2 is a schematic depicting a heterologous biosynthetic pathway for production of cannabinoid compounds, including five enzymatic steps mediated by: (R1) acyl activating enzymes (AAE); (R2) polyketide synthase enzymes (PKS) or bifunctional polyketide synthase-polyketide cyclase enzymes (PKS—PKC); (R3) polyketide cyclase enzymes (PKC) or bifunctional PKS—PKC enzymes; (R4) prenyltransferase enzymes (PT); and (R5) terminal synthase enzymes (TS). Any carboxylic acid of varying chain lengths, structures (e.g., aliphatic, alicyclic, or aromatic) and functionalization (e.g., hydroxylic-, keto-, amino-, thiol-, aryl-, or alogeno-) may also be used as precursor substrates (e.g., thiopropionic acid, hydroxy phenyl acetic acid, norleucine, bromodecanoic acid, butyric acid, isovaleric acid, octanoic acid, decanoic acid, etc).

FIG. 3 is a non-exclusive representation of select putative precursors for the cannabinoid pathway in FIG. 2.

FIG. 4 is a schematic showing a reaction catalyzed by an olivetolic acid cyclase (OAC) enzyme wherein 3,5,7-trioxododecanoyl-CoA (Formula (4a)) is cyclized to form olivetolic acid (OA, Formula (6a)). The spontaneous decarboxylative cyclization of 3,5,7-trioxododecanoyl-CoA to Olivetol (Formula (5a)) is also shown.

FIG. 5 is a schematic showing a plasmid used to express polyketide cyclase enzymes in S. cerevisiae. The coding sequence for the polyketide cyclase enzymes (labeled “Library gene”) was driven by the GAL1 promoter. The plasmid contains markers for both yeast (URA3) and bacteria (ampR), as well as origins of replication for yeast (2 micron), and bacteria (pBR322).

FIG. 6 depicts a graph showing olivetolic acid production in S. cerevisiae strains expressing synthetic OAC polypeptides identified in a screen described in Example 1.

FIGS. 7A-7B depict monomeric crystal structures of CsOAC (SEQ ID NO: 1) with olivetolic acid bound (Protein Data Bank Accession No. 5B09). Olivetolic acid is shown in stick representation in the center. FIG. 7A is a cartoon representation of the CsOAC crystal structure. FIG. 7B is a cartoon representation of the CsOAC crystal structure where positions 73, 74, 80, 82, 84, 86, 87, 88, and 89 are highlighted in sphere representation.

FIG. 8 depicts a graph comparing olivetol and olivetolic acid production in S. cerevisiae strains in a screen as described in Example 2 and Table 5.

FIGS. 9A-9B depict results from a secondary screen of S. cerevisiae strains as described in Example 2 and Table 6. FIG. 9A depicts olivetolic acid production. FIG. 9B shows the ratio of olivetolic acid to olivetol. OACs comprising two (double) or three (triple) mutations relative to wild-type CsOAC are shown.

FIGS. 10A-10B depict results from a screen of S. cerevisiae strains as described in Example 2 and Table 7. FIG. 10A depicts olivetolic acid production. FIG. 10B shows the ratio of olivetolic acid to olivetol.

FIGS. 11A-11B depict results from a screen of S. cerevisiae strains as described in Example 3 and Table 8. FIG. 11A depicts olivetolic acid production. FIG. 11B shows the ratio of olivetolic acid to olivetol.

FIGS. 12A-12D depict results from a screen of S. cerevisiae strains as described in Example 4 and Tables 9A-9B. FIG. 12A depicts olivetolic acid production. FIG. 12B shows the ratio of olivetolic acid (OA) to olivetol (OL). FIG. 12C shows olivetolic acid levels normalized to production by the t815900 strain. FIG. 12D shows the ratio of olivetolic acid (OA) to olivetol (OL) normalized to production by the t815900 strain.

### DETAILED DESCRIPTION

This disclosure provides methods for production of cannabinoids and cannabinoid precursors from fatty acid substrates using genetically modified host cells. Methods include heterologous expression of a polyketide cyclase, such as an olivetolic acid cyclase (OAC). The application describes PKCs that can be functionally expressed in host cells such as S. cerevisiae. As demonstrated in the Examples, PKCs were identified that were capable of producing olivetolic acid (OA) in a host cell. The PKCs described in this disclosure may be useful in increasing the efficiency and purity of cannabinoid production.

### Definitions

While the following terms are believed to be well understood by one of ordinary skill in the art, the following definitions are set forth to facilitate explanation of the disclosed subject matter.

The term “a” or “an” refers to one or more of an entity, i.e., can identify a referent as plural. Thus, the terms “a” or “an,” “one or more” and “at least one” are used interchangeably in this application. In addition, reference to “an element” by the indefinite article “a” or “an” does not exclude the possibility that more than one of the elements is present, unless the context clearly requires that there is one and only one of the elements.

The terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure may refer to the “microorganisms” or “microbes” of lists/tables and figures present in the disclosure. This characterization can refer to not only the identified taxonomic genera of the tables and figures, but also the identified taxonomic species, as well as the various novel and newly identified or designed strains of any organism in the tables or figures. The same characterization holds true for the recitation of these terms in other parts of the specification, such as in the Examples.

The term “prokaryotes” is recognized in the art and refers to cells that contain no nucleus or other cell organelles. The prokaryotes are generally classified in one of two domains, the Bacteria and the Archaea.

“Bacteria” or “eubacteria” refers to a domain of prokaryotic organisms. Bacteria include at least 11 distinct groups as follows: (1) Gram-positive (gram+) bacteria, of which there are two major subdivisions: (a) high G+C group (Actinomycetes, Mycobacteria, Micrococcus, others) and (b) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

The term “Archaea” refers to a taxonomic classification of prokaryotic organisms with certain properties that make them distinct from Bacteria in physiology and phylogeny.

The term “Cannabis” refers to a genus in the family Cannabaceae. Cannabis is a dioecious plant. Glandular structures located on female flowers of Cannabis, called trichomes, accumulate relatively high amounts of a class of terpeno-phenolic compounds known as phytocannabinoids (described in further detail below). Cannabis has conventionally been cultivated for production of fibre and seed (commonly referred to as “hemp-type”), or for production of intoxicants (commonly referred to as “drug-type”). In drug-type Cannabis, the trichomes contain relatively high amounts of tetrahydrocannabinolic acid (THCA), which can convert to tetrahydrocannabinol (THC) via a decarboxylation reaction, for example upon combustion of dried Cannabis flowers, to provide an intoxicating effect. Drug-type Cannabis often contains other cannabinoids in lesser amounts. In contrast, hemp-type Cannabis contains relatively low concentrations of THCA, often less than 0.3% THC by dry weight. Hemp-type Cannabis may contain non-THC and non-THCA cannabinoids, such as cannabidiolic acid (CBDA), cannabidiol (CBD), and other cannabinoids. Presently, there is a lack of consensus regarding the taxonomic organization of the species within the genus. Unless context dictates otherwise, the term “Cannabis” is intended to include all putative species within the genus, such as, without limitation, Cannabis sativa, Cannabis indica, and Cannabis ruderalis and without regard to whether the Cannabis is hemp-type or drug-type.

The term “cyclase activity” in reference to a polyketide synthase (PKS) enzyme (e.g., an olivetol synthase (OLS) enzyme) or a polyketide cyclase (PKC) enzyme (e.g., an olivetolic acid cyclase (OAC) enzyme), refers to the activity of catalyzing the cyclization of an oxo fatty acyl-CoA (e.g., 3,5,7-trioxododecanoyl-COA, 3,5,7-trioxodecanoyl-COA) to the corresponding intramolecular cyclization product (e.g., olivetolic acid, divarinic acid). In some embodiments, the PKS catalyzes the C2-C7 aldol condensation of an acyl-COA with three additional ketide moieties added thereto.

A “cytosolic” or “soluble” enzyme refers to an enzyme that is predominantly localized (or predicted to be localized) in the cytosol of a host cell.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (i.e., bacteria and archaea) is that they have membrane-bound organelles, especially the nucleus, which contains the genetic material, and is enclosed by the nuclear envelope.

The term “host cell” refers to a cell that can be used to express a polynucleotide, such as a polynucleotide that encodes an enzyme used in biosynthesis of cannabinoids or cannabinoid precursors. The terms “genetically modified host cell,” “recombinant host cell,” and “recombinant strain” are used interchangeably and refer to host cells that have been genetically modified by, e.g., cloning and transformation methods, or by other methods known in the art (e.g., selective editing methods, such as CRISPR). Thus, the terms include a host cell (e.g., bacterial cell, yeast cell, fungal cell, insect cell, plant cell, mammalian cell, human cell, etc.) that has been genetically altered, modified, or engineered, so that it exhibits an altered, modified, or different genotype and/or phenotype, as compared to the naturally-occurring cell from which it was derived. It is understood that in some embodiments, the terms refer not only to the particular recombinant host cell in question, but also to the progeny or potential progeny of such a host cell.

The term “control host cell,” or the term “control” when used in relation to a host cell, refers to an appropriate comparator host cell for determining the effect of a genetic modification or experimental treatment. In some embodiments, the control host cell is a wild type cell. In other embodiments, a control host cell is genetically identical to the genetically modified host cell, except for the genetic modification(s) differentiating the genetically modified or experimental treatment host cell. In some embodiments, the control host cell has been genetically modified to express a wild type or otherwise known variant of an enzyme being tested for activity in other test host cells.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

The term “at least a portion” or “at least a fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of an enzyme, such as a catalytic domain. A biologically active portion of a genetic regulatory element may comprise a portion or fragment of a full length genetic regulatory element and have the same type of activity as the full length genetic regulatory element, although the level of activity of the biologically active portion of the genetic regulatory element may vary compared to the level of activity of the full length genetic regulatory element.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence promotes transcription of the coding sequence and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

The terms “link,” “linked,” or “linkage” means two entities (e.g., two polynucleotides or two proteins) are bound to one another by any physicochemical means. Any linkage known to those of ordinary skill in the art, covalent or non-covalent, is embraced. In some embodiments, a nucleic acid sequence encoding an enzyme of the disclosure is linked to a nucleic acid encoding a signal peptide. In some embodiments, an enzyme of the disclosure is linked to a signal peptide. Linkage can be direct or indirect.

The terms “transformed” or “transform” with respect to a host cell refer to a host cell in which one or more nucleic acids have been introduced, for example on a plasmid or vector or by integration into the genome. In some instances where one or more nucleic acids are introduced into a host cell on a plasmid or vector, one or more of the nucleic acids, or fragments thereof, may be retained in the cell, such as by integration into the genome of the cell, while the plasmid or vector itself may be removed from the cell. In such instances, the host cell is considered to be transformed with the nucleic acids that were introduced into the cell regardless of whether the plasmid or vector is retained in the cell or not.

The term “volumetric productivity” or “production rate” refers to the amount of product formed per volume of medium per unit of time. Volumetric productivity can be reported in gram per liter per hour (g/L/h).

The term “specific productivity” of a product refers to the rate of formation of the product normalized by unit volume or mass or biomass and has the physical dimension of a quantity of substance per unit time per unit mass or volume [M•T⁻¹•M⁻¹ or M•T⁻¹•L⁻³, where M is mass or moles, T is time, L is length].

The term “biomass specific productivity” refers to the specific productivity in gram product per gram of cell dry weight (CDW) per hour (g/g CDW/h) or in mmol of product per gram of cell dry weight (CDW) per hour (mmol/g CDW/h). Using the relation of CDW to OD600 for the given microorganism, specific productivity can also be expressed as gram product per liter culture medium per optical density of the culture broth at 600 nm (OD) per hour (g/L/h/OD). Also, if the elemental composition of the biomass is known, biomass specific productivity can be expressed in mmol of product per C-mole (carbon mole) of biomass per hour (mmol/C-mol/h).

The term “yield” refers to the amount of product obtained per unit weight of a certain substrate and may be expressed as g product per g substrate (g/g) or moles of product per mole of substrate (mol/mol). Yield may also be expressed as a percentage of the theoretical yield. “Theoretical yield” is defined as the maximum amount of product that can be generated per a given amount of substrate as dictated by the stoichiometry of the metabolic pathway used to make the product and may be expressed as g product per g substrate (g/g) or moles of product per mole of substrate (mol/mol).

The term “titer” refers to the strength of a solution or the concentration of a substance in solution. For example, the titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

The term “total titer” refers to the sum of all products of interest produced in a process, including but not limited to the products of interest in solution, the products of interest in gas phase if applicable, and any products of interest removed from the process and recovered relative to the initial volume in the process or the operating volume in the process. For example, the total titer of products of interest (e.g., small molecule, peptide, synthetic compound, fuel, alcohol, etc.) in a fermentation broth is described as g of products of interest in solution per liter of fermentation broth or cell-free broth (g/L) or as g of products of interest in solution per kg of fermentation broth or cell-free broth (g/Kg).

The term “amino acid” refers to organic compounds that comprise an amino group, —NH2, and a carboxyl group, —COOH. The term “amino acid” includes both naturally occurring and unnatural amino acids. Nomenclature for the twenty common amino acids is as follows: alanine (ala or A); arginine (arg or R); asparagine (asn or N); aspartic acid (asp or D); cysteine (cys or C); glutamine (gln or Q); glutamic acid (glu or E); glycine (gly or G); histidine (his or H); isoleucine (ile or I); leucine (leu or L); lysine (lys or K); methionine (met or M); phenylalanine (phe or F); proline (pro or P); serine (ser or S); threonine (thr or T); tryptophan (trp or W); tyrosine (tyr or Y); and valine (val or V). Non-limiting examples of unnatural amino acids include homo-amino acids, proline and pyruvic acid derivatives, 3-substituted alanine derivatives, glycine derivatives, ring-substituted phenylalanine derivatives, ring-substituted tyrosine derivatives, linear core amino acids, amino acids with protecting groups including Fmoc, Boc, and Cbz, β-amino acids (β3 and β2), and N-methyl amino acids.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of, or a substituent that is, a straight-chain or branched saturated hydrocarbon group having from 1 to 20 carbon atoms (“C1-20 alkyl”). In certain embodiments, the term “alkyl” refers to a radical of, or a substituent that is, a straight-chain or branched saturated hydrocarbon group having from 1 to 10 carbon atoms (“C1-10 alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 2 to 7 carbon atoms (“C₂₋₇ alkyl”). In some embodiments, an alkyl group has 3 to 7 carbon atoms (“C₃₋₇ alkyl”). In some embodiments, an alkyl group has 1 to 6 carbon atoms (“C₁₋₆ alkyl”). In some embodiments, an alkyl group has 2 to 6 carbon atoms (“C₂₋₆ alkyl”). In some embodiments, an alkyl group has 3 to 5 carbon atoms (“C₃₋₅ alkyl”). In some embodiments, an alkyl group has 5 carbon atoms (“C₅ alkyl”). In some embodiments, the alkyl group has 3 carbon atoms (“C3 alkyl”). In some embodiments, the alkyl group has 7 carbon atoms (“C₇ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C1 alkyl”).

Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, benzyl).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein Rx¹ is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di- aliphaticamino, mono- or di- heteroaliphaticamino, mono- or di- alkylamino, mono- or di- heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described in this application that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkenyl” refers to a radical of, or a substituent that is, a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C2-s alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C2-6 alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of, or a substituent that is, a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C2-6 alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C3-10 carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅6 cycloalkyl”). In some embodiments, a cycloalkyl group has 5 to 10 ring carbon atoms (“C₅₋₁₀ cycloalkyl”). Examples of C₅6 cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl. In certain embodiments, the aralkyl is 7-phenylheptanyl. In certain embodiments, the aralkyl is C7 alkyl substituted by an optionally substituted aryl group (e.g., phenyl). In certain embodiments, the aralkyl is a C7-C10 alkyl group substituted by an optionally substituted aryl group (e.g., phenyl).

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined in this application. Likewise, “saturated” refers to a group that does not contain a double or triple bond, i.e., contains all single bonds.

The term “optionally substituted” means substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described in this application that results in the formation of a stable compound. The present invention contemplates any and all such combinations in order to arrive at a stable compound. For purposes of this invention, heteroatoms such as nitrogen may have hydrogen substituents and/or any suitable substituent as described in this application which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R—, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R—, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{bb})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl;

wherein:
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X— is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH2(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH2, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH2, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion. Alternatively, two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
   wherein:
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{cc}, —SO₂R^{cc}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═)N(R)₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH2(C₁₋₆ alkyl)⁺X⁻, —NH3⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —CO₂(C₁₋₆ alkyl), —C(═O)NH2, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH2, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, B⁻r, I⁻), NO₃⁻, ClO₄⁻, OH⁻, H₂PO₄, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆, B[3,5-(CF₃)₂C₆H3]4₁⁻, B(C₆F₅)₄⁻, BPh₄⁻, Al(OC(CF₃)₃)₄⁻, and carborane anions (e.g., CB₁₁H₁₂⁻ or (HCB₁₁Me₅Br6)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated by reference. Pharmaceutically acceptable salts of the compounds disclosed in this application include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of a compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (1), (9), (10), and (11) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro- forms of phenylnitromethane, which are likewise formed by treatment with acid or base. Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and described by the R- and S-sequencing rules of Cahn and Prelog. An enantiomer can also be characterized by the manner in which the molecule rotates the plane of polarized light, and designated as dextrorotatory or levorotatory (i.e., as (+) or (−)-isomers respectively). A chiral compound can exist as either an individual enantiomer or as a mixture of enantiomers. A mixture containing equal proportions of the enantiomers is called a “racemic mixture.”

The term “co-crystal” refers to a crystalline structure comprising at least two different components (e.g., a compound described in this application and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound described in this application is complexed with the acid in a way that proton transfer (e.g., a complete proton transfer) from the acid to a compound described in this application easily occurs at room temperature. In the co-crystal, however, a compound described in this application is complexed with the acid in a way that proton transfer from the acid to a compound described in this application does not easily occur at room temperature. In certain embodiments, in the co-crystal, there is no proton transfer from the acid to a compound described in this application. In certain embodiments, in the co-crystal, there is partial proton transfer from the acid to a compound described in this application. Co-crystals may be useful to improve the properties (e.g., solubility, stability, and ease of formulation) of a compound described in this application.

The term “polymorphs” refers to a crystalline form of a compound (or a salt, hydrate, or solvate thereof) in a particular crystal packing arrangement. All polymorphs of the same compound have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrug” refers to compounds, including derivatives of the compounds of Formula (X), (8), (9), (10), or (11), that have cleavable groups and become by solvolysis or under physiological conditions the compounds of Formula (X), (8), (9), (10), or (11) and that are pharmaceutically active in vivo. The prodrugs may have attributes such as, without limitation, solubility, bioavailability, tissue compatibility, or delayed release in a mammalian organism. Examples include, but are not limited to, derivatives of compounds described in this application, including derivatives formed from glycosylation of the compounds described in this application (e.g., glycoside derivatives), carrier-linked prodrugs (e.g., ester derivatives), bioprecursor prodrugs (a prodrug metabolized by molecular modification into the active compound), and the like. Non-limiting examples of glycoside derivatives are disclosed in and incorporated by reference from PCT Publication No. WO2018/208875 and U.S. Patent Publication No. 2019/0078168. Non-limiting examples of ester derivatives are disclosed in and incorporated by reference from U.S. Patent Publication No. 2017/0362195.

Other derivatives of the compounds of this invention have activity in both their acid and acid derivative forms, but the acid sensitive form often offers advantages of solubility, bioavailability, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds of Formula (X), (8), (9), (10), or (11) may be preferred.

### Cannabinoids

As used in this application, the term “cannabinoid” includes compounds of Formula (X):

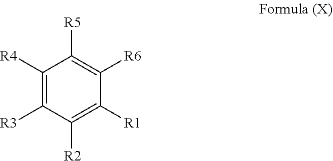

or a pharmaceutically acceptable salt, co-crystal, tautomer, stereoisomer, solvate, hydrate, polymorph, isotopically enriched derivative, or prodrug thereof, wherein R1 is hydrogen, optionally substituted acyl, optionally substituted alkyl, optionally substituted alkenyl, optionally substituted alkynyl, optionally substituted carbocyclyl, or optionally substituted aryl; R2 and R6 are, independently, hydrogen or carboxyl; R3 and R5 are, independently, hydroxyl, halogen, or alkoxy; and R4 is a hydrogen or an optionally substituted prenyl moiety; or optionally R4 and R3 are taken together with their intervening atoms to form a cyclic moiety, or optionally R4 and R5 are taken together with their intervening atoms to form a cyclic moiety, or optionally both 1) R4 and R3 are taken together with their intervening atoms to form a cyclic moiety and 2) R4 and R5 are taken together with their intervening atoms to form a cyclic moiety. In certain embodiments, R4 and R3 are taken together with their intervening atoms to form a cyclic moiety. In certain embodiments, R4 and R5 are taken together with their intervening atoms to form a cyclic moiety. In certain embodiments, “cannabinoid” refers to a compound of Formula (X), or a pharmaceutically acceptable salt thereof. In certain embodiments, both 1) R4 and R3 are taken together with their intervening atoms to form a cyclic moiety and 2) R4 and R5 are taken together with their intervening atoms to form a cyclic moiety.

In some embodiments, cannabinoids may be synthesized via the following steps: a) one or more reactions to incorporate three additional ketone moieties onto an acyl-CoA scaffold, where the acyl moiety in the acyl-CoA scaffold comprises between four and fourteen carbons; b) a reaction cyclizing the product of step (a); and c) a reaction to incorporate a prenyl moiety to the product of step (b) or a derivative of the product of step (b). In some embodiments, non-limiting examples of the acyl-CoA scaffold described in step (a) include hexanoyl-CoA and butyryl-CoA. In some embodiments, non-limiting examples of the product of step (b) or a derivative of the product of step (b) include olivetolic acid, divarinic acid, and sphaerophorolic acid.

In some embodiments, a cannabinoid compound of Formula (X) is of Formula (X-A), (X-B), or (X-C):

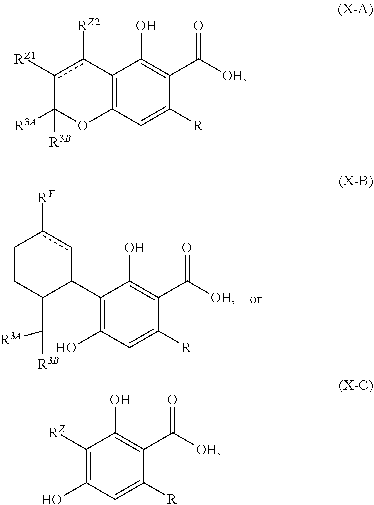

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;
   R is hydrogen, optionally substituted acyl, optionally substituted alkyl, optionally substituted alkenyl, optionally substituted alkynyl, optionally substituted carbocyclyl, or optionally substituted aryl;
   R^{Z1} is hydrogen, optionally substituted acyl, optionally substituted alkyl, optionally substituted alkenyl, optionally substituted alkynyl, optionally substituted carbocyclyl, or optionally substituted aryl;
   R^{Z2} is hydrogen, optionally substituted acyl, optionally substituted alkyl, optionally substituted alkenyl, optionally substituted alkynyl, optionally substituted carbocyclyl, or optionally substituted aryl;
   or optionally, R^{Z1} and R²² are taken together with their intervening atoms to form an optionally substituted carbocyclic ring;
   R^{3A} is hydrogen, optionally substituted acyl, optionally substituted alkyl, optionally substituted alkenyl, or optionally substituted alkynyl;
   R^{3B} is hydrogen, optionally substituted acyl, optionally substituted alkyl, optionally substituted alkenyl, or optionally substituted alkynyl;
   R^{Y} is hydrogen, optionally substituted acyl, optionally substituted alkyl, optionally substituted alkenyl, or optionally substituted alkynyl;
   R^{Z} is hydrogen, optionally substituted acyl, optionally substituted alkyl, optionally substituted alkenyl, or optionally substituted alkynyl.

In certain embodiments, a cannabinoid compound is of Formula (X-A):

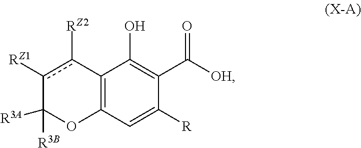

wherein [symbol] is a double bond, and each of R^{Z1} and R^{Z2} is hydrogen, one of R^{3A} and R^{3B} is optionally substituted C₂₋₆ alkenyl, and the other one of R^{3A} and R^{3B} is optionally substituted C₂₋₆ alkyl. In some embodiments, a cannabinoid compound of Formula (X) is of Formula (X-A), wherein each of R^{Z1} and R^{Z2} is hydrogen, one of R^{3A} and R^{3B} is a prenyl group, and the other one of R^{3A} and R^{3B} is optionally substituted methyl.

In certain embodiments, a cannabinoid compound of Formula (X) of Formula (X-A) is of Formula (11-z):

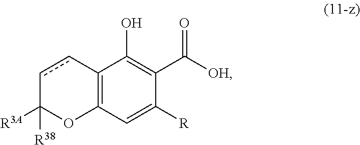

wherein [symbol] is a double bond or single bond, as valency permits; one of R^{3A} and R^{3B} is C₁₋₆ alkyl optionally substituted with alkenyl, and the other of R^{3A} and R^{3B} is optionally substituted C₁₋₆ alkyl. In certain embodiments, in a compound of Formula (11-z), [symbol] is a single bond; one of R^{3A} and R^{3B} is C₁₋₆ alkyl optionally substituted with prenyl; and the other of one of R^{3A} and R^{3B} is unsubstituted methyl; and R is as described in this application. In certain embodiments, in a compound of Formula (11-z), [symbol] is a singe bond; one of R^{3A} and R^{3B} is

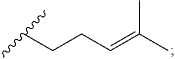 [SMILES: *CCC=C(C)C]

and the other of one of R^{3A} and R^{3B} is unsubstituted methyl; and R is as described in this application. In certain embodiments, a cannabinoid compound of Formula (11-z) is of Formula (11a):

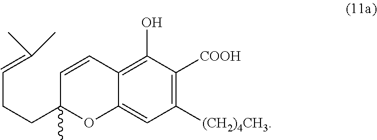

In certain embodiments, a cannabinoid compound of Formula (X) of Formula (X-A) is of Formula (11a):

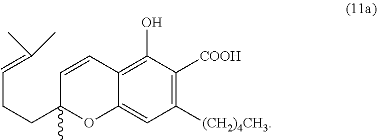

In certain embodiments a cannabinoid compound of Formula (X-A) is of Formula (10-z):

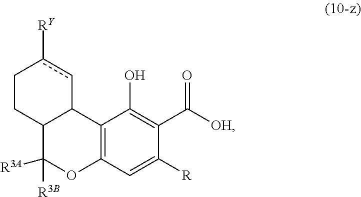

wherein [symbol] is a double bond or single bond, as valency permits; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (10-z), [symbol] is a single bond; each of R^{3A} and R^{3B} is unsubstituted methyl, and R is as described in this application. In certain embodiments, a cannabinoid compound of Formula (10-z) is of Formula (10a):

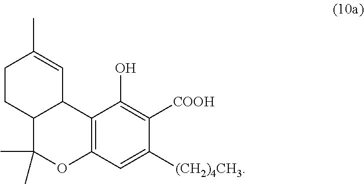

In certain embodiments, a compound of Formula (10a)

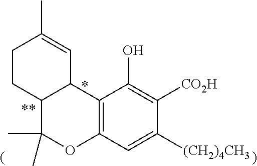

has a chiral atom labeled with * at carbon 10 and a chiral atom labeled with ** at carbon 6. In certain embodiments, in a compound of Formula (10a)

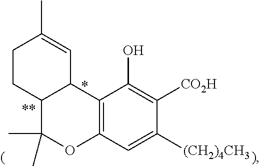

the chiral atom labeled with * at carbon 10 is of the R-configuration or S-configuration; and a chiral atom labeled with ** at carbon 6 is of the R-configuration. In certain embodiments, in a compound of Formula (10a)

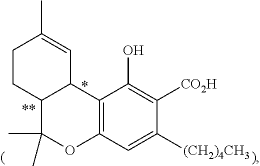

the chiral atom labeled with * at carbon 10 is of the S-configuration; and a chiral atom labeled with ** at carbon 6 is of the R-configuration or S-configuration. In certain embodiments, in a compound of Formula (10a)

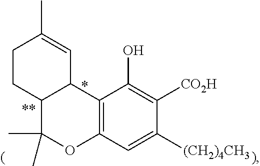

the chiral atom labeled with * at carbon 10 is of the R-configuration and a chiral atom labeled with ** at carbon 6 is of the R-configuration. In certain embodiments, a compound of Formula (10a)

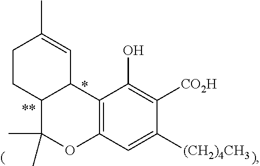

is of the formula:

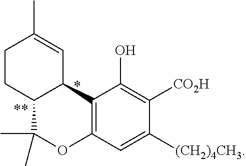

In certain embodiments, in a compound of Formula (10a)

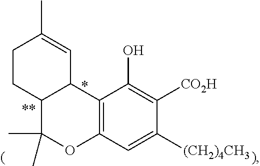

the chiral atom labeled with * at carbon 10 is of the S-configuration and a chiral atom labeled with ** at carbon 6 is of the S-configuration. In certain embodiments, a compound of Formula (10a)

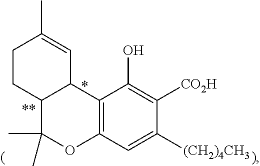

is of the formula:

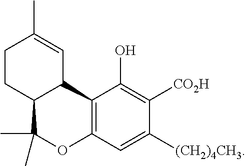

In certain embodiments, a cannabinoid compound is of Formula (X-B):

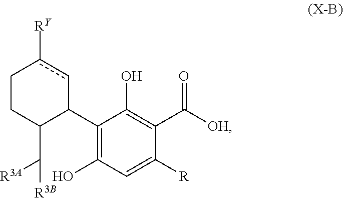

wherein [symbol] is a double bond; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (X-B), R^{Y} is optionally substituted C₁₋₆ alkyl; one of R^{3A} and R^{3B} is

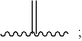

and the other one of R^{3A} and R^{3B} is unsubstituted methyl, and R is as described in this application. In certain embodiments, a compound of Formula (X-B) is of Formula (9a):

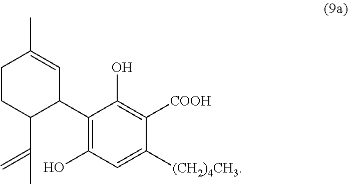

In certain embodiments, a compound of Formula (9a)

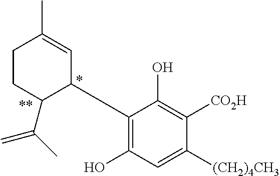

has a chiral atom labeled with * at carbon 3 and a chiral atom labeled with ** at carbon 4. In certain embodiments, in a compound of Formula (9a)

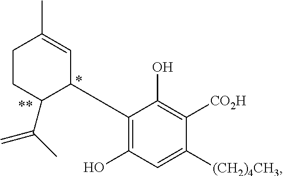

the chiral atom labeled with * at carbon 3 is of the R-configuration or S-configuration; and a chiral atom labeled with ** at carbon 4 is of the R-configuration. In certain embodiments, in a compound of Formula (9a)

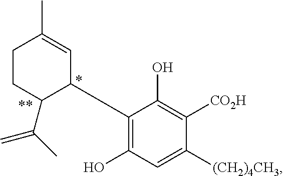

the chiral atom labeled with * at carbon 3 is of the S-configuration; and a chiral atom labeled with ** at carbon 4 is of the R-configuration or S-configuration. In certain embodiments, in a compound of Formula (9a)

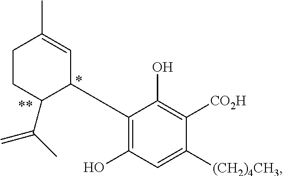

the chiral atom labeled with * at carbon 3 is of the R-configuration and a chiral atom labeled with ** at carbon 4 is of the R-configuration. In certain embodiments, a compound of Formula (9a)

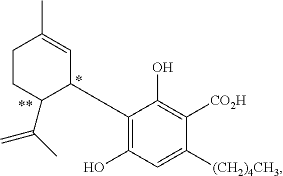

is of the formula:

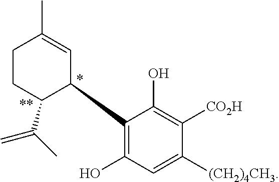

In certain embodiments, in a compound of Formula (9a)

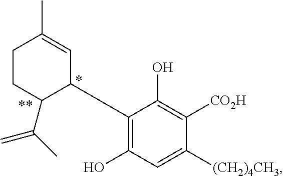

the chiral atom labeled with * at carbon 3 is of the S-configuration and a chiral atom labeled with ** at carbon 4 is of the S-configuration. In certain embodiments, a compound of Formula (9a)

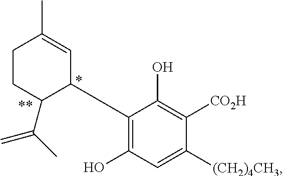

is of the formula:

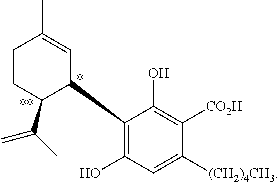

In certain embodiments, a cannabinoid compound is of Formula (X-C):

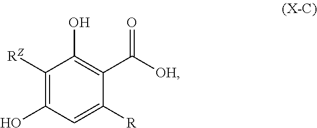

wherein R^{Z} is optionally substituted alkyl or optionally substituted alkenyl. In certain embodiments, a compound of Formula (X-C) is of formula:

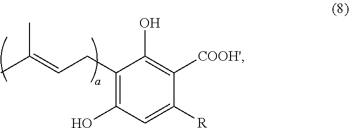

wherein a is 1, 2, 3, 4, 5, 6, 7, 8, 9, or 10. In certain embodiments, a is 1. In certain embodiments, a is 2. In certain embodiments, a is 3. In certain embodiments, a is 1, 2, or 3 for a compound of Formula (X-C). In certain embodiments, a cannabinoid compound is of Formula (X-C), and a is 1, 2, 3, 4, or 5. In certain embodiments, a compound of Formula (X-C) is of Formula (8a):

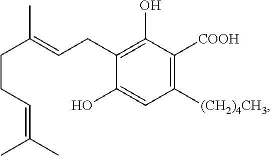

In some embodiments, cannabinoids of the present disclosure comprise cannabinoid receptor ligands. Cannabinoid receptors are a class of cell membrane receptors in the G protein-coupled receptor superfamily. Cannabinoid receptors include the CB₁ receptor and the CB₂ receptor. In some embodiments, cannabinoid receptors comprise GPR18, GPR55, and PPAR. (See Bram et al. “Activation of GPR18 by cannabinoid compounds: a tale of biased agonism” Br J Pharmcol v171 (16) (2014); Shi et al. “The novel cannabinoid receptor GPR55 mediates anxiolytic-like effects in the medial orbital cortex of mice with acute stress” Molecular Brain 10, No. 38 (2017); and O'Sullvan, Elizabeth. “An update on PPAR activation by cannabinoids” Br J Pharmcol v. 173(12) (2016)).

In some embodiments, cannabinoids comprise endocannabinoids, which are substances produced within the body, and phytocannabinoids, which are cannabinoids that are naturally produced by plants of genus Cannabis. In some embodiments, phytocannabinoids comprise the acidic and decarboxylated acid forms of the naturally-occurring plant-derived cannabinoids, and their synthetic and biosynthetic equivalents.

Over 94 phytocannabinoids have been identified to date (Berman, Paula, et al. “A new ESI-LC/MS approach for comprehensive metabolic profiling of phytocannabinoids in Cannabis.” Scientific reports 8.1 (2018): 14280; El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42; Rudolf Brenneisen, 2007, Chemistry and Analysis of Phytocannabinoids, Citti, Cinzia, et al. “A novel phytocannabinoid isolated from Cannabis sativa L. with an in vivo cannabimimetic activity higher than A9-tetrahydrocannabinol: Δ9-Tetrahydrocannabiphorol.” Sci Rep 9 (2019): 20335, each of which is incorporated by reference in this application in its entirety). In some embodiments, cannabinoids comprise Δ⁹-tetrahydrocannabinol (THC) type (e.g., (−)-trans-delta-9-tetrahydrocannabinol or dronabinol, (+)-trans-delta-9-tetrahydrocannabinol, (−)-cis-delta-9-tetrahydrocannabinol, or (+)-cis-delta-9-tetrahydrocannabinol), cannabidiol (CBD) type, cannabigerol (CBG) type, cannabichromene (CBC) type, cannabicyclol (CBL) type, cannabinodiol (CBND) type, or cannabitriol (CBT) type cannabinoids, or any combination thereof (see, e.g., R Pertwee, ed, Handbook of Cannabis (Oxford, UK: Oxford University Press, 2014)), which is incorporated by reference in this application in its entirety). A non-limiting list of cannabinoids comprises: cannabiorcol-C1 (CBNO), CBND-C1 (CBNDO), Δ⁹-trans-Tetrahydrocannabiorcolic acid-C1 (Δ⁹-THCO), Cannabidiorcol-C1 (CBDO), Cannabiorchromene-C1 (CBCO), (−)-Δ⁸-trans-(6aR,10aR)-Tetrahydrocannabiorcol-C1 (Δ⁸-THCO), Cannabiorcyclol C1 (CBLO), CBG-C1 (CBGO), Cannabinol-C₂ (CBN-C2), CBND-C2, Δ⁹-THC-C2, CBD-C2, CBC-C2, Δ⁸-THC-C2, CBL-C2, Bisnor-cannabielsoin-C1 (CBEO), CBG-C2, Cannabivarin-C3 (CBNV), Cannabinodivarin-C3 (CBNDV), (−)-Δ⁹-trans-Tetrahydrocannabivarin-C3 (Δ⁹-THCV), (−)-Cannabidivarin-C3 (CBDV), (±)-Cannabichromevarin-C3 (CBCV), (−)-Δ⁸-trans-THC-C3 (Δ⁸-THCV), (±)-(1aS,3aR,8bR,8cR)-Cannabicyclovarin-C3 (CBLV), 2-Methyl-2-(4-methyl-2-pentenyl)-7-propyl-2H-1-benzopyran-5-ol, Δ⁷-tetrahydrocannabivarin-C3 (Δ⁷-THCV), CBE-C2, Cannabigerovarin-C3 (CBGV), Cannabitriol-C1 (CBTO), Cannabinol-C4 (CBN-C4), CBND-C4, (−)-Δ⁹-trans-Tetrahydrocannabinol-C4 (Δ⁹-THC-C4), Cannabidiol-C4 (CBD-C4), CBC-C4, (−)-trans-Δ⁸-THC-C4, CBL-C4, Cannabielsoin-C3 (CBEV), CBG-C4, CBT-C2, Cannabichromanone-C3, Cannabiglendol-C3 (OH-iso-HHCV-C3), Cannabioxepane-C5 (CBX), Dehydrocannabifuran-C5 (DCBF), Cannabinol-C5 (CBN), Cannabinodiol-C5 (CBND), (−)-Δ⁹-trans-Tetrahydrocannabinol-C5 (Δ⁹-THC), (−)-Δ⁸-trans-(6aR, 10aR)-Tetrahydrocannabinol-C5 (Δ⁹-THC), (±)-Cannabichromene-C5 (CBC), (−)-Cannabidiol-C5 (CBD), (±)-(1aS,3aR,8bR,8cR)-CannabicyclolC5 (CBL), Cannabicitran-C5 (CBR), (−)-Δ⁹-(6aS, 10aR-cis)-Tetrahydrocannabinol-C5 ((−)-cis-Δ⁹-THC), (−)-Δ⁷-trans-(1R,3R,6R)-Isotetrahydrocannabinol-C5 (trans-isoA⁷-THC), CBE-C4, Cannabigerol-C5 (CBG), Cannabitriol-C3 (CBTV), Cannabinol methyl ether-C5 (CBNM), CBNDM-C5, 8-OH—CBN-C5 (OH—CBN), OH—CBND-C5 (OH—CBND), 10-Oxo-A^{6a(10a)}-Tetrahydrocannabinol-C5 (OTHC), Cannabichromanone D-C5, Cannabicoumaronone-C5 (CBCON-C5), Cannabidiol monomethyl ether-C5 (CBDM), Δ⁹-THCM-C5, (±)-3″-hydroxy-Δ⁴″-cannabichromene-C5, (5aS,6S,9R,9aR)-Cannabielsoin-C5 (CBE), 2-geranyl-5-hydroxy-3-n-pentyl-1,4-benzoquinone-C5, 5-geranyl olivetolic acid, 5-geranyl olivetolate, 8α-Hydroxy-Δ⁹-Tetrahydrocannabinol-C5 (8α-OH-Δ⁹-THC), 8β-Hydroxy-Δ⁹-Tetrahydrocannabinol-C5 (80-OH-Δ⁹-THC), 10α-Hydroxy-Δ⁸-Tetrahydrocannabinol-C5 (10α-OH-Δ⁸-THC), 100-Hydroxy-Δ⁸-Tetrahydrocannabinol-C5 (100-OH-Δ⁸-THC), 10α-hydroxy-Δ^{9,11}-hexahydrocannabinol-C5, 9β, 10β-Epoxyhexahydrocannabinol-C5, OH—CBD-C5 (OH—CBD), Cannabigerol monomethyl ether-C5 (CBGM), Cannabichromanone-C5, CBT-C4, (±)-6,7-cis-epoxycannabigerol-C5, (±)-6,7-trans-epoxycannabigerol-C5, (−)-7-hydroxycannabichromane-C5, Cannabimovone-C5, (−)-trans-Cannabitriol-C5 ((−)-trans-CBT), (+)-trans-Cannabitriol-C5 ((+)-trans-CBT), (±)-cis-Cannabitriol-C5 ((±)-cis-CBT), (−)-trans-10-Ethoxy-9-hydroxy-Δ^{6a(10a)}-tetrahydrocannabivarin-C3 [(−)-trans-CBT-OEt], (−)-(6aR,9S,10S,10aR)-9,10-Dihydroxyhexahydrocannabinol-C5 [(−)-Cannabiripsol] (CBR), Cannabichromanone C-C5, (−)-6a,7,10a-Trihydroxy-Δ⁹-tetrahydrocannabinol-C5 [(−)-Cannabitetrol] (CBTT), Cannabichromanone B-C5, 8,9-Dihydroxy-Δ^{6a(10a)}-tetrahydrocannabinol-C5 (8,9-Di-OHCBT), (±)-4-acetoxycannabichromene-C5, 2-acetoxy-6-geranyl-3-n-pentyl-1,4-benzoquinone-C5, 11-Acetoxy-Δ⁹-TetrahydrocannabinolC5 (11-OAc-Δ9-THC), 5-acetyl-4-hydroxycannabigerol-C5, 4-acetoxy-2-geranyl-5-hydroxy-3-npentylphenol-C5, (−)-trans-10-Ethoxy-9-hydroxy-Δ^{6a(10a)}-tetrahydrocannabinol-C5 ((−)-trans-CBTOEt), sesquicannabigerol-C5 (SesquiCBG), carmagerol-C₅, 4-terpenyl cannabinolate-C5, 0-fenchyl-Δ⁹-tetrahydrocannabinolate-C5, α-fenchyl-Δ⁹-tetrahydrocannabinolate-C5, epi-bornyl-Δ⁹-tetrahydrocannabinolate-C5, bornyl-Δ⁹-tetrahydrocannabinolate-C5, α-terpenyl-Δ⁹-tetrahydrocannabinolate-C5, 4-terpenyl-Δ⁹-tetrahydrocannabinolate-C5, 6,6,9-trimethyl-3-pentyl-6H-dibenzo[b,d]pyran-1-ol, 3-(1,1-dimethylheptyl)-6,6a,7, 8, 10,10a-hexahydro-1-hydroxy-6,6-dimethyl-9H-dibenzo[b,d]pyran-9-one, (−)-(3S,4S)-7-hydroxy-A⁶-tetrahydrocannabinol-1,1-dimethylheptyl, (+)-(3S,4S)-7-hydroxy-A⁶-tetrahydrocannabinol-1,1-dimethylheptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(4-morpholinylmethyl)-pyrrolo[1,2,3-de]-1,4-benzoxazin-6-yl]-1-naphthalenyl-methanone), certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′, 3′,4′,5′,6′-hexahydrobiphenyl, tetrahydrocannabiphorol (THCP), cannabidiphorol (CBDP), CBGP, CBCP, their acidic forms, salts of the acidic forms, dimers of any combination of the above, trimers of any combination of the above, polymers of any combination of the above, or any combination thereof.

A cannabinoid described in this application can be a rare cannabinoid. For example, in some embodiments, a cannabinoid described in this application corresponds to a cannabinoid that is naturally produced in conventional Cannabis varieties at concentrations of less than 10%, 9%, 8%, 7%, 6%, 5%, 4%, 3%, 2%,1%, 0.9%, 0.8%, 0.7%, 0.6%, 0.5%, 0.25%, or 0.1% by dry weight of the female flower. In some embodiments, rare cannabinoids include CBGA, CBGVA, THCVA, CBDVA, CBCVA, and CBCA. In some embodiments, rare cannabinoids are cannabinoids that are not THCA, THC, CBDA or CBD.

A cannabinoid described in this application can also be a non-rare cannabinoid.

In some embodiments, the cannabinoid is selected from the cannabinoids listed in Table 1.

TABLE 1
Non-limiting examples of cannabinoids according to the present disclosure

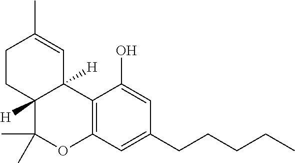 [SMILES: CCCCCc1cc(O)c2c(c1)OC(C)(C)[C@@H]1CCC(C)=C[C@@H]21]

Δ⁹-Tetrahydro-
cannabinol
Δ⁹-THC-C₅

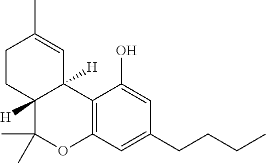 [SMILES: CCCCc1cc(O)c2c(c1)OC(C)(C)[C@@H]1CCC(C)=C[C@@H]21]

Δ⁹-Tetrahydro-
cannabinol-C₄
Δ⁹-THC-C₄

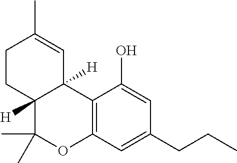 [SMILES: CCCc1cc(O)c2c(c1)OC(C)(C)[C@@H]1CCC(C)=C[C@@H]21]

Δ⁹-Tetrahydro-
cannabivarin
Δ⁹-THCV-C₃

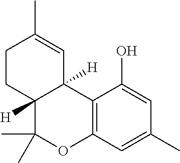

Δ⁹-Tetrahydro-
cannabiorcol
Δ⁹-THCO-C₁

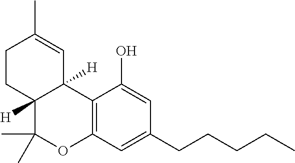 [SMILES: CCCCCc1cc(O)c2c(c1)OC(C)(C)[C@@H]1CCC(C)=C[C@@H]21]

(−)-(6aS,10aR)-Δ⁹-
Tetrahydro-
cannabinol
(−)-cis-Δ⁹-THC-C₅

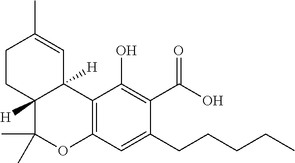 [SMILES: CCCCCc1cc2c(c(O)c1C(=O)O)[C@@H]1C=C(C)CC[C@H]1C(C)(C)O2]

Δ⁹-Tetrahydro-
cannabinolic acid A
Δ⁹-THCA-C₅ A

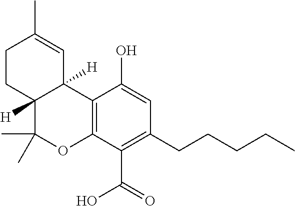 [SMILES: CCCCCc1cc(O)c2c(c1C(=O)O)OC(C)(C)[C@@H]1CCC(C)=C[C@@H]21]

Δ⁹-Tetrahydro-
cannabinolic acid B
Δ⁹-THCA-C₅ B

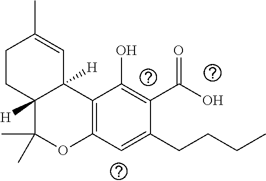 [SMILES: CCCCc1cc2c(c(O)c1C(=O)O)[C@@H]1C=C(C)CC[C@H]1C(C)(C)O2]

Δ⁹-Tetrahydro-
cannabinolic acid-C₄
A and/or B
Δ⁹-THCA-C₄
A and/or B

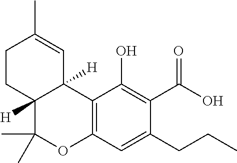 [SMILES: CCCc1cc2c(c(O)c1C(=O)O)[C@@H]1C=C(C)CC[C@H]1C(C)(C)O2]

Δ⁹-Tetrahydro-
cannabivarinic acid A
Δ⁹-THCVA-C₃ A

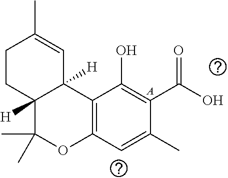

Δ⁹-Tetrahydro-
cannabiorcolic acid
A and/or B
Δ⁹-THCOA-C₁
A and/or B

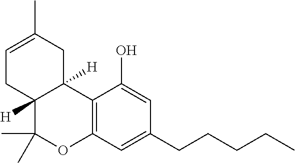 [SMILES: CCCCCc1cc(O)c2c(c1)OC(C)(C)[C@@H]1CC=C(C)C[C@@H]21]

(−)-Δ⁸-trans-
(6aR,10aR)-
Δ⁸-Tetrahydro-
cannabinol
Δ⁸-THC-C₅

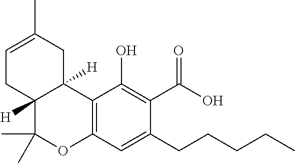 [SMILES: CCCCCc1cc2c(c(O)c1C(=O)O)[C@@H]1CC(C)=CC[C@H]1C(C)(C)O2]

(−)-Δ⁸-trans-
(6aR,10aR)-
Tetrahydro-
cannabinolic
acid A
Δ⁸-THCA-C₅ A

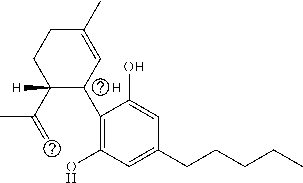

(−)-Cannabidiol
CBD-C5

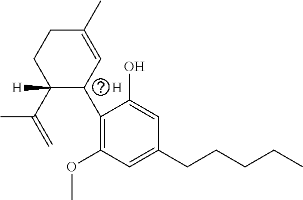

Cannabidiol
momomethyl ether
CBDM-C5

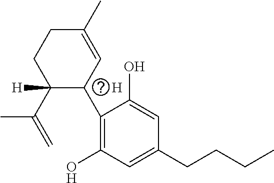

Cannabidiol-C4
CBD-C4

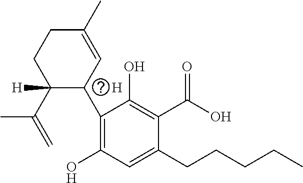

Cannabidiolic acid
CBDA-C5

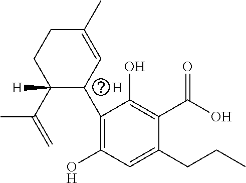

Cannabidivarinic acid
CBDVA-C3

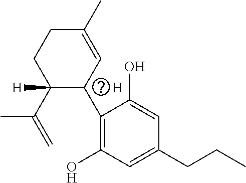

(−)-Cannabidivarin
CBDV-C3

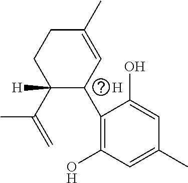

Cannabidiorcol
CBD-C1

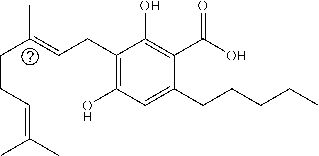 [SMILES: CCCCCc1cc(O)c(C/C=C(\C)CCC=C(C)C)c(O)c1C(=O)O]

Cannabigerolic acid
A
(E)-CBGA-C₅ A

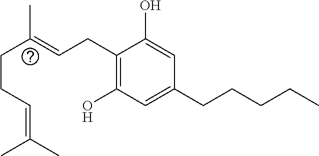 [SMILES: CCCCCc1cc(O)c(C/C=C(\C)CCC=C(C)C)c(O)c1]

Cannabigerol
(E)-CBG-C₅

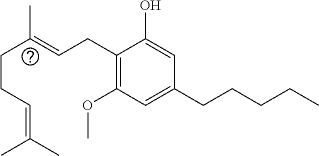 [SMILES: CCCCCc1cc(O)c(C/C=C(\C)CCC=C(C)C)c(OC)c1]

Cannabigerol
monomethyl ether
(E)-CBGM-C₅ A

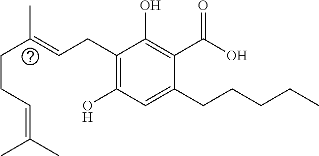 [SMILES: CCCCCc1cc(O)c(C/C=C(\C)CCC=C(C)C)c(O)c1C(=O)O]

Cannabinerolic acid
A
(Z)-CBGA-C₅ A

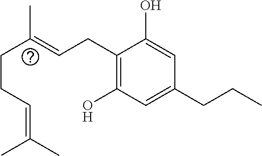 [SMILES: CCCc1cc(O)c(C/C=C(\C)CCC=C(C)C)c(O)c1]

Cannabigerovarin
(E)-CBGV-C₃

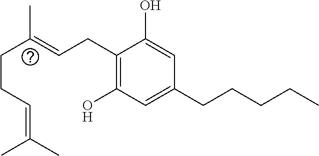 [SMILES: CCCCCc1cc(O)c(C/C=C(\C)CCC=C(C)C)c(O)c1]

Cannabigerol
(E)-CBG-C₅

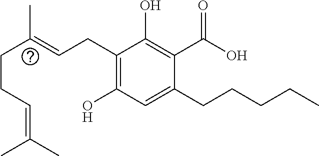 [SMILES: CCCCCc1cc(O)c(C/C=C(\C)CCC=C(C)C)c(O)c1C(=O)O]

Cannabigerolic acid A
(E)-CBGA-C₅ A

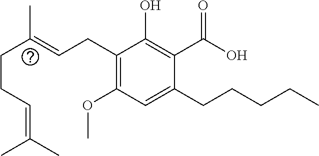 [SMILES: CCCCCc1cc(OC)c(C/C=C(\C)CCC=C(C)C)c(O)c1C(=O)O]

Cannabigerolic acid A
monomethyl ether
(E)-CBGAM-C₅ A

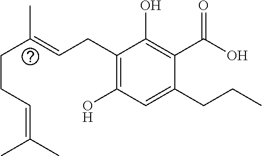 [SMILES: CCCc1cc(O)c(C/C=C(\C)CCC=C(C)C)c(O)c1C(=O)O]

Cannabigerovarinic
acid A
(E)-CBGVA-C₃ A

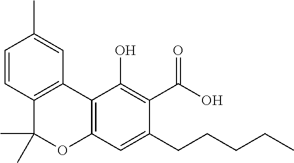 [SMILES: CCCCCc1cc2c(c(O)c1C(=O)O)-c1cc(C)ccc1C(C)(C)O2]

Cannabinolic acid A
CBNA-C5 A

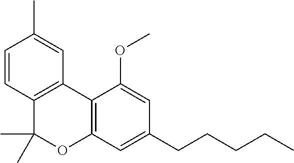 [SMILES: CCCCCc1cc(OC)c2c(c1)OC(C)(C)c1ccc(C)cc1-2]

Cannabinol methyl
ether
CBNM-C5

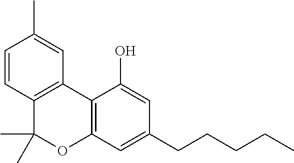 [SMILES: CCCCCc1cc(O)c2c(c1)OC(C)(C)c1ccc(C)cc1-2]

Cannabinol
CBN-C5

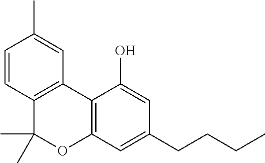 [SMILES: CCCCc1cc(O)c2c(c1)OC(C)(C)c1ccc(C)cc1-2]

Cannabinol-C4
CBN-C4

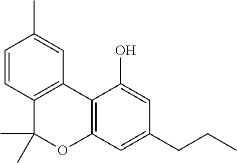 [SMILES: CCCc1cc(O)c2c(c1)OC(C)(C)c1ccc(C)cc1-2]

Cannabivarin
CBN-C3

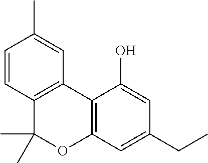 [SMILES: CCc1cc(O)c2c(c1)OC(C)(C)c1ccc(C)cc1-2]

Cannabinol-C2
CBN-C2

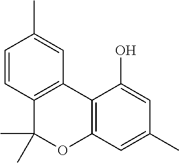

Cannabiorcol
CBN-C1

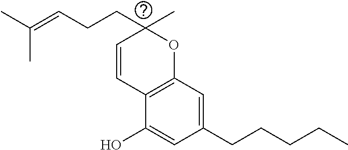 [SMILES: CCCCCc1cc(O)c2c(c1)OC(C)(CCC=C(C)C)C=C2]

(±)-Cannabichromene
CBC-C₅

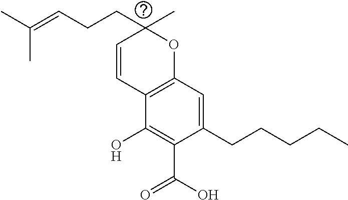 [SMILES: CCCCCc1cc2c(c(O)c1C(=O)O)C=CC(C)(CCC=C(C)C)O2]

(±)-Cannabichromenic
acid A
CBCA-C₅ A

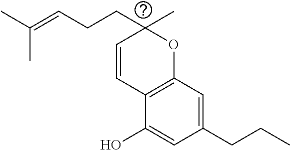 [SMILES: CCCc1cc(O)c2c(c1)OC(C)(CCC=C(C)C)C=C2]

(±)-Cannabivarichro-
mene,
(±)-
Cannabichromevarin
CBCV-C₃

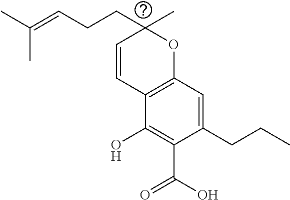 [SMILES: CCCc1cc2c(c(O)c1C(=O)O)C=CC(C)(CCC=C(C)C)O2]

(±)-Cannabichro-
mevarinic
acid A
CBCVA-C₃ A

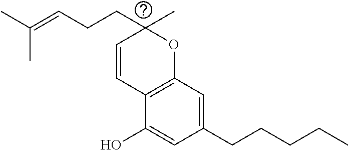 [SMILES: CCCCCc1cc(O)c2c(c1)OC(C)(CCC=C(C)C)C=C2]

(±)-
Cannabichromene
CBC-C₅

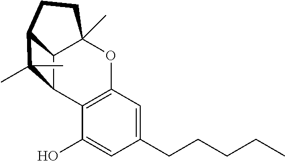

(±)-
(1aS,3aR,8bR,8cR)-
Cannabicyclol
CBL-C₅

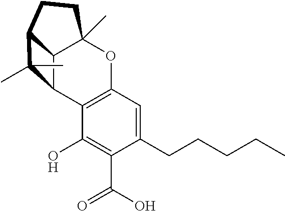

(±)-(1aS,3aR,8bR,8cR)-
Cannabicyclolic acid A
CBLA-C₅ A

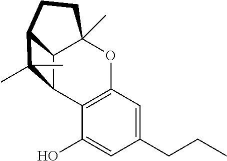

(±)-
(1aS,3aR,8bR,8cR)-
Cannabicyclovarin
CBLV-C₃

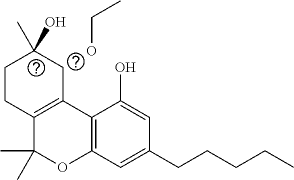

(−)-(9R,10R)-trans-
10-O-Ethyl-
cannabitriol
(−)-trans-CBT-OEt-C5

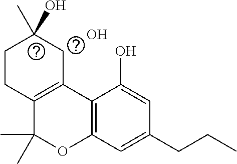

(±)-(9R,10R/9S,10S)-
Cannabitriol-C3
(±)-trans-CBT-C3

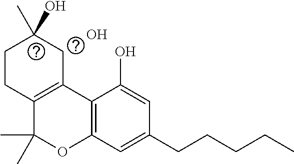

(−)-(9R,10R)-trans-
Cannabitriol
(−)-trans-CBT-C5

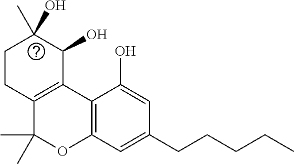 [SMILES: CCCCCc1cc(O)c2c(c1)OC(C)(C)C1=C2[C@H](O)[C@](C)(O)CC1]

(+)-(9S,10S)-
Cannabitriol
(+)-trans-CBT-C5

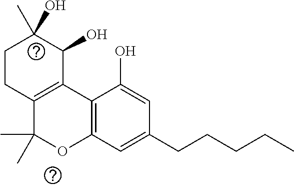 [SMILES: CCCCCc1cc(O)c2c(c1)OC(C)(C)C1=C2[C@H](O)[C@](C)(O)CC1]

(±)-(9R,10S/9S,10R)-
Cannabitriol
(±)-cis-CBT-C5

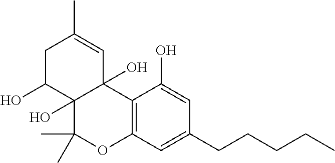 [SMILES: CCCCCc1cc(O)c2c(c1)OC(C)(C)C1(O)C(O)CC(C)=CC21O]

(−)-6a,7,10a-
Trihydroxy-
Δ9-tetrahydro-
cannabinol
(−)-Cannabitetrol

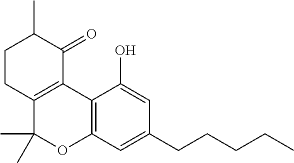 [SMILES: CCCCCc1cc(O)c2c(c1)OC(C)(C)C1=C2C(=O)C(C)CC1]

10-Oxo-Δ6a(10a)-
tetrahydro-
cannabinol
OTHC

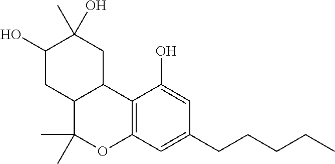 [SMILES: CCCCCc1cc(O)c2c(c1)OC(C)(C)C1CC(O)C(C)(O)CC21]

8,9-Dihydroxy-
Δ6a(10a)-
tetrahydro-
cannabinol
8,9-Di-OH-CBT-C5

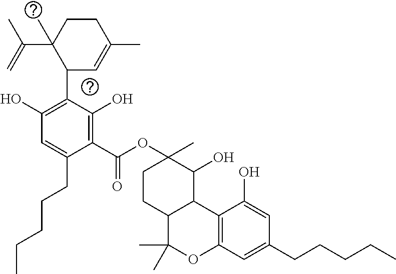 [SMILES: C=C(C)C1(C)CCC(C)=CC1c1c(O)cc(CCCCC)c(C(=O)OC2(C)CCC3C(c4c(O)cc(CCCCC)cc4OC3(C)C)C2O)c1O]

Cannabidiolic acid A
cannabitriol ester
CBDA-C5 9-OH-CBT-C5
ester

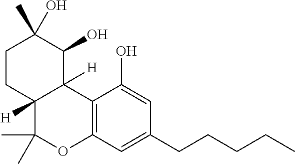 [SMILES: CCCCCc1cc(O)c2c(c1)OC(C)(C)[C@@H]1CC[C@](C)(O)[C@@H](O)C21]

(−)-
(6aR,9S,10S,10aR)-
9,10-Dihydroxy-
hexahydro-
cannabinol,
Cannabiripsol
Cannabiripsol-C5

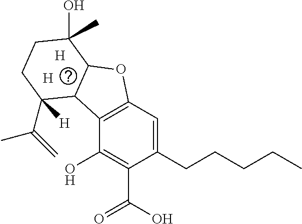

(5aS,6S,9R,9aR)-
Cannabielsoic acid B
CBEA-C5 B

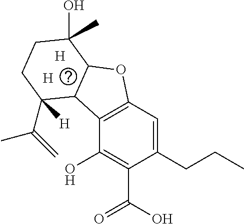

(5aS,6S,9R,9aR)-
C3-Cannabielsoic
acid B
CBEA-C3 B

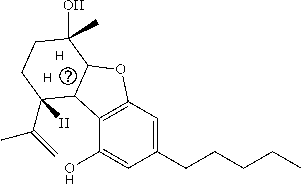

(5aS,6S,9R,9aR)-
Cannabielsoin
CBE-C5

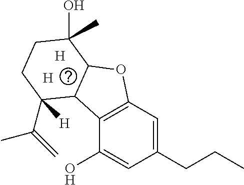

(5aS,6S,9R,9aR)-
C3-Cannabielsoin
CBE-C3

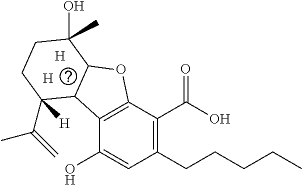

(5aS,6S,9R,9aR)-
Cannabielsoic acid A
CBEA-C5 A

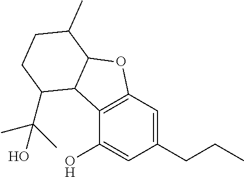 [SMILES: CCCc1cc(O)c2c(c1)OC1C(C)CCC(C(C)(C)O)C21]

Cannabiglendol-C3
OH-iso-HHCV-C3

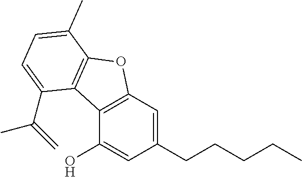 [SMILES: C=C(C)c1ccc(C)c2oc3cc(CCCCC)cc(O)c3c12]

Dehydro-
cannabifuran
DCBF-C5

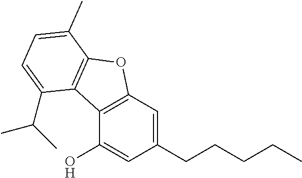 [SMILES: CCCCCc1cc(O)c2c(c1)oc1c(C)ccc(C(C)C)c12]

Cannabifuran
CBF-C5

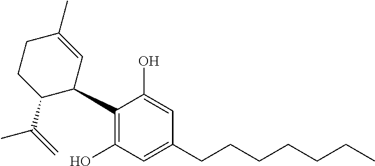 [SMILES: C=C(C)[C@@H]1CCC(C)=C[C@H]1c1c(O)cc(CCCCCCC)cc1O]

Cannabidiphorol
(CBDP)

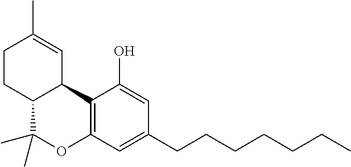 [SMILES: CCCCCCCc1cc(O)c2c(c1)OC(C)(C)[C@@H]1CCC(C)=C[C@@H]21]

Tetrahydro-
cannabiphorol
(THCP)

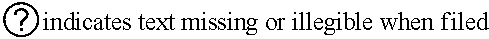

“Cannabinoids are often classified by “type,” i.e., by the topological arrangement of their prenyl moieties (See, for example, M. A. Elsohly and D. Slade, Life Sci., 2005, 78, 539-548; and L. O. Hanus et al. Nat. Prod. Rep., 2016, 33, 1357). Generally, each “type” of cannabinoid includes the variations possible for ring substitutions of the resorcinol moiety at the position meta to the two hydroxyl moieties. As used herein, a “CBG-type” cannabinoid is a 3-[(2E)-3,7-dimethylocta-2,6-dienyl]-2,4-dihydroxybenzoic acid optionally substituted at the 6 position of the benzoic acid moiety. As used herein, “CBC-type” cannabinoids refer to 5-hydroxy-2-methyl-2-(4-methylpent-3-enyl)-chromene-6-carboxylic acid optionally substituted at the 7 position of the chromene moiety. As used herein, a “THC-type”cannabinoid is a (6aR,10aR)-1-hydroxy-6,6,9-trimethyl-6a,7,8,10a-tetrahydrobenzo[c]chromene-2-carboxylic acid optionally substituted at the 3 position of the benzo[c]chromene moiety. As used herein, a “CBD-type” cannabinoid is a 2,4-dihydroxy-3-[(1R,6R)-3-methyl-6-prop-1-en-2-ylcyclohex-2-en-1-yl]-benzoic acid optionally substituted at the 6 position of the benzoic acid moiety. In some embodiments, the optional ring substitution for each “type” is an optionally substituted C1-C11 alkyl, an optionally substituted C1-C11 alkenyl, an optionally substituted C1-C11 alkynyl, or an optionally substituted C1-C11 aralkyl.

### Biosynthesis of Cannabinoids and Cannabinoid Precursors

Aspects of the present disclosure provide tools, sequences, and methods for the biosynthetic production of cannabinoids in host cells. In some embodiments, the present disclosure teaches expression of enzymes that are capable of producing cannabinoids by biosynthesis.

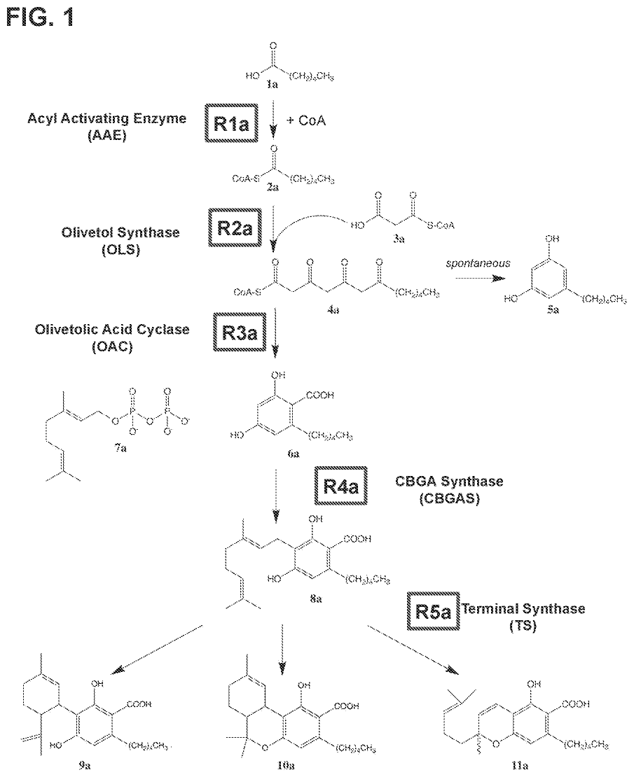

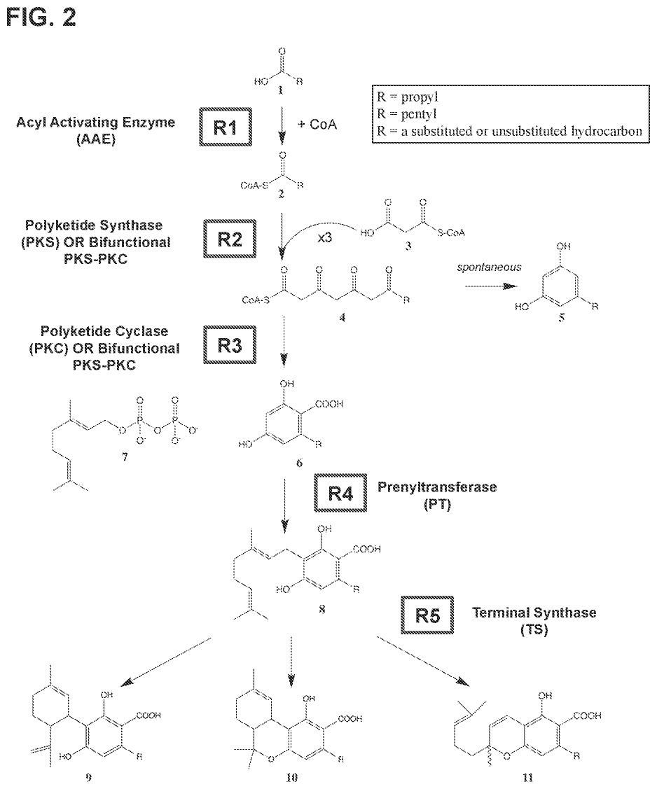

As a non-limiting example, one or more of the enzymes depicted in FIG. 2 may be used to produce a cannabinoid or cannabinoid precursor of interest. FIG. 1 shows a cannabinoid biosynthesis pathway for the most abundant phytocannabinoids found in Cannabis. See also, de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112), and Carvalho et al. “Designing Microorganisms for Heterologous Biosynthesis of Cannabinoids” (2017) FEMS Yeast Research Jun. 1;17(4), each of which is incorporated by reference in this application in its entirety for all purposes.

It should be appreciated that a precursor substrate for use in cannabinoid biosynthesis is generally selected based on the cannabinoid of interest. Non-limiting examples of cannabinoid precursors include compounds of Formulae (1)-(8) in FIG. 2. In some embodiments, polyketides, including compounds of Formula (5), could be prenylated. In certain embodiments, the precursor is a precursor compound shown in FIGS. 1, 2, or 3. Substrates in which R contains 1-40 carbon atoms are preferred. In some embodiments, substrates in which R contains 3-8 carbon atoms are most preferred.

As used in this application, a cannabinoid or a cannabinoid precursor may comprise an R group. See, e.g., FIG. 2. In some embodiments, R may be a hydrogen. In certain embodiments, R is optionally substituted alkyl. In certain embodiments, R is optionally substituted C1-40 alkyl. In certain embodiments, R is optionally substituted C2-40 alkyl. In certain embodiments, R is optionally substituted C2-40 alkyl, which is straight chain or branched alkyl. In certain embodiments, R is optionally substituted C3-8 alkyl. In certain embodiments, R is optionally substituted C₁-C₄₀ alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C10 alkyl. In certain embodiments, R is optionally substituted C1-C8 alkyl. In certain embodiments, R is optionally substituted C1-C5 alkyl. In certain embodiments, R is optionally substituted C1-C7 alkyl. In certain embodiments, R is optionally substituted C3-C5 alkyl. In certain embodiments, R is optionally substituted C3 alkyl. In certain embodiments, R is unsubstituted C3 alkyl. In certain embodiments, R is n-C3 alkyl. In certain embodiments, R is n-propyl. In certain embodiments, R is n-butyl. In certain embodiments, R is n-pentyl. In certain embodiments, R is n-hexyl. In certain embodiments, R is n-heptyl. In certain embodiments, R is of formula:

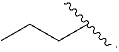 [SMILES: *CCC]

In certain embodiments, R is optionally substituted C4 alkyl. In certain embodiments, R is unsubstituted C4 alkyl. In certain embodiments, R is optionally substituted C5 alkyl. In certain embodiments, R is unsubstituted C5 alkyl. In certain embodiments, R is optionally substituted C6 alkyl. In certain embodiments, R is unsubstituted C6 alkyl. In certain embodiments, R is optionally substituted C₇ alkyl. In certain embodiments, R is unsubstituted C₇ alkyl. In certain embodiments, R is of formula:

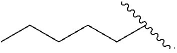 [SMILES: *CCCCC]

In certain embodiments, R is of formula:

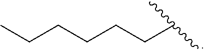 [SMILES: *CCCCCC]

In certain embodiments, R is of formula:

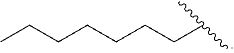 [SMILES: *CCCCCCC]

In certain embodiments, R is of formula:

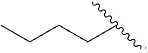 [SMILES: *CCCC]

In certain embodiments, R is of formula:

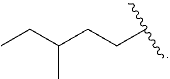 [SMILES: *CCC(C)CC]

In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

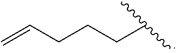 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

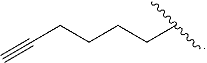 [SMILES: *CCCCC#C]

In certain embodiments, R is optionally substituted carbocyclyl. In certain embodiments, R is optionally substituted aryl (e.g., phenyl or napthyl).

The chain length of a precursor substrate can be from C1-C40. Those substrates can have any degree and any kind of branching or saturation or chain structure, including, without limitation, aliphatic, alicyclic, and aromatic. In addition, they may include any functional groups including hydroxy, halogens, carbohydrates, phosphates, methyl-containing or nitrogen-containing functional groups.

In some embodiments, R is H, an optionally substituted C1-C11 alkyl, an optionally substituted C1-C11 alkenyl, an optionally substituted C1-C11 alkynyl, or an optionally substituted C1-C11 aralkyl.

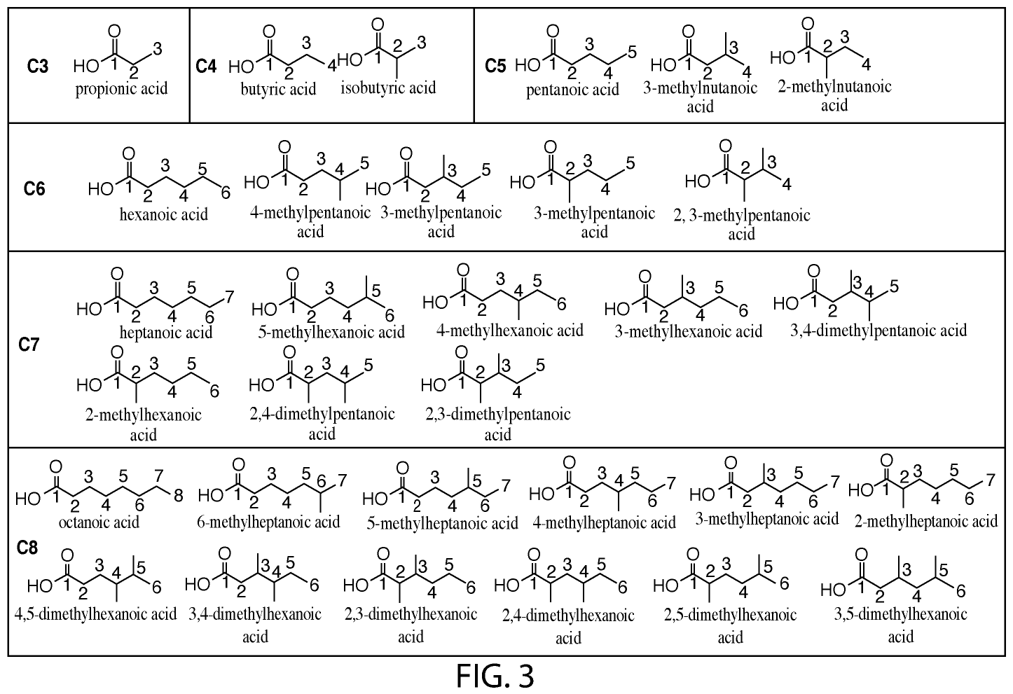

For example, FIG. 3 shows a non-exclusive set of putative precursors for the cannabinoid pathway. Aliphatic carboxylic acids including four to eight total carbons (“C4”-“C8” in FIG. 3) and up to 10-12 total carbons with either linear or branched chains may be used as precursors for the heterologous pathway. Non-limiting examples include methanoic acid, butyric acid, pentanoic acid, hexanoic acid, heptanoic acid, isovaleric acid, octanoic acid, and decanoic acid. Additional precursors may include ethanoic acid and propanoic acid. In some embodiments, in addition to acids, the ester, salt, and acid forms may all be used as substrates. Substrates may have any degree and any kind of branching, saturation, and chain structure, including, without limitation, aliphatic, alicyclic, and aromatic. In addition, they may include any functional modifications or combination of modifications including, without limitation, halogenation, hydroxylation, amination, acylation, alkylation, phenylation, and/or installation of pendant carbohydrates, phosphates, sulfates, heterocycles, or lipids, or any other functional groups.

Substrates for any of the enzymes disclosed in this application may be provided exogenously or may be produced endogenously by a host cell. In some embodiments, the cannabinoids are produced from a glucose substrate, so that compounds of Formula 1 shown in FIG. 2 and CoA precursors are synthesized by the cell. In other embodiments, a precursor is fed into the reaction. In some embodiments, a precursor is a compound selected from Formulae 1-8 in FIG. 2.

Cannabinoids produced by methods disclosed in this application include rare cannabinoids. Due to the low concentrations at which cannabinoids, including rare cannabinoids, occur in nature, producing industrially significant amounts of isolated or purified cannabinoids from the Cannabis plant may become prohibitive, especially in the case of rare cannabinoids, due to, e.g., the large volumes of Cannabis plants, and the large amounts of space, labor, time, and capital requirements to grow, harvest, and/or process the plant materials (see, for example, Crandall, K., 2016. A Chronic Problem: Taming Energy Costs and Impacts from Marijuana Cultivation. EQ Research; Mills, E., 2012. The carbon footprint of indoor Cannabis production. Energy Policy, 46, pp. 58-67; Jourabchi, M. and M. Lahet. 2014. Electrical Load Impacts of Indoor Commercial Cannabis Production. Presented to the Northwest Power and Conservation Council; O'Hare, M., D. Sanchez, and P. Alstone. 2013. Environmental Risks and Opportunities in Cannabis Cultivation. Washington State Liquor and Cannabis Board; 2018. Comparing Cannabis Cultivation Energy Consumption. New Frontier Data; and Madhusoodanan, J., 2019. Can cannabis go green? Nature Outlook: Cannabis; all of which are incorporated by reference in this disclosure). The disclosure provided in this application represents a potentially efficient method for producing high yields of cannabinoids, including rare cannabinoids. The disclosure provided in this application also represents a potential method for addressing concerns related to agricultural practices and water usage associated with traditional methods of cannabinoid production (Dillis et al. “Water storage and irrigation practices for cannabis drive seasonal patterns of water extraction and use in Northern California.” Journal of Environmental Management 272 (2020): 110955, incorporated by reference in this disclosure).

Cannabinoids produced by the disclosed methods also include non-rare cannabinoids. Without being bound by a particular theory, the methods described in this application may be advantageous compared with traditional plant-based methods for producing non-rare cannabinoids. For example, methods provided in this application represent potentially efficient means for producing consistent and high yields of non-rare cannabinoids. With traditional methods of cannabinoid production, in which cannabinoids are harvested from plants, maintaining consistent and uniform conditions, including airflow, nutrients, lighting, temperature, and humidity, can be difficult. For example, with plant-based methods, there can be microclimates created by branching, which can lead to inconsistent yields and by-product formation. In some embodiments, the methods described in this application are more efficient at producing a cannabinoid of interest as compared to harvesting cannabinoids from plants. For example, with plant-based methods, seed-to-harvest can take up to half a year, while cutting-to-harvest usually takes about 4 months. Additional steps including drying, curing, and extraction are also usually needed with plant-based methods. In contrast, in some embodiments, the fermentation-based methods described in this application only take about 1, 2, 3, 4, 5, 6, 7, 8, 9, or 10 days. In some embodiments, the fermentation-based methods described in this application only take about 3-5 days. In some embodiments, the fermentation-based methods described in this application only take about 5 days. In some embodiments, the methods provided in this application reduce the amount of security needed to comply with regulatory standards. For example, a smaller secured area may be needed to be monitored and secured to practice the methods described in this application as compared to the cultivation of plants. In some embodiments, the methods described in this application are advantageous over plant-sourced cannabinoids.

### Polyketide Cyclase (PKC)

A host cell described in this application may comprise a PKC. As used in this application, a “PKC” refers to an enzyme that is capable of cyclizing a polyketide. Without being bound by a particular theory, a PKC may be advantageous in cannabinoid production because it promotes the formation of compounds of Formula 6 (a non-decarboxylated compound). Since prenyltransferases predominantly and often preferentially prenylate compounds of Formula 6, the cyclase activity of PKCs may be advantageous in increasing the efficiency of cannabinoid production by providing a preferred substrate for a downstream prenyltransferase.

In certain embodiments, a polyketide cyclase (PKC) catalyzes the cyclization of an oxo fatty acyl-CoA (e.g., a compound of Formula (4)):

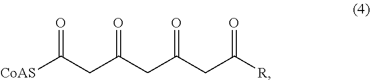

or 3,5,7-trioxododecanoyl-COA, 3,5,7-trioxodecanoyl-COA) to the corresponding intramolecular cyclization product (e.g., compound of Formula (6), including olivetolic acid and divarinic acid). A compound of Formula (6) may also be referred to as a 2,4-dihydroxybenzoic acid. In some embodiments, a PKC catalyzes the formation of a compound which occurs in the presence of a PKS. PKC substrates include trioxoalkanol-CoA, such as 3,5,7-Trioxododecanoyl-CoA, or a compound of Formula (4):

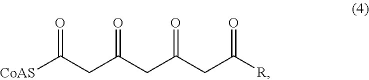

wherein R is hydrogen, optionally substituted acyl, optionally substituted alkyl, optionally substituted alkenyl, optionally substituted alkynyl, optionally substituted carbocyclyl, or optionally substituted aryl. In certain embodiments, a PKC catalyzes a compound of Formula (4):

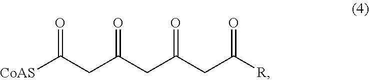

wherein R is hydrogen, optionally substituted acyl, optionally substituted alkyl, optionally substituted alkenyl, optionally substituted alkynyl, optionally substituted carbocyclyl, or optionally substituted aryl; to form a compound of Formula (6):

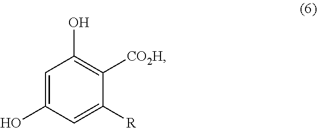

wherein R is hydrogen, optionally substituted acyl, optionally substituted alkyl, optionally substituted alkenyl, optionally substituted alkynyl, optionally substituted carbocyclyl, or optionally substituted aryl; as substrates. R is as definedprenyltransferase (PT). As used in this application. In some embodiments, R is a C₂-C₆ optionally substituted alkyl. In some embodiments, R is a propyl or pentyl. In some embodiments, R is pentyl. In some embodiments, R is propyl.

As one of ordinary skill in the art would appreciate, a PKC could be obtained from any source, including naturally occurring sources and synthetic sources (e.g., a non-naturally occurring PKC). In some embodiments, a PKC is from Cannabis. Non-limiting examples of PKCs include those disclosed in U.S. Pat. Nos. 9,611,460; 10,059,971; and U.S. Patent Application Publication No. 2019/0169661, which are incorporated by reference in this application in their entireties.

In some embodiments, a PKC is an olivetolic acid cyclase (OAC). In other embodiments, a PKC is a divarinic acid cyclase (DAC).

As used in this application, an “OAC” refers to an enzyme that is capable of catalyzing the formation of olivetolic acid (OA). In some embodiments, an OAC is capable of using a substrate of Formula (4a) (3,5,7-trioxododecanoyl-CoA):

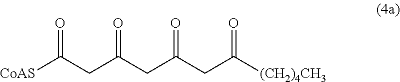

to form a compound of Formula (6a) (olivetolic acid):

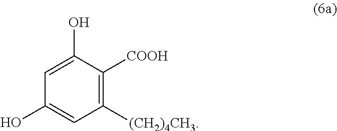

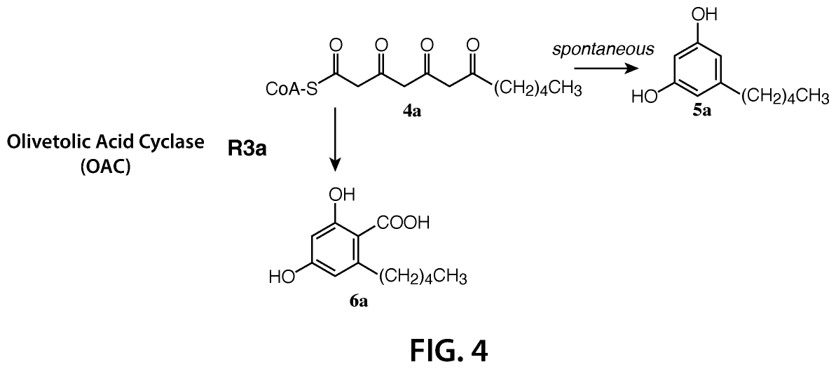

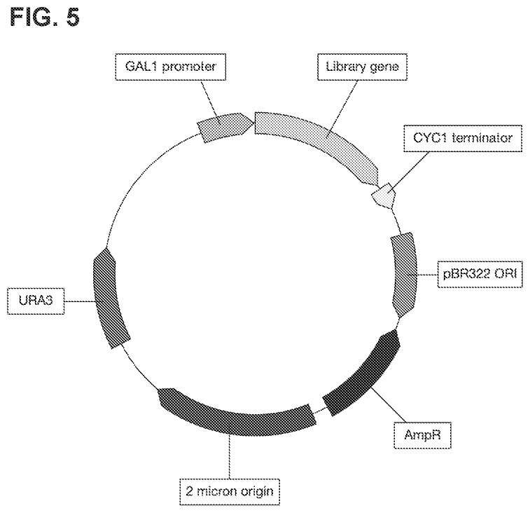

Olivetolic acid cyclase from Cannabis sativa (CsOAC) is a 101 amino acid enzyme that performs non-decarboxylative cyclization of the tetraketide product of olivetol synthase (FIG. 4 Structure 4a) via aldol condensation to form olivetolic acid (FIG. 4 Structure 6a). CsOAC was identified and characterized by Gagne et al. (Proc Natl Acad Sci USA. 2012 Jul. 31; 109(31):12811-6) via transcriptome mining, and its cyclization function was recapitulated in vitro to demonstrate that CsOAC is required for formation of olivetolic acid in Cannabis sativa. A crystal structure of the enzyme was published by Yang et al. (FEBS J. 2016 Mar.;283(6):1088-106), which revealed that the enzyme is a homodimer and belongs to the α+β barrel (DABB) superfamily of protein folds. CsOAC is the only known sequenced plant polyketide cyclase. Multiple fungal Type III polyketide synthases have been identified that perform both polyketide synthase and cyclization functions (Funa et al., J Biol Chem. 2007 May 11;282(19):14476-81); however, in plants such a dual function enzyme has not yet been discovered.

The wild-type amino acid sequence of CsOAC, which catalyzes the formation of olivetolic acid (OA) from 3,5,7-Trioxododecanoyl-CoA, is provided by UniProtKB-I 6WU39 (SEQ ID NO: 1):

(SEQ ID NO: 1)
MAVKHLIVLKFKDEITEAQKEEFFKTYVNLVNIIPAMKDVYWGKDVTQKNK
EEGYTHIVEVTFESVETIQDYIIHPAHVGFGDVYRSFWEKLLIFDYTPRK

A nucleic acid sequence encoding wild-type CsOAC is provided by SEQ ID NO: 2:

(SEQ ID NO: 2)
atggcagtgaagcatttgattgtattgaagttcaaagatgaaatcacagaa
gcccaaaaggaagaatttttcaagacgtatgtgaatcttgtgaatatcatc
ccagccatgaaagatgtatactggggtaaagatgtgactcaaaagaataag
gaagaagggtacactcacatagttgaggtaacatttgagagtgtggagact
attcaggactacattattcatcctgcccatgttggatttggagatgtctat
cgttctttctgggaaaaacttctcatttttgactacacaccacgaaag

A non-limiting additional example of a nucleic acid sequence encoding wild-type CsOAC includes:

(SEQ ID NO: 3)
atggccgtcaagcatctgattgtattgaaatttaaggacgagatcacggaa
gcacaaaaggaagaatttttcaaaacttatgttaatttagttaacataatc
cctgctatgaaggatgtctactggggtaaggatgtgacacagaaaaacaag
gaagaaggctacactcacattgtggaagttaccttcgagagcgttgaaact
attcaagactacatcatccaccccgctcatgtcggattcggtgatgtctat
cgttccttttgggaaaaattgttgattttcgactataccccaagaaagtaa

Multiple structural domains have been identified in the CsOAC protein. See, e.g., Table 2, which is based on output from the DSSP program, Tou et al., Nucleic Acids Res. 2015 Jan.;43(Database issue):D364-8 and Kabsch and Sander Biopolymers. 1983 Dec.;22(12):2577-637. In some embodiments, a PKC comprises one or more (e.g., at least 1, at least 2, at least 3, at least 4, at least 5, at least 6, at least 7, at least 8, at least 9, at least 10, at least 11, at least 12, at least 13, at least 14, or at least 15) of the domains listed in Table 2. As one of ordinary skill in the art would appreciate, multiple different computational analysis programs may be used to determine secondary structures in proteins, such as the CsOAC protein. Different computational analysis programs may define the boundaries of the secondary structures differently. Non-limiting examples of secondary structure analysis of wild-type CsOAC (SEQ ID NO: 1) may be found in Yang et al., FEBS J. 2016 Mar.;283(6):1088-106; Gagne et al., Proc Natl Acad Sci USA. 2012 Jul. 31; 109(31):12811-6; and under UniProtKB Accession No. I6WU39. It should be appreciated that throughout this disclosure, reference to structural domains in CsOAC are based on the structural domains as described in Table 2.

TABLE 2
Structural domains in CsOAC

| | Corresponding |
|---|---|
| | positions in |
| Domain | SEQ ID NO: 1 |
| beta sheet 1 | 1-11 |
| loop 1 | 12-16 |
| helix 1 | 17-29 |
| 310 helix 1 | 30-32 |
| loop 2 | 33-38 |
| beta sheet 2 | 39-44 |
| loop 3 | 45-56 |
| beta sheet 3 | 57-62 |
| loop 4 | 63-65 |
| helix 2 | 66-73 |
| turn 1 | 74-75 |
| helix 3 | 76-85 |
| 310 helix 2 | 86-88 |
| beta sheet 4 | 89-97 |
| loop 5 | 98-101 |

Residues H5, 17, L9, F₂₃, F₂₄, Y27, V28, L30, V40, V59, Y72, 173, H₇₈, F₈₁, G82, W89, L92, and 194 form the active site of wild-type CsOAC (SEQ ID NO: 1), with residues Y72 and H₇₈ being catalytic residues. See, e.g., Yang et al. (FEBS J. 2016 March;283(6):1088-106). In some embodiments, a PKC comprises the amino acid Y at a residue corresponding to position 72 in SEQ ID NO: 1 and comprises the amino acid H at a residue corresponding to position 78 in SEQ ID NO: 1.

In some embodiments, a PKC comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 1-3, 19-106, 107-194, 196-388, 389-581, 582-669, 671-742, 745-948, 949-1036, 1038-1109, or 1112-1315 or a sequence in Table 11. In some embodiments, the reference sequence is SEQ ID NO: 62. In some embodiments, the reference sequence is SEQ ID NO: 87. In some embodiments, the reference sequence is SEQ ID NO: 150. In some embodiments, the reference sequence is SEQ ID NO: 175. In some embodiments, the reference sequence is SEQ ID NO: 220. In some embodiments, the reference sequence is SEQ ID NO: 255. In some embodiments, a PKC comprises a sequence that is at most 5%, at most 10%, at most 15%, at most 20%, at most 25%, at most 30%, at most 35%, at most 40%, at most 45%, at most 50%, at most 55%, at most 60%, at most 65%, at most 70%, at most 71%, at most 72%, at most 73%, at most 74%, at most 75%, at most 76%, at most 77%, at most 78%, at most 79%, at most 80%, at most 81%, at most 82%, at most 83%, at most 84%, at most 85%, at most 86%, at most 87%, at most 88%, at most 89%, at most 90%, at most 91%, at most 92%, at most 93%, at most 94%, at most 95%, at most 96%, at most 97%, at most 98%, at most 99%, or is 100% identical, including all values in between, to one or more of SEQ ID NO: 1-3, 19-106, 107-194, 196-388, 389-581, 582-669, 671-742, 745-948, 949-1036, 1038-1109, or 1112-1315 or a sequence in Table 11. In some embodiments, a PKC comprises a sequence that is 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, including all values in between, to one or more of SEQ ID NOs: 1-3, 19-106, 107-194, 196-388, 389-581, 582-669, 671-742, 745-948, 949-1036, 1038-1109, or 1112-1315 or a sequence in Table 11. In some embodiments, the reference sequence is SEQ ID NO: 62. In some embodiments, the reference sequence is SEQ ID NO: 87. In some embodiments, the reference sequence is SEQ ID NO: 150. In some embodiments, the reference sequence is SEQ ID NO: 175. In some embodiments, the reference sequence is SEQ ID NO: 220. In some embodiments, the reference sequence is SEQ ID NO: 255.

In some embodiments, the reference sequence is SEQ ID NO: 19, 21, 23, 24, 26, 27, 29, 32, 33, 34, 35, 38, 40, 42, 43, 45, 48, 49, 50, 51, 53, 58, 60, 62, 65, 66, 67, 68, 78, 87, 91, 92, 94, 99, 103, 105, 219, 220, 231, 234, 240, 254, 255, 258 283, 289, 294, 344, 345, 349, 365, 582-603, 605, 606, 607, 608, 610, 611, 612, 614, 616, 617, 618, 622, 625, 627, 629, 631, 632, 633, 634, 637, 638, 639, 640, 641, 642, 643, 644, 646, 648, 651, 653, 654, 655, 656, 657, 658, 659, 660, 661, 662, 663, 666, 667, 668, 672, 673, 674, 675, 676, 679, 680, 683, 684, 685, 686, 691, 693, 694, 695, 696, 697, 699, 700, 703, 707, 711, 713, 714, 715, 724, 732, 734, 735, 736, 737, 738, 739, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777. In some embodiments, the reference sequence is SEQ ID NO: 255. In some embodiments, the reference sequence is SEQ ID NO: 220. In some embodiments, the reference sequence is SEQ ID NO: 707. In some embodiments, the reference sequence is selected from the group consisting of SEQ ID NOs: 255, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

In some embodiments, the reference sequence is selected from any one of SEQ ID NOs: 107-194, 389-581, 949-1036, 1038-1109, and 1112-1315. In some embodiments, the reference sequence is selected from any one of SEQ ID NOs: 107, 109, 111, 112, 114, 115, 117, 120, 121, 122, 123, 126, 128, 130, 131, 133, 136, 137, 138, 139, 141, 146, 148, 150, 153, 154, 155, 156, 166, 175, 179, 180, 182, 187, 191, 193, 412, 413, 424, 427, 433, 447, 448, 451, 476, 482, 487, 537, 538, 542, 558, 949-970, 1074, 972, 973, 974, 975, 977, 978, 979, 981, 983, 984, 985, 989, 992, 994, 996, 998, 999, 1000, 1001, 1004, 1005, 1006, 1007, 1008, 1009, 1010, 1011, 1013, 1015, 1018, 1020, 1021, 1022, 1023, 1024, 1025, 1026, 1027, 1028, 1029, 1030, 1033, 1034, 1035, 1039, 1040, 1041, 1042, 1043, 1046, 1047, 1050, 1051, 1052, 1053, 1058, 1060, 1061, 1062, 1063, 1064, 1066, 1067, 1070, 1078, 1080, 1081, 1082, 1091, 1099, 1101, 1102, 1103, 1104, 1105, 1106, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144. In some embodiments, the reference sequence is selected from the group consisting of SEQ ID NOs: 448, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

In some embodiments, the reference sequence is selected from any one of SEQ ID NOs: 283, 289, 294, 344, 345, 349, and 365. In some embodiments, the reference sequence is selected from any one of SEQ ID NOs: 62, 87, 220, 234, 219, 258, 231, 240, and 255. In some embodiments, the reference sequence is selected from any one of SEQ ID NOs: 62, 87, 220, 234, 219, 258, 231, 240, 255, 707, 749, 751, 765, 768, 790, 808, 810, 822, 828, 840, 846, 857, 862, 865, 921, and 922.

In some embodiments, a PKC comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 101 amino acid substitutions, deletions, or insertions relative to SEQ ID NO: 1, 19-106, 196-388, 582-669, 671-742, or 745-948 or an amino acid sequence in Table 11.

In some embodiments, a PKC consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 101 amino acid substitutions, deletions, or insertions relative to SEQ ID NO: 1, 19-106, or 196-388, 582-669, 671-742, or 745-948 or an amino acid sequence in Table 11.

In some embodiments, a PKC comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, or at most 101 amino acid substitutions, deletions, and/or insertions relative to SEQ ID NO: 1, 19-106, 196-388, 582-669, 671-742, or 745-948 or an amino acid sequence in Table 11.

In some embodiments, a PKC comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, or 303 nucleic acid substitutions, deletions, and/or insertions relative to one or more of SEQ ID NOs: 2-3, 107-194, 389-581, 949-1036, 1038-1109 and 1112-1315 or a nucleic acid sequence in Table 11.

In some embodiments, a PKC consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, or 303 nucleic acid substitutions, deletions, and/or insertions relative to one or more of SEQ ID NOs: 2-3, 107-194, 389-581, 949-1036, 1038-1109 and 1112-1315 or a nucleic acid sequence in Table 11.

In some embodiments, a PKC comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, at most 250, at most 251, at most 252, at most 253, at most 254, at most 255, at most 256, at most 257, at most 258, at most 259, at most 260, at most 261, at most 262, at most 263, at most 264, at most 265, at most 266, at most 267, at most 268, at most 269, at most 270, at most 271, at most 272, at most 273, at most 274, at most 275, at most 276, at most 277, at most 278, at most 279, at most 280, at most 281, at most 282, at most 283, at most 284, at most 285, at most 286, at most 287, at most 288, at most 289, at most 290, at most 291, at most 292, at most 293, at most 294, at most 295, at most 296, at most 297, at most 298, at most 299, at most 300, at most 301, at most 302, at most 303 nucleic acid substitutions, deletions, and/or insertions relative to one or more of SEQ ID NOs: 2-3, 107-194, 389-581, 949-1036, 1038-1109, or 1112-1315 or a nucleic acid sequence in Table 11.

In some embodiments, a PKC comprises an amino acid substitution, deletion, or insertion at a residue corresponding to position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, and/or 101 in SEQ ID NO: 1. As a non-limiting example, position 83 in SEQ ID NO: 1 has the amino acid “D.” A PKC that comprises an amino acid substitution, deletion, or insertion at a residue corresponding to position 83 in SEQ ID NO: 1 would not comprise the amino acid “D” at the corresponding position. In some embodiments, a PKC comprises one or more amino acid substitutions or deletions selected from the amino acid substitutions or deletions provided in Tables 4-8, 9A-9B, and/or 10.

In some embodiments, a PKC comprises one or more amino acid substitutions at a residue corresponding to position 18, 29, 53, 70, 82, and/or 83 in SEQ ID NO: 1. In some embodiments, the PKC comprises the amino acid H at a residue corresponding to position 53 in SEQ ID NO: 1, the amino acid K at a residue corresponding to position 82 in SEQ ID NO: 1, and/or the amino acid L at a residue corresponding to position 70 in SEQ ID NO: 1. In some embodiments, a PKC comprises the amino acid substitution E53H, G82K, and/or Q70L relative to SEQ ID NO: 1.

In some embodiments, the amino acid substitution G82K may increase the abundance of a PKC (e.g., by at least 10%, 20%, 30%, 40%, or 50%) relative to its wild-type counterpart (e.g., SEQ ID NO: 1), while substantially maintaining olivetolic acid production activity relative to olivetolic acid production by its wild-type counterpart (e.g., at least 80%, 85%, 90%, or 95% of wild-type activity). It is unexpected that a PKC comprising the amino acid substitution G82K relative to SEQ ID NO: 1 would be capable of producing at least 80%, 85%, 90%, or 95% of the amount of olivetolic acid produced by its wild-type counterpart because the amino acid substitution G82K is a non-conservative amino acid substitution of a residue that is in the active site with close proximity to a bound substrate. Glycine includes hydrogen only in the “R” group, whereas lysine includes 4 carbons and a positively charged amine group. Without being bound by a particular theory, the amine group of lysine may interact with the hydroxyl or carboxyl groups on olivetolic acid. In some embodiments, without being bound by a particular theory, the effect of the G82K amino acid substitution alone may be minimal in terms of increasing olivetolic acid production of a PKC (e.g., SEQ ID NO: 1), but the G82K amino acid substitution may be advantageous in increasing olivetolic acid production of a PKC when used in combination with other amino acid substitutions or deletions. In some of those embodiments where the PKC comprises the amino acid substitution G82K relative to SEQ ID NO: 1, the PKC further comprises A18D, D83E or D83K, E22K, N29S, Q70L, S87K, and/or T26A.

In some embodiments, a PKC comprises an amino acid substitution or deletion at a residue corresponding to position 2, 18, 22, 24, 25, 26, 28, 29, 33, 52, 53, 57, 60, 62, 70, 82, 83, 84, 87, and/or 88 in SEQ ID NO: 1. In some embodiments, the PKC comprises an amino acid substitution at a residue corresponding to position 18, 29, 53, 70, 82 and/or 83 in SEQ ID NO:1.In some embodiments, a PKC comprises: a deletion at a residue corresponding to position 2 in SEQ ID NO: 1; the amino acid D or R at a residue corresponding to position 18 in SEQ ID NO: 1; the amino acid K at a residue corresponding to position 22 in SEQ ID NO: 1; the amino acid M at a residue corresponding to position 24 in SEQ ID NO: 1; the amino acid N at a residue corresponding to position 25 in SEQ ID NO: 1; the amino acid A or H at a residue corresponding to position 26 in SEQ ID NO: 1; the amino acid E at a residue corresponding to position 28 in SEQ ID NO: 1; the amino acid E or S at a residue corresponding to position 29 in SEQ ID NO: 1; the amino acid H or R at a residue corresponding to position 33 in SEQ ID NO: 1; the amino acid H at a residue corresponding to position 52 in SEQ ID NO: 1; the amino acid H at a residue corresponding to position 53 in SEQ ID NO: 1; the amino acid Y at a residue corresponding to position 57 in SEQ ID NO: 1; the amino acid I at a residue corresponding to position 60 in SEQ ID NO: 1; the amino acid M at a residue corresponding to position 62 in SEQ ID NO: 1; the amino acid L at a residue corresponding to position 70 in SEQ ID NO: 1; the amino acid K at a residue corresponding to position 82 in SEQ ID NO: 1; the amino acid A, K, or R at a residue corresponding to position 83 in SEQ ID NO: 1; the amino acid F or K at a residue corresponding to position 84 in SEQ ID NO: 1; the amino acid C or K at a residue corresponding to position 87 in SEQ ID NO: 1; and/or the amino acid Y at a residue corresponding to position 88 in SEQ ID NO: 1. In some embodiments, a PKC comprises a combination of amino acid substitutions or deletions relative to SEQ ID NO: 1 that is in Table 4. In some embodiments, relative to SEQ ID NO: 1, the PKC comprises: E53H, G82K, and Q70L. As a non-limiting example, relative to SEQ ID NO: 1, a PKC may comprise A18D, T26A, V28E, N29S, E53H, E601, Q70L, G82K, D83A, and S87K; A18D, E22K, N29S, E53H, Q70L, G82K, D83R, S87K, and F₈₈Y; A18D, E22K, N29S, E53H, Q70L, G82K, D83K, S87K, and F₈₈Y; A18D, E22K, N29S, I33R, E53H, Q70L, G82K, D83A, S87K, and F₈₈Y; A18D, E22K, K25N, T26A, N29S, E53H, Q70L, G82K, D83A, and S87K; A18D, E22K, N29S, E53H, Q70L, G82K, D83A, V84K, S87K, and F₈₈Y; A18D, E22K, T26A, N29S, E53H, Q70L, G82K, D83A, S87K, and F₈₈Y; A18D, E22K, T26H, N29S, E53H, Q70L, G82K, D83A, S87K, and F₈₈Y; A18D, E22K, N29S, I33H, E53H, Q70L, G82K, D83A, S87K, and F₈₈Y; A18D, E22K, N29S, E53H, Q70L, G82K, D83A, V84F, S87K, and F₈₈Y; A18D, T26A, N29S, E53H, Q70L, G82K, D83A, and F₈₈Y; A18D, E22K, T26A, N29S, E53H, Q70L, G82K, D83A, and S87K; A18D, T26A, N29S, E53H, Q70L, G82K, D83A, S87K, and F₈₈Y; A18D, T26A, N29S, E53H, Q70L, G82K, and D83A; A18D, E22K, T26A, N29S, E53H, Q70L, G82K, D83A, and F₈₈Y; A18D, E22K, N29S, E53H, Q70L, G82K, D83A, S87K, and F₈₈Y; A18D, T26A, V28E, N29S, E53H, Q70L, G82K, D83A, and S87K; A18R, E22K, N29S, E53H, Q70L, G82K, D83A, S87K, and F₈₈Y; deletion of a residue corresponding to position 2 in SEQ ID NO: 1, A18D, E22K, N29S, E53H, Q70L, G82K, D83A, S87K, and F₈₈Y; A18D, E22K, N29S, E53H, T62M, Q70L, G82K, D83A, S87K, and F₈₈Y; A18D, E22K, T26A, N29E, E53H, Q70L, G82K, D83A, and S87K; A18D, E22K, N29S, E52H, E53H, Q70L, G82K, D83A, S87K, and F₈₈Y; A18D, E22K, N29S, E53H, Q70L, G82K, D83A, S87C, and F₈₈Y; A18D, E22K, F₂₄M, N29S, E53H, Q70L, G82K, D83A, S87K, and F₈₈Y; A18D, N29S, E53H, Q70L, G82K, D83A, and F₈₈Y; A18D, N29S, E53H, Q70L, G82K, D83A, S87K, and F₈₈Y; or A18D, E22K, T26A, N29S, E53H, H₅₇Y, Q70L, G82K, D83A, and S87K. In some embodiments, a PKC comprises A18D, E22K, T26A, N29S, E53H, H₅₇Y, Q70L, G82K, D83A, and S87K. In some embodiments, a PKC comprises A18D, E22K, K25N, T26A, N29S, E53H, Q70L, G82K, D83A, and S87K.

In some embodiments, the PKC comprises: A18D, T26A, V28E, N29S, E53H, E601, Q70L, G82K, D83A, and S87K; A18D, E22K, N29S, E53H, Q70L, G82K, D83R, S87K, and F₈₈Y; A18D, E22K, N29S, E53H, Q70L, G82K, D83K, S87K, and F₈₈Y; A18D, E22K, N29S, I33R, E53H, Q70L, G82K, D83A, S87K, and F₈₈Y; A18D, E22K, K25N, T26A, N29S, E53H, Q70L, G82K, D83A, and S87K; A18D, E22K, N29S, E53H, Q70L, G82K, D83A, V84K, S87K, and F₈₈Y; A18D, E22K, T26A, N29S, E53H, Q70L, G82K, D83A, S87K, and F₈₈Y; A18D, E22K, T26H, N29S, E53H, Q70L, G82K, D83A, S87K, and F₈₈Y; A18D, E22K, N29S, I33H, E53H, Q70L, G82K, D83A, S87K, and F₈₈Y; A18D, E22K, N29S, E53H, Q70L, G82K, D83A, V84F, S87K, and F₈₈Y; A18D, T26A, N29S, E53H, Q70L, G82K, D83A, and F₈₈Y; A18D, E22K, T26A, N29S, E53H, Q70L, G82K, D83A, and S87K; A18D, T26A, N29S, E53H, Q70L, G82K, D83A, S87K, and F₈₈Y; A18D, T26A, N29S, E53H, Q70L, G82K, and D83A; A18D, E22K, T26A, N29S, E53H, Q70L, G82K, D83A, and F₈₈Y; A18D, E22K, N29S, E53H, Q70L, G82K, D83A, S87K, and F₈₈Y; A18D, T26A, V28E, N29S, E53H, Q70L, G82K, D83A, and S87K; A18R, E22K, N29S, E53H, Q70L, G82K, D83A, S87K, and F₈₈Y; deletion of a residue corresponding to position 2 in SEQ ID NO: 1, A18D, E22K, N29S, E53H, Q70L, G82K, D83A, S87K, and F₈₈Y; A18D, E22K, N29S, E53H, T62M, Q70L, G82K, D83A, S87K, and F₈₈Y; A18D, E22K, T26A, N29E, E53H, Q70L, G82K, D83A, and S87K; A18D, E22K, N29S, E52H, E53H, Q70L, G82K, D83A, S87K, and F₈₈Y; A18D, E22K, N29S, E53H, Q70L, G82K, D83A, S87C, and F₈₈Y; A18D, E22K, F₂₄M, N29S, E53H, Q70L, G82K, D83A, S87K, and F₈₈Y; A18D, N29S, E53H, Q70L, G82K, D83A, and F₈₈Y; A18D, N29S, E53H, Q70L, G82K, D83A, S87K, and F₈₈Y; A18D, E22K, T26A, N29S, E53H, H₅₇Y, Q70L, G82K, D83A, and S87K; I33R, E52H, Y55F, Q70A, G80A, D83K, and V84K; D13R, A18D, N29S, Q70L, D71Q, G80A, and D83K; I33R, E52H, Y55F, Q70A, I74G, and D83K; T26R, I33H, E52H, Y55F, Q70A, and D83K; A18D, T26R, E52H, Y55F, Q70A, G80A, D83N, V84K, and Y85A; T26R, I33H, Y55F, Q70A, G80A, D83K, and Y85A; E22K, T26H, N29S, D39G, K49Q, E52H, D83N, and V84K; E17N, A18D, V31L, D71S, D83A, and V84F; or A18D, V31L, D39Q, D71S, D83A, and V84F.

In some embodiments, the PKC comprises an amino acid substitution at a residue corresponding to position 17, 18, 26, 29, 31, 33, 37, 39, 71, 80, 83, 84, and/or 95 in SEQ ID NO: 1. In some embodiments, the PKC comprises: the amino acid N at a residue corresponding to position 17 in SEQ ID NO: 1; the amino acid D at a residue corresponding to position 18 in SEQ ID NO: 1; the amino acid A or H at a residue corresponding to position 26 in SEQ ID NO: 1; the amino acid T at a residue corresponding to position 29 in SEQ ID NO: 1; the amino acid L at a residue corresponding to position 31 in SEQ ID NO: 1; the amino acid R, H, or N at a residue corresponding to position 33 in SEQ ID NO: 1; the amino acid L at a residue corresponding to position 37 in SEQ ID NO: 1; the amino acid Q or G at a residue corresponding to position 39 in SEQ ID NO: 1; the amino acid S at a residue corresponding to position 71 in SEQ ID NO: 1; the amino acid A at a residue corresponding to position 80 in SEQ ID NO: 1; the amino acid Q, N, or A at a residue corresponding to position 83 in SEQ ID NO: 1; the amino acid F at a residue corresponding to position 84 in SEQ ID NO: 1; and/or the amino acid M at a residue corresponding to position 95 in SEQ ID NO: 1. In some embodiments, relative to SEQ ID NO: 1, the PKC comprises: E17N, A18D, and D39G; D39Q, and F₉₅M; I33N and F₉₅M; E17N, I33R, and F₉₅M; T26H, D83Q, and F₉₅M; I33H, D39Q, and D71S; N29T, I33N, and D71S; I33N, D39Q, and F₉₅M; D71S, D83N, and F₉₅M; D39Q and D83Q; A18D and M37L; E17N, A18D, and I33N; D39G, D71S, and V84F; N29T and D39G; A18D, D71S, and G80A; N29T, V31L, and D39Q; A18D, V31L, and D39G; T26H, N29T, and D39Q; T26A, N29T, and I33H; D39Q, D71S, and G80A; A18D, D39Q, and V84F; E17N, I33R, and D39Q; E17N, D71S, and F₉₅M; E17N, I33N, and F₉₅M; T26A, N29T, and V31L; E17N, T26A, and D39G; A18D, D39Q, and D71S; V31L, D39G, and V84F; N29T, I33R, and D39Q; D39G, D71S, and D83N; V31L, D71S, and D83Q; 133R, D39Q, and V84F; D39G and D71S; E17N, I33H, and D71S; T26H, D39G, and V84F; A18D, 133R, and D39G; T26A, D83N, and F₉₅M; V31L, I33H, and D71S; V31L, D39G, and D71S; A18D and D39Q; I33R, D39G, and D71S; E17N, G80A, and D83N; A18D, N29T, and V31L; D39Q, D71S, and D83A; A18D, T26H, and D39Q; T26A and F₉₅M; I33H, D39G, and D83Q; T26H, 133N, and G80A; I33R, D39G, and F₉₅M; V31L, I33R, and D83N; N29T, D39G, and V84F; D71S and D83Q; I33H, D39Q, and G80A; E17N, D39G, and D83A; E17N, 133N, and V84F; N29T, I33N, and D83Q; I33N, D71S, and G80A; I33R, D39G, and D83N; T26A and N29T; I33H, D83Q, and F₉₅M; T26A, V31L, and D39Q; E17N, V31L, and I33N; E17N, T26A, and M37L; or N29T and I33N.

In some embodiments, the PKC comprises an amino acid substitution at a residue corresponding to position 13, 18, 22, 26, 29, 33, 39, 49, 52, 55, 61, 64, 65, 66, 68, 70, 71, 74, 80, 83, 84, 85, and/or 98 in SEQ ID NO: 1. In some embodiments, the PKC comprises: the amino acid R at a residue corresponding to position 13 in SEQ ID NO: 1; the amino acid D at a residue corresponding to position 18 in SEQ ID NO: 1; the amino acid K at a residue corresponding to position 22 in SEQ ID NO: 1; the amino acid R or H at a residue corresponding to position 26 in SEQ ID NO: 1; the amino acid S at a residue corresponding to position 29 in SEQ ID NO: 1; the amino acid R or H at a residue corresponding to position 33 in SEQ ID NO: 1; the amino acid G at a residue corresponding to position 39 in SEQ ID NO: 1; the amino acid Q at a residue corresponding to position 49 in SEQ ID NO: 1; the amino acid H at a residue corresponding to position 52 in SEQ ID NO: 1; the amino acid F at a residue corresponding to position 55 in SEQ ID NO: 1; the amino acid D at a residue corresponding to position 61 in SEQ ID NO: 1; the amino acid D at a residue corresponding to position 64 in SEQ ID NO: 1; the amino acid N at a residue corresponding to position 65 in SEQ ID NO: 1; the amino acid Y at a residue corresponding to position 66 in SEQ ID NO: 1; the amino acid G at a residue corresponding to position 68 in SEQ ID NO: 1; the amino acid A or L at a residue corresponding to position 70 in SEQ ID NO: 1; the amino acid E or Q at a residue corresponding to position 71 in SEQ ID NO: 1; the amino acid G at a residue corresponding to position 74 in SEQ ID NO: 1; the amino acid N or A at a residue corresponding to position 80 in SEQ ID NO: 1; the amino acid K or N at a residue corresponding to position 83 in SEQ ID NO: 1; the amino acid K at a residue corresponding to position 84 in SEQ ID NO: 1; the amino acid F or A at a residue corresponding to position 85 in SEQ ID NO: 1; and/or the amino acid E or K at a residue corresponding to position 98 in SEQ ID NO: 1. In some embodiments, relative to SEQ ID NO: 1, the PKC comprises: Y85F; E64D, D71E, and T98K; S65N; V66Y; G80N; T68G; D71E; E64D and D71E; D71E and Y85F; I33R, E52H, Y55F, Q70A, G80A, D83K, and V84K; D13R, A18D, N29S, Q70L, D71Q, G80A, and D83K; I33R, E52H, Y55F, Q70A, I74G, and D83K; Y55F, G80A, and D83K; T26R, I33H, E52H, Y55F, Q70A, and D83K; A18D, T26R, E52H, Y55F, Q70A, G80A, D83N, V84K, and Y85A; T98E; T26R, I33H, Y55F, Q70A, G80A, D83K, and Y85A; V61D; or E22K, T26H, N29S, D39G, K49Q, E52H, D83N, and V84K.

In some embodiments, the PKC comprises an amino acid substitution at a residue corresponding to position 13, 17, 18, 22, 26, 29, 31, 33, 39, 52, 55, 61, 64, 65, 66, 70, 71, 74, 80, 82, 83, 84, 85, 88, 95, and/or 98 in SEQ ID NO: 1. In some embodiments, the PKC comprises: the amino acid R at a residue corresponding to position 13 in SEQ ID NO: 1; the amino acid N at a residue corresponding to position 17 in SEQ ID NO: 1; the amino acid D at a residue corresponding to position 18 in SEQ ID NO: 1; the amino acid K at a residue corresponding to position 22 in SEQ ID NO: 1; the amino acid R at a residue corresponding to position 26 in SEQ ID NO: 1; the amino acid S at a residue corresponding to position 29 in SEQ ID NO: 1; the amino acid L at a residue corresponding to position 31 in SEQ ID NO: 1; the amino acid H, N, or R at a residue corresponding to position 33 in SEQ ID NO: 1; the amino acid G or Q at a residue corresponding to position 39 in SEQ ID NO: 1; the amino acid H at a residue corresponding to position 52 in SEQ ID NO: 1; the amino acid F at a residue corresponding to position 55 in SEQ ID NO: 1; the amino acid S at a residue corresponding to position 61 in SEQ ID NO: 1; the amino acid D at a residue corresponding to position 64 in SEQ ID NO: 1; the amino acid N at a residue corresponding to position 65 in SEQ ID NO: 1; the amino acid Y at a residue corresponding to position 66 in SEQ ID NO: 1; the amino acid A or L at a residue corresponding to position 70 in SEQ ID NO: 1; the amino acid E, Q, or S at a residue corresponding to position 71 in SEQ ID NO: 1; the amino acid G at a residue corresponding to position 74 in SEQ ID NO: 1; the amino acid A at a residue corresponding to position 80 in SEQ ID NO: 1; the amino acid R at a residue corresponding to position 82 in SEQ ID NO: 1; the amino acid A, E, K, or N at a residue corresponding to position 83 in SEQ ID NO: 1; the amino acid F or K at a residue corresponding to position 84 in SEQ ID NO: 1; the amino acid A or F at a residue corresponding to position 85 in SEQ ID NO: 1; the amino acid N at a residue corresponding to position 88 in SEQ ID NO: 1; the amino acid M at a residue corresponding to position 95 in SEQ ID NO: 1; and/or the amino acid K or Y at a residue corresponding to position 98 in SEQ ID NO: 1.

In some embodiments, the PKC comprises an amino acid substitution at a residue corresponding to position 13, 17, 18, 22, 26, 31, 33, 39, 52, 55, 64, 65, 66, 70, 71, 74, 80, 82, 83, 84, 85, 88, and/or 98 in SEQ ID NO: 1. In some embodiments, the PKC comprises: the amino acid R at a residue corresponding to position 13 in SEQ ID NO:1; the amino acid N at a residue corresponding to position 17 in SEQ ID NO:1; the amino acid D at a residue corresponding to position 18 in SEQ ID NO:1; the amino acid K at a residue corresponding to position 22 in SEQ ID NO:1; the amino acid R at a residue corresponding to position 26 in SEQ ID NO:1; the amino acid L at a residue corresponding to position 31 in SEQ ID NO:1; the amino acid R, H, or N at a residue corresponding to position 33 in SEQ ID NO:1; the amino acid G at a residue corresponding to position 39 in SEQ ID NO:1; the amino acid H at a residue corresponding to position 52 in SEQ ID NO:1; the amino acid F at a residue corresponding to position 55 in SEQ ID NO:1; the amino acid D at a residue corresponding to position 64 in SEQ ID NO:1; the amino acid N at a residue corresponding to position 65 in SEQ ID NO:1; the amino acid Y at a residue corresponding to position 66 in SEQ ID NO:1; the amino acid A at a residue corresponding to position 70 in SEQ ID NO:1; the amino acid S or E at a residue corresponding to position 71 in SEQ ID NO:1; the amino acid R at a residue corresponding to position 82 in SEQ ID NO: 1; the amino acid G at a residue corresponding to position 74 in SEQ ID NO:1; the amino acid A at a residue corresponding to position 80 in SEQ ID NO:1; the amino acid E, A, K, or N at a residue corresponding to position 83 in SEQ ID NO:1; the amino acid K or F at a residue corresponding to position 84 in SEQ ID NO:1; the amino acid F at a residue corresponding to position 85 in SEQ ID NO:1; the amino acid N at a residue corresponding to position 88 in SEQ ID NO: 1; and/or the amino acid K or Y at a residue corresponding to position 98 in SEQ ID NO:1. In some embodiments, relative to SEQ ID NO: 1, the PKC comprises: A18D, T26R, I33R, D39G, E52H, Y55F, E64D, Q70A, G80A, D83N, and V84K; E17N, T26R, V31L, D71S, D83E, and V84F; E17N, V31L, I33N, E64D, D71S, and V84F; E17N, A18D, V31L, D71S, D83A, and V84F; E17N, E22K, V31L, I33N, D71E, and V84F; E17N, A18D, V31L, D71E, D83A, and V84F; E17N, V31L, D71S, D83E, V84F, and T98Y; V84K, T26R, D39G, D83E, T98K, and S65N; E17N, A18D, V31L, I33N, D71S, D83E, and V84K; E17N, A18D, V31L, D71S, I74G, D83E, and V84F; E17N, V31L, I33N, D71S, V84K, and Y85F; A18D, T26R, I33R, E52H, Y55F, V66Y, Q70A, G80A, D83N, V84K, and Y85F; E17N, A18D, V31L, I33N, D71S, D83K, and V84K; E17N, A18D, T26R, V31L, I33N, D71S, and V84K; V84K, T26R, D39G, D13R, T98K, and S65N; E17N, V31L, I33N, S65N, D71S, and V84K; E17N, A18D, E22K, V31L, I33N, D71E, and V84F; D13R, E17N, A18D, V31L, D71S, D83A, and V84K; E17N, V31L, D39G, D71S, D83A, and V84K; D13R, A18D, T26R, E52H, Y55F, Q70A, G80A, D83N, V84K, and Y85F; E17N, A18D, T26R, V31L, D71S, D83A, and V84K; E17N, V31L, I33N, D71S, G82R, and V84F; E17N, V31L, I33N, D71S, V84K, and T98K; V84K, T26R, D39G, D83E, D13R, and T98K; V84K, T26R, D83E, D13R, T98K, and S65N; E17N, V31L, I33N, V66Y, D71E, and V84F; E17N, A18D, V31L, I33N, D71E, and V84F; E17N, A18D, V31L, I33N, D71S, G80A, and V84F; A18D, T26R, E52H, Y55F, Q70A, G80A, D83N, V84K, Y85F, and F₈₈N; E17N, A18D, V31L, I33N, D71S, V84K, and T98K; E17N, A18D, V31L, I33N, S65N, D71S, and V84K; T26R, I33H, Y55F, Q70A, G80A, D83K, V84K, and F₈₈N; A18D, E22K, T26R, E52H, Y55F, Q70A, D71E, G80A, D83N, and V84K; or E17N, V31L, I33N, D71E, G82R, and V84F.

In some embodiments, a PKC comprises an amino acid substitution at one or more residues corresponding to one or more positions selected from the group consisting of position 13, 17, 18, 22, 26, 31, 33, 39, 52, 55, 64, 65, 66, 70, 71, 74, 80, 82, 83, 84, 85, 88, and/or 98 in SEQ ID NO: 1. In some embodiments, a PKC comprising a sequence with at least one amino acid substitution relative to SEQ ID NO: 1 and with at least 90% identity to SEQ ID NO: 62, 87, 220, 234, 219, 258, 231, 240, 255, 707, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, or 777.

In some embodiments, one or more amino acid substitutions relative to SEQ ID NO: 1 are selected from the group consisting of: A2G; V3M or V3Y; D13R or D13K; E17N; A18D or A18R; Q19E or Q19D; K20A; E21K; E22T or E22K; K25A or K25N; T26H, T26R, T26A, or T26E; V28A, V28S, V28E, or V28H; N29D, N29S, N29E, or N29T; V31L or V31A; I33H, I33R, I33D, 133E, I33K, I33Q, or 133N; M37L; K38L; D39G, D39S, or D39Q; Y41K or Y41R; D45I; N50K; E52H; E53H; Y55F; T56Y; H₅₇Y; E601 or E60L; T62M; E64Q or E64D; S65N; E67K; T68E, T68G, or T68H; Q70A or Q70L; D71Q or D71S; I74D, 174E, I74H, or I74Q; D83A or D83G; V84F, V84K, V84M, or V84Q; Y85A or Y85F; R86G; S87C, S87D, or S87K; F₈₈Y, F₈₈N, or F₈₈S; F₉₅M or F₉₅I; Y97F; T98E, T98K, or T98Y; R100A, R100G, R100K, R100N, R100Q, or R100S; and K101A or K101E.

In some embodiments, a PKC comprises one or more of the following amino acid substitutions relative to SEQ ID NO: 1: F₂₄M; V46F; Q48A; K49Q; N50F or N50W; V59I or V59F; V61T, V61D, or V61S; V66L, V66M, or V66Y; I69L or I69Y; D71E; 173L; I74S, I74G, or I74L; G80V, G80A, G80E, G80S, G80D, or G80N; F₈₁Y; G82K, G82V, or G82R; D83E, D83K, D83N, D83Q, D83R, or D83S; R86N or R86S; W89V or W89M; and/or I94V.

In some embodiments, a PKC comprises an amino acid deletion or substitution at a residue corresponding to position 2, 3, 13, 17, 18, 19, 20, 21, 22, 24, 25, 26, 28, 29, 31, 33, 37, 38, 39, 41, 45, 46, 48, 49, 50, 52, 53, 55, 56, 57, 59, 60, 61, 62, 64, 65, 66, 67, 68, 69, 70, 71, 73, 74, 80, 81, 82, 83, 84, 85, 86, 87, 88, 89, 94, 95, 97, 98, 100, and/or 101 in SEQ ID NO: 1. In some embodiments, a PKC comprises one, two, three, four, five, six, seven or more amino acid substitutions from Table 10. Without being bound by a particular theory, an amino acid deletion or substitution at a residue corresponding to position 2, 3, 13, 17, 18, 19, 20, 21, 22, 24, 25, 26, 28, 29, 31, 33, 37, 38, 39, 41, 45, 46, 48, 49, 50, 52, 53, 55, 56, 57, 59, 60, 61, 62, 64, 65, 66, 67, 68, 69, 70, 71, 73, 74, 80, 81, 82, 83, 84, 85, 86, 87, 88, 89, 94, 95, 97, 98, 100, and/or 101 in SEQ ID NO: 1 may increase protein abundance of the PKC and/or increase production of olivetolic acid by the PKC relative to a wild-type counterpart. In some embodiments, a PKC comprises: the amino acid G at a residue corresponding to position 2 in SEQ ID NO: 1 or a deletion of a residue corresponding to position 2 in SEQ ID NO: 1; the amino acid M or Y at a residue corresponding to position 3 in SEQ ID NO: 1; the amino acid R or K at a residue corresponding to position 13 in SEQ ID NO: 1; the amino acid N at a residue corresponding to position 17 in SEQ ID NO: 1; the amino acid D or R at a residue corresponding to position 18 in SEQ ID NO: 1; the amino acid E or D at a residue corresponding to position 19 in SEQ ID NO: 1; the amino acid A at a residue corresponding to position 20 in SEQ ID NO: 1; the amino acid K at a residue corresponding to position 21 in SEQ ID NO: 1; the amino acid T or K at a residue corresponding to position 22 in SEQ ID NO: 1; the amino acid M at a residue corresponding to position 24 in SEQ ID NO: 1; the amino acid A or N at a residue corresponding to position 25 in SEQ ID NO: 1; the amino acid H, R, A, or E at a residue corresponding to position 26 in SEQ ID NO: 1; the amino acid A, S, E, or H at a residue corresponding to position 28 in SEQ ID NO: 1; the amino acid D, S, E, or T at a residue corresponding to position 29 in SEQ ID NO: 1; the amino acid L or A at a residue corresponding to position 31 in SEQ ID NO: 1; the amino acid H, R, D, E, K, Q, or N at a residue corresponding to position 33 in SEQ ID NO: 1; the amino acid L at a residue corresponding to position 37 in SEQ ID NO: 1; the amino acid L at a residue corresponding to position 38 in SEQ ID NO: 1; the amino acid G, S, or Q at a residue corresponding to position 39 in SEQ ID NO: 1; the amino acid K or R at a residue corresponding to position 41 in SEQ ID NO: 1; the amino acid I at a residue corresponding to position 45 in SEQ ID NO: 1; the amino acid F at a residue corresponding to position 46 in SEQ ID NO: 1; the amino acid A at a residue corresponding to position 48 in SEQ ID NO: 1; the amino acid Q at a residue corresponding to position 49 in SEQ ID NO: 1; the amino acid K, F, or W at a residue corresponding to position 50 in SEQ ID NO: 1; the amino acid H at a residue corresponding to position 52 in SEQ ID NO: 1; the amino acid H at a residue corresponding to position 53 in SEQ ID NO: 1; the amino acid F at a residue corresponding to position 55 in SEQ ID NO: 1; the amino acid Y at a residue corresponding to position 56 in SEQ ID NO: 1; the amino acid Y at a residue corresponding to position 57 in SEQ ID NO: 1; the amino acid I or F at a residue corresponding to position 59 in SEQ ID NO: 1; the amino acid I or L at a residue corresponding to position 60 in SEQ ID NO: 1; the amino acid T, D, or S at a residue corresponding to position 61 in SEQ ID NO: 1; the amino acid M at a residue corresponding to position 62 in SEQ ID NO: 1; the amino acid Q or D at a residue corresponding to position 64 in SEQ ID NO: 1; the amino acid N at a residue corresponding to position 65 in SEQ ID NO: 1; the amino acid L, M, or Y at a residue corresponding to position 66 in SEQ ID NO: 1; the amino acid K at a residue corresponding to position 67 in SEQ ID NO: 1; the amino acid E, G, or H at a residue corresponding to position 68 in SEQ ID NO: 1; the amino acid L or Y at a residue corresponding to position 69 in SEQ ID NO: 1; the amino acid A or L at a residue corresponding to position 70 in SEQ ID NO: 1; the amino acid Q, S, or E at a residue corresponding to position 71 in SEQ ID NO: 1; the amino acid L at a residue corresponding to position 73 in SEQ ID NO: 1; the amino acid D, E, H, Q, S, G, or L at a residue corresponding to position 74 in SEQ ID NO: 1; the amino acid V, A, E, S, D, or N at a residue corresponding to position 80 in SEQ ID NO: 1; the amino acid Y at a residue corresponding to position 81 in SEQ ID NO: 1; the amino acid K, V, or R at a residue corresponding to position 82 in SEQ ID NO: 1; the amino acid A, E, G, K, N, Q, R, or S at a residue corresponding to position 83 in SEQ ID NO: 1; the amino acid F, K, M, or Q at a residue corresponding to position 84 in SEQ ID NO: 1; the amino acid A or F at a residue corresponding to position 85 in SEQ ID NO: 1; the amino acid N, G, or S at a residue corresponding to position 86 in SEQ ID NO: 1; the amino acid C, D, or K at a residue corresponding to position 87 in SEQ ID NO: 1; the amino acid Y, N, or S at a residue corresponding to position 88 in SEQ ID NO: 1; the amino acid V or M at a residue corresponding to position 89 in SEQ ID NO: 1; the amino acid V at a residue corresponding to position 94 in SEQ ID NO: 1; the amino acid M or I at a residue corresponding to position 95 in SEQ ID NO: 1; the amino acid F at a residue corresponding to position 97 in SEQ ID NO: 1; the amino acid E, K, or Y at a residue corresponding to position 98 in SEQ ID NO: 1; the amino acid A, G, K, N, Q, or S at a residue corresponding to position 100 in SEQ ID NO: 1; and/or the amino acid A or E at a residue corresponding to position 101 in SEQ ID NO: 1.

In some embodiments, the PKC comprises an amino acid deletion or substitution at one or more residues corresponding to position 2, 3, 13, 17, 18, 19, 20, 21, 22, 24, 25, 26, 28, 29, 31, 33, 37, 38, 39, 45, 46, 48, 49, 50, 52, 53, 55, 56, 57, 59, 60, 61, 62, 64, 65, 66, 67, 68, 70, 71, 74, 80, 82, 83, 84, 85, 86, 87, 88, 89, 94, 95, 97, 98, 100, and 101 in SEQ ID NO: 1. In some embodiments, the PKC comprises: the amino acid G at a residue corresponding to position 2 in SEQ ID NO: 1 or a deletion of a residue corresponding to position 2 in SEQ ID NO: 1; the amino acid Y at a residue corresponding to position 3 in SEQ ID NO: 1; the amino acid R or K at a residue corresponding to position 13 in SEQ ID NO: 1; the amino acid N at a residue corresponding to position 17 in SEQ ID NO: 1; the amino acid D or R at a residue corresponding to position 18 in SEQ ID NO: 1; the amino acid D at a residue corresponding to position 19 in SEQ ID NO: 1; the amino acid A at a residue corresponding to position 20 in SEQ ID NO: 1; the amino acid K at a residue corresponding to position 21 in SEQ ID NO: 1; the amino acid K at a residue corresponding to position 22 in SEQ ID NO: 1; the amino acid M at a residue corresponding to position 24 in SEQ ID NO: 1; the amino acid A or N at a residue corresponding to position 25 in SEQ ID NO: 1; the amino acid H, R, or A at a residue corresponding to position 26 in SEQ ID NO: 1; the amino acid E or H at a residue corresponding to position 28 in SEQ ID NO: 1; the amino acid S, E, or T at a residue corresponding to position 29 in SEQ ID NO: 1; the amino acid L at a residue corresponding to position 31 in SEQ ID NO: 1; the amino acid H, R, N at a residue corresponding to position 33 in SEQ ID NO: 1; the amino acid L at a residue corresponding to position 37 in SEQ ID NO: 1; the amino acid L at a residue corresponding to position 38 in SEQ ID NO: 1; the amino acid G or Q at a residue corresponding to position 39 in SEQ ID NO: 1; the amino acid I at a residue corresponding to position 45 in SEQ ID NO: 1; the amino acid F at a residue corresponding to position 46 in SEQ ID NO: 1; the amino acid A at a residue corresponding to position 48 in SEQ ID NO: 1; the amino acid Q at a residue corresponding to position 49 in SEQ ID NO: 1; the amino acid F, W, or K at a residue corresponding to position 50 in SEQ ID NO: 1; the amino acid H at a residue corresponding to position 52 in SEQ ID NO: 1; the amino acid H at a residue corresponding to position 53 in SEQ ID NO: 1; the amino acid F at a residue corresponding to position 55 in SEQ ID NO: 1; the amino acid Y at a residue corresponding to position 56 in SEQ ID NO: 1; the amino acid Y at a residue corresponding to position 57 in SEQ ID NO: 1; the amino acid F at a residue corresponding to position 59 in SEQ ID NO: 1; the amino acid I or L at a residue corresponding to position 60 in SEQ ID NO: 1; the amino acid D, S, or T at a residue corresponding to position 61 in SEQ ID NO: 1; the amino acid M at a residue corresponding to position 62 in SEQ ID NO: 1; the amino acid D at a residue corresponding to position 64 in SEQ ID NO: 1; the amino acid N at a residue corresponding to position 65 in SEQ ID NO: 1; the amino acid Y at a residue corresponding to position 66 in SEQ ID NO: 1; the amino acid K at a residue corresponding to position 67 in SEQ ID NO: 1; the amino acid G or H at a residue corresponding to position 68 in SEQ ID NO: 1; the amino acid A or L at a residue corresponding to position 70 in SEQ ID NO: 1; the amino acid Q, S, or E at a residue corresponding to position 71 in SEQ ID NO: 1; the amino acid G at a residue corresponding to position 74 in SEQ ID NO: 1; the amino acid V, A, D, or N at a residue corresponding to position 80 in SEQ ID NO: 1; the amino acid K or R at a residue corresponding to position 82 in SEQ ID NO: 1; the amino acid A, E, G, K, N, Q, R, or S at a residue corresponding to position 83 in SEQ ID NO: 1; the amino acid F or K at a residue corresponding to position 84 in SEQ ID NO: 1; the amino acid A or F at a residue corresponding to position 85 in SEQ ID NO: 1; the amino acid S at a residue corresponding to position 86 in SEQ ID NO: 1; the amino acid C or K at a residue corresponding to position 87 in SEQ ID NO: 1; the amino acid Y, N, or S at a residue corresponding to position 88 in SEQ ID NO: 1; the amino acid M at a residue corresponding to position 89 in SEQ ID NO: 1; the amino acid V at a residue corresponding to position 94 in SEQ ID NO: 1; the amino acid M or I at a residue corresponding to position 95 in SEQ ID NO: 1; the amino acid F at a residue corresponding to position 97 in SEQ ID NO: 1; the amino acid E, K, or Y at a residue corresponding to position 98 in SEQ ID NO: 1; the amino acid A, G, K, N, Q, or S at a residue corresponding to position 100 in SEQ ID NO: 1; and/or the amino acid A or E at a residue corresponding to position 101 in SEQ ID NO: 1.

In some embodiments, the PKC comprises an amino acid substitution at a residue corresponding to position 2, 3, 13, 17, 18, 19, 20, 21, 22, 25, 26, 28, 29, 31, 33, 37, 38, 39, 41, 45, 50, 52, 53, 55, 56, 57, 60, 62, 64, 65, 67, 68, 70, 71, 74, 83, 84, 85, 86, 87, 88, 95, 97, 98, 100, and/or 101 in SEQ ID NO: 1 wherein the PKC is capable of producing olivetolic acid from 3,5,7-trioxododecanoyl-CoA, and wherein, if present: the amino acid substitution at the residue corresponding to position 13 is D13R or D13K; the amino acid substitution at the residue corresponding to position 21 is E21K; the amino acid substitution at the residue corresponding to position 39 is D39G, D39S, or D39Q; the amino acid substitution at the residue corresponding to position 50 is N50K; the amino acid substitution at the residue corresponding to position 52 is E52H; the amino acid substitution at the residue corresponding to position 67 is E67K; the amino acid substitution at the residue corresponding to position 70 is Q70A or Q70L; the amino acid substitution at the residue corresponding to position 71 is D71Q or D71S; the amino acid substitution at the residue corresponding to position 74 is I74D, 174E, I74H, or I74Q; the amino acid substitution at the residue corresponding to position 83 is D83A or D83G; the amino acid substitution at the residue corresponding to position 86 is R86G; the amino acid substitution at the residue corresponding to position 87 is S87D or S87K; and the amino acid substitution at the residue corresponding to position 100 is R100A, R100G, R100K, R100N, R100Q, or R100S.

In some embodiments, the PKC comprises an amino acid substitution at a residue corresponding to position 2, 3, 13, 17, 18, 19, 20, 21, 22, 25, 26, 28, 29, 31, 33, 37, 38, 39, 45, 50, 52, 53, 55, 56, 57, 60, 62, 64, 65, 67, 68, 70, 71, 83, 84, 85, 87, 88, 95, 97, 98, 100, and/or 101 in SEQ ID NO: 1, and wherein, if present: the amino acid substitution at the residue corresponding to position 13 is D13R or D13K; the amino acid substitution at the residue corresponding to position 21 is E21K; the amino acid substitution at the residue corresponding to position 39 is D39G or D39Q; the amino acid substitution at the residue corresponding to position 50 is N50K; the amino acid substitution at the residue corresponding to position 52 is E52H; the amino acid substitution at the residue corresponding to position 67 is E67K; the amino acid substitution at the residue corresponding to position 70 is Q70A or Q70L; the amino acid substitution at the residue corresponding to position 71 is D71Q or D71S; the amino acid substitution at the residue corresponding to position 83 is D83A or D83G; the amino acid substitution at the residue corresponding to position 87 is S87K; and the amino acid substitution at the residue corresponding to position 100 is R100S.

In some embodiments, the PKC comprises: the amino acid G at a residue corresponding to position 2 in SEQ ID NO: 1; the amino acid M or Y at a residue corresponding to position 3 in SEQ ID NO: 1; the amino acid R or K at a residue corresponding to position 13 in SEQ ID NO: 1; the amino acid N at a residue corresponding to position 17 in SEQ ID NO: 1; the amino acid D or R at a residue corresponding to position 18 in SEQ ID NO: 1; the amino acid E or D at a residue corresponding to position 19 in SEQ ID NO: 1; the amino acid A at a residue corresponding to position 20 in SEQ ID NO: 1; the amino acid K at a residue corresponding to position 21 in SEQ ID NO: 1; the amino acid T or K at a residue corresponding to position 22 in SEQ ID NO: 1; the amino acid A or N at a residue corresponding to position 25 in SEQ ID NO: 1; the amino acid H, R, A, or E at a residue corresponding to position 26 in SEQ ID NO: 1; the amino acid A, S, E, or H at a residue corresponding to position 28 in SEQ ID NO: 1; the amino acid D, S, E, or T at a residue corresponding to position 29 in SEQ ID NO: 1; the amino acid L or A at a residue corresponding to position 31 in SEQ ID NO: 1; the amino acid H, R, D, E, K, Q, or N at a residue corresponding to position 33 in SEQ ID NO: 1; the amino acid L at a residue corresponding to position 37 in SEQ ID NO: 1; the amino acid L at a residue corresponding to position 38 in SEQ ID NO: 1; the amino acid G, S, or Q at a residue corresponding to position 39 in SEQ ID NO: 1; the amino acid K or R at a residue corresponding to position 41 in SEQ ID NO: 1; the amino acid I at a residue corresponding to position 45 in SEQ ID NO: 1; the amino acid K at a residue corresponding to position 50 in SEQ ID NO: 1; the amino acid H at a residue corresponding to position 52 in SEQ ID NO: 1; the amino acid H at a residue corresponding to position 53 in SEQ ID NO: 1; the amino acid F at a residue corresponding to position 55 in SEQ ID NO: 1; the amino acid Y at a residue corresponding to position 56 in SEQ ID NO: 1; the amino acid Y at a residue corresponding to position 57 in SEQ ID NO: 1; the amino acid I or L at a residue corresponding to position 60 in SEQ ID NO: 1; the amino acid M at a residue corresponding to position 62 in SEQ ID NO: 1; the amino acid Q or D at a residue corresponding to position 64 in SEQ ID NO: 1; the amino acid N at a residue corresponding to position 65 in SEQ ID NO: 1; the amino acid K at a residue corresponding to position 67 in SEQ ID NO: 1; the amino acid E, G, or H at a residue corresponding to position 68 in SEQ ID NO: 1; the amino acid A or L at a residue corresponding to position 70 in SEQ ID NO: 1; the amino acid Q or S, at a residue corresponding to position 71 in SEQ ID NO: 1; the amino acid D, E, H, or Q at a residue corresponding to position 74 in SEQ ID NO: 1; the amino acid A or G at a residue corresponding to position 83 in SEQ ID NO: 1; the amino acid F, K, M, or Q at a residue corresponding to position 84 in SEQ ID NO: 1; the amino acid A or F at a residue corresponding to position 85 in SEQ ID NO: 1; the amino acid G at a residue corresponding to position 86 in SEQ ID NO: 1; the amino acid D, or K at a residue corresponding to position 87 in SEQ ID NO: 1; the amino acid Y, N, or S at a residue corresponding to position 88 in SEQ ID NO: 1; the amino acid M or I at a residue corresponding to position 95 in SEQ ID NO: 1; the amino acid F at a residue corresponding to position 97 in SEQ ID NO: 1; the amino acid E, K, or Y at a residue corresponding to position 98 in SEQ ID NO: 1; the amino acid A, G, K, N, Q, or S at a residue corresponding to position 100 in SEQ ID NO: 1; and/or the amino acid A or E at a residue corresponding to position 101 in SEQ ID NO: 1.

In some embodiments, the PKC comprises: the amino acid M at a residue corresponding to position 24 in SEQ ID NO: 1; the amino acid F at a residue corresponding to position 46 in SEQ ID NO: 1; the amino acid A at a residue corresponding to position 48 in SEQ ID NO: 1; the amino acid Q at a residue corresponding to position 49 in SEQ ID NO: 1; the amino acid I or F at a residue corresponding to position 59 in SEQ ID NO: 1; the amino acid T, D, or S at a residue corresponding to position 61 in SEQ ID NO: 1; the amino acid L, M, or Y at a residue corresponding to position 66 in SEQ ID NO: 1; the amino acid L or Y at a residue corresponding to position 69 in SEQ ID NO: 1; the amino acid L at a residue corresponding to position 73 in SEQ ID NO: 1; 34.the amino acid S at a residue corresponding to position 74 in SEQ ID NO: 1; the amino acid V, A, E, S, D, or N at a residue corresponding to position 80 in SEQ ID NO: 1; the amino acid Y at a residue corresponding to position 81 in SEQ ID NO: 1; the amino acid K, V, or R at a residue corresponding to position 82 in SEQ ID NO: 1; the amino acid V or M at a residue corresponding to position 89 in SEQ ID NO: 1; and/or the amino acid V at a residue corresponding to position 94 in SEQ ID NO: 1.

A PKC may comprise one or more amino acid substitutions, deletions, and/or insertions in domain(s) corresponding to any of the domains set forth in Table 2. For example, a PKC may comprise one or more amino acid substitutions, deletions, and/or insertions in a domain corresponding to beta sheet 1 (positions 1-11 in SEQ ID NO:1); loop 1 (positions 12-16 in SEQ ID NO:1); helix 1 (positions 17-29 in SEQ ID NO:1); loop 2 (positions 33-38 in SEQ ID NO:1); loop 3 (positions 45-56 in SEQ ID NO:1); beta sheet 3 (positions 57-62 in SEQ ID NO:1); helix 2 (positions 66-73 in SEQ ID NO:1); helix 3 (positions 76-85 in SEQ ID NO:1); 310 helix 2 (positions 86-88 in SEQ ID NO: 1). In some embodiments, a PKC comprises one or more amino acid substitutions, deletions, and/or insertions in a domain corresponding to beta sheet 1 (positions 1-11 in SEQ ID NO: 1); loop 1 (positions 12-16 in SEQ ID NO: 1); helix 1 (positions 17-29 in SEQ ID NO: 1); 310 helix 1 (positions 30-32 in SEQ ID NO: 1); loop 2 (positions 33-38 in SEQ ID NO: 1); beta sheet 2 (positions 39-44 in SEQ ID NO: 1); loop 3 (positions 45-56 in SEQ ID NO: 1); beta sheet 3 (positions 57-62 in SEQ ID NO: 1); loop 4 (positions 63-65 in SEQ ID NO: 1); helix 2 (positions 66-73 in SEQ ID NO: 1); turn 1 (positions 74-75 in SEQ ID NO: 1); helix 3 (positions 76-85 in SEQ ID NO: 1); 310 helix 2 (positions 86-88 in SEQ ID NO: 1); and/or beta sheet 4 (positions 89-97 in SEQ ID NO: 1). In some embodiments, one or more amino acid substitutions are located in a domain corresponding to: helix 1 (positions 17-29 in SEQ ID NO: 1); loop 3 (positions 45-56 in SEQ ID NO: 1); helix 2 (positions 66-73 in SEQ ID NO: 1); and/or helix 3 (positions 76-85 in SEQ ID NO: 1).

In some embodiments, a PKC comprises one or more amino acid substitutions or deletions located in a domain corresponding to: beta sheet 1 (positions 1-11 in SEQ ID NO: 1); loop 1 (positions 12-16 in SEQ ID NO: 1); helix 1 (positions 17-29 in SEQ ID NO: 1); 310 helix 1 (positions 30-32 in SEQ ID NO: 1); loop 2 (positions 33-38 in SEQ ID NO: 1); beta sheet 2 (positions 39-44 in SEQ ID NO: 1); loop 3 (positions 45-56 in SEQ ID NO: 1); beta sheet 3 (positions 57-62 in SEQ ID NO: 1); loop 4 (positions 63-65 in SEQ ID NO: 1); helix 2 (positions 66-73 in SEQ ID NO: 1); turn 1 (positions 74-75 in SEQ ID NO: 1); helix 3 (positions 76-85 in SEQ ID NO: 1); 310 helix 2 (positions 86-88 in SEQ ID NO: 1); beta sheet 4 (positions 89-97 in SEQ ID NO: 1); and/or loop 5 (positions 98-101 in SEQ ID NO: 1).

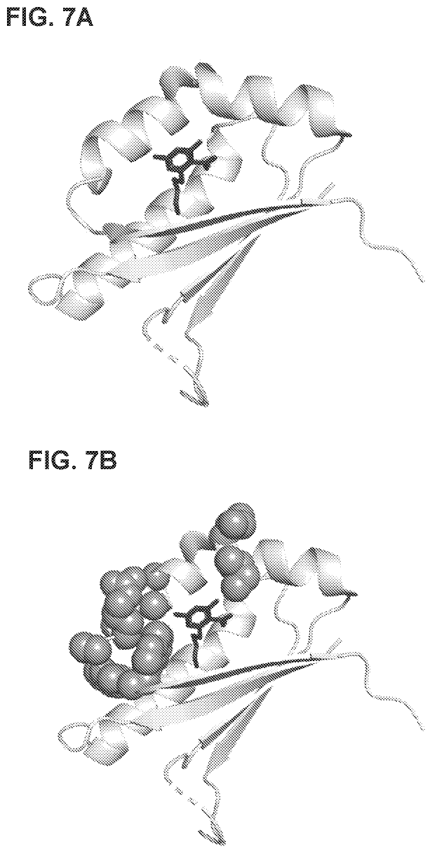

In some embodiments, a PKC comprises one or more amino acid substitutions at a residue in domain(s) corresponding to helix 2 (positions 66-73 in SEQ ID NO:1); turn 1 (positions 74-75 in SEQ ID NO:1); helix 3 (positions 76-85 in SEQ ID NO:1); 310 helix 2 (positions 86-88 in SEQ ID NO:1); and/or beta sheet 4 (positions 89-97 in SEQ ID NO:1). In some embodiments, a PKC comprises one or more amino acid substitutions at a residue corresponding to position 73, 74, 80, 82, 84, 86, 87, 88, and/or 89 in SEQ ID NO: 1. Without being bound by a particular theory, residues corresponding to position 73, 74, 80, 82, 84, 86, 87, 88, and/or 89 in SEQ ID NO: 1 may affect the expression and/or catalytic activity of a PKC because these residues are near or are in the substrate/product binding pocket of a PKC. Many of these positions are near turn 1 (positions 74 and 75), which forms a kink that separates helix 2 and helix 3. As can be observed from FIG. 7A and Table 2, above, the kink between helices 2 and 3 shapes the binding pocket and catalytic sites around the substrate. Without being bound by a particular theory, modification of a PKC at or near a helix kink may affect the catalytic function of a PKC, for example, by changing the shape of the binding pocket to better conform to the substrate, thereby enhancing the catalytic parameters (K_{M} and/or k_{cat}) of the PKC. As used herein, a “binding pocket” comprises the residues in an enzyme that directly contact the substrate and/or product. In some embodiments, one or more (e.g. at least 2, at least 3, at least 4, amino acid substitutions increase the stability of a kinked helix and/or improve conformation of a binding pocket of a PKC to the compound of Formula 4a and/or the compound of Formula 6a. In some embodiments, the kinked helix is defined by the domains corresponding to helix 2 (positions 66-73 in SEQ ID NO: 1), turn 1 (positions 74-75 in SEQ ID NO: 1), and helix 3 (positions 76-85 in SEQ ID NO: 1). In some embodiments, the kinked helix corresponds to positions 66-85 of SEQ ID NO: 1. In some embodiments, improving the conformation of a binding pocket of a PKC to a substrate increases the binding affinity of the PKC for the substrate. In some embodiments, improving the conformation of a binding pocket of a PKC to a substrate and/or product, increases the turnover number of the PKC.

In some embodiments, a PKC comprises one or more amino acid substitutions located in a domain corresponding to: loop 3 (positions 45-56 in SEQ ID NO: 1); helix 2 (positions 66-73 in SEQ ID NO: 1); and helix 3 (positions 76-85 in SEQ ID NO: 1). In some embodiments, a PKC comprises at least two amino acid substitutions in a domain corresponding to helix 3 (positions 76-85 in SEQ ID NO: 1). In some embodiments, a PKC comprises at least 6 amino acid substitutions or deletions relative to SEQ ID NO: 1, wherein the PKC comprises at least two amino acid substitutions in a domain corresponding to helix 3 (positions 76-85 in SEQ ID NO: 1), and wherein the PKC is capable of producing olivetolic acid from 3,5,7-trioxododecanoyl-CoA. In some embodiments, a PKC comprises at least two amino acid substitutions in a domain corresponding to helix 3 (positions 76-85 in SEQ ID NO: 1), wherein the at least two amino acid includes an amino acid substitution at position 84 relative to SEQ ID NO: 1. In some embodiments, the at least two amino acid substitutions in the domain corresponding to helix 3 (positions 76-85 in SEQ ID NO: 1) are at residues corresponding to positions 80 and 82 in SEQ ID NO: 1. In some embodiments, the PKC comprises: the amino acid E, A, or S at the residue corresponding to position 80 in SEQ ID NO: 1; and/or the amino acid V or K at the residue corresponding to position 82 in SEQ ID NO: 1. In some embodiments, the PKC comprises an amino acid substitution at a residue corresponding to position 73 or 74 in SEQ ID NO: 1. In some embodiments, the PKC comprises the amino acid L at the residue corresponding to position 73 in SEQ ID NO: 1; and/or the PKC comprises the amino acid S or L at the residue corresponding to position 74 in SEQ ID NO: 1. In some embodiments, the PKC comprises an amino acid substitution at a residue corresponding to position 84 and/or 86-89. In some embodiments, the PKC comprises: the amino acid M at the residue corresponding to position 84 in SEQ ID NO: 1; the amino acid G at the residue corresponding to position 86 in SEQ ID NO: 1; the amino acid D at the residue corresponding to position 87 in SEQ ID NO: 1; the amino acid Y at the residue corresponding to position 88 in SEQ ID NO: 1; and/or the amino acid V at the residue corresponding to position 89 in SEQ ID NO: 1.

In some embodiments, relative to SEQ ID NO: 1, a PKC comprises I73L, and/or G80E, G80A, or G80S, and/or G82V or G82K. In some embodiments, relative to SEQ ID NO: 1, a PKC comprises I74S or I74L and/or G80E and/or G82V or G82K. Without being bound by a particular theory, amino acid substitutions at residues corresponding to position 80 and position 82 in SEQ ID NO: 1 may increase the expression of a particular PKC relative to a wild-type PKC. In some embodiments, relative to SEQ ID NO: 1, a PKC further comprises W89V, F₈₈Y, S87D, R86G, and/or V84M.

Without being bound by a particular theory, a kink that separates helices is generally expected to be energetically unfavorable unless the kink is stabilized by surrounding amino acids. In some embodiments, an amino acid substitution disclosed in this application either alone or in combination with another amino acid substitution may stabilize a kink between two helices. In some embodiments, an amino acid substitution disclosed in this application either alone or in combination with another amino acid substitution may promote the folding of the kink and stabilize the kink. In some embodiments, amino acid substitutions including: I73L; one of G80E, G80A, or G80S; and one of G82V or G82K, relative to SEQ ID NO: 1, increase the stability or relieve the constraint of the kink between helices 2 and 3 in a PKC. In some embodiments, amino acid substitutions including: one of 174S or I74L; G80E; and one of G82V or G82K, relative to SEQ ID NO: 1, increase the stability or relieve the constraint of the kink between helices 2 and 3 in a PKC.

In some embodiments, a PKC comprises an amino acid substitution at a residue corresponding to position 26, 73, and/or 80 in SEQ ID NO: 1. In some embodiments, relative to SEQ ID NO: 1, a PKC comprises T26A, I73L, and/or G80A.

In some embodiments, relative to SEQ ID NO: 1, a PKC comprises one or more amino acid substitutions or deletions from Tables 4-8, 9A-9B, and/or 10. In some embodiments, a PKC comprises one or more amino acid substitutions at one or more positions corresponding to one or more positions selected from: position 17, 18, 29, 31, 33, 39, 71, 83, and/or 84 in SEQ ID NO: 1. In some embodiments, a PKC is capable of producing olivetolic acid from 3,5,7-trioxododecanoyl-CoA. In some embodiments, a PKC comprises one or more of the following amino acid substitutions: E17N, A18D, N29T, V31L, I33N, D39Q, D71S, D83A, and/or V84F relative to SEQ ID NO: 1. In some embodiments, the PKC comprises: a) E17N, A18D, V31L, D39Q, D71S, and D83A relative to SEQ ID NO: 1; b) E17N, A18D, V31L, D71S, D83A, and V84F relative to SEQ ID NO: 1; or c) A18D, V31L, D39Q, D71S, D83A, and V84F relative to SEQ ID NO: 1. See also, e.g., amino acid substitutions in Table 7.

In some embodiments, a PKC comprises an amino acid substitutions at one or more positions corresponding to one or more positions selected from: position 13, 18, 22, 26, 29, 33, 39, 49, 52, 55, 70, 71, 74, 80, 83, 84, and/or 85 in SEQ ID NO: 1. In some embodiments, the PKC comprises amino acid substitutions relative to SEQ ID NO: 1 selected from the group consisting of: D13R; A18D; E22K; T26R or T26H; N29S; 133R or 133H; D39G; K49Q; E52H; Y55F; Q70A or Q70L; D71Q; I74G; G80A; D83K or D83N; V84K; and Y85A. In some embodiments, relative to SEQ ID NO: 1, the PKC comprises: I33R, E52H, Y55F, Q70A, G80A, D83K, and V84K; D13R, A18D, N29S, Q70L, D71Q, G80A, and D83K; I33R, E52H, Y55F, Q70A, I74G, and D83K; T26R, I33H, E52H, Y55F, Q70A, and D83K; A18D, T26R, E52H, Y55F, Q70A, G80A, D83N, V84K, and Y85A; T26R, I33H, Y55F, Q70A, G80A, D83K, and Y85A; or E22K, T26H, N29S, D39G, K49Q, E52H, D83N, and V84K. See also, e.g., Table 8.

In some embodiments, the production of a PKC of interest by a host cell may be assessed as production relative to a control host cell. As a non-limiting example, the level of mRNA and/or protein level of a PKC relative may be assessed relative to the level of mRNA and/or protein level of a control PKC. In some embodiments, a control host cell does not comprise the PKC of interest. In some embodiments, a control host cell does not comprise a heterologous polynucleotide encoding a PKC. In some embodiments, the control host cell comprises a PKC that is different from the PKC of interest. In some embodiments, the control host cell comprises a wild-type PKC.

In some embodiments, a host cell produces at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, at least 500%, at least 510%, at least 520%, at least 530%, at least 540%, at least 550%, at least 560%, at least 570%, at least 580%, at least 590%, at least 600%, at least 610%, at least 620%, at least 630%, at least 640%, at least 650%, at least 660%, at least 670%, at least 680%, at least 690%, at least 700%, at least 710%, at least 720%, at least 730%, at least 740%, at least 750%, at least 760%, at least 770%, at least 780%, at least 790%, at least 800%, at least 810%, at least 820%, at least 830%, at least 840%, at least 850%, at least 860%, at least 870%, at least 880%, at least 890%, at least 900%, at least 910%, at least 920%, at least 930%, at least 940%, at least 950%, at least 960%, at least 970%, at least 980%, at least 990%, or at least 1,000%, including all values in between, of a PKC relative to a control host cell.

In some embodiments, a host cell that comprises a heterologous polynucleotide encoding a PKC described in this application produces increased levels of the PKC relative to a control host cell. As a non-limiting example, one or more amino acid substitutions, insertions, and/or deletions may increase the level of mRNA and/or protein level of a PKC relative to a control PKC. In some embodiments, a host cell produces at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, at least 500%, at least 510%, at least 520%, at least 530%, at least 540%, at least 550%, at least 560%, at least 570%, at least 580%, at least 590%, at least 600%, at least 610%, at least 620%, at least 630%, at least 640%, at least 650%, at least 660%, at least 670%, at least 680%, at least 690%, at least 700%, at least 710%, at least 720%, at least 730%, at least 740%, at least 750%, at least 760%, at least 770%, at least 780%, at least 790%, at least 800%, at least 810%, at least 820%, at least 830%, at least 840%, at least 850%, at least 860%, at least 870%, at least 880%, at least 890%, at least 900%, at least 910%, at least 920%, at least 930%, at least 940%, at least 950%, at least 960%, at least 970%, at least 980%, at least 990%, or at least 1,000%, including all values in between, more of a PKC relative to a control host cell.

In some embodiments, a PKC comprises one or more amino acid substitutions, insertions, and/or deletions that increase expression of the PKC relative to a control PKC. In some embodiments, a PKC comprises one or more amino acid substitutions, insertions, and/or deletions that increase production of a product relative to a control PKC. In some embodiments, a PKC comprises one or more amino acid substitutions, insertions, and/or deletions that increase expression of the PKC and increases of a product relative to a control PKC.

In some embodiments, a PKC catalyzes the formation of a compound which occurs in the presence of a PKS. In some embodiments, a PKC substrate is a tetraketide. PKC substrates include trioxoalkanoyl-CoAs, such as 3,5,7-Trioxododecanoyl-CoA, or a compound of Formula (4):

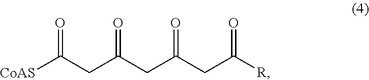

wherein R is hydrogen, optionally substituted acyl, optionally substituted alkyl, optionally substituted alkenyl, optionally substituted alkynyl, optionally substituted carbocyclyl, or optionally substituted aryl. In certain embodiments, a PKC catalyzes a compound of Formula (4):

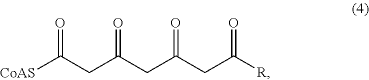

wherein R is hydrogen, optionally substituted acyl, optionally substituted alkyl, optionally substituted alkenyl, optionally substituted alkynyl, optionally substituted carbocyclyl, or optionally substituted aryl; to form a compound of Formula (6):

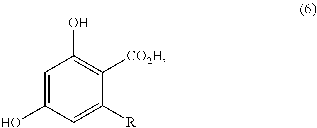

wherein R is hydrogen, optionally substituted acyl, optionally substituted alkyl, optionally substituted alkenyl, optionally substituted alkynyl, optionally substituted carbocyclyl, or optionally substituted aryl; as substrates. R is as defined in this application. In some embodiments, R is a C2-C8 optionally substituted alkyl. In some embodiments, R is a C2-C6 optionally substituted alkyl. In some embodiments, R is a propyl or pentyl. In some embodiments, R is pentyl. In some embodiments, R is propyl.

Any of the enzymes, host cells, and methods described in this application may be used for the production of cannabinoids and cannabinoid precursors, such as those provided in Table 1. In general, the term “production” is used to refer to the generation of one or more products (e.g., products of interest and/or by-products/off-products), for example, from a particular substrate or reactant. The amount of production may be evaluated at any one or more steps of a pathway, such as a final product or an intermediate product, using metrics familiar to one of ordinary skill in the art. For example, the amount of production may be assessed for a single enzymatic reaction (e.g., conversion of a compound of Formula (4) to a compound of Formula (6) by a PKC). Alternatively or in addition, the amount of production may be assessed for a series of enzymatic reactions (e.g., the biosynthetic pathway shown in FIG. 1 and/or FIG. 2). Production may be assessed by any metrics known in the art, for example, by assessing volumetric productivity, enzyme kinetics/reaction rate, specific productivity biomass-specific productivity, titer, yield, and total titer of one or more products (e.g., products of interest and/or by-products/off-products).

In some embodiments, the metric used to measure production may depend on whether a continuous process is being monitored (e.g., several cannabinoid biosynthesis steps are used in combination) or whether a particular end product is being measured. For example, in some embodiments, metrics used to monitor production by a continuous process may include volumetric productivity, enzyme kinetics and reaction rate. In some embodiments, metrics used to monitor production of a particular product may include specific productivity biomass-specific productivity, titer, yield, and total titer of one or more products (e.g., products of interest and/or by-products/off-products).

Production of one or more products (e.g., products of interest and/or by-products/off-products) may be assessed indirectly, for example by determining the amount of a substrate remaining following termination of the reaction/fermentation. For example, for a PKC that catalyzes the formation of products (e.g., a compound of Formula (6), including olivetolic acid (Formula (6a)) from a compound of Formula (4), including 3,5,7-trioxododecanoyl-CoA (Formula (4a)), production of the products may be assessed by quantifying the compound of Formula (6) directly or by quantifying the amount of substrate remaining following the reaction (e.g., amount of the compound of Formula (4)).

In some embodiments, the production of a product (e.g., products of interest and/or by-products/off-products) may be assessed as production relative to a control. In some embodiments, the production of a compound of Formula (6) by a particular PKC may be assessed relative to a control. In some embodiments, the production of CBGA by a particular PKC in a host cell may be assessed relative to a PKC in another host cell. In some embodiments, the production of a compound of Formula (6) from a particular substrate may be assessed relative to a control using a different substrate.

In some embodiments, a PKC may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) the amount of one or more compounds of Formula (6) relative to a control. In some embodiments, the compound of Formula (6) is olivetolic acid (Formula (6a)).

In some embodiments, a PKC may be capable of producing a product at a higher titer or yield relative to a control. In some embodiments, a PKC may be capable of producing a product at a faster rate (e.g., higher productivity) relative to a control. In some embodiments, a PKC may have preferential binding and/or activity towards one substrate relative to another substrate. In some embodiments, a PKC may preferentially produce one product relative to another product.

In some embodiments, a PKC may produce at least 0.0001 μg/L, at least 0.001 μg/L, at least 0.01 μg/L, at least 0.02 μg/L, at least 0.03 μg/L, at least 0.04 μg/L, at least 0.05 μg/L, at least 0.06 μg/L, at least 0.07 μg/L, at least 0.08 μg/L, at least 0.09 μg/L, at least 0.1 μg/L, at least 0.11 μg/L, at least 0.12 μg/L, at least 0.13 μg/L, at least 0.14 μg/L, at least 0.15 μg/L, at least 0.16 μg/L, at least 0.17 μg/L, at least 0.18 μg/L, at least 0.19 μg/L, at least 0.2 μg/L, at least 0.21 μg/L, at least 0.22 μg/L, at least 0.23 μg/L, at least 0.24 μg/L, at least 0.25 μg/L, at least 0.26 μg/L, at least 0.27 μg/L, at least 0.28 μg/L, at least 0.29 μg/L, at least 0.3 μg/L, at least 0.31 μg/L, at least 0.32 μg/L, at least 0.33 μg/L, at least 0.34 μg/L, at least 0.35 μg/L, at least 0.36 μg/L, at least 0.37 μg/L, at least 0.38 μg/L, at least 0.39 μg/L, at least 0.4 μg/L, at least 0.41 μg/L, at least 0.42 μg/L, at least 0.43 μg/L, at least 0.44 μg/L, at least 0.45 μg/L, at least 0.46 μg/L, at least 0.47 μg/L, at least 0.48 μg/L, at least 0.49 μg/L, at least 0.5 μg/L, at least 0.51 μg/L, at least 0.52 μg/L, at least 0.53 μg/L, at least 0.54 μg/L, at least 0.55 μg/L, at least 0.56 μg/L, at least 0.57 μg/L, at least 0.58 μg/L, at least 0.59 μg/L, at least 0.6 μg/L, at least 0.61 μg/L, at least 0.62 μg/L, at least 0.63 μg/L, at least 0.64 μg/L, at least 0.65 μg/L, at least 0.66 μg/L, at least 0.67 μg/L, at least 0.68 μg/L, at least 0.69 μg/L, at least 0.7 μg/L, at least 0.71 μg/L, at least 0.72 μg/L, at least 0.73 μg/L, at least 0.74 μg/L, at least 0.75 μg/L, at least 0.76 μg/L, at least 0.77 μg/L, at least 0.78 μg/L, at least 0.79 μg/L, at least 0.8 μg/L, at least 0.81 μg/L, at least 0.82 μg/L, at least 0.83 μg/L, at least 0.84 μg/L, at least 0.85 μg/L, at least 0.86 μg/L, at least 0.87 μg/L, at least 0.88 μg/L, at least 0.89 μg/L, at least 0.9 μg/L, at least 0.91 μg/L, at least 0.92 μg/L, at least 0.93 μg/L, at least 0.94 μg/L, at least 0.95 μg/L, at least 0.96 μg/L, at least 0.97 μg/L, at least 0.98 μg/L, at least 0.99 μg/L, at least 1 μg/L, at least 1.1 μg/L, at least 1.2 μg/L, at least 1.3 μg/L, at least 1.4 μg/L, at least 1.5 μg/L, at least 1.6 μg/L, at least 1.7 μg/L, at least 1.8 μg/L, at least 1.9 μg/L, at least 2 μg/L, at least 2.1 μg/L, at least 2.2 μg/L, at least 2.3 μg/L, at least 2.4 μg/L, at least 2.5 μg/L, at least 2.6 μg/L, at least 2.7 μg/L, at least 2.8 μg/L, at least 2.9 μg/L, at least 3 μg/L, at least 3.1 μg/L, at least 3.2 μg/L, at least 3.3 μg/L, at least 3.4 μg/L, at least 3.5 μg/L, at least 3.6 μg/L, at least 3.7 μg/L, at least 3.8 μg/L, at least 3.9 μg/L, at least 4 μg/L, at least 4.1 μg/L, at least 4.2 μg/L, at least 4.3 μg/L, at least 4.4 μg/L, at least 4.5 μg/L, at least 4.6 μg/L, at least 4.7 μg/L, at least 4.8 μg/L, at least 4.9 μg/L, at least 5 μg/L, at least 5.1 μg/L, at least 5.2 μg/L, at least 5.3 μg/L, at least 5.4 μg/L, at least 5.5 μg/L, at least 5.6 μg/L, at least 5.7 μg/L, at least 5.8 μg/L, at least 5.9 μg/L, at least 6 μg/L, at least 6.1 μg/L, at least 6.2 μg/L, at least 6.3 μg/L, at least 6.4 μg/L, at least 6.5 μg/L, at least 6.6 μg/L, at least 6.7 μg/L, at least 6.8 μg/L, at least 6.9 μg/L, at least 7 μg/L, at least 7.1 μg/L, at least 7.2 μg/L, at least 7.3 μg/L, at least 7.4 μg/L, at least 7.5 μg/L, at least 7.6 μg/L, at least 7.7 μg/L, at least 7.8 μg/L, at least 7.9 μg/L, at least 8 μg/L, at least 8.1 μg/L, at least 8.2 μg/L, at least 8.3 μg/L, at least 8.4 μg/L, at least 8.5 μg/L, at least 8.6 μg/L, at least 8.7 μg/L, at least 8.8 μg/L, at least 8.9 μg/L, at least 9 μg/L, at least 9.1 μg/L, at least 9.2 μg/L, at least 9.3 μg/L, at least 9.4 μg/L, at least 9.5 μg/L, at least 9.6 μg/L, at least 9.7 μg/L, at least 9.8 μg/L, at least 9.9 μg/L, at least 10 μg/L, at least 10.1 μg/L, at least 10.2 μg/L, at least 10.3 μg/L, at least 10.4 μg/L, at least 10.5 μg/L, at least 10.6 μg/L, at least 10.7 μg/L, at least 10.8 μg/L, at least 10.9 μg/L, at least 11 μg/L, at least 11.1 μg/L, at least 11.2 μg/L, at least 11.3 μg/L, at least 11.4 μg/L, at least 11.5 μg/L, at least 11.6 μg/L, at least 11.7 μg/L, at least 11.8 μg/L, at least 11.9 μg/L, at least 12 μg/L, at least 12.1 μg/L, at least 12.2 μg/L, at least 12.3 μg/L, at least 12.4 μg/L, at least 12.5 μg/L, at least 12.6 μg/L, at least 12.7 μg/L, at least 12.8 μg/L, at least 12.9 μg/L, at least 13 μg/L, at least 13.1 μg/L, at least 13.2 μg/L, at least 13.3 μg/L, at least 13.4 μg/L, at least 13.5 μg/L, at least 13.6 μg/L, at least 13.7 μg/L, at least 13.8 μg/L, at least 13.9 μg/L, at least 14 μg/L, at least 14.1 μg/L, at least 14.2 μg/L, at least 14.3 μg/L, at least 14.4 μg/L, at least 14.5 μg/L, at least 14.6 μg/L, at least 14.7 μg/L, at least 14.8 μg/L, at least 14.9 μg/L, at least 15 μg/L, at least 15.1 μg/L, at least 15.2 μg/L, at least 15.3 μg/L, at least 15.4 μg/L, at least 15.5 μg/L, at least 15.6 μg/L, at least 15.7 μg/L, at least 15.8 μg/L, at least 15.9 μg/L, at least 16 μg/L, at least 16.1 μg/L, at least 16.2 μg/L, at least 16.3 μg/L, at least 16.4 μg/L, at least 16.5 μg/L, at least 16.6 μg/L, at least 16.7 μg/L, at least 16.8 μg/L, at least 16.9 μg/L, at least 17 μg/L, at least 17.1 μg/L, at least 17.2 μg/L, at least 17.3 μg/L, at least 17.4 μg/L, at least 17.5 μg/L, at least 17.6 μg/L, at least 17.7 μg/L, at least 17.8 μg/L, at least 17.9 μg/L, at least 18 μg/L, at least 18.1 μg/L, at least 18.2 μg/L, at least 18.3 μg/L, at least 18.4 μg/L, at least 18.5 μg/L, at least 18.6 μg/L, at least 18.7 μg/L, at least 18.8 μg/L, at least 18.9 μg/L, at least 19 μg/L, at least 19.1 μg/L, at least 19.2 μg/L, at least 19.3 μg/L, at least 19.4 μg/L, at least 19.5 μg/L, at least 19.6 μg/L, at least 19.7 μg/L, at least 19.8 μg/L, at least 19.9 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 105 μg/L, at least 110 μg/L, at least 115 μg/L, at least 120 μg/L, at least 125 μg/L, at least 130 μg/L, at least 135 μg/L, at least 140 μg/L, at least 145 μg/L, at least 150 μg/L, at least 155 μg/L, at least 160 μg/L, at least 165 μg/L, at least 170 μg/L, at least 175 μg/L, at least 180 μg/L, at least 185 μg/L, at least 190 μg/L, at least 195 μg/L, at least 200 μg/L, at least 205 μg/L, at least 210 μg/L, at least 215 μg/L, at least 220 μg/L, at least 225 μg/L, at least 230 μg/L, at least 235 μg/L, at least 240 μg/L, at least 245 μg/L, at least 250 μg/L, at least 255 μg/L, at least 260 μg/L, at least 265 μg/L, at least 270 μg/L, at least 275 μg/L, at least 280 μg/L, at least 285 μg/L, at least 290 μg/L, at least 295 μg/L, at least 300 μg/L, at least 305 μg/L, at least 310 μg/L, at least 315 μg/L, at least 320 μg/L, at least 325 μg/L, at least 330 μg/L, at least 335 μg/L, at least 340 μg/L, at least 345 μg/L, at least 350 μg/L, at least 355 μg/L, at least 360 μg/L, at least 365 μg/L, at least 370 μg/L, at least 375 μg/L, at least 380 μg/L, at least 385 μg/L, at least 390 μg/L, at least 395 μg/L, at least 400 μg/L, at least 405 μg/L, at least 410 μg/L, at least 415 μg/L, at least 420 μg/L, at least 425 μg/L, at least 430 μg/L, at least 435 μg/L, at least 440 μg/L, at least 445 μg/L, at least 450 μg/L, at least 455 μg/L, at least 460 μg/L, at least 465 μg/L, at least 470 μg/L, at least 475 μg/L, at least 480 μg/L, at least 485 μg/L, at least 490 μg/L, at least 495 μg/L, at least 500 μg/L, at least 600 μg/L, at least 700 μg/L, at least 800 μg/L, at least 900 μg/L, at least 1,000 μg/L, at least 2,000 μg/L, at least 3,000 μg/L, at least 4,000 μg/L, at least 5,000 μg/L, at least 6,000 μg/L, at least 7,000 μg/L, at least 8,000 μg/L, at least 9,000 μg/L, at least 10,000 μg/L, at least 11,000 μg/L, at least 12,000 μg/L, at least 13,000 μg/L, at least 14,000 μg/L, at least 15,000 μg/L, at least 16,000 μg/L, at least 17,000 μg/L, at least 18,000 μg/L, at least 19,000 μg/L, at least 20,000 μg/L, at least 21,000 μg/L, at least 22,000 μg/L, at least 23,000 μg/L, at least 24,000 μg/L, at least 25,000 μg/L, at least 26,000 μg/L, at least 27,000 μg/L, at least 28,000 μg/L, at least 29,000 μg/L, at least 30,000 μg/L, at least 31,000 μg/L, at least 32,000 μg/L, at least 33,000 μg/L, at least 34,000 μg/L, at least 35,000 μg/L, at least 36,000 μg/L, at least 37,000 μg/L, at least 38,000 μg/L, at least 39,000 μg/L, at least 40,000 μg/L, at least 41,000 μg/L, at least 42,000 μg/L, at least 43,000 μg/L, at least 44,000 μg/L, at least 45,000 μg/L, at least 46,000 μg/L, at least 47,000 μg/L, at least 48,000 μg/L, at least 49,000 μg/L, at least 50,000 μg/L, at least 51,000 μg/L, at least 52,000 μg/L, at least 53,000 μg/L, at least 54,000 μg/L, at least 55,000 μg/L, at least 56,000 μg/L, at least 57,000 μg/L, at least 58,000 μg/L, at least 59,000 μg/L, at least 60,000 μg/L, at least 61,000 μg/L, at least 62,000 μg/L, at least 63,000 μg/L, at least 64,000 μg/L, at least 65,000 μg/L, at least 66,000 μg/L, at least 67,000 μg/L, at least 68,000 μg/L, at least 69,000 μg/L, at least 70,000 μg/L, at least 71,000 μg/L, at least 72,000 μg/L, at least 73,000 μg/L, at least 74,000 μg/L, at least 75,000 μg/L, at least 76,000 μg/L, at least 77,000 μg/L, at least 78,000 μg/L, at least 79,000 μg/L, at least 80,000 μg/L, at least 81,000 μg/L, at least 82,000 μg/L, at least 83,000 μg/L, at least 84,000 μg/L, at least 85,000 μg/L, at least 86,000 μg/L, at least 87,000 μg/L, at least 88,000 μg/L, at least 89,000 μg/L, at least 90,000 μg/L, at least 91,000 μg/L, at least 92,000 μg/L, at least 93,000 μg/L, at least 94,000 μg/L, at least 95,000 μg/L, at least 96,000 μg/L, at least 97,000 μg/L, at least 98,000 μg/L, at least 99,000 μg/L, at least 100,000 μg/L, at least 105,000 μg/L, at least 110,000 μg/L, at least 115,000 μg/L, at least 120,000 μg/L, at least 125,000 μg/L, at least 130,000 μg/L, at least 135,000 μg/L, at least 140,000 μg/L, at least 145,000 μg/L, at least 150,000 μg/L, at least 155,000 μg/L, at least 160,000 μg/L, at least 165,000 μg/L, at least 170,000 μg/L, at least 175,000 μg/L, at least 180,000 μg/L, at least 185,000 μg/L, at least 190,000 μg/L, at least 195,000 μg/L, at least 200,000 μg/L, at least 205,000 μg/L, at least 210,000 μg/L, at least 215,000 μg/L, at least 220,000 μg/L, at least 225,000 μg/L, at least 230,000 μg/L, at least 235,000 μg/L, at least 240,000 μg/L, at least 245,000 μg/L, at least 250,000 μg/L, at least 255,000 μg/L, at least 260,000 μg/L, at least 265,000 μg/L, at least 270,000 μg/L, at least 275,000 μg/L, at least 280,000 μg/L, at least 285,000 μg/L, at least 290,000 μg/L, at least 295,000 μg/L, at least 300,000 μg/L, at least 305,000 μg/L, at least 310,000 μg/L, at least 315,000 μg/L, at least 320,000 μg/L, at least 325,000 μg/L, at least 330,000 μg/L, at least 335,000 μg/L, at least 340,000 μg/L, at least 345,000 μg/L, at least 350,000 μg/L, at least 355,000 μg/L, at least 360,000 μg/L, at least 365,000 μg/L, at least 370,000 μg/L, at least 375,000 μg/L, at least 380,000 μg/L, at least 385,000 μg/L, at least 390,000 μg/L, at least 395,000 μg/L, at least 400,000 μg/L, at least 405,000 μg/L, at least 410,000 μg/L, at least 415,000 μg/L, at least 420,000 μg/L, at least 425,000 μg/L, at least 430,000 μg/L, at least 435,000 μg/L, at least 440,000 μg/L, at least 445,000 μg/L, at least 450,000 μg/L, at least 455,000 μg/L, at least 460,000 μg/L, at least 465,000 μg/L, at least 470,000 μg/L, at least 475,000 μg/L, at least 480,000 μg/L, at least 485,000 μg/L, at least 490,000 μg/L, at least 495,000 μg/L, at least 500,000 μg/L, at least 600,000 μg/L, at least 700,000 μg/L, at least 800,000 μg/L, at least 900,000 μg/L, or at least 1,000,000 μg/L, including all values in between, of a compound of Formula (6). In some embodiments, the compound of Formula (6) is olivetolic acid (Formula (6a)).

In some embodiments, a PKC may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of one or more compounds of Formula (6) relative to a control. In some embodiments, the compound of Formula (6) is olivetolic acid (Formula (6a)).

In some embodiments, the ratio of two compounds produced by a PKC is assessed. As a non-limiting example, the ratio of the amount of a compound of Formula (6) to the amount of a compound of Formula (5) produced by a PKC can be assessed. In some embodiments, the amount of each product is measured in μg/L. In some embodiments, the compound of Formula (6) is a compound of Formula (6a) (olivetolic acid) and/or the compound of Formula (5) is a compound of Formula (5a) (olivetol). In some embodiments, a PKC produces a ratio of a compound of Formula (6) to a compound of Formula (5) that is at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6, at least 3.7, at least 3.8, at least 3.9, at least 4, at least 4.1, at least 4.2, at least 4.3, at least 4.4, at least 4.5, at least 4.6, at least 4.7, at least 4.8, at least 4.9, at least 5, at least 5.1, at least 5.2, at least 5.3, at least 5.4, at least 5.5, at least 5.6, at least 5.7, at least 5.8, at least 5.9, at least 6, at least 6.1, at least 6.2, at least 6.3, at least 6.4, at least 6.5, at least 6.6, at least 6.7, at least 6.8, at least 6.9, at least 7, at least 7.1, at least 7.2, at least 7.3, at least 7.4, at least 7.5, at least 7.6, at least 7.7, at least 7.8, at least 7.9, at least 8, at least 8.1, at least 8.2, at least 8.3, at least 8.4, at least 8.5, at least 8.6, at least 8.7, at least 8.8, at least 8.9, at least 9, at least 9.1, at least 9.2, at least 9.3, at least 9.4, at least 9.5, at least 9.6, at least 9.7, at least 9.8, at least 9.9, at least 10, at least 10.1, at least 10.2, at least 10.3, at least 10.4, at least 10.5, at least 10.6, at least 10.7, at least 10.8, at least 10.9, at least 11, at least 11.1, at least 11.2, at least 11.3, at least 11.4, at least 11.5, at least 11.6, at least 11.7, at least 11.8, at least 11.9, at least 12, at least 12.1, at least 12.2, at least 12.3, at least 12.4, at least 12.5, at least 12.6, at least 12.7, at least 12.8, at least 12.9, at least 13, at least 13.1, at least 13.2, at least 13.3, at least 13.4, at least 13.5, at least 13.6, at least 13.7, at least 13.8, at least 13.9, at least 14, at least 14.1, at least 14.2, at least 14.3, at least 14.4, at least 14.5, at least 14.6, at least 14.7, at least 14.8, at least 14.9, at least 15, at least 15.1, at least 15.2, at least 15.3, at least 15.4, at least 15.5, at least 15.6, at least 15.7, at least 15.8, at least 15.9, at least 16, at least 16.1, at least 16.2, at least 16.3, at least 16.4, at least 16.5, at least 16.6, at least 16.7, at least 16.8, at least 16.9, at least 17, at least 17.1, at least 17.2, at least 17.3, at least 17.4, at least 17.5, at least 17.6, at least 17.7, at least 17.8, at least 17.9, at least 18, at least 18.1, at least 18.2, at least 18.3, at least 18.4, at least 18.5, at least 18.6, at least 18.7, at least 18.8, at least 18.9, at least 19, at least 19.1, at least 19.2, at least 19.3, at least 19.4, at least 19.5, at least 19.6, at least 19.7, at least 19.8, at least 19.9, or at least 20, including all values in between. In some embodiments, a PKC produces a ratio of a compound of Formula (6) to a compound of a Formula (5) that is between 0.1 and 1, between 1 and 5, between 1 and 10, between 0.5 and 1, between 1 and 2, between 2 and 3, between 3 and 4, between 4 and 5, between 5 and 6, between 6 and 7, between 7 and 8, between 8 and 9, or between 9 and 10.

In some embodiments, a PKC produces a higher ratio of a compound of Formula (6) to a compound of Formula (5) as compared to a control. In some embodiments, the control is a PKC comprising SEQ ID NO: 1. In some embodiments, the control is a PKC that does not comprise one or more amino acid substitutions, deletions, or insertions present in the PKC that is being compared to the control.

In some embodiments, a PKC may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) higher titer or yield of one or more compounds of Formula (6) relative to a control. In some embodiments, the compound of Formula (6) is olivetolic acid (Formula (6a)).

In some embodiments, a PKC may be capable of producing one or more compounds of Formula (6) at a rate that is at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) faster relative to a control. In some embodiments, the compound of Formula (6) is olivetolic acid (Formula (6a)).

In some embodiments, a PKC may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of one or more compounds of Formula (6) relative to a control. In some embodiments, the compound of Formula (6) is olivetolic acid (Formula (6a)).

In some embodiments, a PKC may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) lower titer or yield of one or more compounds of Formula (6) relative to a control. In some embodiments, the compound of Formula (6) is olivetolic acid (Formula (6a)).

In some embodiments, a PKC may be capable of producing one or more compounds of Formula (6) at a rate that is at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) slower relative to a control. In some embodiments, the compound of Formula (6) is olivetolic acid (Formula (6a)).

In some embodiments of methods described herein involving comparison of an experimental PKC enzyme to a control, the control is a wild-type reference PKC. In some embodiments, the control is wild-type CsOAC (SEQ ID NO: 1). In some embodiments, the control PKC is identical to an experimental PKC except for the presence of one or more amino acid substitutions, insertions, or deletions within the experimental PKC.

In some embodiments of methods described herein involving comparison of an experimental host cell to a control host cell, the control host cell is a host cell that does not comprise a heterologous polynucleotide encoding a PKC. In some embodiments, a control host cell is a wild type cell. In some embodiments, a control host cell is a host cell that comprises a heterologous polynucleotide encoding a PKC corresponding to SEQ ID NO: 1. In some embodiments, a control host cell is a host cell that comprises a heterologous polynucleotide comprising SEQ ID NO: 2 or 3. In some embodiments, a control host cell is genetically identical to an experimental host cell except for the presence of one or more amino acid substitutions, insertions, or deletions within a PKC that is heterologously expressed in the experimental host cell.

In some embodiments, a PKC is capable of producing a product mixture comprising a compound of Formula (6a) and one or more other compounds of Formula (6). In some embodiments, at least approximately 50-100%, at least approximately 50-60%, at least approximately 60-70%, at least approximately 70-80%, at least approximately 80-90%, at least approximately 90-100%, of compounds within the product mixture are compounds of Formula (6a). In some embodiments, a PKC is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (6a) than another compound of Formula (6). In some embodiments, a PKC is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (6a) than another compound of Formula (6).

### Additional Cannabinoid Pathway Enzymes

Methods for production of cannabinoids and cannabinoid precursors can further include expression of one or more of: an acyl activating enzyme (AAE); a polyketide synthase (PKS) (e.g., OLS); a polyketide cyclase (PKC); a prenyltransferase (PT); and a terminal synthase (TS).

### Acyl Activating Enzyme (AAE)

A host cell described in this disclosure may comprise an acyl activating enzyme (AAE). As used in this disclosure, an acyl activating enzyme (AAE) refers to an enzyme that is capable of catalyzing the esterification between a thiol and a substrate (e.g., optionally substituted aliphatic or aryl group) that has a carboxylic acid moiety. In some embodiments, an AAE is capable of using Formula (1):

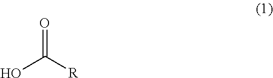

or a salt, solvate, hydrate, polymorph, co-crystal, tautomer, stereoisomer, isotopically labeled derivative thereof to produce a product of Formula (2):

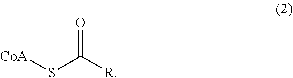

R is as defined in this application. In certain embodiments, R is hydrogen. In certain embodiments, R is optionally substituted alkyl. In certain embodiments, R is optionally substituted C1-40 alkyl. In certain embodiments, R is optionally substituted C₂₋₄₀ alkyl. In certain embodiments, R is optionally substituted C₂₋₄₀ alkyl, which is straight chain or branched alkyl. In certain embodiments, R is optionally substituted C2-10 alkyl, optionally substituted C10-C20 alkyl, optionally substituted C20-C30 alkyl, optionally substituted C30-C40 alkyl, or optionally substituted C40-C50 alkyl, which is straight chain or branched alkyl. In certain embodiments, R is optionally substituted C3-8 alkyl. In certain embodiments, R is optionally substituted C1-C40 alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C20 branched alkyl. In certain embodiments, R is optionally substituted C1-C20 alkyl, optionally substituted C1-C10 alkyl, optionally substituted C10-C20 alkyl, optionally substituted C20-C30 alkyl, optionally substituted C30-C40 alkyl, or optionally substituted C40-C50 alkyl. In certain embodiments, R is optionally substituted C1-C10 alkyl. In certain embodiments, R is optionally substituted C3 alkyl. In certain embodiments, R is optionally substituted n-propyl. In certain embodiments, R is unsubstituted n-propyl. In certain embodiments, R is optionally substituted C1-C8 alkyl. In some embodiments, R is a C2-C6 alkyl. In certain embodiments, R is optionally substituted C1-C5 alkyl. In certain embodiments, R is optionally substituted C3-C5 alkyl. In certain embodiments, R is optionally substituted C3 alkyl. In certain embodiments, R is optionally substituted C5 alkyl. In certain embodiments, R is of formula:

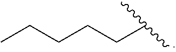 [SMILES: *CCCCC]

In certain embodiments, R is of formula:

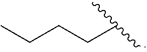 [SMILES: *CCCC]

In certain embodiments, R is of formula:

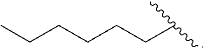 [SMILES: *CCCCCC]

In certain embodiments, R is of formula:

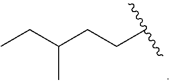 [SMILES: *CCC(C)CC]

In certain embodiments, R is optionally substituted propyl. In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

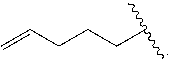 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

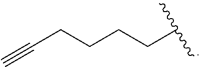 [SMILES: *CCCCC#C]

In certain embodiments, R is optionally substituted carbocyclyl. In certain embodiments, R is optionally substituted aryl (e.g., phenyl or napthyl).

In some embodiments, a substrate for an AAE is produced by fatty acid metabolism within a host cell. In some embodiments, a substrate for an AAE is provided exogenously.

In some embodiments, an AAE is capable of catalyzing the formation of hexanoyl-coenzyme A (hexanoyl-CoA) from hexanoic acid and coenzyme A (CoA). In some embodiments, an AAE is capable of catalyzing the formation of butanoyl-coenzyme A (butanoyl-CoA) from butanoic acid and coenzyme A (CoA).

As one of ordinary skill in the art would appreciate, an AAE could be obtained from any source, including naturally occurring sources and synthetic sources (e.g., a non-naturally occurring AAE). In some embodiments, the AAE is endogenous to the host cell, such as a Saccharomyces cerevisiae. In some embodiments, an AAE is a Cannabis enzyme. Non-limiting examples of AAEs include C. sativa hexanoyl-CoA synthetase 1 (CsHCS1) and C. sativa hexanoyl-CoA synthetase 2 (CsHCS2) as disclosed in U.S. Pat. No. 9,546,362, which is incorporated by reference in this application in its entirety.

CsHCS1 has the sequence:
(SEQ ID NO: 5)
MGKNYKSLDSVVASDFIALGITSEVAETLHGRLAEIVCNYGAATPQTWIN
IANHILSPDLPFSLHQMLFYGCYKDFGPAPPAWIPDPEKVKSTNLGALLE
KRGKEFLGVKYKDPISSFSHFQEFSVRNPEVYWRTVLMDEMKISFSKDPE
CILRRDDINNPGGSEWLPGGYLNSAKNCLNVNSNKKLNDTMIVWRDEGND
DLPLNKLTLDQLRKRVWLVGYALEEMGLEKGCAIAIDMPMHVDAVVIYLA
IVLAGYVVVSIADSFSAPEISTRLRLSKAKAIFTQDHIIRGKKRIPLYSR
VVEAKSPMAIVIPCSGSNIGAELRDGDISWDYFLERAKEFKNCEFTAREQ
PVDAYTNILFSSGTTGEPKAIPWTQATPLKAAADGWSHLDIRKGDVIVWP
TNLGWMMGPWLVYASLLNGASIALYNGSPLVSGFAKFVQDAKVTMLGVVP
SIVRSWKSTNCVSGYDWSTIRCFSSSGEASNVDEYLWLMGRANYKPVIEM
CGGTEIGGAFSAGSFLQAQSLSSFSSQCMGCTLYILDKNGYPMPKNKPGI
GELALGPVMFGASKTLLNGNHHDVYFKGMPTLNGEVLRRHGDIFELTSNG
YYHAHGRADDTMNIGGIKISSIEIERVCNEVDDRVFETTAIGVPPLGGGP
EQLVIFFVLKDSNDTTIDLNQLRLSFNLGLQKKLNPLFKVTRVVPLSSLP
RTATNKIMRRVLRQFSHFE.
CsHCS2 has the sequence:
(SEQ ID NO: 6)
MEKSGYGRDGIYRSLRPPLHLPNNNNLSMVSFLFRNSSSYPQKPALIDSE
TNQILSFSHFKSTVIKVSHGFLNLGIKKNDVVLIYAPNSIHFPVCFLGII
ASGAIATTSNPLYTVSELSKQVKDSNPKLIITVPQLLEKVKGFNLPTILI
GPDSEQESSSDKVMTFNDLVNLGGSSGSEFPIVDDFKQSDTAALLYSSGT
TGMSKGVVLTHKNFIASSLMVTMEQDLVGEMDNVFLCFLPMFHVFGLAII
TYAQLQRGNTVISMARFDLEKMLKDVEKYKVTHLWVVPPVILALSKNSMV
KKFNLSSIKYIGSGAAPLGKDLMEECSKVVPYGIVAQGYGMTETCGIVSM
EDIRGGKRNSGSAGMLASGVEAQIVSVDTLKPLPPNQLGEIWVKGPNMMQ
GYFNNPQATKLTIDKKGWVHTGDLGYFDEDGHLYVVDRIKELIKYKGFQV
APAELEGLLVSHPEILDAVVIPFPDAEAGEVPVAYVVRSPNSSLTENDVK
KFIAGQVASFKRLRKVTFINSVPKSASGKILRRELIQKVRSNM.

### Polyketide Synthases (PKS)

A host cell described in this application may comprise a PKS. As used in this application, a “PKS” refers to an enzyme that is capable of producing a polyketide. In certain embodiments, a PKS converts a compound of Formula (2) to a compound of Formula (4), (5), and/or (6). In certain embodiments, a PKS converts a compound of Formula (2) to a compound of Formula (4). In certain embodiments, a PKS converts a compound of Formula (2) to a compound of Formula (5). In certain embodiments, a PKS converts a compound of Formula (2) to a compound of Formula (4) and/or (5). In certain embodiments, a PKS converts a compound of Formula (2) to a compound of Formula (5) and/or (6).

In some embodiments, a PKS is a tetraketide synthase (TKS). In certain embodiments, a PKS is an olivetol synthase (OLS). As used in this application, an “OLS” refers to an enzyme that is capable of using a substrate of Formula (2a) to form a compound of Formula (4a), (5a) or (6a) as shown in FIG. 1.

In certain embodiments, a PKS is a divarinic acid synthase (DVS).

In certain embodiments, polyketide synthases can use hexanoyl-CoA or any acyl-CoA (or a product of Formula (2):

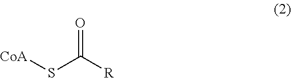

and three malonyl-CoAs as substrates to form 3,5,7-trioxododecanoyl-CoA or other 3,5,7-trioxo-acyl-CoA derivatives; or to form a compound of Formula (4):

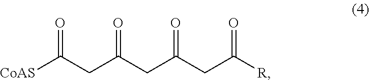

wherein R is hydrogen, optionally substituted acyl, optionally substituted alkyl, optionally substituted alkenyl, optionally substituted alkynyl, optionally substituted carbocyclyl, or optionally substituted aryl; depending on substrate. R is as defined in this application. In some embodiments, R is a C2-C6 optionally substituted alkyl. In some embodiments, R is a propyl or pentyl. In some embodiments, R is pentyl. In some embodiments, R is propyl. A PKS may also bind isovaleryl-CoA, octanoyl-CoA, hexanoyl-CoA, and butyryl-CoA. In some embodiments, a PKS is capable of catalyzing the formation of a 3,5,7-trioxoalkanoyl-CoA (e.g. 3,5,7-trioxododecanoyl-CoA). In some embodiments, an OLS is capable of catalyzing the formation of a 3,5,7-trioxoalkanoyl-CoA (e.g. 3,5,7-trioxododecanoyl-CoA).

In some embodiments, a PKS uses a substrate of Formula (2) to form a compound of Formula (4):

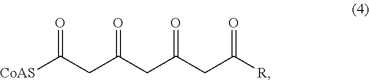

wherein R is unsubstituted pentyl.

As one of ordinary skill in the art would appreciate a PKS, such as an OLS, could be obtained from any source, including naturally occurring sources and synthetic sources (e.g., a non-naturally occurring PKS). In some embodiments a PKS is from Cannabis. In some embodiments, a PKS is from Dictyostelium. In some embodiments, a PKS comprises SEQ ID NO: 195. See, e.g., Example 1. Non-limiting examples of PKS enzymes may be found in U.S. Pat. No. 6,265,633; WO 2018/148848 A1; WO 2018/148849 A1; U.S. Patent Application Publication No. 2018/155748; and WO 2020/176547, which are incorporated by reference in this application in their entireties.

A non-limiting example of an OLS is provided by UniProtKB-B1Q2B6 from C. sativa. In C. sativa, this OLS uses hexanoyl-CoA and malonyl-CoA as substrates to form 3,5,7-trioxododecanoyl-CoA. OLS (e.g., UniProtKB-B1Q2B6) in combination with olivetolic acid cyclase (OAC) produces olivetolic acid (OA) in C. sativa.

The amino acid sequence of UniProtKB-B1Q2B6 is:

(SEQ ID NO: 7)
MNHLRAEGPASVLAIGTANPENILLQDEFPDYYFRVTKSEHMTQLKEKFR
KICDKSMIRKRNCFLNEEHLKQNPRLVEHEMQTLDARQDMLVVEVPKLGK
DACAKAIKEWGQPKSKITHLIFTSASTTDMPGADYHCAKLLGLSPSVKRV
MMYQLGCYGGGTVLRIAKDIAENNKGARVLAVCCDIMACLFRGPSESDLE
LLVGQAIFGDGAAAVIVGAEPDESVGERPIFELVSTGQTILPNSEGTIGG
HIREAGLIFDLHKDVPMLISNNIEKCLIEAFTPIGISDWNSIFWITHPGG
KAILDKVEEKLHLKSDKFVDSRHVLSEHGNMSSSTVLFVMDELRKRSLEE
GKSTTGDGFEWGVLFGFGPGLTVERVVVRSVPIKY.

PKS enzymes described in this application may or may not have cyclase activity. In some embodiments where the PKS enzyme does not have cyclase activity, one or more exogenous polynucleotides that encode a polyketide cyclase (PKC) enzyme may also be co-expressed in the same host cells to enable conversion of hexanoic acid or butyric acid or other fatty acid conversion into olivetolic acid or divarinolic acid or other precursors of cannabinoids. In some embodiments, the PKS enzyme and a PKC enzyme are expressed as separate distinct enzymes. In some embodiments, a PKS enzyme that lacks cyclase activity and a PKC are linked as part of a fusion polypeptide that is a bifunctional PKS. In some embodiments, a bifunctional PKC is referred to as a bifunctional PKS-PKC. In some embodiments, a bifunctional PKC is a bifunctional tetraketide synthase (TKS-TKC). As used in this application, a bifunctional PKS is an enzyme that is capable of producing a compound of Formula (6):

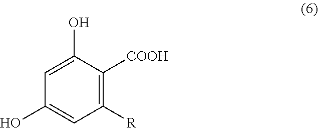

from a compound of Formula (2):

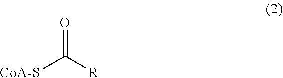

and a compound of Formula (3):

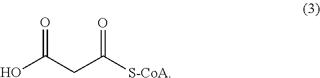

In some embodiments, a PKS produces more of a compound of Formula (6):

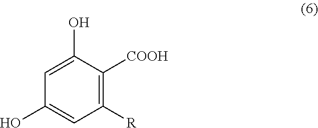

as compared to a compound of Formula (5):

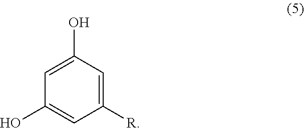

As a non-limiting example, a compound of Formula (6):

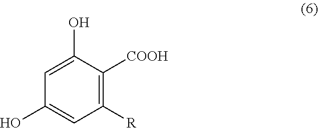

is olivetolic acid (Formula (6a)):

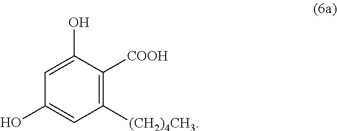

As a non-limiting example, a compound of Formula (5):

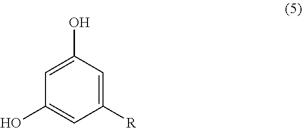

is olivetol (Formula (5a)):

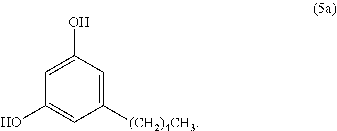

In some embodiments, a polyketide synthase of the present disclosure is capable of catalyzing a compound of Formula (2):

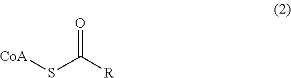

and a compound of Formula (3):

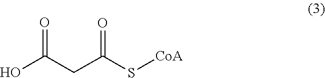

to produce a compound of Formula (4):

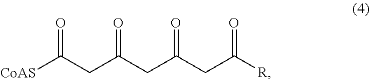

and also further catalyzes a compound of Formula (4):

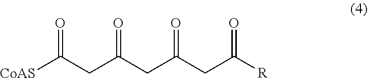

to produce a compound of Formula (6):

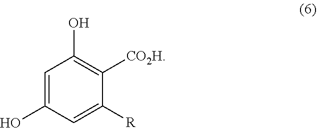

In some embodiments, the PKS is not a fusion protein. In some embodiments, a PKS that is capable of catalyzing a compound of Formula (2):

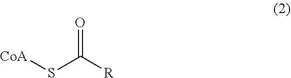

and a compound of Formula (3):

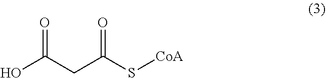

to produce a compound of Formula (4):

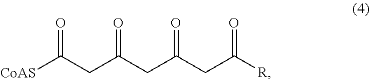

and is also capable of further catalyzing the production of a compound of Formula (6):

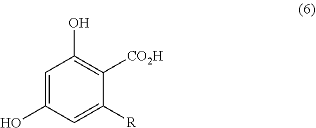

from the compound of Formula (4):

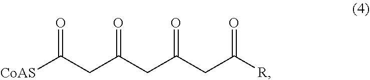

is preferred because it avoids the need for an additional polyketide cyclase to produce a compound of Formula (6):

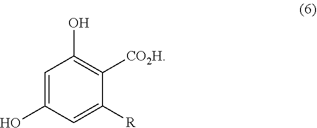

In some embodiments, such an enzyme that is a bifunctional PKS eliminates the transport considerations needed with addition of a polyketide cyclase, whereby the compound of Formula (4), being the product of the PKS, must be transported to the PKS for use as a substrate to be converted into the compound of Formula (6).

In some embodiments, a PKS is capable of producing olivetolic acid in the presence of a compound of Formula (2a):

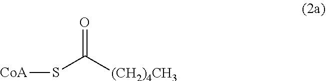

### and Formula (3a):

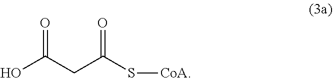

In some embodiments, an OLS is capable of producing olivetolic acid in the presence of a compound of Formula (2a):

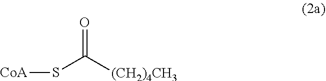

### and Formula (3a):

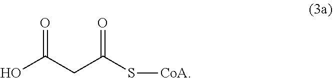

### Prenyltransferase (PT)

A host cell described in this application may comprise a prenyltransferase (PT). As used in this application, a “PT” refers to an enzyme that is capable of transferring prenyl groups to acceptor molecule substrates. Non-limiting examples of prenyltransferases are described in U.S. Pat. No. 7,544,498 and Kumano et al., Bioorg Med Chem. 2008 Sep. 1; 16(17): 8117-8126 (e.g., NphB), PCT Publication No. WO 2018/200888 (e.g., CsPT4), U.S. Pat. No. 8,884,100 (e.g., CsPT1); CA2718469; Valliere et al., Nat Commun. 2019 Feb. 4; 10(1):565 (e.g., NphB variants); PCT Publication Nos: WO2019/173770, WO2019/183152, and WO2020/210810 (e.g., NphB variants); Luo et al., Nature 2019 March;567(7746):123-126 (e.g., CsPT4); WO 2021/034848; U.S. 63/091,292 and U.S. 63/188,442 (e.g., CsPT variants and chimeras), which are incorporated by reference in their entireties. In some embodiments, a PT is capable of producing cannabigerolic acid (CBGA), cannabigerophorolic acid (CBGPA), cannabigerovarinic acid (CBGVA), a CBG-type cannabinoid, or other cannabinoids or cannabinoid-like substances. In some embodiments, a PT is cannabigerolic acid synthase (CBGAS). In some embodiments, a PT is cannabigerovarinic acid synthase (CBGVAS).

In some embodiments, the PT is an NphB prenyltransferase. See, e.g., U.S. Pat. No. 7,544,498; and Kumano et al., Bioorg Med Chem. 2008 Sep. 1; 16(17): 8117-8126, which are incorporated by reference in this application in their entireties. In some embodiments, a PT corresponds to NphB from Streptomyces sp. (see, e.g., UniprotKB Accession No. Q4R2T2; see also SEQ ID NO: 2 of U.S. Pat. No. 7,361,483). The protein sequence corresponding to UniprotKB Accession No. Q4R2T2 is provided by SEQ ID NO: 8:

(SEQ ID NO: 8)
MSEAADVERVYAAMEEAAGLLGVACARDKIYPLLSTFQDTLVEGGSVVVF
SMASGRHSTELDFSISVPTSHGDPYATVVEKGLFPATGHPVDDLLADTQK
HLPVSMFAIDGEVTGGFKKTYAFFPTDNMPGVAELSAIPSMPPAVAENAE
LFARYGLDKVQMTSMDYKKRQVNLYFSELSAQTLEAESVLALVRELGLHV
PNELGLKFCKRSFSVYPTLNWETGKIDRLCFAVISNDPTLVPSSDEGDIE
KFHNYATKAPYAYVGEKRTLVYGLTLSPKEEYYKLGAYYHITDVQRGLLK
AFDSLED.

A non-limiting example of a nucleic acid sequence encoding NphB is:

(SED ID NO: 9)
atgtcagaagccgcagatgtcgaaagagtttacgccgctatggaagaagc
cgccggtttgttaggtgttgcctgtgccagagataagatctacccattgt
tgtctacttttcaagatacattagttgaaggtggttcagttgttgttttc
tctatggcttcaggtagacattctacagaattggatttctctatctcagt
tccaacatcacatggtgatccatacgctactgttgttgaaaaaggtttat
ttccagcaacaggtcatccagttgatgatttgttggctgatactcaaaag
catttgccagtttctatgtttgcaattgatggtgaagttactggtggttt
caagaaaacttacgctttctttccaactgataacatgccaggtgttgcag
aattatctgctattccatcaatgccaccagctgttgcagaaaatgcagaa
ttatttgctagatacggtttggataaggttcaaatgacatctatggatta
caagaaaagacaagttaatttgtacttttctgaattatcagcacaaactt
tggaagctgaatcagttttggcattagttagagaattgggtttacatgtt
ccaaacgaattgggtttgaagttttgtaaaagatctttctcagtttatcc
aactttaaactgggaaacaggcaagatcgatagattatgtttcgcagtta
tctctaacgatccaacattggttccatcttcagatgaaggtgatatcgaa
aagtttcataactacgctactaaagcaccatatgcttacgttggtgaaaa
gagaacattagtttatggtttgactttatcaccaaaggaagaatactaca
agttgggtgcttactaccacattaccgacgtacaaagaggtttattgaaa
gcattcgatagtttagaagactaa.

In other embodiments, a PT corresponds to CsPT1, which is disclosed as SEQ ID NO:2 in U.S. Pat. No. 8,884,100 (C. sativa; corresponding to SEQ ID NO: 10 in this application):

(SEQ ID NO: 10)
MGLSSVCTFSFQTNYHTLLNPHNNNPKTSLLCYRHPKTPIKYSYNNFPSK
HCSTKSFHLQNKCSESLSIAKNSIRAATTNQTEPPESDNHSVATKILNFG
KACWKLQRPYTIIAFTSCACGLFGKELLHNTNLISWSLMFKAFFFLVAIL
CIASFTTTINQIYDLHIDRINKPDLPLASGEISVNTAWIMSIIVALFGLI
ITIKMKGGPLYIFGYCFGIFGGIVYSVPPFRWKQNPSTAFLLNFLAHIIT
NFTFYYASRAALGLPFELRPSFTFLLAFMKSMGSALALIKDASDVEGDTK
FGISTLASKYGSRNLTLFCSGIVLLSYVAAILAGIIWPQAFNSNVMLLSH
AILAFWLILQTRDFALTNYDPEAGRRFYEFMWKLYYAEYLVYVFI.

In some embodiments, a PT corresponds to CsPT4, which is disclosed as SEQ ID NO:1 in WO 2019/071000, corresponding to SEQ ID NO: 11 in this application:

(SEQ ID NO: 11)
MGLSLVCTFSFQTNYHTLLNPHNKNPKNSLLSYQHPKTPIIKSSYDNFPS
KYCLTKNFHLLGLNSHNRISSQSRSIRAGSDQIEGSPHHESDNSIATKIL
NFGHTCWKLQRPYVVKGMISIACGLFGRELFNNRHLFSWGLMWKAFFALV
PILSFNFFAAIMNQIYDVDIDRINKPDLPLVSGEMSIETAWILSIIVALT
GLIVTIKLKSAPLFVFIYIFGIFAGFAYSVPPIRWKQYPFTNFLITISSH
VGLAFTSYSATTSALGLPFVWRPAFSFIIAFMTVMGMTIAFAKDISDIEG
DAKYGVSTVATKLGARNMTFVVSGVLLLNYLVSISIGIIWPQVFKSNIMI
LSHAILAFCLIFQTRELALANYASAPSRQFFEFIWLLYYAEYFVYVFI.

In some embodiments, a PT corresponds to a truncated CsPT4, which is provided as SEQ ID NO: 12 herein:

MSAGSDQIEGSPHHESDNSIATKILNFGHTCWKLQRPYVVKGMISIACGL
FGRELFNNRHLFSWGLMWKAFFALVPILSFNFFAAIMNQIYDVDIDRINK
PDLPLVSGEMSIETAWILSIIVALTGLIVTIKLKSAPLFVFIYIFGIFAG
FAYSVPPIRWKQYPFTNFLITISSHVGLAFTSYSATTSALGLPFVWRPAF
SFIIAFMTVMGMTIAFAKDISDIEGDAKYGVSTVATKLGARNMTFVVSGV
LLLNYLVSISIGIIWPQVFKSNIMILSHAILAFCLIFQTRELALANYASA
PSRQFFEFIWLLYYAEYFVYVFI.

Functional expression of paralog C. sativa CBGAS enzymes in S. cerevisiae and production of the major cannabinoid CBGA has been reported (Page and Boubakir U.S. Patent Application Publication No. 2012/0144523, 2012, and Luo et al. Nature, 2019). Luo et al. reported the production of CBGA in S. cerevisiae by expressing a truncated version of a C. sativa CBGAS, CsPT4, with its native signal peptide removed (Luo et al. Nature, 2019). Without being bound by a particular theory, the integral-membrane nature of C. sativa CBGAS enzymes may render functional expression of C. sativa CBGAS enzymes in heterologous hosts challenging. Removal of transmembrane domain(s) or signal sequences or use of prenyltransferases that are not associated with the membrane and are not integral membrane proteins may facilitate increased interaction between the enzyme and available substrate, for example in the cellular cytosol and/or in organelles that may be targeted using peptides that confer localization.

In some embodiments, the PT is a soluble PT. In some embodiments, the PT is a cytosolic PT. In some embodiments, the PT is a secreted protein. In some embodiments, the PT is not a membrane-associated protein. In some embodiments, the PT is not an integral membrane protein. In some embodiments, the PT does not comprise a transmembrane domain or a predicted transmembrane. In some embodiments, the PT may be primarily detected in the cytosol (e.g., detected in the cytosol to a greater extent than detected associated with the cell membrane). In some embodiments, the PT is a protein from which one or more transmembrane domains have been removed and/or mutated (e.g., by truncation, deletions, substitutions, insertions, and/or additions) so that the PT localizes or is predicted to localize in the cytosol of the host cell, or to cytosolic organelles within the host cell, or, in the case of bacterial hosts, in the periplasm. In some embodiments, the PT is a protein from which one or more transmembrane domains have been removed or mutated (e.g., by truncation, deletions, substitutions, insertions, and/or additions) so that the PT has increased localization to the cytosol, organelles, or periplasm of the host cell, as compared to membrane localization.

Within the scope of the term “transmembrane domains” are predicted or putative transmembrane domains in addition to transmembrane domains that have been empirically determined. In general, transmembrane domains are characterized by a region of hydrophobicity that facilitates integration into the cell membrane. Methods of predicting whether a protein is a membrane protein or a membrane-associated protein are known in the art and may include, for example amino acid sequence analysis, hydropathy plots, and/or protein localization assays.

In some embodiments, the PT is a protein from which a signal sequence has been removed and/or mutated so that the PT is not directed to the cellular secretory pathway. In some embodiments, the PT is a protein from which a signal sequence has been removed and/or mutated so that the PT is localized to the cytosol or has increased localization to the cytosol (e.g., as compared to the secretory pathway).

In some embodiments, the PT is a secreted protein. In some embodiments, the PT contains a signal sequence.

In some embodiments, a PT is a fusion protein. For example, a PT may be fused to one or more genes in the metabolic pathway of a host cell. In certain embodiments, a PT may be fused to mutant forms of one or more genes in the metabolic pathway of a host cell.

In some embodiments, a PT described in this application transfers one or more prenyl groups to any of positions 1, 2, 3, 4, or 5 in a compound of Formula (6), shown below:

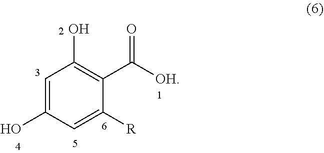

In some embodiments, the PT transfers a prenyl group to any of positions 1, 2, 3, 4, or 5 in a compound of Formula (6), shown below:

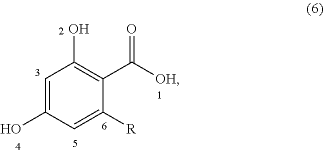

to form a compound of one or more of Formula (8w), Formula (8x), Formula (8′), Formula (8y), Formula (8z):

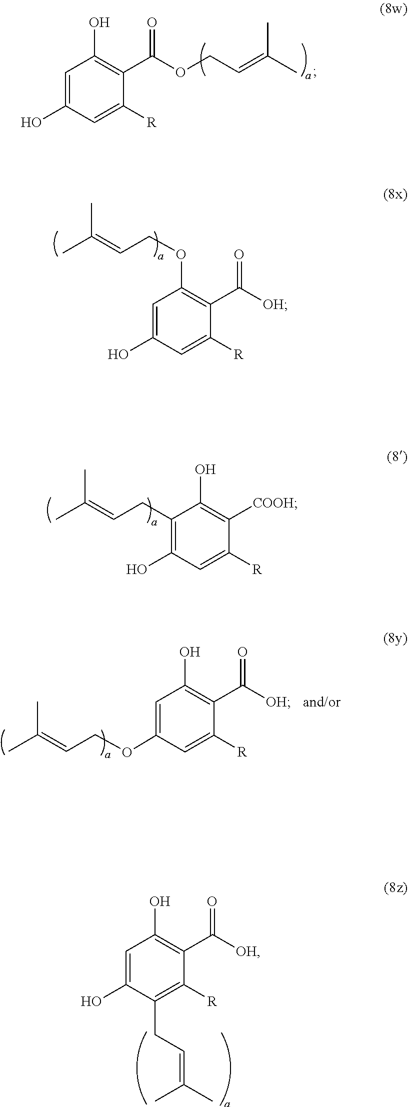

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

### Terminal Synthases (TS)

A host cell described in this application may comprise a terminal synthase (TS). As used in this application, a “TS” refers to an enzyme that is capable of catalyzing oxidative cyclization of a prenyl moiety (e.g., terpene) to produce a ring-containing product (e.g., heterocyclic ring-containing product). In certain embodiments, a TS is capable of catalyzing oxidative cyclization of a prenyl moiety (e.g., terpene) to produce a carbocyclic-ring containing product (e.g., cannabinoid). In certain embodiments, a TS is capable of catalyzing oxidative cyclization of a prenyl moiety (e.g., terpene) to produce a heterocyclic-ring containing product (e.g., cannabinoid). In certain embodiments, a TS is capable of catalyzing oxidative cyclization of a prenyl moiety (e.g., terpene) to produce a cannabinoid. In some embodiments, a terminal synthase is a terpene cyclase that uses a terpenophenolic compound as a substrate.

In some embodiments, a TS is a tetrahydrocannabinolic acid synthase (THCAS), a cannabidiolic acid synthase (CBDAS), and/or a cannabichromenic acid synthase (CBCAS). As one of ordinary skill in the art would appreciate a TS could be obtained from any source, including naturally occurring sources and synthetic sources (e.g., a non-naturally occurring TS).

a. Substrates

A TS may be capable of using one or more substrates. In some instances, the location of the prenyl group and/or the R group differs between TS substrates. For example, a TS may be capable of using as a substrate one or more compounds of Formula (8w), Formula (8x), Formula (8′), Formula (8y), and/or Formula (8z):

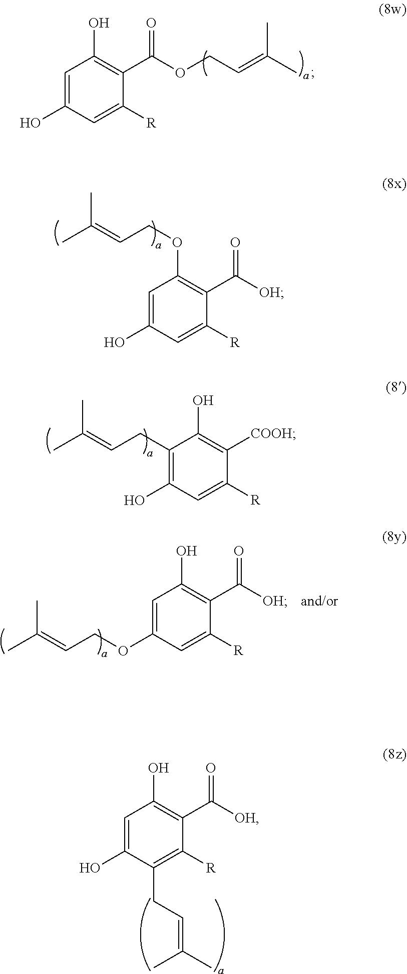

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

In certain embodiments, a compound of Formula (8′) is a compound of Formula

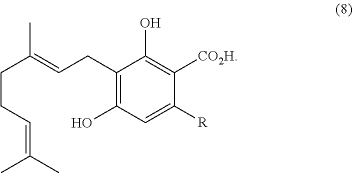

In some embodiments, a TS catalyzes oxidative cyclization of the prenyl moiety (e.g., terpene) of a compound of Formula (8) described in this application and shown in FIG. 2. In certain embodiments, a compound of Formula (8) is a compound of Formula (8a):

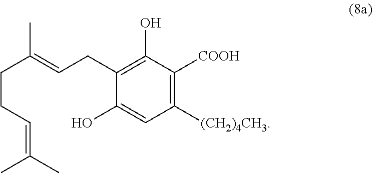

b. Products

In embodiments wherein CBGA is the substrate, the TS enzymes CBDAS, THCAS and CBCAS would generally catalyze the formation of cannabidiolic acid (CBDA), A9-tetrahydrocannabinolic acid (THCA) and cannabichromenic acid (CBCA), respectively. However, in some embodiments, a TS can produce more than one different product depending on reaction conditions. For example, the pH of the reaction environment may cause a THCAS or a CBDAS to produce CBCA in greater proportions than THCA or CBDAS, respectively (see, for example, U.S. Pat. No. 9,359,625 to Winnicki and Donsky, incorporated by reference in its entirety). In some embodiments, a TS has a predetermined product specificity in intracellular conditions, such as cytosolic conditions or organelle conditions. By expressing a TS with a predetermined product specificity based on intracellular conditions, in vivo products produced by a cell expressing the TS may be more predictably produced. In some embodiments, a TS produces a desired product at a pH of 5.5. In some embodiments, a TS produces a desired product at a pH of 1, 2, 3, 4, 5, 6, 7, 8, 9, 10, 11, 12, 13 or 14. In some embodiments, a TS produces a desired product at a pH that is between 4.5 and 8.0. In some embodiments, a TS produces a desired product at a pH that is between 5 and 6. In some embodiments, a TS produces a desired product at a pH that is around 4.5, 4.6, 4.7, 4.8, 4.9, 5.0, 5,1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, including all values in between. In some embodiments, the product profile of a TS is dependent on the TS's signal peptide because the signal peptide targets the TS to a particular intracellular location having particular intracellular conditions (e.g. a particular organelle) that regulate the type of product produced by the TS.

A TS may be capable of using one or more substrates described in this application to produce one or more products. Non-limiting example of TS products are shown in Table 1. In some instances, a TS is capable of using one substrate to produce 1, 2, 3, 4, 5, 6, 7, 8, 9, or 10 different products. In some embodiments, a TS is capable of using more than one substrate to produce 1, 2, 3, 4, 5, 6, 7, 8, 9, or 10 different products.

In some embodiments, a TS is capable of producing a compound of Formula (X-A) and/or a compound of Formula (X-B):

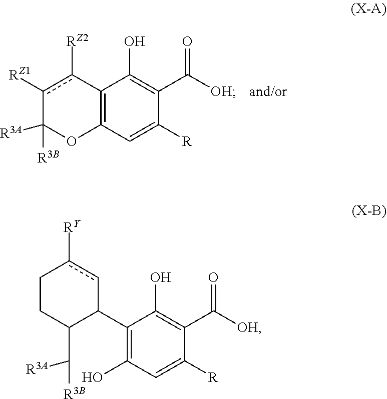

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;
R is hydrogen, optionally substituted acyl, optionally substituted alkyl, optionally substituted alkenyl, optionally substituted alkynyl, optionally substituted carbocyclyl, or optionally substituted aryl;
R^{Z1} is hydrogen, optionally substituted acyl, optionally substituted alkyl, optionally substituted alkenyl, optionally substituted alkynyl, optionally substituted carbocyclyl, or optionally substituted aryl;
R^{Z2} is hydrogen, optionally substituted acyl, optionally substituted alkyl, optionally substituted alkenyl, optionally substituted alkynyl, optionally substituted carbocyclyl, or optionally substituted aryl;
or optionally, R^{Z1} and R^{Z2} are taken together with their intervening atoms to form an optionally substituted carbocyclic ring;
R^{3A} is hydrogen, optionally substituted acyl, optionally substituted alkyl, optionally substituted alkenyl, or optionally substituted alkynyl;
R^{3B} is hydrogen, optionally substituted acyl, optionally substituted alkyl, optionally substituted alkenyl, or optionally substituted alkynyl; and/or
R^{Y} is hydrogen, optionally substituted acyl, optionally substituted alkyl, optionally substituted alkenyl, or optionally substituted alkynyl.

In some embodiments, a compound of Formula (X-A) is:

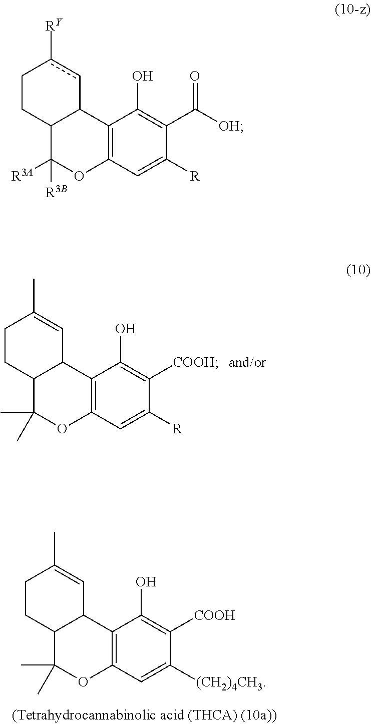

In certain embodiments, a compound of Formula (10)

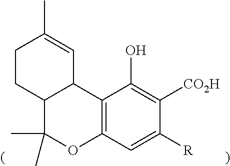

has a chiral atom labeled with * at carbon 10 and a chiral atom labeled with ** at carbon 6. In certain embodiments, in a compound of Formula (10)

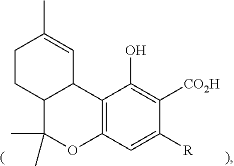

the chiral atom labeled with * at carbon 10 is of the R-configuration or S-configuration; and a chiral atom labeled with ** at carbon 6 is of the R-configuration. In certain embodiments, in a compound of Formula (10)

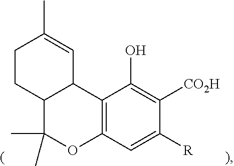

the chiral atom labeled with * at carbon 10 is of the S-configuration; and a chiral atom labeled with ** at carbon 6 is of the R-configuration or S-configuration. In certain embodiments, in a compound of Formula (10)

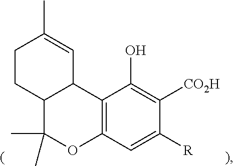

the chiral atom labeled with * at carbon 10 is of the R-configuration and a chiral atom labeled with ** at carbon 6 is of the R-configuration. In certain embodiments, a compound of Formula (10)

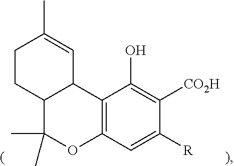

is of the formula:

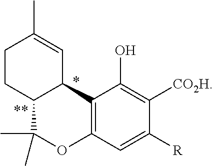

In certain embodiments, in a compound of Formula (10)

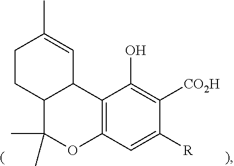

the chiral atom labeled with * at carbon 10 is of the S-configuration and a chiral atom labeled with ** at carbon 6 is of the S-configuration. In certain embodiments, a compound of Formula (10)

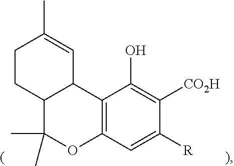

is of the formula:

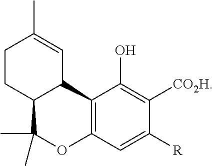

In certain embodiments, a compound of Formula (10a)

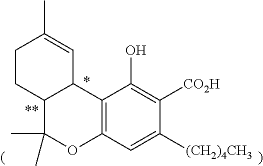

has a chiral atom labeled with * at carbon 10 and a chiral atom labeled with ** at carbon 6. In certain embodiments, in a compound of Formula (10a)

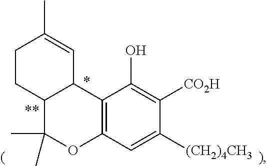

the chiral atom labeled with * at carbon 10 is of the R-configuration or S-configuration; and a chiral atom labeled with ** at carbon 6 is of the R-configuration. In certain embodiments, in a compound of Formula (10a)

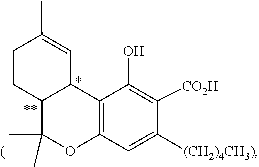

the chiral atom labeled with * at carbon 10 is of the S-configuration; and a chiral atom labeled with ** at carbon 6 is of the R-configuration or S-configuration. In certain embodiments, in a compound of Formula (10a)

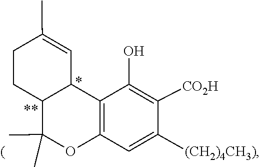

the chiral atom labeled with * at carbon 10 is of the R-configuration and a chiral atom labeled with ** at carbon 6 is of the R-configuration. In certain embodiments, a compound of Formula (10a)

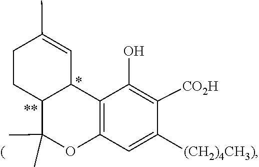

is of the formula:

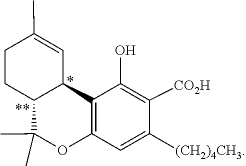

In certain embodiments, in a compound of Formula (10a)

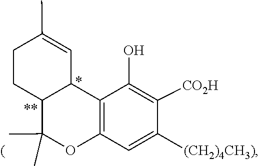

the chiral atom labeled with * at carbon 10 is of the S-configuration and a chiral atom labeled with ** at carbon 6 is of the S-configuration. In certain embodiments, a compound of Formula (10a)

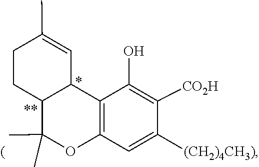

is of the formula:

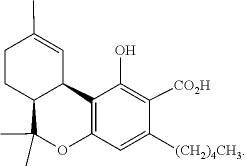

In some embodiments, a compound of Formula (X-A) is:

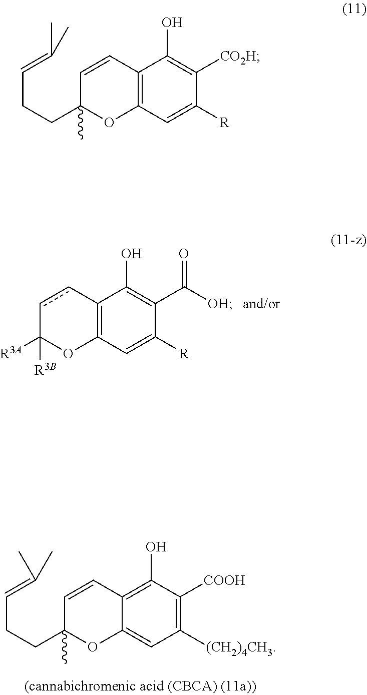

In some embodiments, a compound of Formula (X-A) is:

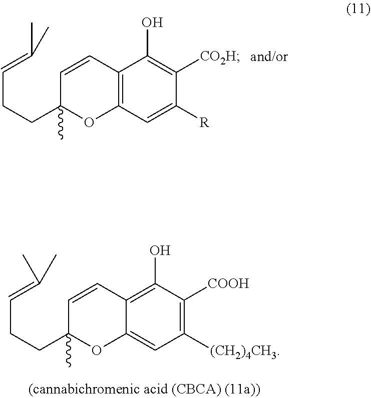

In some embodiments a compound of Formula (X-B) is:

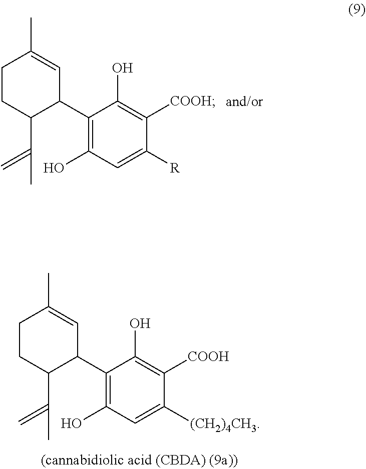

In certain embodiments, a compound of Formula (9)

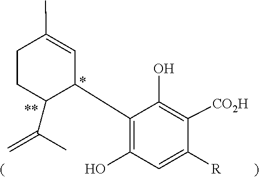

has a chiral atom labeled with * at carbon 3 and a chiral atom labeled with ** at carbon 4. In certain embodiments, in a compound of Formula (9)

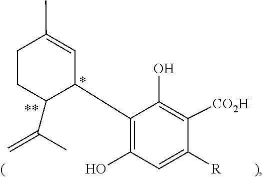

the chiral atom labeled with * at carbon 3 is of the R-configuration or S-configuration; and a chiral atom labeled with ** at carbon 4 is of the R-configuration. In certain embodiments, in a compound of Formula (9)

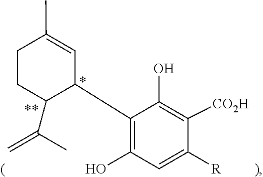

the chiral atom labeled with * at carbon 3 is of the S-configuration; and a chiral atom labeled with ** at carbon 4 is of the R-configuration or S-configuration. In certain embodiments, in a compound of Formula (9)

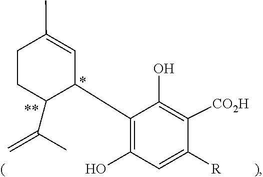

the chiral atom labeled with * at carbon 3 is of the R-configuration and a chiral atom labeled with ** at carbon 4 is of the R-configuration. In certain embodiments, a compound of Formula (9)

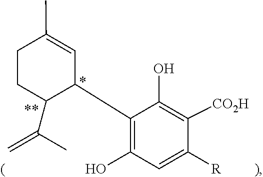

is of the formula:

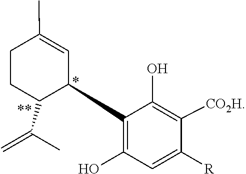

In certain embodiments, in a compound of Formula (9)

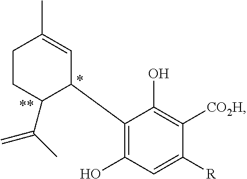

the chiral atom labeled with * at carbon 3 is of the S-configuration and a chiral atom labeled with ** at carbon 4 is of the S-configuration. In certain embodiments, a compound of Formula (9)

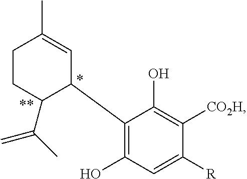

is of the formula:

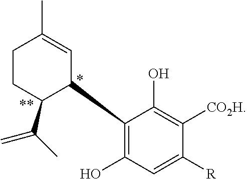

In certain embodiments, a compound of Formula (9a) (CBDA)

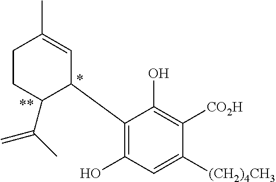

has a chiral atom labeled with * at carbon 3 and a chiral atom labeled with ** at carbon 4. In certain embodiments, in a compound of Formula (9a)

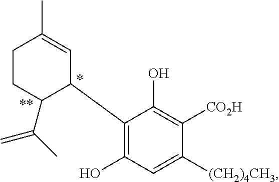

the chiral atom labeled with * at carbon 3 is of the R-configuration or S-configuration; and a chiral atom labeled with ** at carbon 4 is of the R-configuration. In certain embodiments, in a compound of Formula (9a)

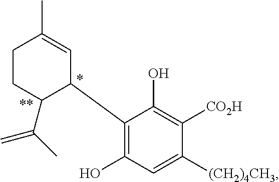

the chiral atom labeled with * at carbon 3 is of the S-configuration; and a chiral atom labeled with ** at carbon 4 is of the R-configuration or S-configuration. In certain embodiments, in a compound of Formula (9a)

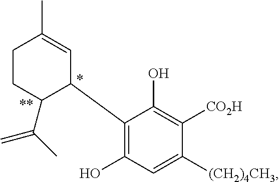

the chiral atom labeled with * at carbon 3 is of the R-configuration and a chiral atom labeled with ** at carbon 4 is of the R-configuration. In certain embodiments, a compound of Formula (9a)

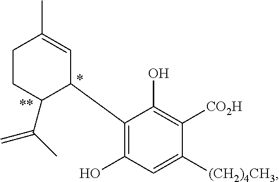

is of the formula:

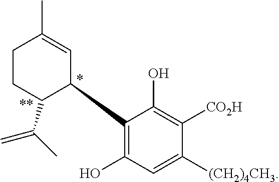

In certain embodiments, in a compound of Formula (9a)

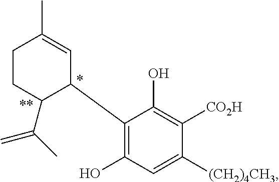

the chiral atom labeled with * at carbon 3 is of the S-configuration and a chiral atom labeled with ** at carbon 4 is of the S-configuration. In certain embodiments, a compound of Formula (9a)

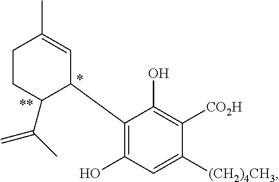

is of the formula:

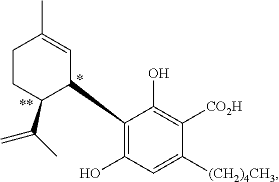

In some embodiments, as shown in FIG. 2, a TS is capable of producing a cannabinoid from the product of a PT, including, without limitation, an enzyme capable of producing a compound of Formula (9), (10), or (11):

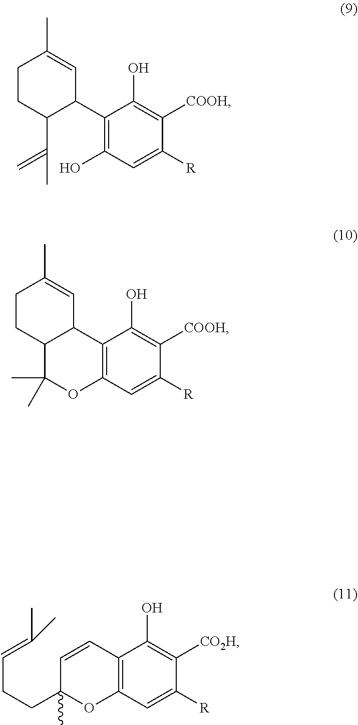

or a pharmaceutically acceptable salt, solvate, hydrate, polymorph, co-crystal, tautomer, stereoisomer, isotopically labeled derivative, or prodrug thereof, wherein R is hydrogen, optionally substituted acyl, optionally substituted alkyl, optionally substituted alkenyl, optionally substituted alkynyl, optionally substituted carbocyclyl, or optionally substituted aryl; produced from a compound of Formula (8′):

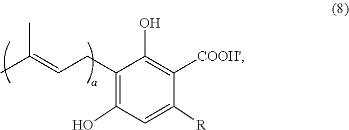

wherein a is 1, 2, 3, 4, 5, 6, 7, 8, 9, or 10; and R is hydrogen, optionally substituted acyl, optionally substituted alkyl, optionally substituted alkenyl, optionally substituted alkynyl, optionally substituted carbocyclyl, or optionally substituted aryl; or using any other substrate. In certain embodiments, a compound of Formula (8′) is a compound of Formula (8):

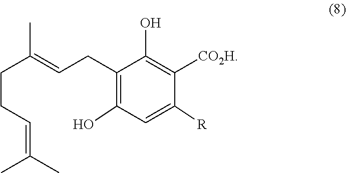

In certain embodiments, a compound of Formula (9), (10), or (11) is produced using a TS from a substrate compound of Formula (8′) (e.g., compound of Formula (8)), for example. Non-limiting examples of substrate compounds of Formula (8′) include but are not limited to cannabigerolic acid (CBGA), cannabigerovarinic acid (CBGVA), or cannabinerolic acid. In certain embodiments, at least one of the hydroxyl groups of the product compounds of Formula (9), (10), or (11) is further methylated. In certain embodiments, a compound of Formula (9) is methylated to form a compound of Formula (12):

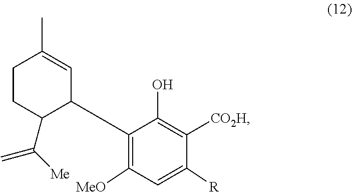

or a pharmaceutically acceptable salt, solvate, hydrate, polymorph, co-crystal, tautomer, stereoisomer, isotopically labeled derivative, or prodrug thereof.

### Tetrahydrocannabinolic Acid Synthase (THCAS)

A host cell described in this application may comprise a TS that is a tetrahydrocannabinolic acid synthase (THCAS). As used in this application “tetrahydrocannabinolic acid synthase (THCAS)” or “Δ¹-tetrahydrocannabinolic acid (THCA) synthase” refers to an enzyme that is capable of catalyzing oxidative cyclization of a prenyl moiety (e.g., terpene) of a compound of Formula (8) to produce a ring-containing product (e.g., heterocyclic ring-containing product, carbocyclic-ring containing product) of Formula (10). In certain embodiments, a THCAS refers to an enzyme that is capable of producing Δ9-tetrahydrocannabinolic acid (Δ9-THCA, THCA, Δ9-Tetrahydro-cannabivarinic acid A (Δ9-THCVA-C3 A), THCVA, THCP, or a compound of Formula 10(a), from a compound of Formula (8). In certain embodiments, a THCAS is capable of producing Δ⁹-tetrahydrocannabinolic acid (Δ⁹-THCA, THCA, or a compound of Formula 10(a)). In certain embodiments, a THCAS is capable of producing Δ9-tetrahydrocannabivarinic acid (Δ9-THCVA, THCVA, or a compound of Formula 10 where R is n-propyl).

In some embodiments, a THCAS may catalyze the oxidative cyclization of substrates, such as 3-prenyl-2,4-dihydroxy-6-alkylbenzoic acids. In some embodiments, a THCAS may use cannabigerolic acid (CBGA) as a substrate. In some embodiments, the THCAS produces A9-THCA from CBGA. In some embodiments, a THCAS may catalyze the oxidative cyclization of cannabigerovarinic acid (CBGVA). In some embodiments, a THCAS exhibits specificity for CBGA substrates as compared to other substrates. In some embodiments, a THCAS may use a compound of Formula (8) of FIG. 2 where R is C4 alkyl (e.g., n-butyl) or R is C7 alkyl (e.g., n-heptyl) as a substrate. In some embodiments, a THCAS may use a compound of Formula (8) where R is C4 alkyl (e.g., n-butyl) as a substrate. In some embodiments, a THCAS may use a compound of Formula (8) of FIG. 2 where R is C7 alkyl (e.g., n-heptyl) as a substrate. In some embodiments, the THCAS exhibits specificity for substrates that can result in THCP as a product.

In some embodiments, a THCAS is from C. sativa. C. sativa THCAS performs the oxidative cyclization of the geranyl moiety of Cannabigerolic Acid (CBGA) (FIG. 4 Structure 8a) to form Tetrahydrocannabinolic Acid (FIG. 4 Structure 10a) using covalently bound flavin adenine dinucleotide (FAD) as a cofactor and molecular oxygen as the final electron acceptor. THCAS was first discovered and characterized by Taura et al. (JACS. 1995) following extraction of the enzyme from the leaf buds of C. sativa and confirmation of its THCA synthase activity in vitro upon the addition of CBGA as a substrate. Additional analysis indicated that the enzyme is a monomer and possesses FAD binding and Berberine Bridge Enzyme (BBE) sequence motifs. A crystal structure of the enzyme published by Shoyama et al. (J Mol Biol. 2012 Oct. 12; 423(1):96-105) revealed that the enzyme covalently binds to a molecule of the cofactor FAD. See also, e.g., Sirikantarams et al., J. Biol. Chem. 2004 September 17; 279(38):39767-39774. There are several THCAS isozymes in Cannabis sativa.

In some embodiments, a C. sativa THCAS (Uniprot KB Accession No.: I1V0C5) comprises the amino acid sequence shown below, in which the signal peptide is underlined and bolded:

(SEQ ID NO: 13)
MNCSAFSFWFVCKIIFFFLSFNIQISIANPQENFLKCFSEYIPNNPANPK
FIYTQHDQLYMSVLNSTIQNLRFTSDTTPKPLVIVTPSNVSHIQASILCS
KKVGLQIRTRSGGHDAEGMSYISQVPFVVVDLRNMHSIKIDVHSQTAWVE
AGATLGEVYYWINEKNENFSFPGGYCPTVGVGGHFSGGGYGALMRNYGLA
ADNIIDAHLVNVDGKVLDRKSMGEDLFWAIRGGGGENFGIIAAWKIKLVA
VPSKSTIFSVKKNMEIHGLVKLFNKWQNIAYKYDKDLVLMTHFITKNITD
NHGKNKTTVHGYFSSIFHGGVDSLVDLMNKSFPELGIKKTDCKEFSWIDT
TIFYSGVVNFNTANFKKEILLDRSAGKKTAFSIKLDYVKKPIPETAMVKI
LEKLYEEDVGVGMYVLYPYGGIMEEISESAIPFPHRAGIMYELWYTASWE
KQEDNEKHINWVRSVYNFTTPYVSQNPRLAYLNYRDLDLGKTNPESPNNY
TQARIWGEKYFGKNFNRLVKVKTKADPNNFFRNEQSIPPLPPHHH.

In some embodiments, a THCAS comprises the sequence shown below:

(SEQ ID NO: 14)
NPQENFLKCFSEYIPNNPANPKFIYTQHDQLYMSVLNSTIQNLRFTSDTT
PKPLVIVTPSNVSHIQASILCSKKVGLQIRTRSGGHDAEGMSYISQVPFV
VVDLRNMHSIKIDVHSQTAWVEAGATLGEVYYWINEKNENFSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLFNKWQN
IAYKYDKDLVLMTHFITKNITDNHGKNKTTVHGYFSSIFHGGVDSLVDLM
NKSFPELGIKKTDCKEFSWIDTTIFYSGVVNFNTANFKKEILLDRSAGKK
TAFSIKLDYVKKPIPETAMVKILEKLYEEDVGVGMYVLYPYGGIMEEISE
SAIPFPHRAGIMYELWYTASWEKQEDNEKHINWVRSVYNFTTPYVSQNPR
LAYLNYRDLDLGKTNPESPNNYTQARIWGEKYFGKNFNRLVKVKTKADPN
NFFRNEQSIPPLPPHHH.

A non-limiting example of a nucleotide sequence encoding SEQ ID NO: 14 is:

(SEQ ID NO: 15)
aacccgcaagaaaactttctaaaatgcttttctgaatacattcctaacaa
ccctgccaacccgaagtttatctacacacaacacgatcaattgtatatga
gcgtgttgaatagtacaatacagaacctgaggtttacatccgacacaacg
ccgaaaccgctagtgatcgtcacaccctccaacgtaagccacattcaggc
aagcattttatgcagcaagaaagtcggactgcagataaggacgaggtccg
gaggacacgacgccgaagggatgagctatatctcccaggtaccttttgtg
gtggtagacttgagaaatatgcactctatcaagatagacgttcactccca
aaccgcttgggttgaggcgggagccacccttggtgaggtctactactgga
tcaacgaaaagaatgaaaattttagctttcctgggggatattgcccaact
gtaggtgttggcggccacttctcaggaggcggttatggggccttgatgcg
taactacggacttgcggccgacaacattatagacgcacatctagtgaatg
tagacggcaaagttttagacaggaagagcatgggtgaggatcttttttgg
gcaattagaggcggagggggagaaaattttggaattatcgctgcttggaa
aattaagctagttgcggtaccgagcaaaagcactatattctctgtaaaaa
agaacatggagatacatggtttggtgaagctttttaataagtggcaaaac
atcgcgtacaagtacgacaaagatctggttctgatgacgcattttataac
gaaaaatatcaccgacaaccacggaaaaaacaaaaccacagtacatggct
acttctctagtatatttcatgggggagtcgattctctggttgatttaatg
aacaaatcattcccagagttgggtataaagaagacagactgtaaggagtt
ctcttggattgacacaactatattctattcaggcgtagtcaactttaaca
cggcgaatttcaaaaaagagatccttctggacagatccgcaggtaagaaa
actgcgttctctatcaaattggactatgtgaagaagcctattcccgaaac
cgcgatggtcaagatacttgagaaattatacgaggaagatgtgggagttg
gaatgtacgtactttatccctatggtgggataatggaagaaatcagcgag
agcgccattccatttccccatcgtgccggcatcatgtacgagctgtggta
tactgcgagttgggagaagcaagaagacaacgaaaagcacattaactggg
tcagatcagtttacaatttcaccaccccatacgtgtcccagaatccgcgt
ctggcttacttgaactaccgtgatcttgacctgggtaaaacgaacccgga
gtcacccaacaattacactcaagctagaatctggggagagaaatactttg
ggaagaacttcaacaggttagtaaaggttaaaaccaaggcagatccaaac
aacttttttagaaatgaacaatccattcccccgctacccccgcaccatca
c.

In some embodiments, a C. sativa THCAS comprises the amino acid sequence set forth in UniProtKB-Q8GTB6 (SEQ ID NO: 16):

MNCSAFSFWFVCKIIFFFLSFHIQISIANPRENFLKCFSKHIPNNVANPK
LVYTQHDQLYMSILNSTIQNLRFISDTTPKPLVIVTPSNNSHIQATILCS
KKVGLQIRTRSGGHDAEGMSYISQVPFVVVDLRNMHSIKIDVHSQTAWVE
AGATLGEVYYWINEKNENLSFPGGYCPTVGVGGHFSGGGYGALMRNYGLA
ADNIIDAHLVNVDGKVLDRKSMGEDLFWAIRGGGGENFGIIAAWKIKLVA
VPSKSTIFSVKKNMEIHGLVKLFNKWQNIAYKYDKDLVLMTHFITKNITD
NHGKNKTTVHGYFSSIFHGGVDSLVDLMNKSFPELGIKKTDCKEFSWIDT
TIFYSGVVNFNTANFKKEILLDRSAGKKTAFSIKLDYVKKPIPETAMVKI
LEKLYEEDVGAGMYVLYPYGGIMEEISESAIPFPHRAGIMYELWYTASWE
KQEDNEKHINWVRSVYNFTTPYVSQNPRLAYLNYRDLDLGKTNHASPNNY
TQARIWGEKYFGKNFNRLVKVKTKVDPNNFFRNEQSIPPLPPHHH.

Additional non-limiting examples of THCAS enzymes may also be found in U.S. Pat. No. 9,512,391 and U.S. Patent Application Publication No. 2018/0179564, which are incorporated by reference in this application in their entireties.

Cannabidiolic acid synthase (CBDAS)

A host cell described in this application may comprise a TS that is a cannabidiolic acid synthase (CBDAS). As used in this application, a “CBDAS” refers to an enzyme that is capable of catalyzing oxidative cyclization of a prenyl moiety (e.g., terpene) of a compound of Formula (8) to produce a compound of Formula (9). In some embodiments, a compound of Formula 9 is a compound of Formula (9a) (cannabidiolic acid (CBDA)), CBDVA, or CBDP. A CBDAS may use cannabigerolic acid (CBGA) or cannabinerolic acid as a substrate. In some embodiments, a cannabidiolic acid synthase is capable of oxidative cyclization of cannabigerolic acid (CBGA) to produce cannabidiolic acid (CBDA). In some embodiments, the CBDAS may catalyze the oxidative cyclization of other substrates, such as 3-geranyl-2,4-dihydro-6-alkylbenzoic acids like cannabigerovarinic acid (CBGVA) or a substrate of Formula (8) with R as a C7 alkyl (heptyl) group (cannabigerophorolic acid (CBGPA)). In some embodiments, the CBDAS exhibits specificity for CBGA substrates.

In some embodiments, a CBDAS is from Cannabis. In C. sativa, CBDAS is encoded by the CBDAS gene and is a flavoenzyme. A non-limiting example of a CBDAS is provided by UniProtKB-A6P6V9 (SEQ ID NO: 17) from C. sativa:

MKCSTFSFWFVCKIIFFFFSFNIQTSIANPRENFLKCFSQYIPNNATNLK
LVYTQNNPLYMSVLNSTIHNLRFTSDTTPKPLVIVTPSHVSHIQGTILCS
KKVGLQIRTRSGGHDSEGMSYISQVPFVIVDLRNMRSIKIDVHSQTAWVE
AGATLGEVYYWVNEKNENLSLAAGYCPTVCAGGHFGGGGYGPLMRNYGLA
ADNIIDAHLVNVHGKVLDRKSMGEDLFWALRGGGAESFGIIVAWKIRLVA
VPKSTMFSVKKIMEIHELVKLVNKWQNIAYKYDKDLLLMTHFITRNITDN
QGKNKTAIHTYFSSVFLGGVDSLVDLMNKSFPELGIKKTDCRQLSWIDTI
IFYSGVVNYDTDNFNKEILLDRSAGQNGAFKIKLDYVKKPIPESVFVQIL
EKLYEEDIGAGMYALYPYGGIMDEISESAIPFPHRAGILYELWYICSWEK
QEDNEKHLNWIRNIYNFMTPYVSKNPRLAYLNYRDLDIGINDPKNPNNYT
QARIWGEKYFGKNFDRLVKVKTLVDPNNFFRNEQSIPPLPRHRH.

Additional non-limiting examples of CBDAS enzymes may also be found in U.S. Pat. No. 9,512,391 and U.S. Patent Application Publication No. 2018/0179564, which are incorporated by reference in this application in their entireties.

### Cannabichromenic Acid Synthase (CBCAS)

A host cell described in this application may comprise a TS that is a cannabichromenic acid synthase (CBCAS). As used in this application, a “CBCAS” refers to an enzyme that is capable of catalyzing oxidative cyclization of a prenyl moiety (e.g., terpene) of a compound of Formula (8) to produce a compound of Formula (11). In some embodiments, a compound of Formula (11) is a compound of Formula (11a) (cannabichromenic acid (CBCA)), CBCVA, or a compound of Formula (8) with R as a C7 alkyl (heptyl) group. A CBCAS may use cannabigerolic acid (CBGA) as a substrate. In some embodiments, a CBCAS produces cannabichromenic acid (CBCA) from cannabigerolic acid (CBGA). In some embodiments, the CBCAS may catalyze the oxidative cyclization of other substrates, such as 3-geranyl-2,4-dihydro-6-alkylbenzoic acids like cannabigerovarinic acid (CBGVA), or a substrate of Formula (8) with R as a C7 alkyl (heptyl) group. In some embodiments, the CBCAS exhibits specificity for CBGA substrates.

In some embodiments, a CBCAS is from Cannabis. In C. sativa, an amino acid sequence encoding CBCAS is provided by, and incorporated by reference from, SEQ ID NO:2 disclosed in U.S. Patent Publication No. 2017/0211049. In other embodiments, a CBCAS may be a THCAS described in and incorporated by reference from U.S. Pat. No. 9,359,625. SEQ ID NO:2 disclosed in U.S. Patent Application Publication No. 2017/0211049 (corresponding to SEQ ID NO: 4 in this application) has the amino acid sequence:

MNCSTFSFWFVCKIIFFFLSFNIQISIANPQENFLKCFSEYIPNNPANPK
FIYTQHDQLYMSVLNSTIQNLRFTSDTTPKPLVIVTPSNVSHIQASILCS
KKVGLQIRTRSGGHDAEGLSYISQVPFAIVDLRNMHTVKVDIHSQTAWVE
AGATLGEVYYWINEMNENFSFPGGYCPTVGVGGHFSGGGYGALMRNYGLA
ADNIIDAHLVNVDGKVLDRKSMGEDLFWAIRGGGGENFGIIAACKIKLVV
VPSKATIFSVKKNMEIHGLVKLFNKWQNIAYKYDKDLMLTTHFRTRNITD
NHGKNKTTVHGYFSSIFLGGVDSLVDLMNKSFPELGIKKTDCKELSWIDT
TIFYSGVVNYNTANFKKEILLDRSAGKKTAFSIKLDYVKKLIPETAMVKI
LEKLYEEEVGVGMYVLYPYGGIMDEISESAIPFPHRAGIMYELWYTATWE
KQEDNEKHINWVRSVYNFTTPYVSQNPRLAYLNYRDLDLGKTNPESPNNY
TQARIWGEKYFGKNFNRLVKVKTKADPNNFFRNEQSIPPLPPRHH.

Additional CBCASs are disclosed in and incorporated by reference from PCT/US21/24398.

### Variants

Aspects of the disclosure relate to nucleic acids encoding any of the polypeptides (e.g., AAE, PKS, PKC, PT, or TS) described in this application. In some embodiments, a nucleic acid encompassed by the disclosure is a nucleic acid that hybridizes under high or medium stringency conditions to a nucleic acid encoding an AAE, PKS, PKC, PT, or TS and is biologically active. For example, high stringency conditions of 0.2 to 1×SSC at 65° C. followed by a wash at 0.2×SSC at 65° C. can be used. In some embodiments, a nucleic acid encompassed by the disclosure is a nucleic acid that hybridizes under low stringency conditions to a nucleic acid encoding an AAE, PKS, PKC, PT, or TS and is biologically active. For example, low stringency conditions of 6×SSC at room temperature followed by a wash at 2×SSC at room temperature can be used. Other hybridization conditions include 3×SSC at 40 or 50° C., followed by a wash in 1 or 2×SSC at 20, 30, 40, 50, 60, or 65° C.

Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

Variants of enzyme sequences described in this application (e.g., AAE, PKS, PKC, PT, or TS, including nucleic acid or amino acid sequences) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” which is used interchangeably in this disclosure with the term “percent identity,” as known in the art, refers to a relationship between the sequences of two polypeptides or polynucleotides, as determined by sequence comparison (alignment). In some embodiments, sequence identity is determined across the entire length of a sequence (e.g., AAE, PKS, PKC, PT, or TS sequence). In some embodiments, sequence identity is determined over a region (e.g., a stretch of amino acids or nucleic acids, e.g., the sequence spanning an active site) of a sequence (e.g., AAE, PKS, PKC, PT, or TS sequence). For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natd. Acad. Sci. USA 87:2264-68, 1990, modified as in Karlin and Altschul Proc. Natd. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

As used in this application, variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., AAE, PKS, PKC, PT, or TS enzyme variant) comprises a domain that shares a secondary structure (e.g., alpha helix, beta sheet) with a reference polypeptide (e.g., a reference AAE, PKS, PKC, PT, or TS enzyme). In some embodiments, a polypeptide variant (e.g., AAE, PKS, PKC, PT, or TS enzyme variant) shares a tertiary structure with a reference polypeptide (e.g., a reference AAE, PKS, PKC, PT, or TS enzyme). As a non-limiting example, a polypeptide variant (e.g., AAE, PKS, PKC, PT, or TS enzyme) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of the recombinant AAE, PKS, PKC, PT, or TS enzyme disclosed in this application are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July;28(3):405-20) may be used to identify polypeptides with a particular domain.

Homology modeling may also be used to identify amino acid residues that are amenable to mutation (e.g., substitution, deletion, and/or insertion) without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11;10(9):2997-3011. The likelihood of observing a particular residue at a given position can be calculated. Without being bound by a particular theory, positions in sequences with high variability may be amenable to mutation (e.g., substitution, deletion, and/or insertion; e.g., PSSM score≥0) to produce functional homologs.

PSSM may be paired with calculation of a Rosetta energy function, which determines the difference between the wild-type and the single-point mutant. The Rosetta energy function calculates this difference as (ΔΔ_{calc}). With the Rosetta function, the bonding interactions between a mutated residue and the surrounding atoms are used to determine whether an amino acid substitution, deletion, or insertion increases or decreases protein stability. For example, an amino acid substitution, deletion, or insertion that is designated as favorable by the PSSM score (e.g. PSSM score≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔ_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, an AAE, PKS, PKC, PT, or TS coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions relative to a reference (e.g., AAE, PKS, PKC, PT, or TS) coding sequence. In some embodiments, the AAE, PKS, PKC, PT, or TS coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference (e.g., AAE, PKS, PKC, PT, or TS) coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., AAE, PKS, PKC, PT, or TS) relative to the amino acid sequence of a reference polypeptide (e.g., AAE, PKS, PKC, PT, or TS).

It should be appreciated that sequences disclosed herein may or may not contain signal sequences. The sequences disclosed herein encompass versions with or without signal sequences.

It should also be understood that protein sequences disclosed herein may be depicted with or without a start codon (M). Accordingly, in some instances amino acid numbering may correspond to protein sequences containing a start codon, while in other instances, amino acid numbering may correspond to protein sequences that do not contain a start codon. As a non-limiting example, a PKC may comprise residues 1-X of SEQ ID NOs: 1, 19-106 196-388, 582-669, 671-742, or 745-948, or an amino acid sequence in Table 11, in which X is the last amino acid of the reference sequence. As a non-limiting example, a PKC may comprise residues 2-X of SEQ ID NOs: 1, 19-106 196-388, 582-669, 671-742, or 745-948 or an amino acid sequence in Table 11, in which X is the last amino acid of the reference sequence. Where a PKC comprise residues 2-X of SEQ ID NOs: 1, 19-106, or 196-388, 582-669, 671-742, or 745-948 or an amino acid sequence in Table 11, such PKC may, for example, be used with a signal sequence. Aspects of the disclosure encompass host cells comprising any of the sequences described herein, including the sequences within Table 11 and fragments thereof.

In some embodiments, the one or more mutations in a recombinant coding sequence (e.g., AAE, PKS, PKC, PT, or TS coding sequence) do alter the amino acid sequence of the corresponding polypeptide (e.g., AAE, PKS, PKC, PT, or TS) relative to the amino acid sequence of a reference polypeptide (e.g., AAE, PKS, PKC, PT, or TS). In some embodiments, the one or more mutations alters the amino acid sequence of the polypeptide (e.g., AAE, PKS, PKC, PT, or TS) relative to the amino acid sequence of a reference polypeptide (e.g., AAE, PKS, PKC, PT, or TS) and alters (enhances or reduces) an activity of the polypeptide relative to the reference polypeptide.

The activity (e.g., specific activity) of any of the recombinant polypeptides described in this application (e.g., AAE, PKS, PKC, PT, or TS) may be measured using routine methods. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide (e.g., AAE, PKS, PKC, PT, or TS) coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 3). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 3.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions. In some embodiments, amino acids are replaced by non-conservative amino acid substitutions.

TABLE 3
Conservative Amino Acid Substitutions

| | | Conservative Amino |
|---|---|---|
| Original Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide (e.g., AAE, PKS, PKC, PT, or TS) variant having a desired property and/or activity can be made by alteration of the coding sequence of the polypeptide (e.g., AAE, PKS, PKC, PT, or TS). Similarly, conservative amino acid substitutions in the amino acid sequence of a polypeptide to produce functionally equivalent variants of the polypeptide typically are made by alteration of the coding sequence of the recombinant polypeptide (e.g., AAE, PKS, PKC, PT, or TS).

Mutations (e.g., substitutions, insertions, additions, or deletions) can be made in a nucleic acid sequence by a variety of methods known to one of ordinary skill in the art. For example, mutations (e.g., substitutions, insertions, additions, or deletions) can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by CRISPR, or by insertions, such as insertion of a tag (e.g., a HIS tag or a GFP tag). Mutations can include, for example, substitutions, insertions, additions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January;29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January;29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

### Expression of Nucleic Acids in Host Cells

Aspects of the present disclosure relate to recombinant enzymes, functional modifications and variants thereof, as well as their uses. For example, the methods described in this application may be used to produce cannabinoids and/or cannabinoid precursors. The methods may comprise using a host cell comprising an enzyme disclosed in this application, cell lysate, isolated enzymes, or any combination thereof. Methods comprising recombinant expression of genes encoding an enzyme disclosed in this application in a host cell are encompassed by the present disclosure. In vitro methods comprising reacting one or more cannabinoid precursors or cannabinoids in a reaction mixture with an enzyme disclosed in this application are also encompassed by the present disclosure. In some embodiments, the enzyme is a PKC.

A nucleic acid encoding any of the recombinant polypeptides (e.g., AAE, PKS, PKC, PT, or TS enzyme) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

A vector encoding any of the recombinant polypeptides (e.g., AAE, PKS, PKC, PT, or TS enzyme) described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is hereby incorporated by reference in its entirety. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described in this application is inserted into a cloning vector so that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome. In some embodiments, the nucleic acid sequence of a gene described in this application is recoded. Recoding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not recoded.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TP11, GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. This may be used, for example, to controllably induce the expression of an enzyme. In some embodiments, an inducible promoter linked to a PKC, a PT and/or a TS may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). In some embodiments, an inducible promoter linked to a CBGAS and/or a TS, the CBGAS and/or TS may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, an amino acid, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TP11, HXT3, HXT7, ACT1, ADH1, ADH2, ENO2, and SOD1.

Other inducible promoters or constitutive promoters, including synthetic promoters, that may be known to one of ordinary skill in the art are also contemplated.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

The disclosed cannabinoid biosynthetic methods and host cells are exemplified with S. cerevisiae, but are also applicable to other host cells, as would be understood by one of ordinary skill in the art.

Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuffle™ competent E. coli available from New England BioLabs in Ipswich, Mass.).

Other suitable host cells of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host cell of the present disclosure is C. glutamicum.

Suitable host cells of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types. In some embodiments, the plant is of the Cannabis genus in the family Cannabaceae. In certain embodiments, the plant is of the species Cannabis sativa, Cannabis indica, or Cannabis ruderalis. In other embodiments, the plant is of the genus Nicotiana in the family Solanaceae. In certain embodiments, the plant is of the species Nicotiana rustica.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart. Reduction of gene expression and/or gene inactivation in a host cell may be achieved through any suitable method, including but not limited to, deletion of the gene, introduction of a point mutation into the gene, selective editing of the gene and/or truncation of the gene. For example, polymerase chain reaction (PCR)-based methods may be used (see, e.g., Gardner et al., Methods Mol Biol. 2014; 1205:45-78). As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). A gene may also be edited through of the use of gene editing technologies known in the art, such as CRISPR-based technologies.

### Culturing of Host Cells

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermentor is used to culture the cell. Thus, in some embodiments, the cells are used in fermentation. As used in this application, the terms “bioreactor” and “fermentor” are interchangeably used and refer to an enclosure, or partial enclosure, in which a biological, biochemical and/or chemical reaction takes place that involves a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., cannabinoid or cannabinoid precursor) may display some differences from the substrate in terms of solubility, toxicity, cellular accumulation and secretion and in some embodiments can have different fermentation kinetics.

In some embodiments, the cells of the present disclosure are adapted to produce cannabinoids or cannabinoid precursors in vivo. In some embodiments, the cells are adapted to secrete one or more enzymes for cannabinoid synthesis (e.g., AAE, PKS, PKC, PT, or TS). In some embodiments, the cells of the present disclosure are lysed, and the lysate is recovered for subsequent use. In such embodiments, the secreted or lysed enzyme can catalyze reactions for the production of a cannabinoid or precursor by bioconversion in an in vitro or ex vivo process. In some embodiments, any and all conversions described in this application can be conducted chemically or enzymatically, in vitro or in vivo.

### Purification and Further Processing

In some embodiments, any of the methods described in this application may include isolation and/or purification of the cannabinoids and/or cannabinoid precursors produced (e.g., produced in a bioreactor). For example, the isolation and/or purification can involve one or more of cell lysis, centrifugation, extraction, column chromatography, distillation, crystallization, and lyophilization.

The methods described in this application encompass production of any cannabinoid or cannabinoid precursor known in the art. Cannabinoids or cannabinoid precursors produced by any of the recombinant cells disclosed in this application or any of the in vitro methods described in this application may be identified and extracted using any method known in the art. Mass spectrometry (e.g., LC-MS, GC-MS) is a non-limiting example of a method for identification and may be used to extract a compound of interest.

In some embodiments, any of the methods described in this application further comprise decarboxylation of a cannabinoid or cannabinoid precursor. As a non-limiting example, the acid form of a cannabinoid or cannabinoid precursor may be heated (e.g., at least 90° C.) to decarboxylate the cannabinoid or cannabinoid precursor. See, e.g., U.S. Pat. Nos. 10,159,908, 10,143,706, 9,908,832 and 7,344,736. See also, e.g., Wang et al., Cannabis Cannabinoid Res. 2016; 1(1): 262-271.

### Compositions, Kits, and Administration

The present disclosure provides compositions, including pharmaceutical compositions, comprising a cannabinoid or a cannabinoid precursor, or pharmaceutically acceptable salt thereof, produced by any of the methods described in this application, and optionally a pharmaceutically acceptable excipient.

In certain embodiments, a cannabinoid or cannabinoid precursor described in this application is provided in an effective amount in a composition, such as a pharmaceutical composition. In certain embodiments, the effective amount is a therapeutically effective amount. In certain embodiments, the effective amount is a prophylactically effective amount.

Compositions, such as pharmaceutical compositions, described in this application can be prepared by any method known in the art. In general, such preparatory methods include bringing a compound described in this application (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described in this application will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. Exemplary excipients include diluents, dispersing and/or granulating agents, surface active agents and/or emulsifiers, disintegrating agents, binding agents, preservatives, buffering agents, lubricating agents, and/or oils (e.g., synthetic oils, semi-synthetic oils) as disclosed in this application.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic or semi-synthetic oils include, but are not limited to, butyl stearate, medium chain triglycerides (such as caprylic triglyceride and capric triglyceride), cyclomethicone, diethyl sebacate, dimethicone 360, isopropyl myristate, mineral oil, octyldodecanol, oleyl alcohol, silicone oil, and mixtures thereof. In certain embodiments, exemplary synthetic oils comprise medium chain triglycerides (such as caprylic triglyceride and capric triglyceride).

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described in this application are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described in this application with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described in this application may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described in this application include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described in this application.

A pharmaceutical composition described in this application can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided in this application are typically formulated in dosage unit form for ease of administration and uniformity of dosage. It will be understood, however, that the total daily usage of the compositions described in this application will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided in this application can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In some embodiments, compounds or compositions disclosed in this application are formulated and/or administered in nanoparticles. Nanoparticles are particles in the nanoscale. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described in this application. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described in this application includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described in this application. In certain embodiments, a dose described in this application includes independently between 1 mg and 3 mg, inclusive, of a compound described in this application. In certain embodiments, a dose described in this application includes independently between 3 mg and 10 mg, inclusive, of a compound described in this application. In certain embodiments, a dose described in this application includes independently between 10 mg and 30 mg, inclusive, of a compound described in this application. In certain embodiments, a dose described in this application includes independently between 30 mg and 100 mg, inclusive, of a compound described in this application.

Dose ranges as described in this application provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described in this application, can be administered in combination with one or more additional pharmaceutical agents (e.g., therapeutically and/or prophylactically active agents). The compounds or compositions can be administered in combination with additional pharmaceutical agents that improve their activity, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described in this application including a compound described in this application and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described in this application in a single dose or administered separately in different doses. The particular combination to employ in a regimen will take into account compatibility of the compound described in this application with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In some embodiments, one or more of the compositions described in this application are administered to a subject. In certain embodiments, the subject is an animal. The animal may be of either sex and may be at any stage of development. In certain embodiments, the subject is a human. In other embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

Also encompassed by the disclosure are kits (e.g., pharmaceutical packs). The kits provided may comprise a composition, such as a pharmaceutical composition, or a compound described in this application and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described in this application. In some embodiments, the pharmaceutical composition or compound described in this application provided in the first container and the second container a combined to form one unit dosage form.

Thus, in one aspect, provided are kits including a first container comprising a compound or composition described in this application. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described in this application further includes instructions for using the kit. A kit described in this application may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. A kit described in this application may include one or more additional pharmaceutical agents described in this application as a separate composition.

The present invention is further illustrated by the following Examples, which in no way should be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. However, mention of any reference, article, publication, patent, patent publication, and patent application cited in this disclosure is not, and should not be taken as an acknowledgment or any form of suggestion that they constitute valid prior art or form part of the common general knowledge in any country in the world.

### EXAMPLES

### Example 1: Functional Expression of Synthetic OAC Polypeptides

A library of synthetic OAC polypeptides was generated and screened in vivo in S. cerevisiae to identify polypeptides that were capable of producing olivetolic acid (OA). Each polypeptide comprised multiple amino acid substitutions or deletions relative to wild-type CsOAC (SEQ ID NO: 1).

For expression in S. cerevisiae, nucleotide sequences were recoded, synthesized, and cloned into a non-replicative yeast expression vector. Each candidate OAC expression construct was transformed and integrated into the genome of an S. cerevisiae CEN.PK strain previously engineered to produce olivetol by carrying a genomically integrated olivetol synthase (OLS).

The nucleic acid sequence of the genomically integrated OLS is provided by SEQ ID NO: 1316:

atgcccagtttagagtcagttaagaaatccaatcgtgccgacggcttcgc
atcgattctggctataggtagagctaaccctgaaaactttatcgaacagt
ctacatatccagatttctttttcagagtcaccaatagcgaacaccttgta
aacctaaagaaaaagttccaaagaatttgcgacaagactgctatcaggaa
gcgtcattttgtgtggaacgaagaattgttgaatgccaacccatgtttgg
gtacgtttatggataactcattaaacgtcagacaagaatttgctattaga
gagattccaaaactaggtgctgaagctgccactaaggcaatccaagaatg
gggtcaaccaaagtccagaataacccacttgatcttctgtactacctctg
gaatggatttgccaggtgctgactaccaattgacccaaattctgggtttg
aatcctaatattgagagggttatgttataccagcaaggttgtttcgctgg
tggtactactttgagattggccaaatgtttagccgaatctcgtaagggag
ctagagttttggttgtctgtgctgaaacaaccgctgttctattcagagca
ccttccgaagaacatcaagatgatttagtaactcaagctttgttcgccga
cggtgcttctgctcttatcgttggtgcagacccagacgagactgcccacg
aaagagctagttttgttattgtctctacatctcaagtcttgttaccagat
agcgctggtgctatcggcggtcatgtgtccgaaggtggtttgatcgccac
tttgcacagagatgttccacagatagttagcaaaaatgtcggtaagtgct
tggaagaagcattcacccccttgggtattagtgattggaacagtattttt
tgggttccacacccaggaggtagagctattcttgaccaagtggaagaaag
agtcggtttaaagcctgagaagttgatcgtatccagacatgtgttagccg
aatatggcaacatgtcttctgtttgtgttcactttgctctggatgaaatg
aggaagagatctaaaaaagaaggtaaggctacaaccggtgagggtttaga
ctggggtgttttgttcggcttcggtccaggattaactgtcgaaaccgtcg
ttttgcactctgttccaatataa.

The protein sequence of the genomically integrated OLS is provided by SEQ ID NO: 195:

MPSLESVKKSNRADGFASILAIGRANPENFIEQSTYPDFFFRVTNSEHLV
NLKKKFQRICDKTAIRKRHFVWNEELLNANPCLGTFMDNSLNVRQEFAIR
EIPKLGAEAATKAIQEWGQPKSRITHLIFCTTSGMDLPGADYQLTQILGL
NPNIERVMLYQQGCFAGGTTLRLAKCLAESRKGARVLVVCAETTAVLFRA
PSEEHQDDLVTQALFADGASALIVGADPDETAHERASFVIVSTSQVLLPD
SAGAIGGHVSEGGLIATLHRDVPQIVSKNVGKCLEEAFTPLGISDWNSIF
WVPHPGGRAILDQVEERVGLKPEKLIVSRHVLAEYGNMSSVCVHFALDEM
RKRSKKEGKATTGEGLDWGVLFGFGPGLTVETVVLHSVPI.

An assay for measuring production of olivetolic acid was conducted as follows: each thawed glycerol stock of OAC transformants was stamped into a well of YPD+4% dextrose media. Samples were incubated at 30° C. in a shaking incubator for 2 days. A portion of each of the resulting cultures was stamped into a well of YEP+2% raffinose+2% galactose+1 mM hexanoate (C6). Samples were incubated at 30° C. in a shaking incubator for 4 days. Additional spike-ins of 1 mM hexanoate and 2% galactose were added at 24 and 48 hours for a total of 3 mM hexanoate and 6% galactose feed. A portion of each of the resulting production cultures was stamped into a well of Phosphate Buffered Saline (PBS). Optical measurements were taken on a plate reader, with absorbance measured at 600 nm and fluorescence at 528 nm with 485 nm excitation. A portion of each of the production cultures was stamped into a well of 100% methanol in half-height deepwell plates. Plates were heat sealed and frozen at −80° C. for two hours. Samples were then thawed and spun down. A portion of the supernatant was stamped into half-area 96 well plates. Olivetolic acid production in the samples was measured via liquid chromatography-mass spectrometry (LC-MS) by measuring relative peak areas.

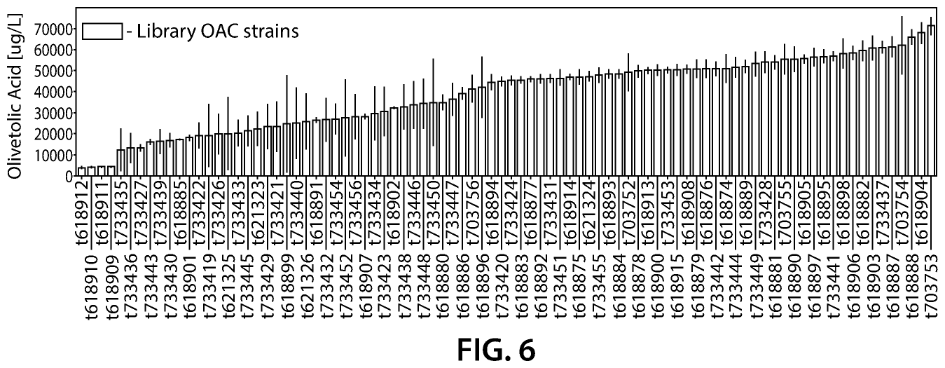

As shown in Table 4 and FIG. 6, multiple synthetic OAC polypeptides produced olivetolic acid (OA). For example, strains t703753, t618904, t618888, t618887, t618903, t618882, t733437, t618906, t618898, t733441, t618895, t618897, t618905, t618890, t703755, t618881, t733428, t733449, t618889, t733444, t703754, t618874, t733442, t618876, t618879, t618908, t618915, t733453, t618900, t618913, t618878, t703752, t618884, t618893, t733455, and t621324 produced at least 47,000 μg/L olivetolic acid. Surprisingly, strains comprising several non-conservative amino acid substitutions were capable of producing olivetolic acid. Non-conservative amino acid substitutions are indicated in bold font and are underlined in Table 4.

TABLE 4
Activity data for synthetic OAC polypeptides in S. cerevisiae

| | | Average | Standard Deviation |
|---|---|---|---|
| | Amino acid substitutions relative to | Olivetolic Acid | Olivetolic Acid |
| Strain | SEQ ID NO: 1 | [μg/L] | [μg/L] |
| t621327 | — | 83112.99 | 3180.61 |
| t703753 | A18D T26A V28E N29S E53H E60I | 71051.94 | 4357.89 |
| | Q70L G82K D83A S87K | | |
| t618904 | A18D E22K N29S E53H Q70L G82K | 67803.84 | 5130.069 |
| | D83R S87K F88Y | | |
| t618888 | A18D E22K N29S E53H Q70L G82K | 65943.84 | 3771.639 |
| | D83R S87K F88Y | | |
| t703754 | A18D E22K T26A N29S E53H Q70L | 61898.15 | 13873.5 |
| | G82K D83A F88Y | | |
| t618887 | A18D E22K N29S E53H Q70L G82K | 61157.09 | 4988.12 |
| | D83K S87K F88Y | | |
| t733437 | A18D E22K K25N T26A N29S E53H | 60924.63 | 3069.983 |
| | Q70L G82K D83A S87K | | |
| t618903 | A18D E22K N29S E53H Q70L G82K | 60664.32 | 6048.762 |
| | D83K S87K F88Y | | |
| t618882 | A18D E22K N29S I33R E53H Q70L | 59371.51 | 4972.507 |
| | G82K D83A S87K F88Y | | |
| t618906 | A18D E22K N29S E53H Q70L G82K | 58293.9 | 3409.644 |
| | D83A V84K S87K F88Y | | |
| t618898 | A18D E22K N29S I33R E53H Q70L | 57987.09 | 7114.668 |
| | G82K D83A S87K F88Y | | |
| t733441 | A18D E22K T26A N29S E53H Q70L | 56814.63 | 2333.956 |
| | G82K D83A S87K F88Y | | |
| t618895 | A18D E22K T26H N29S E53H Q70L | 56466.43 | 3350.318 |
| | G82K D83A S87K F88Y | | |
| t618897 | A18D E22K N29S I33H E53H Q70L | 56240.63 | 4550.125 |
| | G82K D83A S87K F88Y | | |
| t618905 | A18D E22K N29S E53H Q70L G82K | 55458.68 | 2234.887 |
| | D83A V84F S87K F88Y | | |
| t618890 | A18D E22K N29S E53H Q70L G82K | 55344.53 | 6117.93 |
| | D83A V84K S87K F88Y | | |
| t703755 | A18D T26A N29S E53H Q70L G82K | 55243.23 | 7240.143 |
| | D83A F88Y | | |
| t618881 | A18D E22K N29S I33H E53H Q70L | 53817.65 | 3411.585 |
| | G82K D83A S87K F88Y | | |
| t733428 | A18D E22K T26A N29S E53H Q70L | 53764.98 | 5289.474 |
| | G82K D83A S87K | | |
| t733449 | A18D T26A N29S E53H Q70L G82K | 53104.9 | 5995.439 |
| | D83A S87K F88Y | | |
| t618889 | A18D E22K N29S E53H Q70L G82K | 51897.8 | 3218.588 |
| | D83A V84F S87K F88Y | | |
| t733444 | A18D T26A N29S E53H Q70L G82K | 51530.41 | 4992.193 |
| | D83A | | |
| t618874 | A18D E22K N29S E53H Q70L G82K | 50868.29 | 7037.235 |
| | D83A S87K F88Y | | |
| t733442 | A18D T26A V28E N29S E53H Q70L | 50754.38 | 3412.802 |
| | G82K D83A S87K | | |
| t618876 | A18R E22K N29S E53H Q70L G82K | 50737.94 | 4477.936 |
| | D83A S87K F88Y | | |
| t618879 | A18D E22K T26H N29S E53H Q70L | 50677.48 | 4527.628 |
| | G82K D83A S87K F88Y | | |
| t618908 | d2 A18D E22K N29S E53H Q70L | 50660.38 | 2210.721 |
| | G82K D83A S87K F88Y | | |
| t618915 | A18D E22K N29S E53H T62M Q70L | 50223.45 | 2855.212 |
| | G82K D83A S87K F88Y | | |
| t733453 | A18D E22K T26A N29E E53H Q70L | 50179.68 | 1547.743 |
| | G82K D83A S87K | | |
| t618900 | A18D E22K N29S E52H E53H Q70L | 50081.71 | 2759.643 |
| | G82K D83A S87K F88Y | | |
| t618913 | A18D E22K N29S E53H Q70L G82K | 49877.07 | 1495.849 |
| | D83A S87C F88Y | | |
| t618878 | A18D E22K F24M N29S E53H Q70L | 49593.19 | 3181.27 |
| | G82K D83A S87K F88Y | | |
| t703752 | A18D N29S E53H Q70L G82K D83A | 49167.24 | 9067.041 |
| | F88Y | | |
| t618884 | A18D E22K N29S E52H E53H Q70L | 48455.36 | 2418.536 |
| | G82K D83A S87K F88Y | | |
| t618893 | A18R E22K N29S E53H Q70L G82K | 48255.78 | 2355.544 |
| | D83A S87K F88Y | | |
| t733455 | A18D N29S E53H Q70L G82K D83A | 47534.07 | 3782.694 |
| | S87K F88Y | | |
| t621324 | A18D E22K T26A N29S E53H H57Y | 47010.07 | 2707.421 |
| | Q70L G82K D83A S87K | | |
| t618875 | D13R A18D E22K N29S E53H Q70L | 46880.2 | 3653.498 |
| | G82K D83A S87K F88Y | | |
| t618914 | A18D E22K N29S E53H Q70L G82K | 46846.9 | 1542.364 |
| | D83A S87C F88Y | | |
| t733451 | A18D N29S E53H Q70L G82K D83A | 46536.01 | 3940.882 |
| | S87K | | |
| t733431 | A18D T26A N29S E53H Q70L G82K | 46094.45 | 2385.012 |
| | D83A S87K | | |
| t618892 | D13R A18D E22K N29S E53H Q70L | 45895.72 | 2437.177 |
| | G82K D83A S87K F88Y | | |
| t618877 | A18D K20A E22K N29S E53H Q70L | 45775.07 | 1698.94 |
| | G82K D83A S87K F88Y | | |
| t618883 | A18D E22K N29S N50K E53H Q70L | 45393.22 | 1956.514 |
| | G82K D83A S87K F88Y | | |
| t733424 | A18D E22K N29S E53H Q70L G82K | 45209.24 | 2590.566 |
| | D83A S87K | | |
| t733420 | A18D T26A V28E N29S E53H Q70L | 44644.47 | 2371.943 |
| | G82K D83A S87K F88Y | | |
| t618894 | A18D K20A E22K N29S E53H Q70L | 44242.37 | 4051.608 |
| | G82K D83A S87K F88Y | | |
| t618896 | A18D E22K T26R N29S E53H Q70L | 41915.47 | 14867.75 |
| | G82K D83A S87K F88Y | | |
| t703756 | A18D N29S E53H H57Y Q70L G82K | 41188.71 | 6817.98 |
| | D83A S87K F88Y | | |
| t618886 | A18D E22K N29S E53H Q70L G80V | 39008.2 | 3100.816 |
| | G82K D83A S87K F88Y | | |
| t733447 | A18D E22K T26A N29S E53H Q70L | 36277.48 | 7873.193 |
| | G82K D83A | | |
| t618880 | A18D E22K T26R N29S E53H Q70L | 34821.62 | 3825.626 |
| | G82K D83A S87K F88Y | | |
| t733450 | A18D E22K N29S E53H E60I Q70L | 34644.28 | 20796.55 |
| | G82K D83A S87K | | |
| t733448 | A18D E22K T26A N29S E53H E60I | 34154.85 | 11909.31 |
| | Q70L G82K D83A | | |
| t733446 | A18D E22K T26A N29E E53H E60I | 33517.33 | 11449.4 |
| | Q70L G82K D83A S87K | | |
| t733438 | A18D K25N T26A N29S E53H E60I | 32638.74 | 10813.33 |
| | Q70L G82K D83A F88Y | | |
| t618902 | A18D E22K N29S E53H Q70L G80V | 32116.82 | 614.0084 |
| | G82K D83A S87K F88Y | | |
| t733423 | A18D E22K N29S E53H E60I Q70L | 30463.15 | 11691.4 |
| | G82K D83A S87K F88Y | | |
| t733434 | A18D T26A N29S E53H E60I Q70L | 29507.28 | 12923.24 |
| | G82K D83A F88Y | | |
| t618907 | A18D E22K N29S E53H Q70L G82K | 28120.06 | 1367.604 |
| | D83A Y85A S87K F88Y | | |
| t733456 | A18D E22K K25N T26A N29S E53H | 27966.55 | 10615.38 |
| | E60I Q70L G82K D83A F88Y | | |
| t733452 | A18D T26A V28E N29S E53H E60I | 27469.17 | 18457.2 |
| | Q70L G82K D83A S87K F88Y | | |
| t733454 | A18D E22K T26A N29S E53H E60I | 26853.01 | 7215.242 |
| | Q70L G82K D83A S87K | | |
| t733432 | A18D E22K T26A V28E N29S E53H | 26554.45 | 10446.36 |
| | E60I Q70L G82K D83A S87K F88Y | | |
| t618891 | A18D E22K N29S E53H Q70L G82K | 26231.71 | 1524.903 |
| | D83A Y85A S87K F88Y | | |
| t621326 | A18D K25N T26A N29S E53H E60I | 25650.15 | 13084.44 |
| | Q70L G82K D83A S87K F88Y | | |
| t733440 | A18D T26A N29S E53H E60I Q70L | 24860.86 | 17040.81 |
| | G82K D83A | | |
| t618899 | A18D E22K N29S N50K E53H Q70L | 24523.8 | 23062.39 |
| | G82K D83A S87K F88Y | | |
| t733421 | A18D N29S E53H E60I Q70L G82K | 23409.49 | 11929.06 |
| | D83A F88Y | | |
| t733429 | A18D N29S E53H E60I Q70L G82K | 23282.18 | 10836.94 |
| | D83A S87K | | |
| t621323 | A18D E22K T26A V28E N29S E53H | 22223.72 | 8285.182 |
| | E60I Q70L G82K D83A S87K | | |
| t733445 | A18D E22K K25N T26A N29S E53H | 21167.38 | 7497.625 |
| | E60I Q70L G82K D83A S87K F88Y | | |
| t733433 | A18D E22K T26A N29S E53H E60I | 20135.18 | 6451.948 |
| | Q70L G82K D83A F88Y | | |
| t621325 | A18D K25N T26A N29S E53H E60I | 19974.38 | 17388.96 |
| | Q70L G82K D83A S87K | | |
| t733426 | A18D N29S E53H E60I Q70L G82K | 19865.77 | 9768.158 |
| | D83A S87K F88Y | | |
| t733419 | A18D T26A V28E N29S E53H E60I | 19051.88 | 15141.84 |
| | Q70L G82K D83A F88Y | | |
| t733422 | A18D T26A N29S E53H E60I Q70L | 18987.74 | 6139.826 |
| | G82K D83A S87K I94V | | |
| t618901 | A18D E22K N29S E53H T56Y Q70L | 17945.17 | 1633.593 |
| | G82K D83A S87K F88Y | | |
| t618885 | A18D E22K N29S E53H T56Y Q70L | 17226.74 | 670.601 |
| | G82K D83A S87K F88Y | | |
| t733430 | A18D T26A N29S E53H E60I Q70L | 16745.01 | 3271.252 |
| | G82K D83A S87K | | |
| t733439 | A18D E22K K25N T26A N29S E53H | 16244.41 | 5970.461 |
| | E60I Q70L G82K D83A S87K | | |
| t733443 | A18D E22K T26A V28E N29S E53H | 15979.92 | 1466.996 |
| | E60I Q70L G82K D83A F88Y | | |
| t733427 | A18D T26A N29S E53H E60I Q70L | 13124.14 | 1823.281 |
| | G82K D83A S87K F88Y | | |
| t733436 | A18D V28E N29S E53H E60I Q70L | 13096.49 | 7004.152 |
| | G82K D83A S87K | | |
| t733435 | A18D E22K N29S E53H H57Y E60I | 12261.54 | 10276.08 |
| | Q70L G82K D83A S87K | | |
| t618909 | A18R K20A E22K T26R N29S I33R | 4214.472 | 429.7709 |
| | N50K E53H T56Y Q70L G82K D83K | | |
| | V84K Y85A S87K F88Y | | |
| t618911 | A18R K20A E22K T26R N29S I33R | 4191.066 | 433.3006 |
| | N50K E53H T56Y Q70L G82K D83K | | |
| | V84K Y85A S87K F88Y | | |
| t618910 | A18R K20A E22K T26R N29S I33R | 4030.05 | 468.0208 |
| | N50K E53H T56Y Q70L G82K D83K | | |
| | V84K Y85A S87K F88Y | | |
| t618912 | A18R K20A E22K T26R N29S I33R | 3845.85 | 709.8007 |
| | N50K E53H T56Y Q70L G82K D83K | | |
| | V84K Y85A S87K F88Y | | |

### Example 2: Identification of Combinatorially Mutated OAC Polypeptides that are Capable of Producing Olivetolic Acid

Example 1 describes a library of synthetic OAC polypeptides that demonstrated production of olivetolic acid (OA) in vivo in S. cerevisiae. However olivetol (OL), a by-product generated by the spontaneous, decarboxylative cyclization of 3,5,7-trioxododecanoyl-CoA generated by olivetol synthase (FIG. 1 R2a), was also generated in vivo in S. cerevisiae strains incorporating the synthetic OAC polypeptides. No known enzyme has been demonstrated to utilize OL in meaningful amounts to generate the decarboxylated variants of other cannabinoids (e.g., a prenyltransferase capable of prenylating OL with geranyl diphosphate to form cannabigerol). Consequently, OL is considered an unwanted carbon sink for the biosynthesis of cannabinoids and its production should be minimized.

A library of synthetic OAC polypeptides was generated in which the polypeptides included multiple point mutations selected from a set of 230 different point mutations relative to the wild type C. sativa OAC. Strains expressing the point mutations were assessed in vivo in S. cerevisiae for both the generation of OA and the minimization of OL production. This latter metric was assessed by calculating the OA/OL ratio of each point mutant. For expression in S. cerevisiae, nucleotide sequences were recoded, synthesized, and cloned into a 2p replicative yeast expression vector. Each candidate OAC expression construct was transformed into a S. cerevisiae CEN.PK strain previously engineered to produce olivetol by carrying a genomically integrated olivetol synthase as in Example 1. Strains t442707, t442710, and t454529, expressing three different recoded variants of CsOAC, were included in the library as positive controls. Strain t442712, expressing a fluorescent protein (GFP), was included in the library as a negative control for enzyme activity.

An assay for measuring production of olivetolic acid was conducted as follows: each thawed glycerol stock of OAC transformants was stamped into a well of YPD+4% dextrose media. Samples were incubated at 30° C. in a shaking incubator for 2 days. A portion of each of the resulting cultures was stamped into a well of YEP+2% raffinose+2% galactose+1 mM hexanoate (C6). Samples were incubated at 30° C. in a shaking incubator for 4 days. Additional spike-ins of 1 mM hexanoate and 2% galactose were added at 24 and 48 hours for a total of 3 mM hexanoate and 6% galactose feed. A portion of each of the resulting production cultures was stamped into a well of PBS. Optical measurements were taken on a plate reader, with absorbance measured at 600 nm and fluorescence at 528 nm with 485 nm excitation. A portion of each of the production cultures was stamped into a well of 100% methanol in half-height deepwell plates. Plates were heat sealed and frozen at −80° C. for two hours. Samples were then thawed and spun down. A portion of the supernatant was stamped into half-area 96 well plates. Olivetolic acid and olivetol production in the samples were quantified via LC-MS.

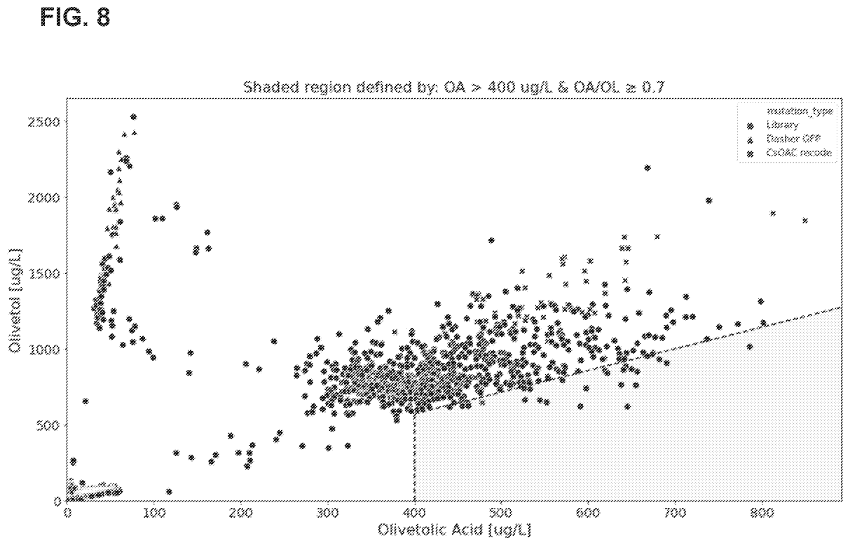

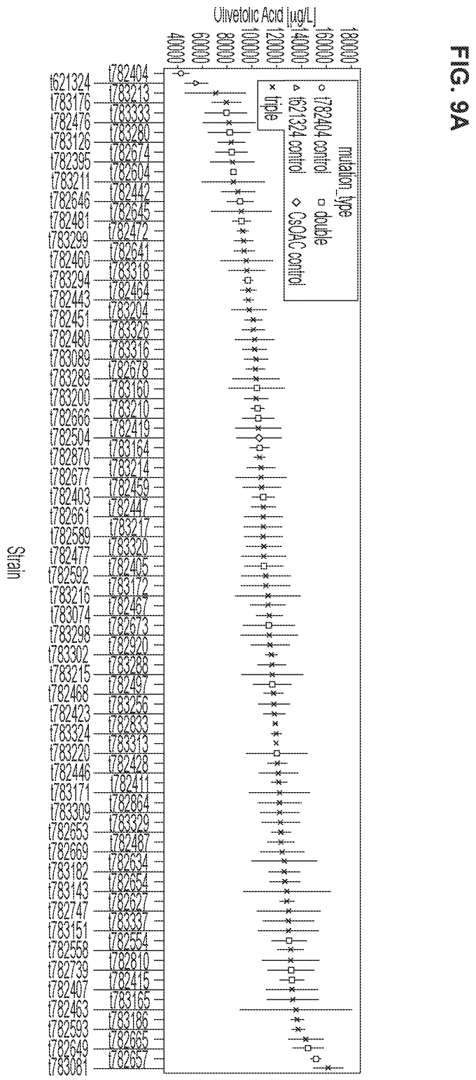

Two criteria were applied to the results of this assay to determine hits: the generation of an OA titer greater than 400 μg/L, and an OA/OL ratio greater than or equal to 0.7 (shaded region FIG. 8). Strains incorporating OAC polypeptides with mutations at 13 different residues, representing 19 point mutations (e.g. E17N, A18D, T26A, T26H, N29T, V31L, I33R, I33H, I33N, M37L, D39Q, D39G, D71S, G80A, D83Q, D83N, D83A, V84F, and F₉₅M) satisfied both criteria (Table 5). These point mutations were combined into CsOAC variants having multiple mutations in a library containing 937 synthetic OACs which was screened both for production of OA and OA/OL titer greater than or equal to 70%. For expression in S. cerevisiae, nucleotide sequences were recoded, synthesized, and cloned into an integrative yeast expression vector. Each candidate OAC expression construct was transformed into a S. cerevisiae CEN.PK strain previously engineered to produce olivetol by carrying a genomically integrated olivetol synthase as in Example 1. Strains t782504, expressing wild-type CsOAC (SEQ ID NO: 1), was included in the library as a positive control. Strain t621324, expressing a CsOAC variant containing a combination of 10 point mutations relative to the CsOAC sequence, was included in the library as positive control for hit ranking. Strain t782404, expressing a fluorescent protein (GFP), was included in the library as a negative control for enzyme activity.

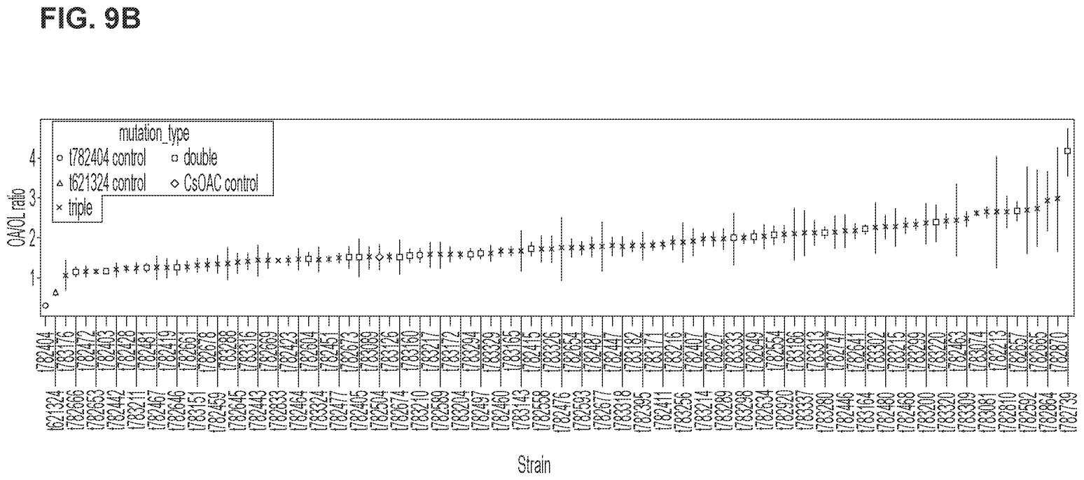

99 of the multiply-mutated CsOAC variants were selected and a secondary screen performed to confirm their activity (FIGS. 9A-9B and with mutations shown in Table 6). FIG. 9A shows olivetolic acid production by each synthetic OAC and FIG. 9B shows the ratio of OA:OL. 65% of the library demonstrated a mean OA titer, and 70% demonstrated a mean OA/OL ratio, greater than that of the t782504 positive control. To determine which individual mutations within the multiply-mutated CsOAC variants increased OA titer and/or the OA/OL ratio, hits were first segregated into groups. Each CsOAC variant was represented as a 1×20 vector containing mixed categorical and numerical data. Dimensions of each vector represented whether an OAC carried the wild-type residue at each of the possible mutated positions or one of the possible mutations. 2 dimensions of each vector represented the mean olivetolic acid titer and OA/OL ratio associated with a specific OAC.

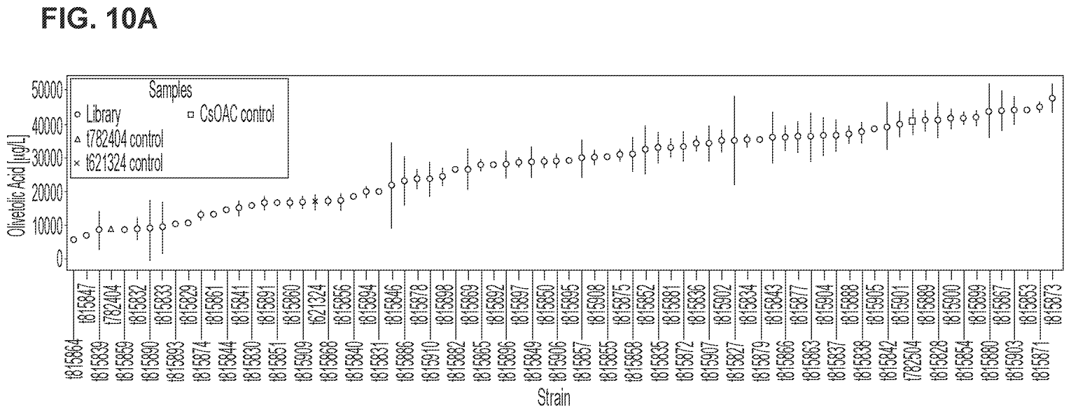

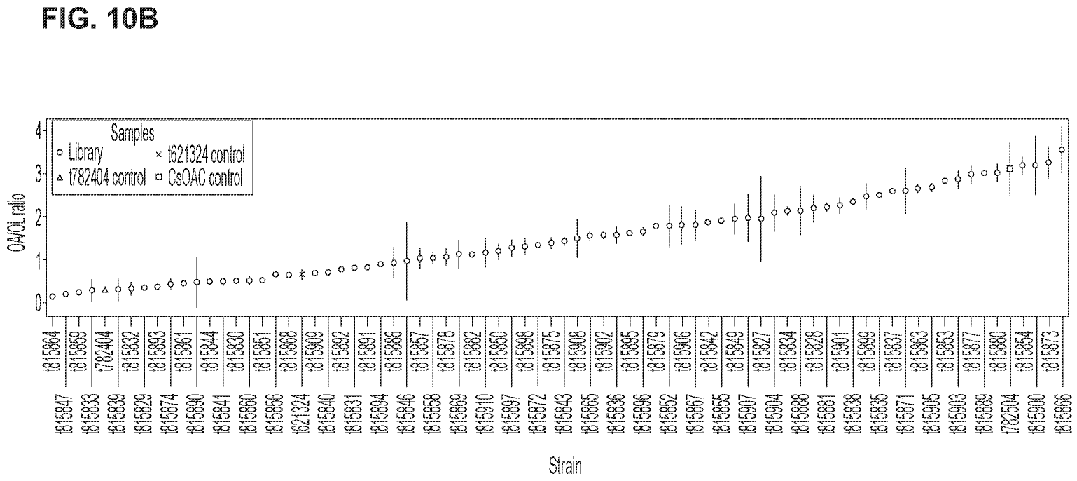

A library of 75 synthetic OACs was generated from a combination of nine mutations (E17N, A18D, N29T, V31L, I33N, D39Q, D71S, D83A, and V84F). Each strain tested in the library contained 6 of these amino acid substitutions. An assay for measuring production of olivetolic acid was conducted as follows: each thawed glycerol stock of OAC transformants was stamped into a well of YPD+4% dextrose media. Samples were incubated at 30° C. in a shaking incubator for 2 days. A portion of each of the resulting cultures was stamped into a well of YEP+2% raffinose+2% galactose+1 mM hexanoate (C₆). Samples were incubated at 30° C. in a shaking incubator for 4 days. Additional spike-ins of 1 mM hexanoate and 2% galactose were added at 24 and 48 hours for a total of 3 mM hexanoate and 6% galactose feed. A portion of each of the resulting production cultures was stamped into a well of PBS. Optical measurements were taken on a plate reader, with absorbance measured at 600 nm and fluorescence at 528 nm with 485 nm excitation. A portion of each of the production cultures was stamped into a well of 100% methanol in half-height deepwell plates. Plates were heat sealed and frozen at −80° C. for two hours. Samples were then thawed and spun down. A portion of the supernatant was stamped into half-area 96 well plates. Olivetolic acid and olivetol production in the samples were quantified via LC-MS. Strains t815873, t815900 and t815854 produced a mean OA titer that is greater than the CsOAC positive control. Strains t815873, t815900 and t815854 generated a mean OA titer greater than 41,000 μg/L and a mean OA/OL ratio greater than 3.1 (FIGS. 10A-10B and Table 7).

TABLE 5
Activity data for synthetic OAC polypeptides in
S. cerevisiae described in Example 2 and FIG. 8.

| | | Mutations relative to | Average | Average |
|---|---|---|---|---|
| | Strain | CsOAC | Olivetolic Acid | OA/OL |
| Strain | type | (SEQ ID NO: 1) | [μg/L] | ratio |
| t500610 | Library | I33R | 650.21 | 0.95 |
| t500726 | Library | D83Q | 552.41 | 0.85 |
| t500710 | Library | V84F | 544.78 | 0.83 |
| t500269 | Library | A18D | 618.04 | 0.82 |
| t500435 | Library | D83N | 686.58 | 0.82 |
| t500492 | Library | F95M | 527.47 | 0.80 |
| t500431 | Library | G80A | 670.57 | 0.76 |
| t500267 | Library | D83A | 631.72 | 0.74 |
| t500294 | Library | D39Q | 462.16 | 0.74 |
| t500693 | Library | I33H | 665.47 | 0.74 |
| t500192 | Library | I33N | 462.07 | 0.73 |
| t500544 | Library | N29T | 533.34 | 0.73 |
| t500289 | Library | D39G | 631.43 | 0.72 |
| t500701 | Library | V31L | 570.68 | 0.72 |
| t500588 | Library | D71S | 459.91 | 0.72 |
| t500274 | Library | T26A | 455.98 | 0.72 |
| t500213 | Library | E17N | 440.70 | 0.72 |
| t500440 | Library | M37L | 526.31 | 0.71 |
| t500137 | Library | T26H | 668.56 | 0.70 |
| t454529 | CsOAC | — | 245.69 | 0.67 |
| | positive | | | |
| | control | | | |
| t442707 | CsOAC | — | 299.15 | 0.61 |
| | positive | | | |
| | control | | | |
| t442710 | CsOAC | — | 286.12 | 0.58 |
| | positive | | | |
| | control | | | |
| t442712 | GFP | — | 29.18 | 0.03 |
| | negative | | | |
| | control | | | |

TABLE 6
Activity data for synthetic OAC polypeptides in S. cerevisiae
described in Example 2 and FIGS. 9A-9B.

| | | | Standard | | |
|---|---|---|---|---|---|
| | | | Deviation | | Standard |
| | | | Olivetolic | Average | Deviation |
| | Mutations relative to | Average Olivetolic | Acid | OA/OL | OA/OL |
| Strain | CsOAC (SEQ ID NO: 1) | Acid [ug/L] | [ug/L] | ratio | ratio |
| t782739 | [A18D, M37L] | 131500 | 26162.95 | 4.15 | 0.82 |
| t782870 | [E17N, T26A, M37L] | 106000 | 5830.95 | 2.96 | 1.48 |
| t782864 | [V31L, D39G, V84F] | 122000 | 20412.41 | 2.93 | 0.87 |
| t782665 | [E17N, I33R, F95M] | 142750 | 16997.55 | 2.73 | 1.11 |
| t782592 | [N29T, D39G, V84F] | 110800 | 21982.42 | 2.69 | 1.25 |
| t782657 | [D39Q, F95M] | 151500 | 5446.71 | 2.66 | 0.27 |
| t782810 | [E17N, A18D, I33N] | 130500 | 31819.81 | 2.65 | 0.57 |
| t783213 | [E17N, M37L, D83Q] | 70425 | 27893.65 | 2.64 | 1.58 |
| t783081 | [E17N, A18D, D39G] | 161500 | 16263.46 | 2.63 | 0.17 |
| t783074 | [I33H, D39G, D83Q] | 113500 | 14849.24 | 2.62 | 0.09 |
| t783309 | [A18D, D39Q, D71S] | 122375 | 18634.09 | 2.48 | 0.21 |
| t782463 | [N29T, I33N, D71S] | 134975 | 51662.39 | 2.45 | 1.05 |
| t783320 | [E17N, D39G, D83A] | 109450 | 16133.09 | 2.42 | 0.22 |
| t783220 | [D39G, D71S] | 119900 | 28999.54 | 2.38 | 0.52 |
| t783200 | [E17N, D83N, F95M] | 103375 | 11190.58 | 2.37 | 0.57 |
| t783299 | [E17N, T26H, N29T] | 93050 | 8327.66 | 2.33 | 0.15 |
| t782468 | [V31L, D39G, D71S] | 117000 | 9380.83 | 2.31 | 0.22 |
| t783215 | [I33R, D39G, D71S] | 115925 | 28962.89 | 2.27 | 0.54 |
| t782480 | [N29T, I33H, D71S] | 101775 | 18170.01 | 2.27 | 0.33 |
| t783302 | [A18D, N29T, V31L] | 115500 | 5196.15 | 2.24 | 0.73 |
| t783164 | [N29T, I33N] | 105750 | 11667.26 | 2.21 | 0.16 |
| t782641 | [E17N, D71S, G80A] | 93875 | 9591.79 | 2.17 | 0.21 |
| t782446 | [V31L, D71S, D83Q] | 121375 | 17594.39 | 2.17 | 0.47 |
| t782747 | [A18D, V31L, D39G] | 128650 | 29506.33 | 2.15 | 0.47 |
| t783280 | [E17N, D39G] | 81550 | 19849.52 | 2.14 | 0.15 |
| t783313 | [E17N, I33H, D71S] | 119000 | 2160.25 | 2.13 | 0.35 |
| t783337 | [N29T, V31L, D39Q] | 129250 | 23414.74 | 2.12 | 0.65 |
| t783186 | [I33H, D39Q, D71S | 136250 | 6184.66 | 2.10 | 0.73 |
| t782920 | [D39Q, D71S, D83A] | 114000 | 18565.20 | 2.09 | 0.24 |
| t782554 | [N29T, D39G] | 129750 | 15924.30 | 2.06 | 0.27 |
| t782634 | [E17N, I33R, D39Q] | 125425 | 30202.47 | 2.05 | 0.35 |
| t782649 | [I33N, F95M] | 145500 | 14364.31 | 2.03 | 0.19 |
| t783298 | [A18D, T26H, D39Q] | 113575 | 26853.72 | 1.99 | 0.13 |
| t783333 | [T26H, D39Q] | 79200 | 19386.77 | 1.98 | 0.74 |
| t783289 | [V31L, D71S, D83N] | 103125 | 22393.95 | 1.98 | 0.29 |
| t782627 | [T26H, N29T, D39Q] | 128000 | 8485.28 | 1.97 | 0.25 |
| t783214 | [E17N, V31L, I33N] | 106875 | 12300.51 | 1.97 | 0.18 |
| t782407 | [D71S, D83N, F95M] | 132250 | 24088.38 | 1.91 | 0.40 |
| t783256 | [V31L, I33H, D71S] | 117500 | 14200.94 | 1.89 | 0.55 |
| t783216 | [I33R, D39G, F95M] | 112650 | 30138.96 | 1.88 | 0.20 |
| t782411 | [D39G, D71S, D83N] | 121750 | 7410.58 | 1.84 | 0.13 |
| t783171 | [N29T, I33R, D39Q] | 121850 | 27560.06 | 1.83 | 0.13 |
| t782395 | [T26A, D39Q, D83Q] | 84175 | 20696.11 | 1.80 | 0.39 |
| t783182 | [A18D, D39Q, V84F] | 126000 | 13735.60 | 1.80 | 0.17 |
| t783318 | [A18D, T26H, F95M] | 95400 | 16849.93 | 1.80 | 0.28 |
| t782447 | [I33R, D39G, D83N] | 109025 | 11593.21 | 1.79 | 0.26 |
| t782677 | [T26A, V31L, D39Q] | 106950 | 23892.19 | 1.79 | 0.69 |
| t782487 | [E17N, I33N, F95M] | 123500 | 14849.24 | 1.78 | 0.36 |
| t782593 | [T26H, D83Q, F95M] | 137250 | 5500.00 | 1.76 | 0.19 |
| t782654 | [D39Q, D71S, G80A] | 126250 | 13913.42 | 1.75 | 0.26 |
| t782476 | [E17N, N29T, G80A] | 81300 | 23963.03 | 1.74 | 0.91 |
| t783326 | [V31L, D39G, F95M] | 100825 | 10236.66 | 1.74 | 0.37 |
| t782558 | [D39G, D71S, V84F] | 130500 | 11818.07 | 1.73 | 0.38 |
| t782415 | [D39Q, D83Q] | 131750 | 10436.31 | 1.73 | 0.19 |
| t783143 | [T26A, N29T, I33H] | 127900 | 42706.32 | 1.68 | 0.60 |
| t783165 | [I33N, D39Q, F95M] | 132500 | 23388.03 | 1.67 | 0.13 |
| t782460 | [V31L, D83N, V84F] | 95275 | 23936.77 | 1.66 | 0.13 |
| t783329 | [E17N, T26A, D39G] | 122750 | 18589.87 | 1.62 | 0.23 |
| t782497 | [A18D, D39Q] | 116125 | 17712.40 | 1.61 | 0.16 |
| t783294 | [V31L, I33R] | 96066.66667 | 4833.56 | 1.60 | 0.19 |
| t783204 | [T26H, N29T, V84F] | 97400 | 19233.30 | 1.59 | 0.13 |
| t783172 | [V31L, I33R, D83N] | 111075 | 21233.21 | 1.59 | 0.21 |
| t782589 | [E17N, I33N, V84F] | 109450 | 18432.13 | 1.58 | 0.37 |
| t783217 | [N29T, I33N, D83Q] | 109125 | 18220.02 | 1.58 | 0.35 |
| t783210 | [N29T, V31L] | 104300 | 8061.02 | 1.57 | 0.14 |
| t783160 | [I33R, F95M] | 103350 | 25286.29 | 1.56 | 0.11 |
| t782674 | [A18D, T26H] | 83475 | 14618.34 | 1.53 | 0.49 |
| t783126 | [T26H, I33H, D83Q] | 83350 | 15627.06 | 1.53 | 0.15 |
| t782504 | N/A (Wild-type Control) | 105480 | 18803.59 | 1.52 | 0.32 |
| t783089 | [T26A, N29T, I33R] | 102925 | 11378.74 | 1.51 | 0.34 |
| t782405 | [D71S, D83Q] | 109650 | 18192.58 | 1.51 | 0.54 |
| t782673 | [T26A, F95M] | 113575 | 23737.22 | 1.51 | 0.30 |
| t782477 | [I33H, D39Q, G80A] | 109600 | 20465.09 | 1.50 | 0.12 |
| t782451 | [I33N, D39G, D83Q] | 100625 | 8775.11 | 1.47 | 0.10 |
| t783324 | [T26H, D39G, V84F] | 119000 | 4898.98 | 1.47 | 0.35 |
| t782604 | [D39G, D83A] | 84975 | 2382.40 | 1.47 | 0.18 |
| t782464 | [E17N, T26A, N29T] | 96625 | 7191.84 | 1.46 | 0.30 |
| t782423 | [T26A, D83N, F95M] | 118000 | 9933.11 | 1.45 | 0.13 |
| t782833 | [A18D, I33R, D39G] | 118500 | 2121.32 | 1.44 | 0.06 |
| t782669 | [E17N, D71S, F95M] | 124325 | 21002.12 | 1.44 | 0.21 |
| t782443 | [T26H, V31L, D83A] | 96975 | 4885.61 | 1.44 | 0.43 |
| t783316 | [T26H, D39Q, D83Q] | 102250 | 10803.24 | 1.42 | 0.23 |
| t782645 | [N29T, V31L, D83N] | 91175 | 27712.26 | 1.38 | 0.27 |
| t783288 | [E17N, G80A, D83N] | 115750 | 13225.61 | 1.37 | 0.46 |
| t782459 | [I33H, D83Q, F95M] | 107650 | 17249.83 | 1.36 | 0.25 |
| t782678 | [N29T, D39G, D83N] | 103000 | 11313.71 | 1.33 | 0.22 |
| t783151 | [A18D, D71S, G80A] | 129300 | 28311.83 | 1.32 | 0.20 |
| t782661 | [I33N, D71S, G80A] | 109100 | 17630.66 | 1.29 | 0.16 |
| t782646 | [I33R, D83A] | 90475 | 11315.88 | 1.27 | 0.21 |
| t782419 | [T26H, N29T, I33R] | 105225 | 21279.16 | 1.26 | 0.27 |
| t782467 | [T26H, I33N, G80A] | 113100 | 16245.20 | 1.26 | 0.34 |
| t782481 | [D83N, F95M] | 91175 | 7944.13 | 1.26 | 0.12 |
| t783211 | [E17N, T26A, G80A] | 85150 | 28828.05 | 1.25 | 0.16 |
| t782428 | [I33R, D39Q, V84F] | 120250 | 9287.09 | 1.24 | 0.11 |
| t782442 | [T26H, I33R, G80A] | 88550 | 15858.02 | 1.21 | 0.19 |
| t782403 | [T26A, N29T] | 108925 | 10684.37 | 1.18 | 0.04 |
| t782653 | [T26A, N29T, V31L] | 123250 | 9912.11 | 1.17 | 0.07 |
| t782472 | [T26H, V31L, D83N] | 92400 | 5273.83 | 1.16 | 0.16 |
| t782666 | [T26H, D83N] | 104925 | 13907.64 | 1.15 | 0.14 |
| t783176 | [E17N, T26H, M37L] | 79175 | 12900.48 | 1.07 | 0.43 |
| t621324 | [A18D E22K T26A N29S | 55637.5 | 9750.01 | 0.66 | 0.09 |
| | E53H H57Y Q70L G82K | | | | |
| | D83A S87K] | | | | |
| t782404 | N/A (GFP control) | 42837.5 | 6759.11 | 0.32 | 0.04 |

TABLE 7
Activity data for synthetic OAC polypeptides in S. cerevisiae
described in Example 2 and FIGS. 10A-10B.

| | | Average | | | |
|---|---|---|---|---|---|
| | Mutations relative | Olivetolic | Standard Deviation | Average | Standard |
| | to CsOAC | Acid | Olivetolic Acid | OA/OL | Deviation OA/OL |
| Strain | (SEQ ID NO: 1) | [μg/L] | [μg/L] | ratio | ratio |
| t815866 | [A18D, N29T, | 35749.00 | 4645.51 | 3.53 | 0.67 |
| | V31L, I33N, D71S, | | | | |
| | D83A] | | | | |
| t815873 | [E17N, A18D, | 47197.14 | 4955.33 | 3.26 | 0.44 |
| | V31L, D39Q, D71S, | | | | |
| | D83A] | | | | |
| t815900 | [E17N, A18D, | 41329.14 | 3257.12 | 3.19 | 0.82 |
| | V31L, D71S, D83A, | | | | |
| | V84F] | | | | |
| t815854 | [A18D, V31L, | 41449.30 | 1578.33 | 3.18 | 0.28 |
| | D39Q, D71S, D83A, | | | | |
| | V84F] | | | | |
| t782504 | N/A (wild-type | 40344.42 | 4937.39 | 3.09 | 0.75 |
| (CsOAC | CsOAC) | | | | |
| control) | | | | | |
| t815880 | [A18D, N29T, | 43303.03 | 9974.64 | 3.01 | 0.34 |
| | V31L, D71S, D83A, | | | | |
| | V84F] | | | | |
| t815889 | [A18D, V31L, I33N, | 40807.95 | 4219.60 | 3.01 | 0.07 |
| | D71S, D83A, V84F] | | | | |
| t815877 | [A18D, N29T, | 36091.62 | 5306.50 | 2.97 | 0.23 |
| | V31L, D39Q, D71S, | | | | |
| | D83A] | | | | |
| t815903 | [A18D, V31L, I33N, | 43634.75 | 5037.15 | 2.87 | 0.26 |
| | D39Q, D71S, D83A] | | | | |
| t815853 | [A18D, N29T, | 43642.68 | NaN | 2.82 | NaN |
| | V31L, D39Q, D83A, | | | | |
| | V84F] | | | | |
| t815905 | [E17N, A18D, I33N, | 38172.27 | 1406.28 | 2.68 | 0.12 |
| | D39Q, D71S, D83A] | | | | |
| t815863 | [E17N, A18D, | 36094.76 | 8414.97 | 2.64 | 0.10 |
| | V31L, I33N, D71S, | | | | |
| | V84F] | | | | |
| t815871 | [A18D, N29T, I33N, | 44488.79 | 2020.74 | 2.59 | 0.60 |
| | D39Q, D71S, D83A] | | | | |
| t815837 | [E17N, A18D, | 36312.69 | 5578.28 | 2.59 | 0.08 |
| | V31L, I33N, D83A, | | | | |
| | V84F] | | | | |
| t815835 | [E17N, A18D, | 32658.87 | 5410.17 | 2.50 | 0.09 |
| | N29T, V31L, I33N, | | | | |
| | D83A] | | | | |
| t815899 | [E17N, A18D, | 41669.54 | 2020.40 | 2.47 | 0.37 |
| | V31L, D39Q, D83A, | | | | |
| | V84F] | | | | |
| t815838 | A18D, N29T, | 37370.30 | 4081.42 | 2.34 | 0.09 |
| | V31L, I33N, D83A, | | | | |
| | V84F] | | | | |
| t815901 | [E17N, A18D, | 39489.34 | 729.89 | 2.27 | 0.22 |
| | N29T, V31L, D71S, | | | | |
| | V84F] | | | | |
| t815881 | [E17N, A18D, | 32774.59 | 4057.74 | 2.22 | 0.12 |
| | N29T, V31L, D39Q, | | | | |
| | D83A] | | | | |
| t815828 | [E17N, A18D, | 40926.17 | 6005.50 | 2.18 | 0.40 |
| | D39Q, D71S, D83A, | | | | |
| | V84F] | | | | |
| t815888 | [E17N, A18D, I33N, | 36730.64 | 2676.94 | 2.13 | 0.71 |
| | D71S, D83A, V84F] | | | | |
| t815834 | [A18D, N29T, | 35009.31 | 2374.51 | 2.13 | 0.10 |
| | V31L, I33N, D71S, | | | | |
| | V84F] | | | | |
| t815904 | [A18D, I33N, D39Q, | 36221.85 | 6677.50 | 2.09 | 0.52 |
| | D71S, D83A, V84F] | | | | |
| t815827 | [E17N, A18D, | 34860.93 | 15643.87 | 1.97 | 1.23 |
| | V31L, I33N, D71S, | | | | |
| | D83A] | | | | |
| t815907 | [E17N, A18D, | 33995.00 | 6537.45 | 1.96 | 0.65 |
| | V31L, I33N, D39Q, | | | | |
| | D83A] | | | | |
| t815849 | [A18D, N29T, I33N, | 28538.88 | 5644.49 | 1.94 | 0.16 |
| | D39Q, D83A, V84F] | | | | |
| t815855 | [E17N, A18D, I33N, | 30089.72 | 147.45 | 1.91 | 0.01 |
| | D39Q, D83A, V84F] | | | | |
| t815842 | [A18D, N29T, | 38794.41 | 8972.62 | 1.87 | 0.09 |
| | V31L, D39Q, D71S, | | | | |
| | V84F] | | | | |
| t815867 | [E17N, A18D, | 43486.43 | 6654.88 | 1.81 | 0.49 |
| | V31L, D39Q, D71S, | | | | |
| | V84F] | | | | |
| t815906 | [E17N, A18D, | 28859.48 | 2431.80 | 1.80 | 0.52 |
| | V31L, I33N, D39Q, | | | | |
| | D71S] | | | | |
| t815852 | [A18D, N29T, | 32126.56 | 8527.84 | 1.78 | 0.61 |
| | V31L, I33N, D39Q, | | | | |
| | D83A] | | | | |
| t815879 | [E17N, A18D, | 35056.85 | 778.11 | 1.78 | 0.06 |
| | N29T, I33N, D71S, | | | | |
| | D83A] | | | | |
| t815896 | [E17N, A18D, I33N, | 27853.32 | 4862.01 | 1.65 | 0.10 |
| | D39Q, D71S, V84F] | | | | |
| t815895 | [A18D, N29T, | 29013.73 | 776.74 | 1.61 | 0.01 |
| | D39Q, D71S, D83A, | | | | |
| | V84F] | | | | |
| t815836 | [A18D, N29T, I33N, | 33932.07 | 2658.16 | 1.58 | 0.25 |
| | D71S, D83A, V84F] | | | | |
| t815902 | [E17N, A18D, | 34708.90 | 3892.84 | 1.58 | 0.09 |
| | V31L, I33N, D39Q, | | | | |
| | V84F] | | | | |
| t815865 | [E17N, A18D, | 27609.94 | 1441.49 | 1.56 | 0.11 |
| | N29T, 133N, D39Q, | | | | |
| | D83A] | | | | |
| t815908 | [E17N, A18D, | 29852.50 | 2287.07 | 1.51 | 0.54 |
| | N29T, D39Q, D71S, | | | | |
| | D83A] | | | | |
| t815843 | [A18D, N29T, | 35731.01 | 10611.66 | 1.43 | 0.15 |
| | V31L, I33N, D39Q, | | | | |
| | V84F] | | | | |
| t815875 | [E17N, A18D, | 30632.17 | 1861.61 | 1.40 | 0.14 |
| | N29T, D71S, D83A, | | | | |
| | V84F] | | | | |
| t815872 | [N29T, V31L, I33N, | 33086.50 | 5504.44 | 1.35 | 0.03 |
| | D39Q, D71S, D83A] | | | | |
| t815898 | [E17N, A18D, | 24271.32 | 3358.39 | 1.32 | 0.22 |
| | N29T, I33N, D83A, | | | | |
| | V84F] | | | | |
| t815897 | [E17N, A18D, | 28267.49 | 1626.04 | 1.29 | 0.25 |
| | N29T, V31L, I33N, | | | | |
| | D71S] | | | | |
| t815850 | [E17N, N29T, | 28683.03 | 2743.43 | 1.20 | 0.26 |
| | V31L, I33N, D71S, | | | | |
| | D83A] | | | | |
| t815910 | [N29T, I33N, D39Q, | 23567.96 | 6052.88 | 1.17 | 0.42 |
| | D71S, D83A, V84F] | | | | |
| t815882 | [E17N, A18D, | 26275.60 | 218.96 | 1.13 | 0.03 |
| | N29T, V31L, I33N, | | | | |
| | D39Q] | | | | |
| t815869 | [A18D, V31L, I33N, | 26312.14 | 7219.06 | 1.13 | 0.39 |
| | D39Q, D71S, V84F] | | | | |
| t815878 | [E17N, N29T, | 23511.30 | 3103.18 | 1.07 | 0.23 |
| | V31L, I33N, D83A, | | | | |
| | V84F] | | | | |
| t815858 | [E17N, V31L, I33N, | 30845.73 | 6875.73 | 1.05 | 0.17 |
| | D71S, D83A, V84F] | | | | |
| t815857 | [E17N, V31L, I33N, | 29770.65 | 6425.08 | 1.05 | 0.28 |
| | D39Q, D71S, D83A] | | | | |
| t815846 | [E17N, A18D, | 21644.98 | 15432.47 | 0.98 | 1.06 |
| | N29T, V31L, I33N, | | | | |
| | V84F] | | | | |
| t815886 | [E17N, V31L, | 22966.40 | 9020.82 | 0.94 | 0.44 |
| | D39Q, D71S, D83A, | | | | |
| | V84F] | | | | |
| t815894 | [N29T, V31L, I33N, | 19807.04 | 1913.56 | 0.90 | 0.02 |
| | D39Q, D83A, V84F] | | | | |
| t815891 | [E17N, A18D, | 16423.81 | 2292.54 | 0.83 | 0.04 |
| | N29T, I33N, D71S, | | | | |
| | V84F] | | | | |
| t815831 | [E17N, A18D, | 19842.58 | 314.09 | 0.81 | 0.10 |
| | N29T, V31L, D39Q, | | | | |
| | V84F] | | | | |
| t815892 | [E17N, N29T, | 27654.77 | NaN | 0.78 | NaN |
| | V31L, D71S, D83A, | | | | |
| | V84F] | | | | |
| t815840 | [E17N, I33N, D39Q, | 18279.26 | 1046.99 | 0.72 | 0.01 |
| | D71S, D83A, V84F] | | | | |
| t815909 | [E17N, A18D, | 16671.36 | 2033.67 | 0.69 | 0.01 |
| | N29T, I33N, D39Q, | | | | |
| | D71S] | | | | |
| t621324 | [A18D E22K T26A | 16829.38 | 2766.43 | 0.67 | 0.18 |
| control | N29S E53H H57Y | | | | |
| | Q70L G82K D83A | | | | |
| | S87K] | | | | |
| t815868 | [E17N, N29T, | 16907.68 | 1586.46 | 0.66 | 0.03 |
| | V31L, I33N, D39Q, | | | | |
| | D83A] | | | | |
| t815856 | [E17N, N29T, | 17081.44 | 2908.10 | 0.65 | 0.05 |
| | V31L, D39Q, D71S, | | | | |
| | D83A] | | | | |
| t815851 | [N29T, V31L, I33N, | 16447.30 | 295.78 | 0.53 | 0.04 |
| | D39Q, D71S, V84F] | | | | |
| t815860 | [E17N, N29T, I33N, | 16483.78 | 1949.73 | 0.53 | 0.13 |
| | D39Q, D71S, D83A] | | | | |
| t815830 | [A18D, N29T, | 15613.24 | 883.00 | 0.52 | 0.06 |
| | V31L, I33N, D39Q, | | | | |
| | D71S] | | | | |
| t815841 | [E17N, A18D, | 15061.45 | 2395.21 | 0.51 | 0.10 |
| | N29T, I33N, D39Q, | | | | |
| | V84F] | | | | |
| t815844 | [E17N, N29T, | 14346.60 | 563.91 | 0.50 | 0.06 |
| | V31L, I33N, D71S, | | | | |
| | V84F] | | | | |
| t815890 | [E17N, N29T, I33N, | 8905.77 | 11353.83 | 0.48 | 0.71 |
| | D39Q, D83A, V84F] | | | | |
| t815861 | [E17N, N29T, | 13072.13 | 566.05 | 0.45 | 0.06 |
| | V31L, D39Q, D83A, | | | | |
| | V84F] | | | | |
| t815874 | [E17N, A18D, | 12902.23 | 1626.79 | 0.45 | 0.14 |
| | N29T, D39Q, D71S, | | | | |
| | V84F] | | | | |
| t815893 | [E17N, N29T, I33N, | 10230.32 | 811.02 | 0.38 | 0.01 |
| | D71S, D83A, V84F] | | | | |
| t815829 | [E17N, V31L, I33N, | 10524.50 | 1187.27 | 0.36 | 0.03 |
| | D39Q, D71S, V84F] | | | | |
| t815832 | [E17N, N29T, | 8802.50 | 4580.88 | 0.34 | 0.20 |
| | V31L, I33N, D39Q, | | | | |
| | V84F] | | | | |
| t815839 | [E17N, A18D, | 8414.07 | 6954.17 | 0.32 | 0.31 |
| | N29T, V31L, D39Q, | | | | |
| | D71S] | | | | |
| t782404 | N/A (GFP) | 8573.22 | 1281.74 | 0.31 | 0.00 |
| control | | | | | |
| t815833 | [E17N, V31L, I33N, | 9417.30 | 9216.60 | 0.30 | 0.31 |
| | D39Q, D83A, V84F] | | | | |
| t815859 | [E17N, N29T, | 8631.14 | 112.37 | 0.24 | 0.04 |
| | V31L, I33N, D39Q, | | | | |
| | D71S] | | | | |
| t815847 | [E17N, N29T, | 6905.20 | 803.92 | 0.20 | 0.01 |
| | V31L, D39Q, D71S, | | | | |
| | V84F] | | | | |
| t815864 | [E17N, N29T, I33N, | 5576.78 | 830.47 | 0.16 | 0.03 |
| | D39Q, D71S, V84F] | | | | |

### Example 3: Identification of Additional Synthetic OAC Polypeptides that are Capable of Producing Olivetolic Acid

An additional library of synthetic OAC polypeptides was designed using a combination of: (1) statistical information derived from homologous proteins; (2) variant abundance data derived from a multiplexed assay; (3) variant stability differences predicted from structures; and (4) activity data from previous designs obtained via an in vivo activity assay. Statistical information from homologous proteins was obtained from two models both derived from a multiple sequence alignment of several thousand homologs to CsOAC: a position-specific scoring matrix, which scores amino acids at each position as either being present more often or less often than one would expect from chance; and a second-order Potts model which incorporates both position-specific information as well as interactions between positions in the multiplex sequence alignment, similarly scoring full-length sequences as either more or less likely to occur. Variant abundance data were derived from both a site-scanning library presenting all possible single mutations of CsOAC as well as a combinatorial library in which previously identified beneficial mutations were recombined together. The variant abundance was determined in a multiplexed assay wherein synthetic OAC polypeptides were expressed as genetic fusions to GFP and isolated based upon their fluorescent signal at a single-cell level. Variants that were brighter were assumed to be able to be expressed at a high level, due to some combination of increased thermal and colloidal stability. Variant stability predictions were computed using structural models of mutants of CsOAC and a scoring algorithm combining physics-based and statistically-based features of protein structures. Scores are assumed to be correlative with protein stability. Activity data from previous designs were obtained via the in vivo assays described above. Previous designs incorporated portions of the scoring systems proposed above. These data were combined and filtered across a collection of thresholds to obtain corresponding collections of point-mutations. These point-mutations were then combinatorially evaluated via a structure-guided scoring algorithm.

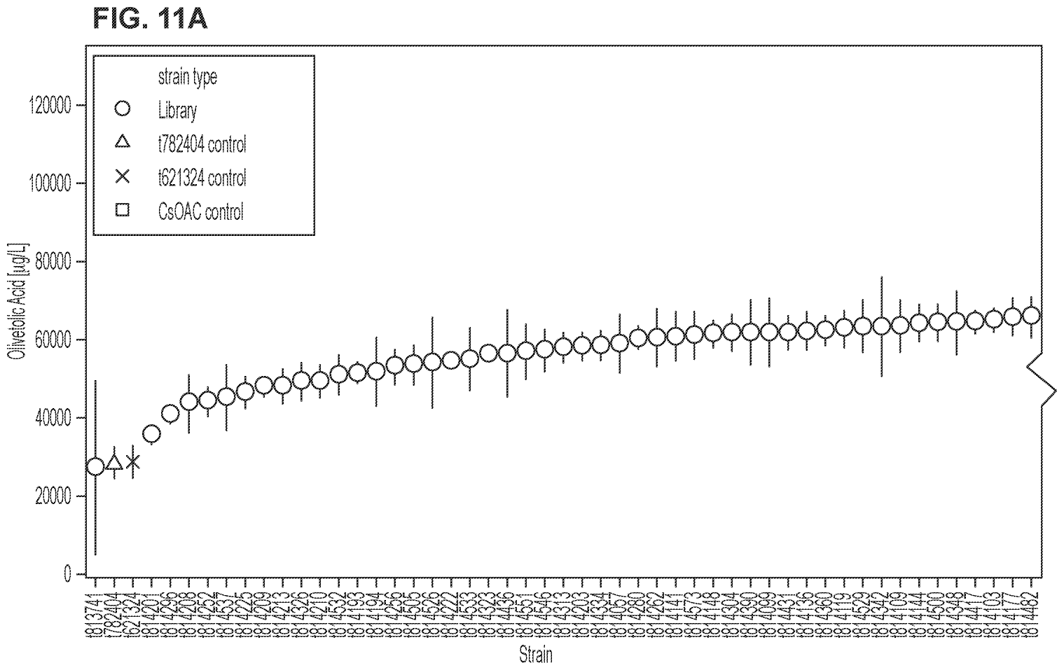

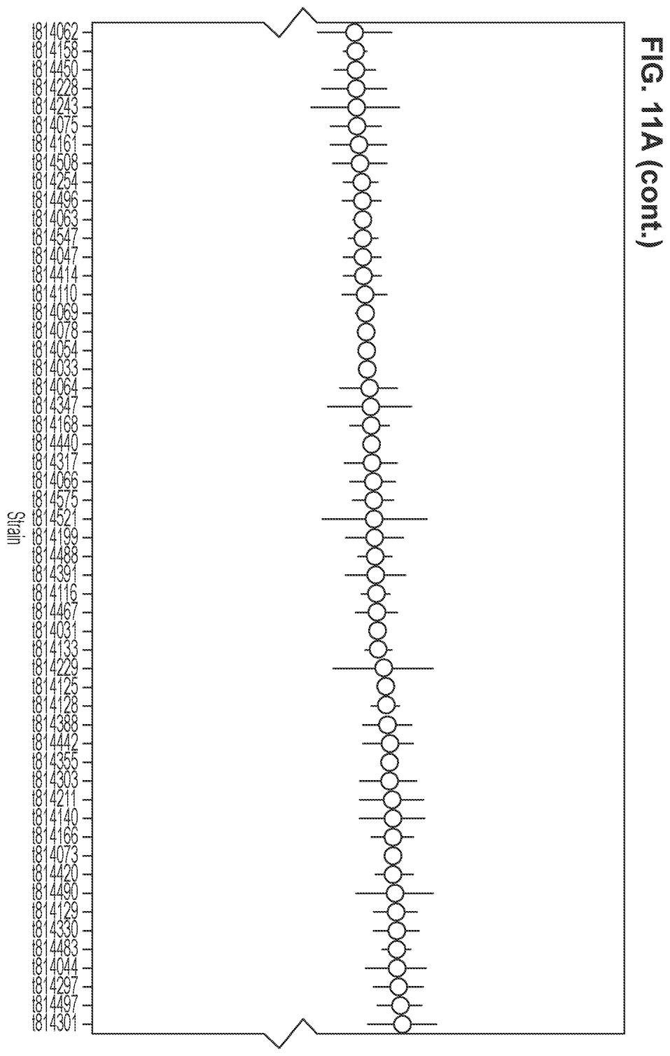

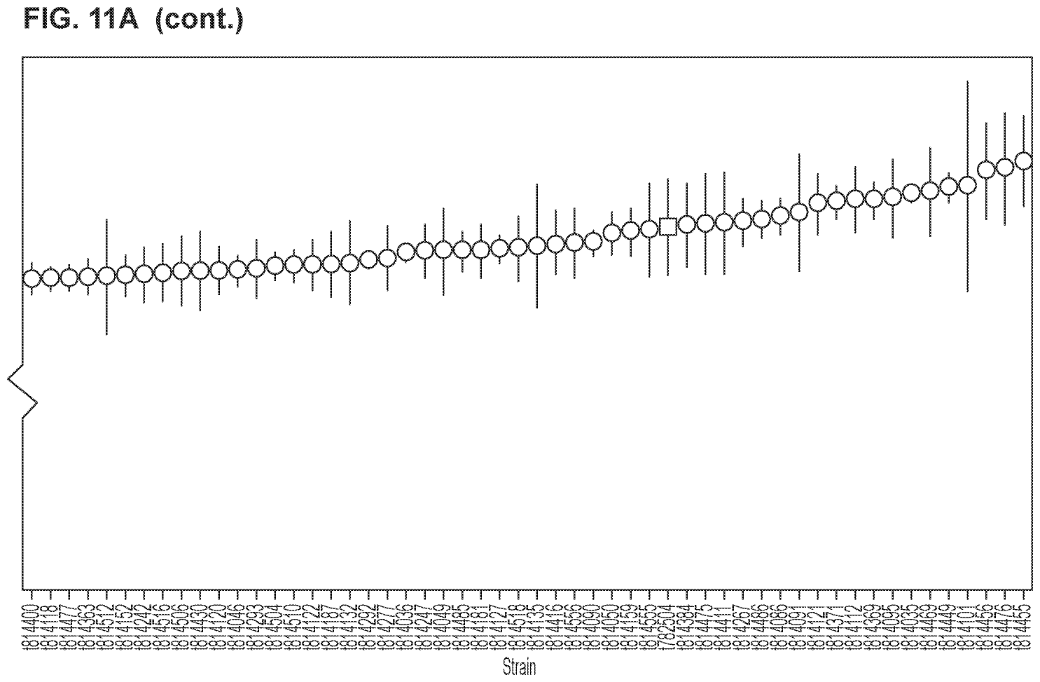

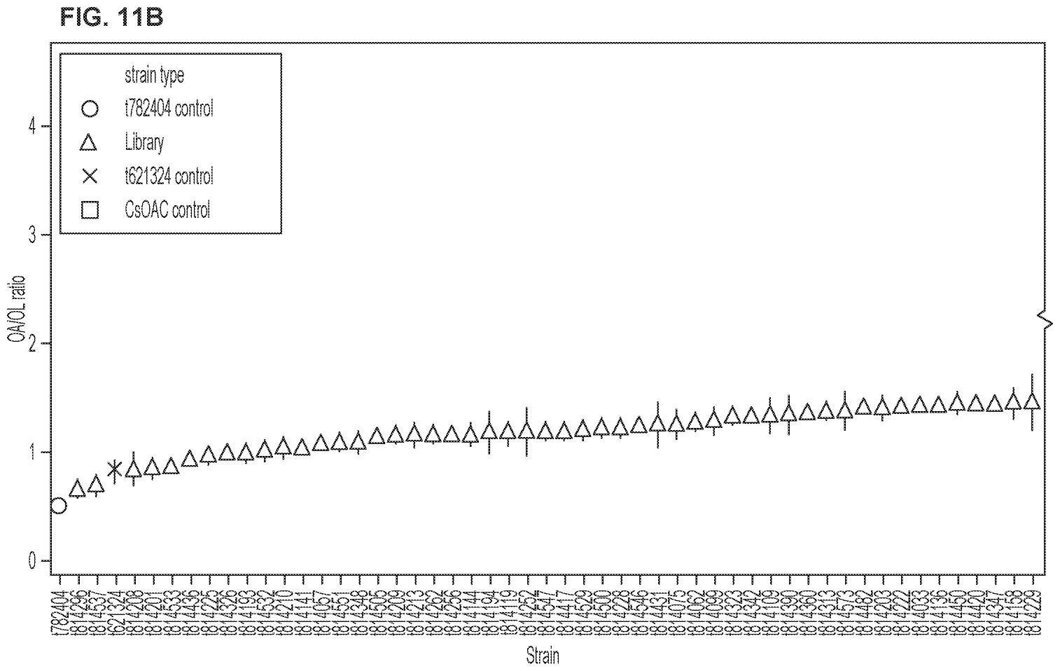

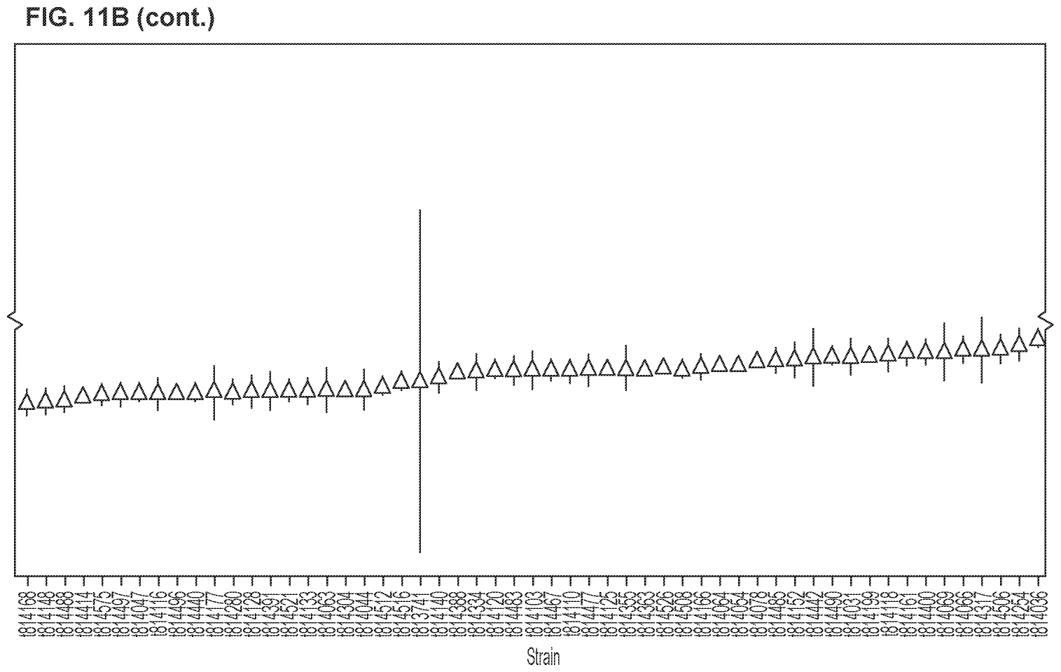

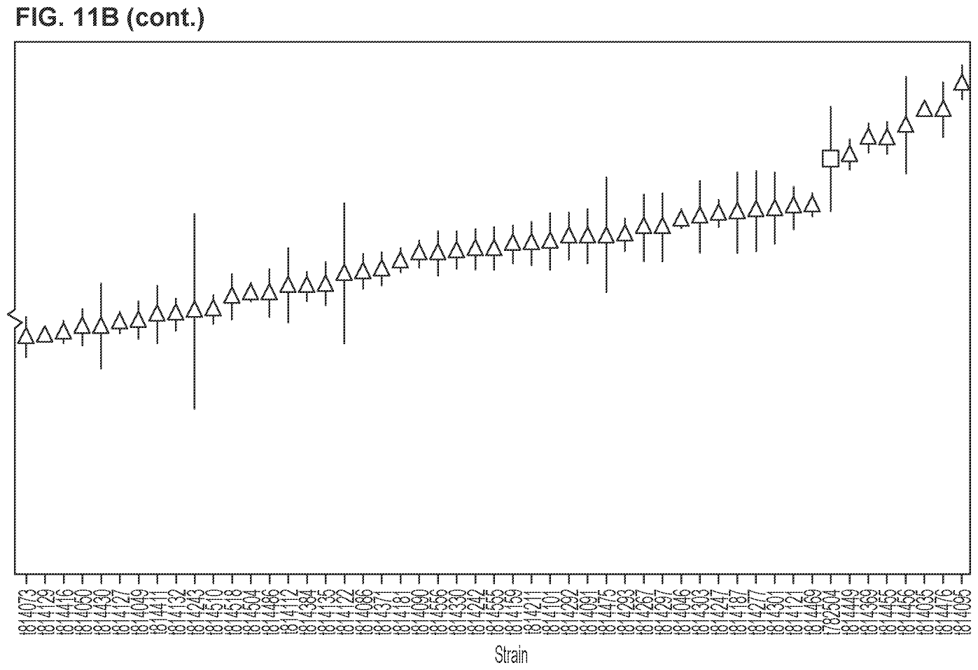

900 synthetic OAC polypeptides generated via these various design strategies were assembled into a library. For expression in S. cerevisiae, nucleotide sequences were recoded, synthesized, and cloned into an integrative yeast expression vector. Each candidate OAC expression construct was transformed into a S. cerevisiae CEN.PK strain previously engineered to produce olivetol by carrying a genomically integrated olivetol synthase as in Example 1. Strain t782504, expressing CsOAC, was included in the library as a positive control. Strain t621324, expressing a CsOAC variant containing a combination of 10 point mutations relative to the CsOAC peptide sequence, was included in the library as positive control for hit ranking. Strain t782404, expressing a fluorescent protein (GFP), was included in the library as a negative control for enzyme activity. 156 hit strains were elevated to a secondary screen to verify OA and OL production (FIGS. 11A-11B and Table 8).

TABLE 8
Activity data for synthetic OAC polypeptides in S. cerevisiae
described in Example 3 and FIGS. 11A-11B.

| | | | Standard | | Standard |
|---|---|---|---|---|---|
| | Mutations relative to | | Deviation | Average | Deviation |
| | CsOAC | Average Olivetolic | Olivetolic Acid | OA/OL | OA/OL |
| Strain | (SEQ ID NO: 1) | Acid [ug/L] | [ug/L] | ratio | ratio |
| t814095 | [E64D, D71E] | 99259.28 | 11458.83 | 4.39 | 0.16 |
| t814476 | [E64D, D71E, T98K] | 106932.56 | 16461.36 | 4.15 | 0.28 |
| t814035 | [D71E] | 100324.04 | 3036.19 | 4.15 | 0.01 |
| t814456 | [S65N] | 106438.46 | 14038.86 | 4.00 | 0.51 |
| t814455 | [Y85F] | 108539.98 | 13276.90 | 3.89 | 0.17 |
| t814369 | [D71E, Y85F] | 98995.28 | 5512.66 | 3.89 | 0.14 |
| t814449 | [G80N] | 102027.20 | 4092.05 | 3.74 | 0.16 |
| CsOAC | N/A (CsOAC control) | 92056.51 | 12562.84 | 3.69 | 0.51 |
| control | | | | | |
| t814469 | [T68G] | 101196.40 | 13043.65 | 3.26 | 0.12 |
| t814121 | [I33R, E52H, Y55F, | 97896.01 | 9101.88 | 3.25 | 0.23 |
| | Q70A, I74G, D83K] | | | | |
| t814301 | [F88N] | 78443.74 | 10220.50 | 3.24 | 0.37 |
| t814277 | [T98K] | 83784.09 | 9470.12 | 3.22 | 0.43 |
| t814187 | [K101A] | 82473.77 | 9474.23 | 3.19 | 0.43 |
| t814247 | [N50F] | 85833.59 | 7842.01 | 3.19 | 0.13 |
| t814303 | [R100A] | 75141.18 | 8308.64 | 3.16 | 0.38 |
| t814046 | [D71E, G80D, R100S] | 80790.96 | 5672.39 | 3.14 | 0.11 |
| t814297 | [R100G] | 77544.52 | 7072.56 | 3.06 | 0.36 |
| t814267 | [T98E] | 93281.71 | 6950.41 | 3.06 | 0.36 |
| t814293 | [F88S] | 81206.40 | 8900.53 | 2.99 | 0.17 |
| t814475 | [V61D] | 92817.30 | 14833.56 | 2.98 | 0.60 |
| t814091 | [Y55F, G80A, D83K] | 95682.15 | 17299.12 | 2.97 | 0.29 |
| t814292 | [Y97F] | 83386.13 | 2712.81 | 2.97 | 0.24 |
| t814101 | [V66Y] | 102326.29 | 30888.79 | 2.93 | 0.30 |
| t814211 | [Q48A] | 75848.61 | 9379.21 | 2.91 | 0.23 |
| t814159 | [F95I] | 90872.11 | 6843.17 | 2.91 | 0.19 |
| t814555 | [V61S] | 91250.85 | 13810.44 | 2.87 | 0.22 |
| t814242 | [R100Q] | 79722.98 | 7831.72 | 2.86 | 0.21 |
| t814330 | [R100N] | 76845.91 | 6667.67 | 2.85 | 0.20 |
| t814556 | [R100K] | 87722.73 | 10131.48 | 2.82 | 0.23 |
| t814090 | [A18D, T26R, I33R, | 87963.02 | 3921.69 | 2.82 | 0.14 |
| | E52H, Y55F, Q70A, | | | | |
| | G80A, D83N, V84K] | | | | |
| t814181 | [G82R] | 86167.85 | 7757.83 | 2.75 | 0.12 |
| t814371 | [D13R, A18D, N29S, | 98412.72 | 5276.22 | 2.68 | 0.17 |
| | Q70L, D71Q, G80A, | | | | |
| | D83K] | | | | |
| t814086 | [T26R, I33H, E52H, | 94827.60 | 5212.14 | 2.66 | 0.18 |
| | Y55F, Q70A, D83K] | | | | |
| t814122 | [E52H, Y55F, I74G, | 82360.04 | 7505.42 | 2.64 | 0.73 |
| | G80A, D83K, Y85A] | | | | |
| t814135 | [A18D, T26R, I33R, | 86978.55 | 18258.92 | 2.53 | 0.23 |
| | E52H, Y55F, Q70A, | | | | |
| | I74G, G80A, D83N] | | | | |
| t814384 | [E22K, T26H, N29S, | 92471.22 | 12266.86 | 2.53 | 0.15 |
| | D39G, K49Q, E52H, | | | | |
| | D83N, V84K] | | | | |
| t814112 | [I33R, E52H, Y55F, | 98929.52 | 9627.43 | 2.52 | 0.38 |
| | Q70A, G80A, D83K, | | | | |
| | V84K] | | | | |
| t814486 | [A18D, T26R, E52H, | 93991.25 | 5641.02 | 2.45 | 0.24 |
| | Y55F, Q70A, G80A, | | | | |
| | D83N, V84K, Y85A] | | | | |
| t814504 | [I33H, E52H, Y55F, | 81892.82 | 4132.88 | 2.45 | 0.09 |
| | Q70A, I74G, G80A, | | | | |
| | D83K] | | | | |
| t814518 | [D45I] | 86508.97 | 9610.99 | 2.42 | 0.25 |
| t814510 | [A18D, T26R, I33H, | 82196.15 | 4928.22 | 2.31 | 0.14 |
| | E52H, Y55F, Q70A, | | | | |
| | I74G, G80A, D83N] | | | | |
| t814243 | [R100S] | 66545.44 | 12933.04 | 2.29 | 1.03 |
| t814132 | [F24M, T26R, E52H, | 82553.61 | 12048.12 | 2.26 | 0.16 |
| | Y55F, G80A, D83K] | | | | |
| t814411 | [T26R, I33H, Y55F, | 92841.09 | 15188.37 | 2.25 | 0.31 |
| | Q70A, G80A, D83K, | | | | |
| | Y85A] | | | | |
| t814049 | [A18D, T26R, I33R, | 85908.13 | 15504.24 | 2.20 | 0.25 |
| | E52H, Y55F, Q70A, | | | | |
| | G80A, D83K] | | | | |
| t814127 | [T26R, E52H, Y55F, | 86305.45 | 3855.22 | 2.18 | 0.11 |
| | Q70A, G80A, D83N, | | | | |
| | V84K, Y85A] | | | | |
| t814430 | [T26R, I33R, E52H, | 80742.44 | 11285.88 | 2.15 | 0.44 |
| | G80A, D83N, Y85A] | | | | |
| t814050 | [T26R, E52H, Y55F, | 90110.23 | 6318.84 | 2.14 | 0.19 |
| | Q70A, G80A, D83K, | | | | |
| | V84F] | | | | |
| t814416 | [A18D, T26R, I33H, | 87700.85 | 9388.18 | 2.09 | 0.11 |
| | E52H, Y55F, Q70A, | | | | |
| | G80A, D83K] | | | | |
| t814129 | [A18D, T26R, E52H, | 76833.34 | 6423.15 | 2.06 | 0.05 |
| | Y55F, Q70A, I74G, | | | | |
| | G80A, D83N, Y85A] | | | | |
| t814073 | [T26R, I33H, E52H, | 76193.56 | 1643.99 | 2.05 | 0.20 |
| | Y55F, Q70A, I74G, | | | | |
| | G80A, D83N, Y85A] | | | | |
| t814036 | [E52H, Y55F, Q70A, | 85381.80 | 2493.49 | 2.04 | 0.07 |
| | G80A, D83K, V84K, | | | | |
| | Y85A] | | | | |
| t814254 | [E22K, T26R, I33H, | 68036.82 | 4857.23 | 2.00 | 0.16 |
| | N50K, D71Q, G80A, | | | | |
| | D83E, T98Y] | | | | |
| t814506 | [T26R, I33R, E52H, | 80706.53 | 10279.55 | 1.96 | 0.15 |
| | Y55F, Q70A, G80A, | | | | |
| | D83K] | | | | |
| t814317 | [A18D, E22K, N29S, | 70516.82 | 7598.78 | 1.95 | 0.34 |
| | Q70A, G82K, D83R, | | | | |
| | S87K, F88Y] | | | | |
| t814066 | [I33H, E52H, Y55F, | 70870.95 | 6613.47 | 1.94 | 0.14 |
| | Q70A, G80A, D83K, | | | | |
| | V84K] | | | | |
| t814069 | [A18D, T26R, N29S, | 68881.87 | 2647.33 | 1.93 | 0.30 |
| | I33H, E52H, Y55F, | | | | |
| | Q70A, I74G, G80A, | | | | |
| | D83N, Y85A] | | | | |
| t814400 | [V59F] | 78649.60 | 4679.03 | 1.93 | 0.13 |
| t814161 | [A18D, E22K, T26A, | 67332.37 | 8497.08 | 1.92 | 0.13 |
| | N29S, Q70L, G82K, | | | | |
| | D83A] | | | | |
| t814118 | [T26R, I33R, E52H, | 78766.93 | 3474.85 | 1.91 | 0.17 |
| | Y55F, Q70A, I74G, | | | | |
| | G80A, D83N, V84K, | | | | |
| | Y85A] | | | | |
| t814199 | [N50W] | 71334.80 | 8348.90 | 1.90 | 0.05 |
| t814031 | [I33H, E52H, Q70A, | 72000.32 | 1054.90 | 1.89 | 0.20 |
| | G80A, D83K, Y85A] | | | | |
| t814490 | [A18D, T26R, I33H, | 76541.21 | 11297.36 | 1.88 | 0.08 |
| | E52H, Y55F, Q70A, | | | | |
| | D71Q, I74G, G80A, | | | | |
| | D83N, V84K, Y85A] | | | | |
| t814442 | [A18D, T26A, N29S, | 75008.38 | 7209.44 | 1.88 | 0.31 |
| | Q70L, G82K, D83E, | | | | |
| | S87K] | | | | |
| t814152 | [A18D, F24M, T26R, | 79395.67 | 5948.92 | 1.86 | 0.18 |
| | I33H, E52H, Y55F, | | | | |
| | Q70A, G80A, D83N] | | | | |
| t814485 | [I33H, E52H, Y55F, | 85922.63 | 5750.37 | 1.85 | 0.12 |
| | Q70A, G80V, D83K] | | | | |
| t814078 | [A18D, T26R, N29S, | 69219.21 | 2118.47 | 1.84 | 0.09 |
| | I33R, E52H, Y55F, | | | | |
| | Q70A, I74G, G80A, | | | | |
| | D83N, Y85A] | | | | |
| t814054 | [T26R, I33R, E52H, | 69535.34 | 531.41 | 1.80 | 0.09 |
| | Y55F, G80A, Y85A] | | | | |
| t814064 | [T26R, I33R, E52H, | 70010.30 | 8211.71 | 1.80 | 0.08 |
| | Q70A, G80A, D83K] | | | | |
| t814166 | [I33H, E52H, Y55F, | 76106.47 | 6106.34 | 1.80 | 0.13 |
| | Q70A, G80A, D83N, | | | | |
| | V84K, Y85A] | | | | |
| t814508 | [A18D, F24M, T26R, | 67651.17 | 7876.19 | 1.78 | 0.09 |
| | I33H, E52H, Y55F, | | | | |
| | Q70A, I74G, G80A, | | | | |
| | D83N, V84K, Y85A] | | | | |
| t814526 | [A18D, T26R, I33R, | 54304.54 | 13362.86 | 1.78 | 0.06 |
| | E52H, Y55F, Q70A, | | | | |
| | G80A, D83N, V84F] | | | | |
| t814363 | [T26R, I33R, Y55F, | 79214.22 | 5130.62 | 1.78 | 0.05 |
| | Q70A, G80A, D83K, | | | | |
| | Y85A] | | | | |
| t814355 | [T26R, I33H, E52H, | 75030.60 | 1368.09 | 1.78 | 0.23 |
| | Y55F, Q70A, G80A, | | | | |
| | D83E, Y85A] | | | | |
| t814125 | [T26R, I33H, E52H, | 73996.36 | 2073.33 | 1.77 | 0.06 |
| | Y55F, Q70A, G80A, | | | | |
| | Y85A] | | | | |
| t814477 | [A18D, T26R, I33H, | 78801.64 | 3693.25 | 1.77 | 0.17 |
| | E52H, Y55F, Q70A, | | | | |
| | G80A, D83E, Y85A] | | | | |
| t814110 | [I33H, E52H, Y55F, | 68786.64 | 6477.05 | 1.76 | 0.12 |
| | G80A, D83K, Y85A] | | | | |
| t814467 | [A18D, E22K, T26A, | 71944.63 | 6198.99 | 1.76 | 0.11 |
| | N29S, E53H, Q70L, | | | | |
| | G82K, D83A] | | | | |
| t814103 | [T26R, E52H, Y55F, | 65161.42 | 4494.72 | 1.76 | 0.22 |
| | G80A, D83K, Y85A] | | | | |
| t814483 | [V28H, I33H, E52H, | 76966.87 | 4601.42 | 1.76 | 0.14 |
| | Y55F, Q70A, G80A, | | | | |
| | D83K] | | | | |
| t814120 | [T26R, I33H, E52H, | 80770.56 | 6868.86 | 1.75 | 0.08 |
| | Y55F, G80A, D83N, | | | | |
| | Y85A] | | | | |
| t814334 | [A18D, T26R, N29S, | 58758.68 | 4476.79 | 1.75 | 0.19 |
| | I33R, E52H, Y55F, | | | | |
| | Q70A, I74G, G80A, | | | | |
| | D83N, V84K, Y85A] | | | | |
| t814388 | [A18D, E22K, N29S, | 74725.60 | 6838.14 | 1.74 | 0.04 |
| | Q70L, G82K, D83K, | | | | |
| | S87K] | | | | |
| t814140 | [A18D, T26R, E52H, | 76018.91 | 9585.13 | 1.69 | 0.16 |
| | Y55F, Q70A, G80A, | | | | |
| | D83K, Y85A] | | | | |
| t813741 | [A2G, V3Y, V46F, E60L, | 27548.07 | 25508.26 | 1.65 | 1.82 |
| | E67K, T68H, G80D, | | | | |
| | D83S, R86S, W89M, | | | | |
| | T98K, R100S, K101E] | | | | |
| t814516 | [I33H, E52H, Y55F, | 79997.35 | 8676.98 | 1.65 | 0.09 |
| | Q70A, G80A, D83K, | | | | |
| | V84F] | | | | |
| t814512 | [T26R, E52H, Y55F, | 79285.61 | 16774.42 | 1.60 | 0.09 |
| | Q70A, G80A, D83K, | | | | |
| | Y85A] | | | | |
| t814044 | [A18D, F24M, T26R, | 77077.52 | 8987.80 | 1.58 | 0.22 |
| | I33H, E52H, Y55F, | | | | |
| | Q70A, G80A, D83N, | | | | |
| | V84K, Y85A] | | | | |
| t814304 | [A18D, Q19D, F24M, | 61871.74 | 5451.10 | 1.58 | 0.03 |
| | N29S, N50K, Y55F, | | | | |
| | Q70A, I74G, G80V, | | | | |
| | D83E, V84K] | | | | |
| t814063 | [F24M, E52H, Q70A, | 68226.32 | 2516.64 | 1.58 | 0.24 |
| | G80A, D83K, Y85A] | | | | |
| t814133 | [A18D, T26R, N29S, | 72427.63 | 3842.75 | 1.57 | 0.13 |
| | I33R, E52H, Y55F, | | | | |
| | Q70A, D71Q, I74G, | | | | |
| | G80A, D83N, V84K, | | | | |
| | Y85A] | | | | |
| t814521 | [A18D, I33R, E52H, | 71320.48 | 15604.75 | 1.57 | 0.08 |
| | Y55F, Q70A, I74G, | | | | |
| | G80A, D83N, V84K, | | | | |
| | Y85A] | | | | |
| t814391 | [A18D, E22K, N29S, | 71625.07 | 8504.96 | 1.57 | 0.19 |
| | Q70L, G82K, D83Q, | | | | |
| | S87K, F88Y] | | | | |
| t814128 | [E52H, Y55F, Q70A, | 74221.09 | 4022.64 | 1.57 | 0.17 |
| | D83K, V84F, Y85A] | | | | |
| t814280 | [A18D, T26A, V28E, | 60686.44 | 3465.46 | 1.56 | 0.13 |
| | N29S, Q70A, G82K, | | | | |
| | D83A, S87K] | | | | |
| t814177 | [A18D, T26R, N29S, | 65847.20 | 5638.88 | 1.56 | 0.28 |
| | I33R, K49Q, E52H, | | | | |
| | Y55F, Q70A, I74G, | | | | |
| | G80A, D83N, V84K, | | | | |
| | Y85A] | | | | |
| t814440 | [A18D, T26A, V28E, | 70484.55 | 2175.67 | 1.55 | 0.10 |
| | N29S, Q70L, G82K, | | | | |
| | D83N, S87K] | | | | |
| t814496 | [F24M, T26R, I33R, | 68138.04 | 5631.80 | 1.55 | 0.05 |
| | E52H, Y55F, Q70A, | | | | |
| | G80A, D83N] | | | | |
| t814116 | [A18D, F24M, T26R, | 71734.50 | 3866.41 | 1.54 | 0.17 |
| | I33R, E52H, Y55F, | | | | |
| | Q70A, G80A, D83E, | | | | |
| | Y85A] | | | | |
| t814047 | [T26R, I33R, E52H, | 68260.55 | 5022.09 | 1.54 | 0.08 |
| | Y55F, Q70A, G80A, | | | | |
| | Y85A] | | | | |
| t814497 | [F24M, I33H, E52H, | 77950.71 | 6689.49 | 1.54 | 0.12 |
| | Y55F, Q70A, G80A, | | | | |
| | D83K] | | | | |
| t814575 | [A18D, T26R, I33R, | 71030.66 | 6079.55 | 1.53 | 0.10 |
| | E52H, Y55F, Q70A, | | | | |
| | G80A, D83N, V84K, | | | | |
| | Y85A] | | | | |
| t814414 | [A18D, T26R, I33R, | 68431.79 | 5346.05 | 1.52 | 0.05 |
| | E52H, Y55F, Q70A, | | | | |
| | G80A, D83K, V84K, | | | | |
| | Y85A] | | | | |
| t814488 | [A18D, E22K, N29S, | 71495.03 | 4874.13 | 1.49 | 0.12 |
| | I33R, Q70L, G82K, | | | | |
| | D83R, S87K] | | | | |
| t814148 | [A18D, T26R, I33H, | 61650.57 | 3906.47 | 1.47 | 0.13 |
| | E52H, Y55F, Q70A, | | | | |
| | G80A, D83K, V84K, | | | | |
| | Y85A] | | | | |
| t814168 | [E22K, K49Q, V61T, | 70325.66 | 5845.58 | 1.47 | 0.14 |
| | Q70A, D83Q, V84F] | | | | |
| t814229 | [A18D, T26A, V28E, | 73566.57 | 14281.63 | 1.46 | 0.29 |
| | N29S, Q70L, G82K, | | | | |
| | D83Q, S87K] | | | | |
| t814158 | [A18D, F24M, T26R, | 66339.50 | 3258.94 | 1.44 | 0.16 |
| | N29S, I33H, E52H, | | | | |
| | Y55F, Q70A, G80A, | | | | |
| | D83N, V84K, Y85A] | | | | |
| t814347 | [A18D, T26R, I33R, | 70304.17 | 12311.55 | 1.44 | 0.06 |
| | E52H, Y55F, Q70A, | | | | |
| | I74G, G80A, D83N, | | | | |
| | V84F, Y85A] | | | | |
| t814420 | [A18D, T26R, I33R, | 76252.04 | 5466.38 | 1.44 | 0.06 |
| | E52H, Y55F, Q70A, | | | | |
| | G80A, D83K, V84F, | | | | |
| | Y85A] | | | | |
| t814450 | [A18D, E22K, N29S, | 66414.74 | 5837.58 | 1.44 | 0.12 |
| | Q70L, G82K, D83G, | | | | |
| | S87K] | | | | |
| t814136 | [A18D, T26R, N29S, | 62421.27 | 5280.15 | 1.42 | 0.07 |
| | I33H, E52H, Y55F, | | | | |
| | Q70A, G80A, D83N, | | | | |
| | V84F, Y85A] | | | | |
| t814033 | [T26R, I33H, E52H, | 69559.56 | 2109.09 | 1.42 | 0.06 |
| | Y55F, Q70A, D71Q, | | | | |
| | G80A, D83N, Y85A] | | | | |
| t814222 | [A18D, T26A, N29S, | 54901.18 | 1403.25 | 1.40 | 0.07 |
| | E53H, Q70L, G82K, | | | | |
| | D83E, S87K] | | | | |
| t814203 | [A18D, F24M, T26R, | 58626.81 | 4369.84 | 1.40 | 0.13 |
| | N29S, I33H, E52H, | | | | |
| | Y55F, Q70A, I74G, | | | | |
| | G80A, D83N, V84K, | | | | |
| | Y85A] | | | | |
| t814482 | [T26R, V28H, E52H, | 66120.60 | 6045.55 | 1.40 | 0.06 |
| | Y55F, Q70A, G80A, | | | | |
| | D83K] | | | | |
| t814573 | [A18D, E22K, N29S, | 61313.57 | 7018.06 | 1.37 | 0.19 |
| | Q70L, G82K, D83N, | | | | |
| | S87K] | | | | |
| t814313 | [F24M, E52H, Y55F, | 58204.75 | 4570.64 | 1.36 | 0.08 |
| | Q70A, G80A, D83K, | | | | |
| | Y85A] | | | | |
| t814360 | [K49Q, E52H, Y55F, | 62506.07 | 4094.06 | 1.35 | 0.03 |
| | G80A, D83K, Y85A] | | | | |
| t814390 | [A18D, T26A, N29S, | 61892.12 | 9412.38 | 1.34 | 0.20 |
| | Q70L, G82K, D83A, | | | | |
| | S87K] | | | | |
| t814109 | [F24M, T26R, I33H, | 63601.88 | 7922.81 | 1.33 | 0.19 |
| | E52H, Y55F, Q70A, | | | | |
| | G80A, D83K, Y85A] | | | | |
| t814342 | [D13K, A18D, E21K, | 63541.75 | 14540.54 | 1.32 | 0.02 |
| | E22K, K25A, N29S, | | | | |
| | K38L, Y55F, V61T, | | | | |
| | Q70L, D71Q, I74G, | | | | |
| | G80A, G82K, D83A, | | | | |
| | V84K] | | | | |
| t814323 | [A18D, T26A, V28E, | 56683.38 | 1400.63 | 1.32 | 0.09 |
| | N29S, E53H, Q70L, | | | | |
| | G82K, D83E, S87K] | | | | |
| t814099 | [A18D, T26R, N29S, | 61958.05 | 9976.94 | 1.28 | 0.13 |
| | I33R, E52H, Y55F, | | | | |
| | Q70A, I74G, G80A, | | | | |
| | D83N, V84F, Y85A] | | | | |
| t814062 | [T26R, I33R, E52H, | 66136.28 | 10948.72 | 1.26 | 0.08 |
| | Y55F, Q70A, G80A, | | | | |
| | D83N, V84K, Y85A] | | | | |
| t814075 | [A18D, T26R, I33R, | 66591.97 | 7562.19 | 1.25 | 0.15 |
| | E52H, Y55F, Q70A, | | | | |
| | G80A, D83N, Y85A] | | | | |
| t814431 | [I33H, E52H, Y55F, | 61959.82 | 5090.07 | 1.25 | 0.24 |
| | Q70A, G80A, D83K, | | | | |
| | Y85A] | | | | |
| t814546 | [T26R, I33R, E52H, | 57603.45 | 6188.55 | 1.24 | 0.03 |
| | Y55F, G80A, D83N, | | | | |
| | Y85A] | | | | |
| t814228 | [A18D, E22K, N29S, | 66480.60 | 9351.15 | 1.22 | 0.09 |
| | I33R, Q70L, G82K, | | | | |
| | D83R, S87K, F88Y] | | | | |
| t814500 | [F24M, T26R, I33H, | 64503.93 | 5349.67 | 1.21 | 0.09 |
| | E52H, Y55F, Q70A, | | | | |
| | G80A, D83N, Y85A] | | | | |
| t814529 | [A18D, E22K, T26H, | 63482.82 | 7674.28 | 1.20 | 0.09 |
| | N29S, Q70L, G82K, | | | | |
| | D83R, S87K, F88Y] | | | | |
| t814417 | [I33R, E52H, Y55F, | 64711.41 | 3242.97 | 1.18 | 0.08 |
| | Q70A, G80A, D83N, | | | | |
| | V84F, Y85A] | | | | |
| t814547 | [T26R, I33H, E52H, | 68250.27 | 4314.36 | 1.18 | 0.11 |
| | Y55F, Q70A, G80A, | | | | |
| | D83K, Y85A] | | | | |
| t814252 | [A18D, T26A, N29S, | 44541.60 | 4363.76 | 1.18 | 0.25 |
| | Q70L, G82K, D83R, | | | | |
| | S87K] | | | | |
| t814119 | [T26R, K49Q, E52H, | 63142.34 | 5495.38 | 1.17 | 0.12 |
| | Y55F, Q70A, G80A, | | | | |
| | D83N, Y85A] | | | | |
| t814194 | [A18D, T26R, N29S, | 51910.53 | 10307.56 | 1.17 | 0.22 |
| | I33R, E52H, Y55F, | | | | |
| | Q70A, D71Q, I74G, | | | | |
| | G80A, G82K, D83N, | | | | |
| | V84F, Y85A] | | | | |
| t814144 | [A18D, T26R, I33H, | 64224.45 | 5491.59 | 1.16 | 0.11 |
| | E52H, Y55F, Q70A, | | | | |
| | G80A, D83K, V84F, | | | | |
| | Y85A] | | | | |
| t814256 | [A18D, T26R, I33R, | 53350.15 | 5315.87 | 1.15 | 0.07 |
| | E52H, Y55F, Q70A, | | | | |
| | D71Q, I74G, G80A, | | | | |
| | D83N, V84K, Y85A] | | | | |
| t814262 | [A18D, F24M, T26R, | 60797.71 | 8668.02 | 1.15 | 0.09 |
| | I33H, E52H, Y55F, | | | | |
| | Q70A, G80A, D83N, | | | | |
| | Y85A] | | | | |
| t814213 | [A18D, F24M, I33R, | 48284.65 | 5005.12 | 1.15 | 0.14 |
| | E52H, Y55F, Q70A, | | | | |
| | I74G, G80A, D83N, | | | | |
| | Y85A] | | | | |
| t814209 | [A18D, T26R, I33H, | 48231.27 | 2973.34 | 1.15 | 0.08 |
| | K49Q, E52H, Y55F, | | | | |
| | Q70A, D71Q, I74G, | | | | |
| | G80A, D83N, V84K, | | | | |
| | Y85A] | | | | |
| t814505 | [A18D, F24M, T26R, | 53861.71 | 5768.97 | 1.13 | 0.05 |
| | I33R, E52H, Y55F, | | | | |
| | Q70A, D71Q, I74G, | | | | |
| | G80A, D83N, V84F, | | | | |
| | Y85A] | | | | |
| t814348 | [T26R, I33R, E52H, | 64580.30 | 9556.06 | 1.08 | 0.11 |
| | Y55F, Q70A, G80A, | | | | |
| | D83K, Y85A] | | | | |
| t814551 | [A18D, T26R, I33R, | 57233.25 | 7854.62 | 1.08 | 0.08 |
| | E52H, Y55F, Q70A, | | | | |
| | D71Q, I74G, G80A, | | | | |
| | D83N, Y85A] | | | | |
| t814057 | [A18D, F24M, T26R, | 59128.02 | 8511.08 | 1.07 | 0.01 |
| | N29S, I33R, E52H, | | | | |
| | Y55F, Q70A, G80A, | | | | |
| | D83N, V84F, Y85A] | | | | |
| t814141 | [T26R, I33H, E52H, | 60932.05 | 7113.88 | 1.03 | 0.08 |
| | Y55F, Q70A, G80A, | | | | |
| | D83N, V84F, Y85A] | | | | |
| t814210 | [N29S, I33R, E52H, | 49548.21 | 4737.42 | 1.03 | 0.11 |
| | Y55F, Q70A, G80A, | | | | |
| | D83K, Y85A] | | | | |
| t814532 | [A18D, I33H, E52H, | 51117.00 | 5721.69 | 1.00 | 0.10 |
| | Y55F, Q70A, G80A, | | | | |
| | D83N, V84F, Y85A] | | | | |
| t814193 | [A18D, F24M, T26R, | 51531.08 | 2806.47 | 0.98 | 0.10 |
| | N29S, I33H, E52H, | | | | |
| | Y55F, Q70A, G80A, | | | | |
| | D83N, V84F, Y85A] | | | | |
| t814326 | [A18D, F24M, T26R, | 49506.68 | 5425.54 | 0.98 | 0.02 |
| | N29S, I33R, E52H, | | | | |
| | Y55F, Q70A, D71Q, | | | | |
| | I74G, G80A, D83N, | | | | |
| | V84K, Y85A] | | | | |
| t814225 | [T26R, I33R, K49Q, | 46671.13 | 4390.75 | 0.96 | 0.09 |
| | E52H, Y55F, Q70A, | | | | |
| | I74G, G80A, D83N, | | | | |
| | Y85A] | | | | |
| t814436 | [F24M, I33R, E52H, | 56696.61 | 12728.60 | 0.92 | 0.03 |
| | Y55F, Q70A, G80A, | | | | |
| | D83K, Y85A] | | | | |
| t814533 | [A18D, T26R, I33H, | 55150.84 | 9098.06 | 0.84 | 0.07 |
| | K49Q, E52H, Y55F, | | | | |
| | Q70A, G80A, D83N, | | | | |
| | Y85A] | | | | |
| t814201 | [A18D, I33H, K49Q, | 35649.52 | 2325.58 | 0.84 | 0.10 |
| | E52H, Y55F, Q70A, | | | | |
| | G80A, D83N, Y85A] | | | | |
| t814208 | [A18D, T26A, V28E, | 44146.79 | 8318.58 | 0.84 | 0.17 |
| | N29S, E53H, Q70L, | | | | |
| | G80A, G82K, D83A, | | | | |
| | S87K] | | | | |
| t621324 | [A18D E22K T26A N29S | 28824.56 | 4324.76 | 0.82 | 0.10 |
| (control) | E53H H57Y Q70L G82K | | | | |
| | D83A S87K] | | | | |
| t814537 | [A18D, F24M, T26R, | 45472.46 | 9370.79 | 0.68 | 0.10 |
| | N29S, I33H, K49Q, | | | | |
| | E52H, Y55F, Q70A, | | | | |
| | G80A, D83N, V84F, | | | | |
| | Y85A] | | | | |
| t814296 | [A18D, T26R, N29S, | 41020.94 | 2545.16 | 0.64 | 0.02 |
| | I33H, K49Q, E52H, | | | | |
| | Y55F, Q70A, G80A, | | | | |
| | D83N, V84F, Y85A] | | | | |
| t782404 | N/A (GFP control) | 28760.66 | 4283.59 | 0.49 | 0.07 |
| (control) | | | | | |

### Example 4: Identification of Additional Synthetic OAC Polypeptides that are Capable of Producing Olivetolic Acid

A collection of CsOAC mutation designs from Examples 1-3 was assessed for activity. These designs spanned a number of positions and included many combinations of mutations. Following screening which evaluated olivetolic acid (OA) and olivetol (OL) production in an in vivo context, data were filtered based upon reliability and a predictive model regressed to predict OA and OL production given a protein sequence. Using this model, a set of template sequences which had been identified in Examples 1-3 as lead candidates was subjected to incorporation of additional mutations as assessed by the model. Designs were made to achieve some combination of predicted OA production increase and/or predicted OL production decrease.

928 synthetic OAC polypeptides generated via these various design strategies were assembled into a library. For expression in S. cerevisiae, nucleotide sequences were recoded, synthesized, and cloned into an integrative yeast expression vector. Each candidate OAC expression construct was transformed into a S. cerevisiae CEN.PK strain previously engineered to produce olivetol by carrying a genomically integrated olivetol synthase as in Example 1. Strain t815900, expressing a CsOAC variant containing a combination of 6 point mutations relative to the CsOAC peptide sequence, was included in the library as positive control for hit ranking. Strain t782404, expressing a fluorescent protein (GFP), was included in the library as a negative control for enzyme activity. Strain t782504, expressing wild-type CsOAC, was also included as a control.

An assay for measuring production of olivetolic acid was conducted as follows: each thawed glycerol stock of OAC transformants was stamped into a well of YPD+4% dextrose media. Samples were incubated at 30° C. in a shaking incubator for 2 days. A portion of each of the resulting cultures was stamped into a well of YEP+2% raffinose+2% galactose+1 mM hexanoate (C6). Samples were incubated at 30° C. in a shaking incubator for 4 days. Additional spike-ins of 1 mM hexanoate and 2% galactose were added at 24 and 48 hours for a total of 3 mM hexanoate and 6% galactose feed. A portion of each of the resulting production cultures was stamped into a well of PBS. Optical measurements were taken on a plate reader, with absorbance measured at 600 nm and fluorescence at 528 nm with 485 nm excitation. A portion of each of the production cultures was stamped into a well of 100% methanol in half-height deepwell plates. Plates were heat sealed and frozen at −80° C. for two hours. Samples were then thawed and spun down. A portion of the supernatant was stamped into half-area 96 well plates. Olivetolic acid and olivetol production in the samples were quantified via LC-MS.

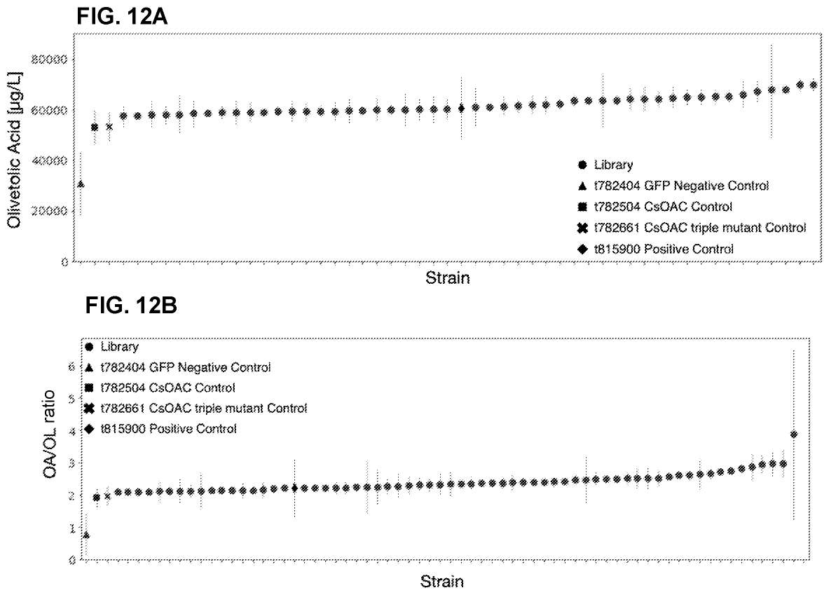

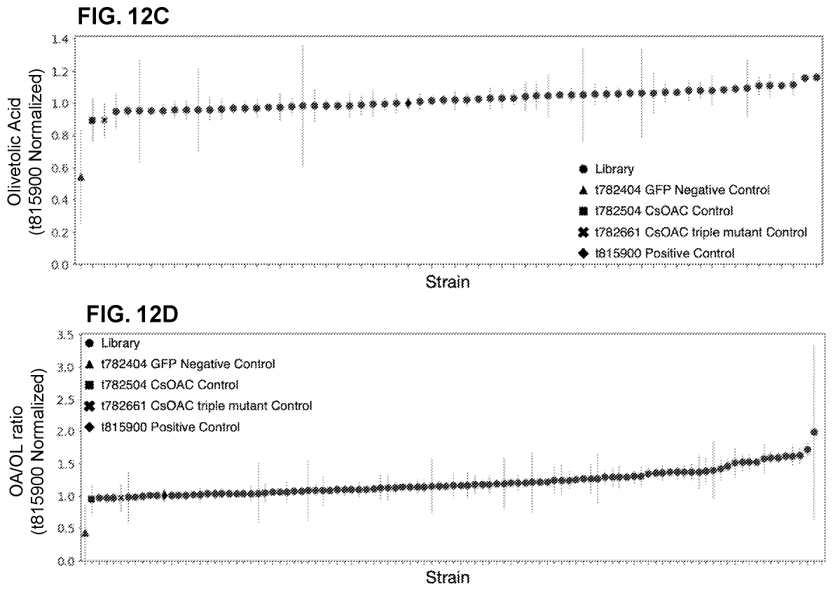

204 library strains were elevated to a secondary screen to verify OA and OL production. The secondary screen was performed as described above with the exception that additional biological replicates were included per strain. All strains were screened in quadruplicate (FIGS. 12A-12D and Tables 9A-9B).

Strains were designated as hits from the secondary screen if they produced a mean Olivetolic Acid titer equal to or greater than 95% of the t815900 positive control. Since minimization of Olivetol production was an explicit design strategy for this library, the ratio of OA to OL (OA/OL) was also examined. Strains were also designated as hits, using OA/OL as a key performance indicator, if they produced a mean OA/OL equal to or greater than 95% of the t815900 positive control. Several strains met both of these criteria, including: t876782, t877510, t877258, t815900, t877645, t877718, t877702, t877534, t876870, t877712, t877449, t877187, t877644, t876749, t876938, t877570, t877598, t877444, t877304, t877667, t877616, t877620, t876806, and t877290. Without wishing to be bound by any theory, the set of mutations harbored by the OAC candidates in this group may impact the apparent cyclase activity of the enzyme by enhancing the enzyme's thermal and colloidal stability and/or positively impact its kinetics against the tetraketide-CoA substrate.

Based on the data, additional strains of interest include: t877091, t876768, t876909, t877536, t877477, t877264, t877554, t876985, t876764, and t877431.

TABLE 9A
Activity data for synthetic OAC polypeptides in S. cerevisiae described in
Example 4 and FIGs. 12A-12B.

| | Mutations | | | | | | |
|---|---|---|---|---|---|---|---|
| | relative to | Olivetolic | Olivetolic | | | | |
| | CsOAC | Acid | Acid | Olivetol | Olivetol | | |
| | (SEQ ID | (ug/L) | (ug/L) | (ug/L) | (ug/L) | OA/OL | OA/OL |
| Strains | NO: 1) | mean | std | mean | std | mean | std |
| t877598 | E17N | 67868.27 | 21691.20 | 21305.15 | 6434.17 | 3.19 | 3.37 |
| | A18D | | | | | | |
| | E22K | | | | | | |
| | V31L | | | | | | |
| | I33N | | | | | | |
| | D71E | | | | | | |
| | V84F | | | | | | |
| t877570 | E17N | 63817.18 | 12078.01 | 21521.78 | 1734.48 | 2.97 | 6.96 |
| | V31L | | | | | | |
| | I33N | | | | | | |
| | S65N | | | | | | |
| | D71S | | | | | | |
| | V84K | | | | | | |
| t877091 | E17N | 50551.07 | 4492.37 | 17251.51 | 1783.09 | 2.93 | 2.52 |
| | V31L | | | | | | |
| | I33N | | | | | | |
| | D71S | | | | | | |
| | V84K | | | | | | |
| | T98K | | | | | | |
| t876768 | E17N | 51029.88 | 2242.17 | 17496.33 | 2480.65 | 2.92 | 0.90 |
| | V31L | | | | | | |
| | D39G | | | | | | |
| | D71S | | | | | | |
| | D83A | | | | | | |
| | V84K | | | | | | |
| t876938 | V84K | 67174.40 | 4646.90 | 24006.48 | 2630.23 | 2.80 | 1.77 |
| | T26R | | | | | | |
| | D39G | | | | | | |
| | D13R | | | | | | |
| | T98K | | | | | | |
| | S65N | | | | | | |
| t876909 | E17N | 33840.19 | 4098.31 | 12254.20 | 3408.22 | 2.76 | 1.20 |
| | A18D | | | | | | |
| | T26R | | | | | | |
| | V31L | | | | | | |
| | D71S | | | | | | |
| | D83A | | | | | | |
| | V84K | | | | | | |
| t877536 | E17N | 54680.05 | 3491.01 | 19992.98 | 1763.47 | 2.73 | 1.98 |
| | A18D | | | | | | |
| | V31L | | | | | | |
| | I33N | | | | | | |
| | D71S | | | | | | |
| | V84K | | | | | | |
| | T98K | | | | | | |
| t876749 | E17N | 64440.98 | 5327.62 | 23578.38 | 1077.22 | 2.73 | 4.95 |
| | A18D | | | | | | |
| | T26R | | | | | | |
| | V31L | | | | | | |
| | I33N | | | | | | |
| | D71S | | | | | | |
| | V84K | | | | | | |
| t877477 | A18D | 54780.30 | 1226.33 | 20699.62 | 1728.08 | 2.65 | 0.71 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| | F88N | | | | | | |
| t877264 | V84K | 57075.58 | 3857.78 | 21848.34 | 2343.84 | 2.61 | 1.65 |
| | T26R | | | | | | |
| | D39G | | | | | | |
| | D83E | | | | | | |
| | D13R | | | | | | |
| | T98K | | | | | | |
| t877554 | E17N | 50002.90 | 2280.08 | 19180.35 | 985.50 | 2.61 | 2.31 |
| | A18D | | | | | | |
| | V31L | | | | | | |
| | I33N | | | | | | |
| | S65N | | | | | | |
| | D71S | | | | | | |
| | V84K | | | | | | |
| t876985 | E17N | 49808.11 | 1879.77 | 19379.58 | 3707.24 | 2.57 | 0.51 |
| | V31L | | | | | | |
| | I33N | | | | | | |
| | D71S | | | | | | |
| | G82R | | | | | | |
| | V84F | | | | | | |
| t877362 | E17N | 55978.26 | 5879.41 | 21883.71 | 2598.33 | 2.56 | 2.26 |
| | A18D | | | | | | |
| | V31L | | | | | | |
| | I33N | | | | | | |
| | V66Y | | | | | | |
| | D71S | | | | | | |
| | V84F | | | | | | |
| t876764 | D13R | 50627.72 | 2274.69 | 20232.64 | 1467.87 | 2.50 | 1.55 |
| | E17N | | | | | | |
| | A18D | | | | | | |
| | V31L | | | | | | |
| | D71S | | | | | | |
| | D83A | | | | | | |
| | V84K | | | | | | |
| t877407 | A18D | 55706.52 | 1736.04 | 22286.50 | 1497.50 | 2.50 | 1.16 |
| | E22K | | | | | | |
| | T26R | | | | | | |
| | I33R | | | | | | |
| | E52H | | | | | | |
| | Y55F | | | | | | |
| | V66Y | | | | | | |
| | Q70A | | | | | | |
| | G80A | | | | | | |
| | D83N | | | | | | |
| | V84K | | | | | | |
| t877429 | E17N | 56547.64 | 1990.83 | 22639.43 | 801.14 | 2.50 | 2.49 |
| | A18D | | | | | | |
| | V31L | | | | | | |
| | I33N | | | | | | |
| | D71S | | | | | | |
| | V84K | | | | | | |
| t877431 | E17N | 54719.34 | 3649.82 | 21964.68 | 2504.58 | 2.49 | 1.46 |
| | A18D | | | | | | |
| | V31L | | | | | | |
| | I33N | | | | | | |
| | D71E | | | | | | |
| | V84F | | | | | | |
| t877109 | E17N | 32989.36 | 2692.01 | 13272.99 | 1374.32 | 2.49 | 1.96 |
| | T26R | | | | | | |
| | V31L | | | | | | |
| | D71S | | | | | | |
| | D83A | | | | | | |
| | V84K | | | | | | |
| t876982 | T26R | 49937.92 | 2166.38 | 20302.76 | 3713.45 | 2.46 | 0.58 |
| | D39G | | | | | | |
| | D83E | | | | | | |
| | E64D | | | | | | |
| | T98K | | | | | | |
| | S65N | | | | | | |
| t877136 | E17N | 53887.86 | 2665.34 | 21946.08 | 1221.45 | 2.46 | 2.18 |
| | A18D | | | | | | |
| | V31L | | | | | | |
| | D39G | | | | | | |
| | D71S | | | | | | |
| | D83A | | | | | | |
| | V84K | | | | | | |
| t877723 | E17N | 47011.33 | 11004.06 | 19283.83 | 6554.43 | 2.44 | 1.68 |
| | A18D | | | | | | |
| | V31L | | | | | | |
| | I33N | | | | | | |
| | D71S | | | | | | |
| | G82R | | | | | | |
| | V84F | | | | | | |
| t877187 | A18D | 59011.05 | 4813.31 | 24309.28 | 377.29 | 2.43 | 12.76 |
| | T26R | | | | | | |
| | I33R | | | | | | |
| | E52H | | | | | | |
| | Y55F | | | | | | |
| | V66Y | | | | | | |
| | Q70A | | | | | | |
| | G80A | | | | | | |
| | D83N | | | | | | |
| | V84K | | | | | | |
| | Y85F | | | | | | |
| t877011 | E17N | 48856.93 | 4810.13 | 20292.70 | 1075.39 | 2.41 | 4.47 |
| | A18D | | | | | | |
| | V31L | | | | | | |
| | I33N | | | | | | |
| | D71S | | | | | | |
| | I74G | | | | | | |
| | V84K | | | | | | |
| t877449 | E17N | 59697.91 | 1646.25 | 24855.90 | 1294.87 | 2.40 | 1.27 |
| | V31L | | | | | | |
| | I33N | | | | | | |
| | D71S | | | | | | |
| | V84K | | | | | | |
| | Y85F | | | | | | |
| t877712 | E17N | 60438.98 | 6217.18 | 25290.87 | 3570.75 | 2.39 | 1.74 |
| | A18D | | | | | | |
| | V31L | | | | | | |
| | D71S | | | | | | |
| | I74G | | | | | | |
| | D83E | | | | | | |
| | V84F | | | | | | |
| t877501 | D39G | 56288.48 | 3133.12 | 23588.23 | 1656.88 | 2.39 | 1.89 |
| | D83E | | | | | | |
| | D13R | | | | | | |
| | E64D | | | | | | |
| | T98K | | | | | | |
| | S65N | | | | | | |
| t876853 | E17N | 51192.93 | 6578.52 | 21555.28 | 2239.36 | 2.37 | 2.94 |
| | A18D | | | | | | |
| | V31L | | | | | | |
| | S65N | | | | | | |
| | D71S | | | | | | |
| | D83A | | | | | | |
| | V84K | | | | | | |
| t877644 | E17N | 58178.95 | 8656.44 | 24506.98 | 4230.45 | 2.37 | 2.05 |
| | A18D | | | | | | |
| | V31L | | | | | | |
| | I33N | | | | | | |
| | D71S | | | | | | |
| | D83K | | | | | | |
| | V84K | | | | | | |
| t877056 | T26R | 49443.43 | 2753.79 | 20946.27 | 1166.74 | 2.36 | 2.36 |
| | D39G | | | | | | |
| | D83E | | | | | | |
| | D13R | | | | | | |
| | T98K | | | | | | |
| | S65N | | | | | | |
| t877014 | E17N | 51670.08 | 3751.55 | 21890.11 | 899.77 | 2.36 | 4.17 |
| | A18D | | | | | | |
| | E22K | | | | | | |
| | V31L | | | | | | |
| | D71E | | | | | | |
| | D83A | | | | | | |
| | V84F | | | | | | |
| t877228 | D13R | 56782.44 | 1036.99 | 24080.67 | 708.77 | 2.36 | 1.46 |
| | A18D | | | | | | |
| | N29S | | | | | | |
| | Q70L | | | | | | |
| | D71Q | | | | | | |
| | G80A | | | | | | |
| | D83E | | | | | | |
| | T98K | | | | | | |
| t876933 | V84K | 50834.86 | 3688.86 | 21721.90 | 486.44 | 2.34 | 7.58 |
| | T26R | | | | | | |
| | D39G | | | | | | |
| | D13R | | | | | | |
| | E64D | | | | | | |
| | S65N | | | | | | |
| t876870 | E17N | 64937.02 | 3842.99 | 27812.42 | 1786.49 | 2.33 | 2.15 |
| | A18D | | | | | | |
| | V31L | | | | | | |
| | I33N | | | | | | |
| | D71S | | | | | | |
| | D83E | | | | | | |
| | V84K | | | | | | |
| t877158 | V84K | 50511.64 | 7121.08 | 21832.81 | 1444.18 | 2.31 | 4.93 |
| | T26R | | | | | | |
| | D83E | | | | | | |
| | D13R | | | | | | |
| | E64D | | | | | | |
| | T98K | | | | | | |
| t877223 | E22K | 54773.70 | 4040.06 | 23764.25 | 1240.33 | 2.30 | 3.26 |
| | T26R | | | | | | |
| | I33H | | | | | | |
| | Y55F | | | | | | |
| | Q70A | | | | | | |
| | D71E | | | | | | |
| | G80A | | | | | | |
| | D83K | | | | | | |
| t877441 | D13R | 52694.50 | 4034.20 | 22896.71 | 2283.07 | 2.30 | 1.77 |
| | A18D | | | | | | |
| | N29S | | | | | | |
| | Q70L | | | | | | |
| | D71Q | | | | | | |
| | I74G | | | | | | |
| | G80A | | | | | | |
| | D83K | | | | | | |
| | T98K | | | | | | |
| t877227 | A18D | 57436.56 | 5876.45 | 25150.13 | 4402.90 | 2.28 | 1.33 |
| | T26R | | | | | | |
| | I33R | | | | | | |
| | E52H | | | | | | |
| | Y55F | | | | | | |
| | Q70A | | | | | | |
| | D71E | | | | | | |
| | G80A | | | | | | |
| | D83N | | | | | | |
| | V84K | | | | | | |
| t877534 | V84K | 60486.62 | 4718.43 | 26604.38 | 1613.50 | 2.27 | 2.92 |
| | T26R | | | | | | |
| | D39G | | | | | | |
| | D83E | | | | | | |
| | T98K | | | | | | |
| | S65N | | | | | | |
| t876945 | E17N | 49344.29 | 3954.50 | 21965.10 | 2541.79 | 2.25 | 1.56 |
| | V31L | | | | | | |
| | I33N | | | | | | |
| | D71S | | | | | | |
| | V84K | | | | | | |
| | T98Y | | | | | | |
| t876987 | E17N | 52352.34 | 6215.69 | 23431.23 | 1821.68 | 2.23 | 3.41 |
| | V31L | | | | | | |
| | D71S | | | | | | |
| | D83A | | | | | | |
| | V84F | | | | | | |
| | T98Y | | | | | | |
| t877645 | E17N | 60952.30 | 8731.29 | 27313.27 | 1880.08 | 2.23 | 4.64 |
| | E22K | | | | | | |
| | V31L | | | | | | |
| | I33N | | | | | | |
| | D71E | | | | | | |
| | V84F | | | | | | |
| t877404 | E17N | 54149.04 | 1829.47 | 24395.83 | 1120.53 | 2.22 | 1.63 |
| | A18D | | | | | | |
| | V31L | | | | | | |
| | D71S | | | | | | |
| | I74G | | | | | | |
| | D83A | | | | | | |
| | V84K | | | | | | |
| t877718 | E17N | 57706.74 | 1367.21 | 26027.09 | 1785.37 | 2.22 | 0.77 |
| | A18D | | | | | | |
| | V31L | | | | | | |
| | D71E | | | | | | |
| | D83A | | | | | | |
| | V84F | | | | | | |
| t877698 | E17N | 55949.94 | 11367.02 | 25333.42 | 5637.25 | 2.21 | 2.02 |
| | A18D | | | | | | |
| | V31L | | | | | | |
| | D71S | | | | | | |
| | D83E | | | | | | |
| | V84F | | | | | | |
| | T98Y | | | | | | |
| t876959 | A18D | 53915.45 | 5563.11 | 24414.71 | 2336.65 | 2.21 | 2.38 |
| | T26R | | | | | | |
| | D39G | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | T98K | | | | | | |
| t877021 | E17N | 52143.99 | 2881.57 | 23657.40 | 908.72 | 2.20 | 3.17 |
| | V31L | | | | | | |
| | I33N | | | | | | |
| | V66Y | | | | | | |
| | D71S | | | | | | |
| | V84F | | | | | | |
| t877702 | E17N | 60323.07 | 5054.18 | 27415.81 | 5956.57 | 2.20 | 0.85 |
| | V31L | | | | | | |
| | D71S | | | | | | |
| | D83E | | | | | | |
| | V84F | | | | | | |
| | T98Y | | | | | | |
| t876990 | V84K | 49616.28 | 1548.70 | 22698.29 | 1513.65 | 2.19 | 1.02 |
| | D39G | | | | | | |
| | D13R | | | | | | |
| | E64D | | | | | | |
| | T98K | | | | | | |
| | S65N | | | | | | |
| t876772 | E17N | 44451.09 | 4539.77 | 20565.64 | 1090.33 | 2.16 | 4.16 |
| | A18D | | | | | | |
| | V31L | | | | | | |
| | I33N | | | | | | |
| | V66Y | | | | | | |
| | D71E | | | | | | |
| | V84F | | | | | | |
| t877248 | E17N | 31807.81 | 15843.50 | 14750.48 | 17077.18 | 2.16 | 0.93 |
| | A18D | | | | | | |
| | V31L | | | | | | |
| | I33N | | | | | | |
| | D71S | | | | | | |
| | I74G | | | | | | |
| | D83K | | | | | | |
| | V84F | | | | | | |
| t877510 | E17N | 59381.25 | 4475.59 | 27720.90 | 1434.25 | 2.14 | 3.12 |
| | T26R | | | | | | |
| | V31L | | | | | | |
| | D71S | | | | | | |
| | D83E | | | | | | |
| | V84F | | | | | | |
| t877258 | E17N | 60136.68 | 7447.61 | 28147.27 | 4203.09 | 2.14 | 1.77 |
| | V31L | | | | | | |
| | I33N | | | | | | |
| | E64D | | | | | | |
| | D71S | | | | | | |
| | V84F | | | | | | |
| t877088 | E17N | 49961.36 | 3968.73 | 23428.45 | 1241.19 | 2.13 | 3.20 |
| | V31L | | | | | | |
| | I33N | | | | | | |
| | D71S | | | | | | |
| | D83K | | | | | | |
| | V84K | | | | | | |
| t877516 | V84K | 52307.39 | 2218.39 | 24548.03 | 1514.85 | 2.13 | 1.46 |
| | D39G | | | | | | |
| | D83E | | | | | | |
| | D13R | | | | | | |
| | T98K | | | | | | |
| | S65N | | | | | | |
| t877306 | A18D | 45652.55 | 3641.24 | 21442.37 | 2263.41 | 2.13 | 1.61 |
| | T26R | | | | | | |
| | I33R | | | | | | |
| | E52H | | | | | | |
| | Y55F | | | | | | |
| | S65N | | | | | | |
| | Q70A | | | | | | |
| | G80A | | | | | | |
| | D83E | | | | | | |
| | V84K | | | | | | |
| t877188 | A18D | 51048.31 | 3689.03 | 24130.48 | 1483.64 | 2.12 | 2.49 |
| | T26R | | | | | | |
| | I33R | | | | | | |
| | E52H | | | | | | |
| | Y55F | | | | | | |
| | V66Y | | | | | | |
| | Q70A | | | | | | |
| | D71E | | | | | | |
| | G80A | | | | | | |
| | D83N | | | | | | |
| | V84K | | | | | | |
| t876766 | E17N | 48231.25 | 3648.00 | 22912.68 | 2213.71 | 2.11 | 1.65 |
| | A18D | | | | | | |
| | V31L | | | | | | |
| | I33N | | | | | | |
| | D39G | | | | | | |
| | D71S | | | | | | |
| | V84K | | | | | | |
| t877629 | E17N | 50566.55 | 2789.66 | 24080.49 | 1476.78 | 2.10 | 1.89 |
| | A18D | | | | | | |
| | V31L | | | | | | |
| | D71S | | | | | | |
| | D83A | | | | | | |
| | V84K | | | | | | |
| | T98Y | | | | | | |
| t876865 | V84K | 45363.52 | 3151.94 | 21623.87 | 525.66 | 2.10 | 6.00 |
| | T26R | | | | | | |
| | D39G | | | | | | |
| | D83E | | | | | | |
| | E64D | | | | | | |
| | S65N | | | | | | |
| t876782 | A18D | 64809.00 | 4704.41 | 30912.59 | 1905.96 | 2.10 | 2.47 |
| | T26R | | | | | | |
| | I33R | | | | | | |
| | D39G | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| t877108 | A18D | 44105.85 | 5897.00 | 21066.34 | 1458.25 | 2.09 | 4.04 |
| | T26R | | | | | | |
| | E52H | | | | | | |
| | Y55F | | | | | | |
| | E64D | | | | | | |
| | Q70A | | | | | | |
| | I74G | | | | | | |
| | G80A | | | | | | |
| | D83N | | | | | | |
| | V84K | | | | | | |
| t877164 | D13R | 52444.53 | 1022.42 | 25060.05 | 1527.90 | 2.09 | 0.67 |
| | A18D | | | | | | |
| | N29S | | | | | | |
| | E64D | | | | | | |
| | Q70L | | | | | | |
| | D71Q | | | | | | |
| | G80A | | | | | | |
| | D83E | | | | | | |
| t877256 | T26R | 46754.65 | 6054.46 | 22368.15 | 1888.78 | 2.09 | 3.21 |
| | I33H | | | | | | |
| | Y55F | | | | | | |
| | Q70A | | | | | | |
| | I74G | | | | | | |
| | G80A | | | | | | |
| | D83K | | | | | | |
| | Y85A | | | | | | |
| t877596 | D13R | 56239.07 | 1947.27 | 26982.19 | 9631.60 | 2.08 | 0.20 |
| | A18D | | | | | | |
| | E22K | | | | | | |
| | N29S | | | | | | |
| | Q70L | | | | | | |
| | D71Q | | | | | | |
| | G80A | | | | | | |
| | D83K | | | | | | |
| t877249 | E17N | 44060.21 | 27194.35 | 21150.39 | 11587.99 | 2.08 | 2.35 |
| | A18D | | | | | | |
| | V31L | | | | | | |
| | D71S | | | | | | |
| | D83A | | | | | | |
| | V84K | | | | | | |
| | Y85F | | | | | | |
| t877439 | D13R | 52912.00 | 3008.81 | 25417.38 | 1169.27 | 2.08 | 2.57 |
| | A18D | | | | | | |
| | N29S | | | | | | |
| | E64D | | | | | | |
| | Q70L | | | | | | |
| | D71Q | | | | | | |
| | G80A | | | | | | |
| | D83K | | | | | | |
| t815900 | E17N | 60692.00 | 12202.43 | 29189.54 | 5746.55 | 2.08 | 2.12 |
| | A18D | | | | | | |
| | V31L | | | | | | |
| | D71S | | | | | | |
| | D83A | | | | | | |
| | V84F | | | | | | |
| t877444 | E17N | 59293.89 | 4550.29 | 28537.59 | 775.95 | 2.08 | 5.86 |
| | A18D | | | | | | |
| | V31L | | | | | | |
| | I33N | | | | | | |
| | D71S | | | | | | |
| | G80A | | | | | | |
| | V84F | | | | | | |
| t877064 | E17N | 46022.26 | 4521.93 | 22155.20 | 2136.18 | 2.08 | 2.12 |
| | A18D | | | | | | |
| | V31L | | | | | | |
| | D71S | | | | | | |
| | D83K | | | | | | |
| | V84F | | | | | | |
| t877229 | V84K | 51766.53 | 581.34 | 25024.06 | 2763.81 | 2.07 | 0.21 |
| | T26R | | | | | | |
| | D39G | | | | | | |
| | D83E | | | | | | |
| | D13R | | | | | | |
| | S65N | | | | | | |
| t876807 | E17N | 54416.78 | 3410.98 | 26387.58 | 1522.51 | 2.06 | 2.24 |
| | V31L | | | | | | |
| | I33N | | | | | | |
| | D39G | | | | | | |
| | D71S | | | | | | |
| | V84K | | | | | | |
| t877304 | E17N | 58019.99 | 6004.18 | 28200.61 | 1010.49 | 2.06 | 5.94 |
| | V31L | | | | | | |
| | I33N | | | | | | |
| | V66Y | | | | | | |
| | D71E | | | | | | |
| | V84F | | | | | | |
| t877320 | D13R | 55475.12 | 5368.94 | 27093.32 | 4082.04 | 2.05 | 1.32 |
| | A18D | | | | | | |
| | T26R | | | | | | |
| | D39G | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t877667 | E17N | 59293.88 | 2274.62 | 28993.64 | 742.66 | 2.05 | 3.06 |
| | V31L | | | | | | |
| | I33N | | | | | | |
| | D71E | | | | | | |
| | G82R | | | | | | |
| | V84F | | | | | | |
| t877625 | E17N | 50975.71 | 3509.66 | 24954.44 | 2374.73 | 2.04 | 1.48 |
| | A18D | | | | | | |
| | T26R | | | | | | |
| | V31L | | | | | | |
| | D71S | | | | | | |
| | D83E | | | | | | |
| | V84F | | | | | | |
| t877354 | E17N | 55675.94 | 6879.13 | 27318.84 | 5363.49 | 2.04 | 1.28 |
| | V31L | | | | | | |
| | I33N | | | | | | |
| | D71S | | | | | | |
| | D83K | | | | | | |
| | V84F | | | | | | |
| | T98Y | | | | | | |
| t877544 | V84K | 51936.13 | 15630.75 | 25563.27 | 1783.85 | 2.03 | 8.76 |
| | D39G | | | | | | |
| | D83E | | | | | | |
| | D13R | | | | | | |
| | E64D | | | | | | |
| | T98K | | | | | | |
| t877616 | T26R | 59339.87 | 2627.33 | 29475.12 | 961.12 | 2.01 | 2.73 |
| | I33H | | | | | | |
| | Y55F | | | | | | |
| | Q70A | | | | | | |
| | G80A | | | | | | |
| | D83K | | | | | | |
| | V84K | | | | | | |
| | F88N | | | | | | |
| t877185 | D13R | 56431.78 | 1953.46 | 28050.45 | 6496.14 | 2.01 | 0.30 |
| | E17N | | | | | | |
| | V31L | | | | | | |
| | D71S | | | | | | |
| | D83A | | | | | | |
| | V84K | | | | | | |
| t877620 | A18D | 58565.43 | 6339.82 | 29145.28 | 1437.66 | 2.01 | 4.41 |
| | E22K | | | | | | |
| | T26R | | | | | | |
| | E52H | | | | | | |
| | Y55F | | | | | | |
| | Q70A | | | | | | |
| | D71E | | | | | | |
| | G80A | | | | | | |
| | D83N | | | | | | |
| | V84K | | | | | | |
| t877709 | E17N | 56976.00 | 6382.90 | 28534.37 | 9507.54 | 2.00 | 0.67 |
| | T26R | | | | | | |
| | V31L | | | | | | |
| | I33N | | | | | | |
| | E64D | | | | | | |
| | D71S | | | | | | |
| | V84F | | | | | | |
| t877073 | V84K | 50256.88 | 4643.33 | 25232.83 | 5471.39 | 1.99 | 0.85 |
| | T26R | | | | | | |
| | D39G | | | | | | |
| | D13R | | | | | | |
| | E64D | | | | | | |
| | T98K | | | | | | |
| t861745 | D39Q | 53231.45 | 4132.82 | 26791.25 | 2049.20 | 1.99 | 2.02 |
| | F95M | | | | | | |
| t876837 | T26R | 53844.56 | 2316.81 | 27183.17 | 3782.35 | 1.98 | 0.61 |
| | I33H | | | | | | |
| | Y55F | | | | | | |
| | Q70A | | | | | | |
| | G80A | | | | | | |
| | D83E | | | | | | |
| | T98K | | | | | | |
| t876806 | D13R | 65340.51 | 2791.81 | 33036.07 | 1511.88 | 1.98 | 1.85 |
| | A18D | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| t876826 | A18D | 48035.13 | 261.40 | 24298.01 | 2308.16 | 1.98 | 0.11 |
| | T26R | | | | | | |
| | D39G | | | | | | |
| | E52H | | | | | | |
| | Y55F | | | | | | |
| | Q70A | | | | | | |
| | I74G | | | | | | |
| | G80A | | | | | | |
| | D83N | | | | | | |
| | V84K | | | | | | |
| t877087 | V84K | 43514.87 | 1691.57 | 22013.71 | 1077.34 | 1.98 | 1.57 |
| | D39G | | | | | | |
| | D83E | | | | | | |
| | E64D | | | | | | |
| | T98K | | | | | | |
| | S65N | | | | | | |
| t877290 | V84K | 58038.07 | 4194.21 | 29367.92 | 1203.75 | 1.98 | 3.48 |
| | T26R | | | | | | |
| | D83E | | | | | | |
| | D13R | | | | | | |
| | T98K | | | | | | |
| | S65N | | | | | | |
| t877100 | T26R | 52206.88 | 4172.80 | 26599.39 | 2164.38 | 1.96 | 1.93 |
| | I33N | | | | | | |
| | Y55F | | | | | | |
| | Q70A | | | | | | |
| | D71S | | | | | | |
| | G80A | | | | | | |
| | D83K | | | | | | |
| t861743 | I33R | 51788.45 | 4620.19 | 26394.32 | 3071.48 | 1.96 | 1.50 |
| | E52H | | | | | | |
| | Y55F | | | | | | |
| | Q70A | | | | | | |
| | I74G | | | | | | |
| | D83K | | | | | | |
| t877269 | E17N | 54266.24 | 2942.08 | 27665.11 | 3953.05 | 1.96 | 0.74 |
| | V31L | | | | | | |
| | D71S | | | | | | |
| | D83A | | | | | | |
| | V84K | | | | | | |
| | T98K | | | | | | |
| t876751 | A18D | 64432.37 | 4566.22 | 32889.15 | 2634.00 | 1.96 | 1.73 |
| | T26R | | | | | | |
| | I33R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t876801 | V84K | 47956.65 | 3456.69 | 24643.11 | 2195.19 | 1.95 | 1.57 |
| | D83E | | | | | | |
| | D13R | | | | | | |
| | E64D | | | | | | |
| | T98K | | | | | | |
| | S65N | | | | | | |
| t877098 | A18D | 45521.98 | 1851.09 | 23469.52 | 2543.73 | 1.94 | 0.73 |
| | E22K | | | | | | |
| | T26R | | | | | | |
| | I33R | | | | | | |
| | E52H | | | | | | |
| | Y55F | | | | | | |
| | Q70A | | | | | | |
| | G80A | | | | | | |
| | G82R | | | | | | |
| | D83N | | | | | | |
| | V84K | | | | | | |
| t782661 | I33N | 53265.20 | 5799.56 | 27507.95 | 4860.83 | 1.94 | 1.19 |
| | D71S | | | | | | |
| | G80A | | | | | | |
| t877282 | E17N | 39931.21 | 6774.40 | 21054.86 | 8239.13 | 1.90 | 0.82 |
| | A18D | | | | | | |
| | V31L | | | | | | |
| | I33N | | | | | | |
| | E64D | | | | | | |
| | D71S | | | | | | |
| | V84K | | | | | | |
| t877612 | A18D | 56659.73 | 2292.11 | 30064.51 | 1808.49 | 1.88 | 1.27 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| t876957 | V84K | 48689.93 | 3150.24 | 25915.87 | 2344.16 | 1.88 | 1.34 |
| | T26R | | | | | | |
| | D13R | | | | | | |
| | E64D | | | | | | |
| | T98K | | | | | | |
| | S65N | | | | | | |
| t782504 | Wild-type | 53125.92 | 6762.30 | 28431.92 | 4401.36 | 1.87 | 1.54 |
| t876956 | T26R | 51382.83 | 1504.71 | 27606.53 | 2389.77 | 1.86 | 0.63 |
| | I33H | | | | | | |
| | Y55F | | | | | | |
| | Q70A | | | | | | |
| | G80A | | | | | | |
| | D83E | | | | | | |
| | F88N | | | | | | |
| t877090 | D13R | 49781.04 | 3138.95 | 26812.85 | 5759.06 | 1.86 | 0.55 |
| | A18D | | | | | | |
| | N29S | | | | | | |
| | Q70L | | | | | | |
| | I74G | | | | | | |
| | G80A | | | | | | |
| | D83K | | | | | | |
| | Y85F | | | | | | |
| t876810 | E17N | 61011.30 | 1321.57 | 32871.62 | 517.81 | 1.86 | 2.55 |
| | A18D | | | | | | |
| | V31L | | | | | | |
| | E64D | | | | | | |
| | D71S | | | | | | |
| | D83A | | | | | | |
| | V84F | | | | | | |
| | T98K | | | | | | |
| t877138 | D13R | 54129.25 | 2629.03 | 29196.65 | 1079.77 | 1.85 | 2.43 |
| | E17N | | | | | | |
| | V31L | | | | | | |
| | I33N | | | | | | |
| | D71S | | | | | | |
| | V84K | | | | | | |
| t876773 | A18D | 63668.51 | 1650.14 | 34474.82 | 946.43 | 1.85 | 1.74 |
| | T26R | | | | | | |
| | I33R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t876968 | D13R | 70112.52 | 3302.04 | 37968.56 | 1586.99 | 1.85 | 2.08 |
| | A18D | | | | | | |
| | T26R | | | | | | |
| | N29S | | | | | | |
| | Q70L | | | | | | |
| | D71Q | | | | | | |
| | G80A | | | | | | |
| | D83E | | | | | | |
| t877255 | A18D | 24979.53 | 28870.66 | 13532.39 | 15656.42 | 1.85 | 1.84 |
| | T26R | | | | | | |
| | I33R | | | | | | |
| | D39G | | | | | | |
| | E52H | | | | | | |
| | Y55F | | | | | | |
| | Q70A | | | | | | |
| | G80A | | | | | | |
| | D83E | | | | | | |
| | V84K | | | | | | |
| t876755 | A18D | 63852.13 | 2030.64 | 34631.82 | 1214.55 | 1.84 | 1.67 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t876943 | E17N | 50230.13 | 2423.56 | 27315.55 | 2299.52 | 1.84 | 1.05 |
| | V31L | | | | | | |
| | D71S | | | | | | |
| | I74G | | | | | | |
| | D83K | | | | | | |
| | V84F | | | | | | |
| t877367 | E17N | 52450.09 | 1782.81 | 28646.64 | 5843.69 | 1.83 | 0.31 |
| | A18D | | | | | | |
| | V31L | | | | | | |
| | I33N | | | | | | |
| | D71S | | | | | | |
| | V84K | | | | | | |
| | T98Y | | | | | | |
| t877437 | T26R | 55489.85 | 885.19 | 30327.34 | 962.27 | 1.83 | 0.92 |
| | D39G | | | | | | |
| | D83E | | | | | | |
| | D13R | | | | | | |
| | E64D | | | | | | |
| | T98K | | | | | | |
| t876988 | D13R | 56602.94 | 1536.17 | 30942.16 | 3192.03 | 1.83 | 0.48 |
| | A18D | | | | | | |
| | N29S | | | | | | |
| | D39G | | | | | | |
| | Q70L | | | | | | |
| | D71Q | | | | | | |
| | G80A | | | | | | |
| | D83K | | | | | | |
| | V84K | | | | | | |
| t877071 | E17N | 50655.59 | 6007.50 | 27721.38 | 4377.50 | 1.83 | 1.37 |
| | A18D | | | | | | |
| | V31L | | | | | | |
| | E64D | | | | | | |
| | D71S | | | | | | |
| | I74G | | | | | | |
| | D83A | | | | | | |
| | V84F | | | | | | |
| t876753 | T26R | 65312.88 | 3222.63 | 35971.07 | 1458.97 | 1.82 | 2.21 |
| | I33H | | | | | | |
| | Y55F | | | | | | |
| | E64D | | | | | | |
| | Q70A | | | | | | |
| | G80A | | | | | | |
| | D83K | | | | | | |
| | V84K | | | | | | |
| t876878 | A18D | 63615.34 | 2845.67 | 35061.22 | 738.36 | 1.81 | 3.85 |
| | T26R | | | | | | |
| | I33R | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| | T98Y | | | | | | |
| t877610 | D13R | 60117.93 | 2260.25 | 33164.85 | 1600.92 | 1.81 | 1.41 |
| | A18D | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t877467 | D13R | 50994.55 | 2091.86 | 28164.52 | 1778.31 | 1.81 | 1.18 |
| | E17N | | | | | | |
| | A18D | | | | | | |
| | V31L | | | | | | |
| | D71S | | | | | | |
| | D83E | | | | | | |
| | V84F | | | | | | |
| t877639 | D13R | 58919.01 | 3226.63 | 32615.60 | 1677.97 | 1.81 | 1.92 |
| | A18D | | | | | | |
| | N29S | | | | | | |
| | S65N | | | | | | |
| | Q70L | | | | | | |
| | D71Q | | | | | | |
| | I74G | | | | | | |
| | G80A | | | | | | |
| | D83K | | | | | | |
| t876936 | D71E | 62002.57 | 4090.71 | 34429.04 | 12548.73 | 1.80 | 0.33 |
| | V66Y | | | | | | |
| | E22K | | | | | | |
| | D83K | | | | | | |
| | T98K | | | | | | |
| | D13R | | | | | | |
| t876845 | V84K | 45943.44 | 3805.34 | 25593.74 | 2731.59 | 1.80 | 1.39 |
| | T26R | | | | | | |
| | D39G | | | | | | |
| | D83E | | | | | | |
| | D13R | | | | | | |
| | E64D | | | | | | |
| t877608 | E17N | 53825.58 | 1437.47 | 30042.53 | 1352.02 | 1.79 | 1.06 |
| | V31L | | | | | | |
| | V66Y | | | | | | |
| | D71S | | | | | | |
| | D83A | | | | | | |
| | V84F | | | | | | |
| | Y85F | | | | | | |
| t876882 | A18D | 62211.05 | 2342.43 | 34935.93 | 3007.76 | 1.78 | 0.78 |
| | T26R | | | | | | |
| | E52H | | | | | | |
| | Y55F | | | | | | |
| | Q70A | | | | | | |
| | D71E | | | | | | |
| | G80A | | | | | | |
| | D83N | | | | | | |
| | V84K | | | | | | |
| t877578 | D13R | 56567.60 | 7352.26 | 32014.26 | 1849.16 | 1.77 | 3.98 |
| | A18D | | | | | | |
| | N29S | | | | | | |
| | Q70L | | | | | | |
| | D71Q | | | | | | |
| | G80A | | | | | | |
| | D83K | | | | | | |
| t877577 | D13R | 51844.69 | 6015.37 | 29372.26 | 1666.04 | 1.77 | 3.61 |
| | A18D | | | | | | |
| | N29S | | | | | | |
| | Q70L | | | | | | |
| | D71Q | | | | | | |
| | G80A | | | | | | |
| | D83K | | | | | | |
| t876784 | D13R | 68077.97 | 2122.95 | 38638.31 | 2228.32 | 1.76 | 0.95 |
| | A18D | | | | | | |
| | E22K | | | | | | |
| | N29S | | | | | | |
| | Q70L | | | | | | |
| | D71E | | | | | | |
| | G80A | | | | | | |
| | D83K | | | | | | |
| t877744 | D13R | 55370.92 | 1769.06 | 31470.83 | 903.65 | 1.76 | 1.96 |
| | A18D | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | T98Y | | | | | | |
| t876980 | D13R | 48818.06 | 1712.04 | 27780.46 | 556.16 | 1.76 | 3.08 |
| | A18D | | | | | | |
| | T26R | | | | | | |
| | I33R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| t877741 | E17N | 54880.83 | 1927.15 | 31402.16 | 3011.95 | 1.75 | 0.64 |
| | V31L | | | | | | |
| | S65N | | | | | | |
| | D71S | | | | | | |
| | D83A | | | | | | |
| | V84K | | | | | | |
| t876914 | E17N | 62033.53 | 4019.09 | 35588.43 | 3070.28 | 1.74 | 1.31 |
| | A18D | | | | | | |
| | V31L | | | | | | |
| | V66Y | | | | | | |
| | D71E | | | | | | |
| | D83A | | | | | | |
| | V84F | | | | | | |
| t876778 | D13R | 65144.75 | 3711.83 | 37388.75 | 2663.52 | 1.74 | 1.39 |
| | A18D | | | | | | |
| | N29S | | | | | | |
| | E64D | | | | | | |
| | Q70L | | | | | | |
| | D71Q | | | | | | |
| | G80A | | | | | | |
| | D83K | | | | | | |
| | V84K | | | | | | |
| t876917 | D13R | 54484.93 | 7486.95 | 31352.32 | 1753.53 | 1.74 | 4.27 |
| | A18D | | | | | | |
| | T26R | | | | | | |
| | N29S | | | | | | |
| | Q70L | | | | | | |
| | D71Q | | | | | | |
| | G80A | | | | | | |
| | D83K | | | | | | |
| t877507 | D13R | 57669.72 | 4764.07 | 33271.40 | 2596.12 | 1.73 | 1.84 |
| | E17N | | | | | | |
| | A18D | | | | | | |
| | V31L | | | | | | |
| | D71S | | | | | | |
| | D83K | | | | | | |
| | V84F | | | | | | |
| t877039 | E17N | 43422.75 | 4055.70 | 25090.39 | 1469.59 | 1.73 | 2.76 |
| | A18D | | | | | | |
| | V31L | | | | | | |
| | E64D | | | | | | |
| | D71S | | | | | | |
| | D83A | | | | | | |
| | V84K | | | | | | |
| t876904 | D13R | 66141.66 | 6038.91 | 38337.34 | 2570.39 | 1.73 | 2.35 |
| | A18D | | | | | | |
| | N29S | | | | | | |
| | Q70L | | | | | | |
| | D71E | | | | | | |
| | G80A | | | | | | |
| | D83K | | | | | | |
| t877615 | E17N | 61434.01 | 4434.67 | 35676.87 | 13790.92 | 1.72 | 0.32 |
| | T26R | | | | | | |
| | V31L | | | | | | |
| | D71S | | | | | | |
| | D83K | | | | | | |
| | V84F | | | | | | |
| t877061 | E17N | 53257.10 | 3324.55 | 30936.45 | 3064.13 | 1.72 | 1.08 |
| | A18D | | | | | | |
| | V31L | | | | | | |
| | D71S | | | | | | |
| | D83E | | | | | | |
| | V84F | | | | | | |
| | T98K | | | | | | |
| t876840 | D13R | 69892.25 | 2966.24 | 40994.08 | 2674.04 | 1.70 | 1.11 |
| | A18D | | | | | | |
| | N29S | | | | | | |
| | Q70L | | | | | | |
| | D71Q | | | | | | |
| | G80A | | | | | | |
| | D83K | | | | | | |
| | V84K | | | | | | |
| | Y85F | | | | | | |
| t877471 | E17N | 43661.98 | 29171.98 | 25645.43 | 17118.60 | 1.70 | 1.70 |
| | V31L | | | | | | |
| | D71S | | | | | | |
| | D83E | | | | | | |
| | V84F | | | | | | |
| | T98K | | | | | | |
| t876815 | V84K | 45825.55 | 4088.83 | 26919.71 | 2484.95 | 1.70 | 1.65 |
| | D39G | | | | | | |
| | D83E | | | | | | |
| | D13R | | | | | | |
| | E64D | | | | | | |
| | S65N | | | | | | |
| t877080 | E17N | 27202.36 | 31490.38 | 16017.57 | 18496.58 | 1.70 | 1.70 |
| | A18D | | | | | | |
| | V31L | | | | | | |
| | E64D | | | | | | |
| | D71S | | | | | | |
| | D83K | | | | | | |
| | V84F | | | | | | |
| t877383 | D13R | 56579.90 | 2839.54 | 33334.05 | 2463.72 | 1.70 | 1.15 |
| | A18D | | | | | | |
| | T26R | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| t876802 | A18D | 47318.59 | 6209.25 | 27962.90 | 2462.72 | 1.69 | 2.52 |
| | T26R | | | | | | |
| | E52H | | | | | | |
| | Y55F | | | | | | |
| | Q70A | | | | | | |
| | G80A | | | | | | |
| | D83E | | | | | | |
| | V84K | | | | | | |
| | T98Y | | | | | | |
| t877150 | A18D | 55112.13 | 1557.50 | 32832.41 | 1290.71 | 1.68 | 1.21 |
| | T26R | | | | | | |
| | I33R | | | | | | |
| | D39G | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| t877662 | A18D | 56767.62 | 1891.78 | 33967.79 | 992.94 | 1.67 | 1.91 |
| | T26R | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| | T98K | | | | | | |
| t877593 | D13R | 61619.50 | 2949.19 | 36934.06 | 1572.95 | 1.67 | 1.87 |
| | A18D | | | | | | |
| | N29S | | | | | | |
| | Q70L | | | | | | |
| | D71Q | | | | | | |
| | G80A | | | | | | |
| | D83E | | | | | | |
| t877319 | E17N | 54369.52 | 15021.04 | 32731.93 | 11595.99 | 1.66 | 1.30 |
| | A18D | | | | | | |
| | V31L | | | | | | |
| | D71S | | | | | | |
| | D83A | | | | | | |
| | V84F | | | | | | |
| t877010 | D13R | 50212.28 | 1463.94 | 30313.87 | 2666.87 | 1.66 | 0.55 |
| | A18D | | | | | | |
| | N29S | | | | | | |
| | E64D | | | | | | |
| | S65N | | | | | | |
| | Q70L | | | | | | |
| | D71Q | | | | | | |
| | G80A | | | | | | |
| | D83K | | | | | | |
| t876911 | E17N | 44933.16 | 2769.64 | 27163.22 | 2271.33 | 1.65 | 1.22 |
| | A18D | | | | | | |
| | V31L | | | | | | |
| | D71S | | | | | | |
| | D83K | | | | | | |
| | V84K | | | | | | |
| t876790 | E17N | 42767.90 | 1422.92 | 25886.38 | 2259.19 | 1.65 | 0.63 |
| | V31L | | | | | | |
| | I33N | | | | | | |
| | V66Y | | | | | | |
| | D71S | | | | | | |
| | G82R | | | | | | |
| | V84F | | | | | | |
| t877352 | A18D | 54491.72 | 3151.88 | 33032.73 | 3790.76 | 1.65 | 0.83 |
| | T26R | | | | | | |
| | I33R | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| | T98K | | | | | | |
| t876781 | A18D | 49062.31 | 2690.53 | 30141.20 | 1762.18 | 1.63 | 1.53 |
| | E22K | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t877581 | T26R | 51086.83 | 790.11 | 31485.27 | 1784.46 | 1.62 | 0.44 |
| | I33H | | | | | | |
| | Y55F | | | | | | |
| | E64D | | | | | | |
| | Q70A | | | | | | |
| | G80A | | | | | | |
| | D83K | | | | | | |
| | T98K | | | | | | |
| t877387 | A18D | 54247.25 | 2901.87 | 33498.88 | 4221.74 | 1.62 | 0.69 |
| | T26R | | | | | | |
| | I33R | | | | | | |
| | E52H | | | | | | |
| | Y55F | | | | | | |
| | Q70A | | | | | | |
| | G80A | | | | | | |
| | D83E | | | | | | |
| | V84K | | | | | | |
| | T98Y | | | | | | |
| t877062 | D13R | 53174.32 | 4952.39 | 32898.77 | 3748.10 | 1.62 | 1.32 |
| | A18D | | | | | | |
| | T26R | | | | | | |
| | N29S | | | | | | |
| | Q70L | | | | | | |
| | D71Q | | | | | | |
| | G80A | | | | | | |
| | D83K | | | | | | |
| | T98K | | | | | | |
| t877397 | D13R | 57542.45 | 10254.51 | 36004.04 | 2810.36 | 1.60 | 3.65 |
| | A18D | | | | | | |
| | N29S | | | | | | |
| | Q70L | | | | | | |
| | D71Q | | | | | | |
| | G80A | | | | | | |
| | D83K | | | | | | |
| | V84K | | | | | | |
| t877031 | D13R | 53912.32 | 2482.86 | 33761.70 | 3118.14 | 1.60 | 0.80 |
| | A18D | | | | | | |
| | T26R | | | | | | |
| | N29S | | | | | | |
| | E64D | | | | | | |
| | Q70L | | | | | | |
| | D71Q | | | | | | |
| | G80A | | | | | | |
| | D83K | | | | | | |
| t877343 | T26R | 59141.53 | 1317.80 | 37312.72 | 3134.04 | 1.59 | 0.42 |
| | D83E | | | | | | |
| | D13R | | | | | | |
| | E64D | | | | | | |
| | T98K | | | | | | |
| | S65N | | | | | | |
| t877356 | A18D | 52102.50 | 4473.06 | 32945.28 | 2417.88 | 1.58 | 1.85 |
| | T26R | | | | | | |
| | I33H | | | | | | |
| | Y55F | | | | | | |
| | Q70A | | | | | | |
| | G80A | | | | | | |
| | D83K | | | | | | |
| | T98K | | | | | | |
| t877374 | D13R | 53161.62 | 3886.75 | 33716.08 | 6694.27 | 1.58 | 0.58 |
| | A18D | | | | | | |
| | N29S | | | | | | |
| | Q70L | | | | | | |
| | G80A | | | | | | |
| | D83E | | | | | | |
| | T98K | | | | | | |
| t877063 | E17N | 45766.54 | 4802.01 | 29062.23 | 1600.35 | 1.57 | 3.00 |
| | A18D | | | | | | |
| | T26R | | | | | | |
| | V31L | | | | | | |
| | D71S | | | | | | |
| | D83K | | | | | | |
| | V84F | | | | | | |
| t877414 | E17N | 49412.38 | 2888.25 | 31403.87 | 3633.12 | 1.57 | 0.79 |
| | V31L | | | | | | |
| | D71S | | | | | | |
| | D83K | | | | | | |
| | V84F | | | | | | |
| | T98K | | | | | | |
| t876776 | E17N | 45250.30 | 1408.77 | 28762.94 | 2425.69 | 1.57 | 0.58 |
| | A18D | | | | | | |
| | V31L | | | | | | |
| | I33N | | | | | | |
| | D71S | | | | | | |
| | V84F | | | | | | |
| t877180 | E17N | 42965.53 | 2933.77 | 27425.53 | 969.64 | 1.57 | 3.03 |
| | V31L | | | | | | |
| | I33N | | | | | | |
| | E64D | | | | | | |
| | D71S | | | | | | |
| | V84K | | | | | | |
| t877503 | A18D | 54727.11 | 838.20 | 35175.07 | 1707.06 | 1.56 | 0.49 |
| | T26R | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| | T98Y | | | | | | |
| t877566 | A18D | 59335.98 | 4118.21 | 38167.15 | 1357.54 | 1.55 | 3.03 |
| | T26R | | | | | | |
| | E52H | | | | | | |
| | Y55F | | | | | | |
| | Q70A | | | | | | |
| | G80A | | | | | | |
| | D83K | | | | | | |
| | V84K | | | | | | |
| | Y85F | | | | | | |
| t877349 | E17N | 55247.91 | 3258.62 | 35623.05 | 413.02 | 1.55 | 7.89 |
| | A18D | | | | | | |
| | V31L | | | | | | |
| | D71S | | | | | | |
| | D83A | | | | | | |
| | V84K | | | | | | |
| t877246 | T26R | 43789.53 | 4556.19 | 28242.40 | 2610.61 | 1.55 | 1.75 |
| | I33H | | | | | | |
| | Y55F | | | | | | |
| | Q70A | | | | | | |
| | G80A | | | | | | |
| | D83E | | | | | | |
| | V84K | | | | | | |
| | Y85A | | | | | | |
| t877626 | A18D | 51276.81 | 3132.15 | 33243.31 | 3045.66 | 1.54 | 1.03 |
| | T26R | | | | | | |
| | I33R | | | | | | |
| | D39G | | | | | | |
| | E52H | | | | | | |
| | Y55F | | | | | | |
| | Q70A | | | | | | |
| | G80A | | | | | | |
| | D83K | | | | | | |
| | V84K | | | | | | |
| t877614 | D13R | 59002.02 | 5566.68 | 38706.25 | 3000.72 | 1.52 | 1.86 |
| | A18D | | | | | | |
| | N29S | | | | | | |
| | S65N | | | | | | |
| | Q70L | | | | | | |
| | D71Q | | | | | | |
| | G80A | | | | | | |
| | D83K | | | | | | |
| | V84K | | | | | | |
| t877511 | D13R | 55323.29 | 1673.57 | 36337.22 | 3803.47 | 1.52 | 0.44 |
| | A18D | | | | | | |
| | N29S | | | | | | |
| | Q70L | | | | | | |
| | G80A | | | | | | |
| | D83K | | | | | | |
| t877705 | D13R | 58682.51 | 1007.68 | 38799.46 | 2267.89 | 1.51 | 0.44 |
| | A18D | | | | | | |
| | T26R | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| t877681 | D13R | 64473.67 | 3661.18 | 42633.55 | 2507.41 | 1.51 | 1.46 |
| | A18D | | | | | | |
| | N29S | | | | | | |
| | Q70L | | | | | | |
| | D71Q | | | | | | |
| | G80A | | | | | | |
| | D83K | | | | | | |
| | T98Y | | | | | | |
| t876883 | T26R | 43635.02 | 6523.95 | 29123.60 | 3584.38 | 1.50 | 1.82 |
| | I33H | | | | | | |
| | Y55F | | | | | | |
| | Q70A | | | | | | |
| | I74G | | | | | | |
| | G80A | | | | | | |
| | D83K | | | | | | |
| | V84K | | | | | | |
| | Y85A | | | | | | |
| t876821 | T26R | 46393.73 | 4393.48 | 31311.04 | 2086.82 | 1.48 | 2.11 |
| | I33H | | | | | | |
| | Y55F | | | | | | |
| | Q70A | | | | | | |
| | G80A | | | | | | |
| | D83K | | | | | | |
| | V84K | | | | | | |
| | Y85A | | | | | | |
| | T98Y | | | | | | |
| t877125 | D13R | 50594.32 | 19519.68 | 34638.15 | 10759.79 | 1.46 | 1.81 |
| | A18D | | | | | | |
| | N29S | | | | | | |
| | Q70L | | | | | | |
| | D71Q | | | | | | |
| | G80A | | | | | | |
| | D83E | | | | | | |
| | T98Y | | | | | | |
| t876760 | D13R | 59978.32 | 5456.33 | 41894.55 | 1871.36 | 1.43 | 2.92 |
| | A18D | | | | | | |
| | N29S | | | | | | |
| | S65N | | | | | | |
| | Q70L | | | | | | |
| | D71Q | | | | | | |
| | G80A | | | | | | |
| | D83K | | | | | | |
| | T98K | | | | | | |
| t877110 | E17N | 44622.97 | 4117.58 | 31752.97 | 2894.48 | 1.41 | 1.42 |
| | V31L | | | | | | |
| | D71S | | | | | | |
| | D83K | | | | | | |
| | V84F | | | | | | |
| | T98Y | | | | | | |
| t877325 | A18D | 53864.14 | 4270.57 | 38400.16 | 1835.27 | 1.40 | 2.33 |
| | T26R | | | | | | |
| | E52H | | | | | | |
| | Y55F | | | | | | |
| | V61S | | | | | | |
| | Q70A | | | | | | |
| | G80A | | | | | | |
| | D83N | | | | | | |
| | V84K | | | | | | |
| | Y85F | | | | | | |
| t877026 | T26R | 41245.64 | 1460.34 | 29622.85 | 1819.79 | 1.39 | 0.80 |
| | I33H | | | | | | |
| | Y55F | | | | | | |
| | S65N | | | | | | |
| | Q70A | | | | | | |
| | I74G | | | | | | |
| | G80A | | | | | | |
| | D83K | | | | | | |
| | Y85A | | | | | | |
| t877654 | D13R | 53277.86 | 2190.22 | 38500.47 | 1193.48 | 1.38 | 1.84 |
| | A18D | | | | | | |
| | N29S | | | | | | |
| | Q70L | | | | | | |
| | D71Q | | | | | | |
| | G80A | | | | | | |
| | D83K | | | | | | |
| | V84K | | | | | | |
| | T98Y | | | | | | |
| t877683 | T26R | 52683.68 | 2906.47 | 38103.66 | 4156.85 | 1.38 | 0.70 |
| | I33H | | | | | | |
| | Y55F | | | | | | |
| | E64D | | | | | | |
| | Q70A | | | | | | |
| | G80A | | | | | | |
| | D83E | | | | | | |
| | Y85A | | | | | | |
| t877152 | E17N | 39982.71 | 3767.63 | 28922.01 | 3159.46 | 1.38 | 1.19 |
| | V31L | | | | | | |
| | I33N | | | | | | |
| | E64D | | | | | | |
| | D71S | | | | | | |
| | V84F | | | | | | |
| | T98Y | | | | | | |
| t877013 | T26R | 50250.86 | 3051.02 | 36921.70 | 3587.18 | 1.36 | 0.85 |
| | I33H | | | | | | |
| | Y55F | | | | | | |
| | E64D | | | | | | |
| | Q70A | | | | | | |
| | G80A | | | | | | |
| | D83K | | | | | | |
| | Y85A | | | | | | |
| t877459 | A18D | 49613.42 | 17677.78 | 36848.27 | 10516.95 | 1.35 | 1.68 |
| | T26R | | | | | | |
| | I33N | | | | | | |
| | E52H | | | | | | |
| | Y55F | | | | | | |
| | Q70A | | | | | | |
| | D71S | | | | | | |
| | G80A | | | | | | |
| | D83N | | | | | | |
| | V84K | | | | | | |
| t877360 | A18D | 55629.74 | 5239.91 | 41344.24 | 3067.91 | 1.35 | 1.71 |
| | T26R | | | | | | |
| | I33H | | | | | | |
| | Y55F | | | | | | |
| | E64D | | | | | | |
| | Q70A | | | | | | |
| | G80A | | | | | | |
| | D83K | | | | | | |
| | Y85A | | | | | | |
| t877323 | T26R | 53155.73 | 2541.27 | 39716.98 | 911.82 | 1.34 | 2.79 |
| | I33H | | | | | | |
| | Y55F | | | | | | |
| | E64D | | | | | | |
| | S65N | | | | | | |
| | Q70A | | | | | | |
| | G80A | | | | | | |
| | D83K | | | | | | |
| t877700 | D13R | 54030.56 | 1091.17 | 40737.22 | 1921.22 | 1.33 | 0.57 |
| | A18D | | | | | | |
| | N29S | | | | | | |
| | V66Y | | | | | | |
| | Q70L | | | | | | |
| | G80A | | | | | | |
| | D83K | | | | | | |
| | Y85F | | | | | | |
| t877114 | E17N | 44154.74 | 5797.62 | 33380.26 | 4354.29 | 1.32 | 1.33 |
| | A18D | | | | | | |
| | V31L | | | | | | |
| | V66Y | | | | | | |
| | D71S | | | | | | |
| | D83A | | | | | | |
| | V84F | | | | | | |
| | Y85F | | | | | | |
| t877743 | D13R | 53182.29 | 3024.92 | 40385.00 | 2265.42 | 1.32 | 1.34 |
| | A18D | | | | | | |
| | E22K | | | | | | |
| | N29S | | | | | | |
| | V66Y | | | | | | |
| | Q70L | | | | | | |
| | G80A | | | | | | |
| | D83K | | | | | | |
| t876903 | D71E | 42448.26 | 2819.85 | 32317.42 | 1854.84 | 1.31 | 1.52 |
| | V66Y | | | | | | |
| | V61S | | | | | | |
| | E22K | | | | | | |
| | D83K | | | | | | |
| | T98K | | | | | | |
| t877509 | T26R | 59558.15 | 6000.20 | 46174.04 | 3308.49 | 1.29 | 1.81 |
| | I33H | | | | | | |
| | Y55F | | | | | | |
| | Q70A | | | | | | |
| | G80A | | | | | | |
| | D83K | | | | | | |
| | Y85A | | | | | | |
| t877072 | E17N | 43184.72 | 13725.25 | 33598.09 | 13119.62 | 1.29 | 1.05 |
| | A18D | | | | | | |
| | V31L | | | | | | |
| | D71S | | | | | | |
| | D83K | | | | | | |
| | V84F | | | | | | |
| | T98Y | | | | | | |
| t877078 | A18D | 46074.38 | 3379.15 | 35855.08 | 1592.37 | 1.29 | 2.12 |
| | T26R | | | | | | |
| | D39G | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| t877167 | E17N | 38952.55 | 26147.79 | 30322.74 | 1220.26 | 1.28 | 21.43 |
| | V31L | | | | | | |
| | D71S | | | | | | |
| | D83A | | | | | | |
| | V84K | | | | | | |
| | Y85F | | | | | | |
| t877275 | D13R | 52433.61 | 2891.17 | 40989.42 | 24382.88 | 1.28 | 0.12 |
| | A18D | | | | | | |
| | N29S | | | | | | |
| | S65N | | | | | | |
| | Q70L | | | | | | |
| | I74G | | | | | | |
| | G80A | | | | | | |
| | D83K | | | | | | |
| t877055 | A18D | 45554.91 | 14223.25 | 35884.72 | 15700.71 | 1.27 | 0.91 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | F88N | | | | | | |
| | T98K | | | | | | |
| t877316 | T26R | 48379.93 | 19967.42 | 40767.54 | 17324.29 | 1.19 | 1.15 |
| | I33H | | | | | | |
| | Y55F | | | | | | |
| | Q70A | | | | | | |
| | G80A | | | | | | |
| | D83K | | | | | | |
| | Y85A | | | | | | |
| t876939 | D13R | 46079.32 | 4457.09 | 40107.14 | 4147.24 | 1.15 | 1.07 |
| | A18D | | | | | | |
| | N29S | | | | | | |
| | Q70L | | | | | | |
| | G80A | | | | | | |
| | D83K | | | | | | |
| | V84K | | | | | | |
| | T98K | | | | | | |
| t877447 | T26R | 52568.18 | 2553.16 | 45938.46 | 3375.15 | 1.14 | 0.76 |
| | I33H | | | | | | |
| | Y55F | | | | | | |
| | S65N | | | | | | |
| | Q70A | | | | | | |
| | G80A | | | | | | |
| | D83E | | | | | | |
| | Y85A | | | | | | |
| t877364 | A18D | 52750.13 | 2411.79 | 46905.95 | 3854.75 | 1.12 | 0.63 |
| | T26R | | | | | | |
| | I33H | | | | | | |
| | Y55F | | | | | | |
| | S65N | | | | | | |
| | Q70A | | | | | | |
| | G80A | | | | | | |
| | D83K | | | | | | |
| | Y85A | | | | | | |
| t877321 | D13R | 53063.05 | 2361.30 | 52368.65 | 5735.38 | 1.01 | 0.41 |
| | A18D | | | | | | |
| | N29S | | | | | | |
| | D39G | | | | | | |
| | Q70L | | | | | | |
| | G80A | | | | | | |
| | D83K | | | | | | |
| | Y85F | | | | | | |
| t877292 | D13R | 56027.12 | 3162.02 | 55882.42 | 7195.46 | 1.00 | 0.44 |
| | A18D | | | | | | |
| | N29S | | | | | | |
| | D39G | | | | | | |
| | S65N | | | | | | |
| | Q70L | | | | | | |
| | D71Q | | | | | | |
| | G80A | | | | | | |
| | D83K | | | | | | |
| t877294 | T26R | 42651.87 | 23963.68 | 47518.82 | 19815.28 | 0.90 | 1.21 |
| | I33H | | | | | | |
| | Y55F | | | | | | |
| | Q70A | | | | | | |
| | G80A | | | | | | |
| | D83K | | | | | | |
| t877230 | E17N | 35114.61 | 18324.52 | 45950.68 | 21048.95 | 0.76 | 0.87 |
| | E22K | | | | | | |
| | V31L | | | | | | |
| | D71E | | | | | | |
| | D83A | | | | | | |
| | V84F | | | | | | |
| t782404 | yZsGreen | 30940.47 | 13011.12 | 51357.25 | 15279.63 | 0.60 | 0.85 |
| t877481 | E17N | 383.97 | 767.95 | 20057.91 | 40115.82 | 0.02 | 0.02 |
| | V31L | | | | | | |
| | E64D | | | | | | |
| | D71S | | | | | | |
| | D83A | | | | | | |
| | V84K | | | | | | |
| t877455 | D71E | 365.04 | 730.08 | 19301.83 | 38603.66 | 0.02 | 0.02 |
| | V66Y | | | | | | |
| | E22K | | | | | | |
| | G82R | | | | | | |
| | T98K | | | | | | |
| | D13R | | | | | | |
| t877023 | D13R | 0.00 | 0.00 | 18625.67 | 37251.34 | 0.00 | 0.00 |
| | E17N | | | | | | |
| | A18D | | | | | | |
| | V31L | | | | | | |
| | I33N | | | | | | |
| | D71S | | | | | | |
| | V84K | | | | | | |
| t877057 | E17N | 0.00 | 0.00 | 17235.94 | 34471.88 | 0.00 | 0.00 |
| | V31L | | | | | | |
| | I33N | | | | | | |
| | D71S | | | | | | |
| | D83E | | | | | | |
| | V84K | | | | | | |

TABLE 9B
Activity data for synthetic OAC polypeptides in S. cerevisiae described in
Example 4 and FIGS. 12C-12D.

| | | Olivetolic | Olivetolic | | | | |
|---|---|---|---|---|---|---|---|
| | | Acid | Acid | Olivetol | Olivetol | | |
| | | (ug/L) | (ug/L) | (ug/L) | (ug/L) | | |
| | Mutations | mean | std | mean | std | OA/OL | OA/OL |
| | relative to | t815900 | t815900 | t815900 | t815900 | mean | std |
| | CsOAC | normalized | normalized | normalized | normalized | normalized | normalized |
| | (SEQ ID | (plate | (plate | (plate | (plate | to | to |
| Strain | NO: 1) | mean) | mean) | mean) | mean) | t815900 | t815900 |
| t877598 | E17N | 1.05 | 0.34 | 0.65 | 0.19 | 1.63 | 1.73 |
| | A18D | | | | | | |
| | E22K | | | | | | |
| | V31L | | | | | | |
| | I33N | | | | | | |
| | D71E | | | | | | |
| | V84F | | | | | | |
| t877570 | E17N | 1.1 | 0.21 | 0.85 | 0.07 | 1.3 | 3.04 |
| | V31L | | | | | | |
| | I33N | | | | | | |
| | S65N | | | | | | |
| | D71S | | | | | | |
| | V84K | | | | | | |
| t877091 | E17N | 0.94 | 0.08 | 0.58 | 0.06 | 1.61 | 1.39 |
| | V31L | | | | | | |
| | I33N | | | | | | |
| | D71S | | | | | | |
| | V84K | | | | | | |
| | T98K | | | | | | |
| t876768 | E17N | 0.89 | 0.04 | 0.64 | 0.09 | 1.4 | 0.43 |
| | V31L | | | | | | |
| | D39G | | | | | | |
| | D71S | | | | | | |
| | D83A | | | | | | |
| | V84K | | | | | | |
| t876938 | V84K | 0.91 | 0.06 | 0.74 | 0.08 | 1.24 | 0.79 |
| | T26R | | | | | | |
| | D39G | | | | | | |
| | D13R | | | | | | |
| | T98K | | | | | | |
| | S65N | | | | | | |
| t876909 | E17N | 0.61 | 0.07 | 0.4 | 0.11 | 1.52 | 0.66 |
| | A18D | | | | | | |
| | T26R | | | | | | |
| | V31L | | | | | | |
| | D71S | | | | | | |
| | D83A | | | | | | |
| | V84K | | | | | | |
| t877536 | E17N | 0.87 | 0.04 | 0.63 | 0.02 | 1.37 | 1.75 |
| | A18D | | | | | | |
| | V31L | | | | | | |
| | I33N | | | | | | |
| | D71S | | | | | | |
| | V84K | | | | | | |
| | T98K | | | | | | |
| t876749 | E17N | 0.88 | 0.07 | 0.72 | 0.03 | 1.21 | 2.2 |
| | A18D | | | | | | |
| | T26R | | | | | | |
| | V31L | | | | | | |
| | I33N | | | | | | |
| | D71S | | | | | | |
| | V84K | | | | | | |
| t877477 | A18D | 1.07 | 0.02 | 0.62 | 0.05 | 1.71 | 0.46 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| | F88N | | | | | | |
| t877264 | V84K | 0.63 | 0.04 | 1.15 | 0.12 | 0.54 | 0.34 |
| | T26R | | | | | | |
| | D39G | | | | | | |
| | D83E | | | | | | |
| | D13R | | | | | | |
| | T98K | | | | | | |
| t877554 | E17N | 0.81 | 0.04 | 0.64 | 0.03 | 1.26 | 1.12 |
| | A18D | | | | | | |
| | V31L | | | | | | |
| | I33N | | | | | | |
| | S65N | | | | | | |
| | D71S | | | | | | |
| | V84K | | | | | | |
| t876985 | E17N | 0.87 | 0.03 | 0.71 | 0.14 | 1.23 | 0.24 |
| | V31L | | | | | | |
| | I33N | | | | | | |
| | D71S | | | | | | |
| | G82R | | | | | | |
| | V84F | | | | | | |
| t877362 | E17N | 0.61 | 0.06 | 1.15 | 0.14 | 0.53 | 0.47 |
| | A18D | | | | | | |
| | V31L | | | | | | |
| | I33N | | | | | | |
| | V66Y | | | | | | |
| | D71S | | | | | | |
| | V84F | | | | | | |
| t876764 | D13R | 0.88 | 0.04 | 0.74 | 0.05 | 1.2 | 0.74 |
| | E17N | | | | | | |
| | A18D | | | | | | |
| | V31L | | | | | | |
| | D71S | | | | | | |
| | D83A | | | | | | |
| | V84K | | | | | | |
| t877407 | A18D | 1.09 | 0.03 | 0.67 | 0.05 | 1.62 | 0.75 |
| | E22K | | | | | | |
| | T26R | | | | | | |
| | I33R | | | | | | |
| | E52H | | | | | | |
| | Y55F | | | | | | |
| | V66Y | | | | | | |
| | Q70A | | | | | | |
| | G80A | | | | | | |
| | D83N | | | | | | |
| | V84K | | | | | | |
| t877429 | E17N | 0.62 | 0.02 | 1.19 | 0.04 | 0.52 | 0.52 |
| | A18D | | | | | | |
| | V31L | | | | | | |
| | I33N | | | | | | |
| | D71S | | | | | | |
| | V84K | | | | | | |
| t877431 | E17N | 1.07 | 0.07 | 0.66 | 0.08 | 1.61 | 0.94 |
| | A18D | | | | | | |
| | V31L | | | | | | |
| | I33N | | | | | | |
| | D71E | | | | | | |
| | V84F | | | | | | |
| t877109 | E17N | 0.59 | 0.05 | 0.43 | 0.04 | 1.37 | 1.08 |
| | T26R | | | | | | |
| | V31L | | | | | | |
| | D71S | | | | | | |
| | D83A | | | | | | |
| | V84K | | | | | | |
| t876982 | T26R | 0.93 | 0.04 | 0.68 | 0.13 | 1.35 | 0.32 |
| | D39G | | | | | | |
| | D83E | | | | | | |
| | E64D | | | | | | |
| | T98K | | | | | | |
| | S65N | | | | | | |
| t877136 | E17N | 1.05 | 0.05 | 0.66 | 0.04 | 1.59 | 1.41 |
| | A18D | | | | | | |
| | V31L | | | | | | |
| | D39G | | | | | | |
| | D71S | | | | | | |
| | D83A | | | | | | |
| | V84K | | | | | | |
| t877723 | E17N | 0.73 | 0.17 | 0.58 | 0.2 | 1.25 | 0.86 |
| | A18D | | | | | | |
| | V31L | | | | | | |
| | I33N | | | | | | |
| | D71S | | | | | | |
| | G82R | | | | | | |
| | V84F | | | | | | |
| t877187 | A18D | 1.11 | 0.09 | 0.69 | 0.01 | 1.6 | 8.39 |
| | T26R | | | | | | |
| | I33R | | | | | | |
| | E52H | | | | | | |
| | Y55F | | | | | | |
| | V66Y | | | | | | |
| | Q70A | | | | | | |
| | G80A | | | | | | |
| | D83N | | | | | | |
| | V84K | | | | | | |
| | Y85F | | | | | | |
| t877011 | E17N | 0.85 | 0.08 | 0.74 | 0.04 | 1.15 | 2.14 |
| | A18D | | | | | | |
| | V31L | | | | | | |
| | I33N | | | | | | |
| | D71S | | | | | | |
| | I74G | | | | | | |
| | V84K | | | | | | |
| t877449 | E17N | 0.66 | 0.02 | 1.31 | 0.07 | 0.5 | 0.26 |
| | V31L | | | | | | |
| | I33N | | | | | | |
| | D71S | | | | | | |
| | V84K | | | | | | |
| | Y85F | | | | | | |
| t877712 | E17N | 0.98 | 0.1 | 0.84 | 0.12 | 1.16 | 0.84 |
| | A18D | | | | | | |
| | V31L | | | | | | |
| | D71S | | | | | | |
| | I74G | | | | | | |
| | D83E | | | | | | |
| | V84F | | | | | | |
| t877501 | D39G | 0.91 | 0.05 | 0.79 | 0.06 | 1.15 | 0.92 |
| | D83E | | | | | | |
| | D13R | | | | | | |
| | E64D | | | | | | |
| | T98K | | | | | | |
| | S65N | | | | | | |
| t876853 | E17N | 0.89 | 0.11 | 0.79 | 0.08 | 1.14 | 1.41 |
| | A18D | | | | | | |
| | V31L | | | | | | |
| | S65N | | | | | | |
| | D71S | | | | | | |
| | D83A | | | | | | |
| | V84K | | | | | | |
| t877644 | E17N | 0.9 | 0.13 | 0.74 | 0.13 | 1.22 | 1.05 |
| | A18D | | | | | | |
| | V31L | | | | | | |
| | I33N | | | | | | |
| | D71S | | | | | | |
| | D83K | | | | | | |
| | V84K | | | | | | |
| t877056 | T26R | 0.92 | 0.05 | 0.71 | 0.04 | 1.3 | 1.3 |
| | D39G | | | | | | |
| | D83E | | | | | | |
| | D13R | | | | | | |
| | T98K | | | | | | |
| | S65N | | | | | | |
| t877014 | E17N | 0.96 | 0.07 | 0.74 | 0.03 | 1.3 | 2.29 |
| | A18D | | | | | | |
| | E22K | | | | | | |
| | V31L | | | | | | |
| | D71E | | | | | | |
| | D83A | | | | | | |
| | V84F | | | | | | |
| t877228 | D13R | 1.11 | 0.02 | 0.73 | 0.02 | 1.53 | 0.95 |
| | A18D | | | | | | |
| | N29S | | | | | | |
| | Q70L | | | | | | |
| | D71Q | | | | | | |
| | G80A | | | | | | |
| | D83E | | | | | | |
| | T98K | | | | | | |
| t876933 | V84K | 0.91 | 0.07 | 0.71 | 0.02 | 1.29 | 4.17 |
| | T26R | | | | | | |
| | D39G | | | | | | |
| | D13R | | | | | | |
| | E64D | | | | | | |
| | S65N | | | | | | |
| t876870 | E17N | 0.88 | 0.05 | 0.85 | 0.05 | 1.04 | 0.96 |
| | A18D | | | | | | |
| | V31L | | | | | | |
| | I33N | | | | | | |
| | D71S | | | | | | |
| | D83E | | | | | | |
| | V84K | | | | | | |
| t877158 | V84K | 1.06 | 0.15 | 0.86 | 0.06 | 1.23 | 2.63 |
| | T26R | | | | | | |
| | D83E | | | | | | |
| | D13R | | | | | | |
| | E64D | | | | | | |
| | T98K | | | | | | |
| t877223 | E22K | 1.03 | 0.08 | 0.68 | 0.04 | 1.52 | 2.14 |
| | T26R | | | | | | |
| | I33H | | | | | | |
| | Y55F | | | | | | |
| | Q70A | | | | | | |
| | D71E | | | | | | |
| | G80A | | | | | | |
| | D83K | | | | | | |
| t877441 | D13R | 1.03 | 0.08 | 0.69 | 0.07 | 1.49 | 1.14 |
| | A18D | | | | | | |
| | N29S | | | | | | |
| | Q70L | | | | | | |
| | D71Q | | | | | | |
| | I74G | | | | | | |
| | G80A | | | | | | |
| | D83K | | | | | | |
| | T98K | | | | | | |
| t877227 | A18D | 1.08 | 0.11 | 0.72 | 0.13 | 1.5 | 0.88 |
| | T26R | | | | | | |
| | I33R | | | | | | |
| | E52H | | | | | | |
| | Y55F | | | | | | |
| | Q70A | | | | | | |
| | D71E | | | | | | |
| | G80A | | | | | | |
| | D83N | | | | | | |
| | V84K | | | | | | |
| t877534 | V84K | 0.94 | 0.07 | 0.81 | 0.05 | 1.17 | 1.5 |
| | T26R | | | | | | |
| | D39G | | | | | | |
| | D83E | | | | | | |
| | T98K | | | | | | |
| | S65N | | | | | | |
| t876945 | E17N | 0.89 | 0.07 | 0.72 | 0.08 | 1.23 | 0.85 |
| | V31L | | | | | | |
| | I33N | | | | | | |
| | D71S | | | | | | |
| | V84K | | | | | | |
| | T98Y | | | | | | |
| t876987 | E17N | 0.91 | 0.11 | 0.85 | 0.07 | 1.07 | 1.63 |
| | V31L | | | | | | |
| | D71S | | | | | | |
| | D83A | | | | | | |
| | V84F | | | | | | |
| | T98Y | | | | | | |
| t877645 | E17N | 1.05 | 0.15 | 1.07 | 0.07 | 0.97 | 2.03 |
| | E22K | | | | | | |
| | V31L | | | | | | |
| | I33N | | | | | | |
| | D71E | | | | | | |
| | V84F | | | | | | |
| t877404 | E17N | 1.02 | 0.03 | 0.7 | 0.03 | 1.46 | 1.07 |
| | A18D | | | | | | |
| | V31L | | | | | | |
| | D71S | | | | | | |
| | I74G | | | | | | |
| | D83A | | | | | | |
| | V84K | | | | | | |
| t877718 | E17N | 0.9 | 0.02 | 0.79 | 0.05 | 1.14 | 0.39 |
| | A18D | | | | | | |
| | V31L | | | | | | |
| | D71E | | | | | | |
| | D83A | | | | | | |
| | V84F | | | | | | |
| t877698 | E17N | 0.9 | 0.18 | 0.84 | 0.19 | 1.07 | 0.98 |
| | A18D | | | | | | |
| | V31L | | | | | | |
| | D71S | | | | | | |
| | D83E | | | | | | |
| | V84F | | | | | | |
| | T98Y | | | | | | |
| t876959 | A18D | 0.94 | 0.1 | 0.89 | 0.09 | 1.06 | 1.14 |
| | T26R | | | | | | |
| | D39G | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | T98K | | | | | | |
| t877021 | E17N | 0.91 | 0.05 | 0.86 | 0.03 | 1.06 | 1.52 |
| | V31L | | | | | | |
| | I33N | | | | | | |
| | V66Y | | | | | | |
| | D71S | | | | | | |
| | V84F | | | | | | |
| t877702 | E17N | 0.94 | 0.08 | 0.83 | 0.18 | 1.13 | 0.44 |
| | V31L | | | | | | |
| | D71S | | | | | | |
| | D83E | | | | | | |
| | V84F | | | | | | |
| | T98Y | | | | | | |
| t876990 | V84K | 0.92 | 0.03 | 0.77 | 0.05 | 1.2 | 0.56 |
| | D39G | | | | | | |
| | D13R | | | | | | |
| | E64D | | | | | | |
| | T98K | | | | | | |
| | S65N | | | | | | |
| t876772 | E17N | 0.78 | 0.08 | 0.75 | 0.04 | 1.04 | 1.99 |
| | A18D | | | | | | |
| | V31L | | | | | | |
| | I33N | | | | | | |
| | V66Y | | | | | | |
| | D71E | | | | | | |
| | V84F | | | | | | |
| t877248 | E17N | 0.67 | 0.33 | 0.58 | 0.68 | 1.15 | 0.49 |
| | A18D | | | | | | |
| | V31L | | | | | | |
| | I33N | | | | | | |
| | D71S | | | | | | |
| | I74G | | | | | | |
| | D83K | | | | | | |
| | V84F | | | | | | |
| t877510 | E17N | 0.92 | 0.07 | 0.84 | 0.04 | 1. 1 | 1.6 |
| | T26R | | | | | | |
| | V31L | | | | | | |
| | D71S | | | | | | |
| | D83E | | | | | | |
| | V84F | | | | | | |
| t877258 | E17N | 0.66 | 0.08 | 1.49 | 0.22 | 0.44 | 0.37 |
| | V31L | | | | | | |
| | I33N | | | | | | |
| | E64D | | | | | | |
| | D71S | | | | | | |
| | V84F | | | | | | |
| t877088 | E17N | 0.9 | 0.07 | 0.77 | 0.04 | 1.17 | 1.76 |
| | V31L | | | | | | |
| | I33N | | | | | | |
| | D71S | | | | | | |
| | D83K | | | | | | |
| | V84K | | | | | | |
| t877516 | V84K | 0.84 | 0.04 | 0.82 | 0.05 | 1.03 | 0.71 |
| | D39G | | | | | | |
| | D83E | | | | | | |
| | D13R | | | | | | |
| | T98K | | | | | | |
| | S65N | | | | | | |
| t877306 | A18D | 0.96 | 0.08 | 0.85 | 0.09 | 1.13 | 0.86 |
| | T26R | | | | | | |
| | I33R | | | | | | |
| | E52H | | | | | | |
| | Y55F | | | | | | |
| | S65N | | | | | | |
| | Q70A | | | | | | |
| | G80A | | | | | | |
| | D83E | | | | | | |
| | V84K | | | | | | |
| t877188 | A18D | 1.08 | 0.08 | 0.95 | 0.06 | 1.13 | 1.32 |
| | T26R | | | | | | |
| | I33R | | | | | | |
| | E52H | | | | | | |
| | Y55F | | | | | | |
| | V66Y | | | | | | |
| | Q70A | | | | | | |
| | D71E | | | | | | |
| | G80A | | | | | | |
| | D83N | | | | | | |
| | V84K | | | | | | |
| t876766 | E17N | 0.84 | 0.06 | 0.83 | 0.08 | 1.01 | 0.79 |
| | A18D | | | | | | |
| | V31L | | | | | | |
| | I33N | | | | | | |
| | D39G | | | | | | |
| | D71S | | | | | | |
| | V84K | | | | | | |
| t877629 | E17N | 0.82 | 0.05 | 0.8 | 0.05 | 1.02 | 0.91 |
| | A18D | | | | | | |
| | V31L | | | | | | |
| | D71S | | | | | | |
| | D83A | | | | | | |
| | V84K | | | | | | |
| | T98Y | | | | | | |
| t876865 | V84K | 0.79 | 0.05 | 0.79 | 0.02 | 1 | 2.87 |
| | T26R | | | | | | |
| | D39G | | | | | | |
| | D83E | | | | | | |
| | E64D | | | | | | |
| | S65N | | | | | | |
| t876782 | A18D | 0.88 | 0.06 | 0.95 | 0.06 | 0.93 | 1.1 |
| | T26R | | | | | | |
| | I33R | | | | | | |
| | D39G | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| t877108 | A18D | 0.93 | 0.12 | 0.83 | 0.06 | 1.12 | 2.15 |
| | T26R | | | | | | |
| | E52H | | | | | | |
| | Y55F | | | | | | |
| | E64D | | | | | | |
| | Q70A | | | | | | |
| | I74G | | | | | | |
| | G80A | | | | | | |
| | D83N | | | | | | |
| | V84K | | | | | | |
| t877164 | D13R | 1.02 | 0.02 | 0.76 | 0.05 | 1.36 | 0.43 |
| | A18D | | | | | | |
| | N29S | | | | | | |
| | E64D | | | | | | |
| | Q70L | | | | | | |
| | D71Q | | | | | | |
| | G80A | | | | | | |
| | D83E | | | | | | |
| t877256 | T26R | 0.99 | 0.13 | 0.88 | 0.07 | 1.11 | 1.71 |
| | I33H | | | | | | |
| | Y55F | | | | | | |
| | Q70A | | | | | | |
| | I74G | | | | | | |
| | G80A | | | | | | |
| | D83K | | | | | | |
| | Y85A | | | | | | |
| t877596 | D13R | 1 | 0.1 | 0.79 | 0.31 | 1.26 | 0.33 |
| | A18D | | | | | | |
| | E22K | | | | | | |
| | N29S | | | | | | |
| | Q70L | | | | | | |
| | D71Q | | | | | | |
| | G80A | | | | | | |
| | D83K | | | | | | |
| t877249 | E17N | 0.83 | 0.51 | 0.6 | 0.33 | 1.37 | 1.54 |
| | A18D | | | | | | |
| | V31L | | | | | | |
| | D71S | | | | | | |
| | D83A | | | | | | |
| | V84K | | | | | | |
| | Y85F | | | | | | |
| t877439 | D13R | 1.03 | 0.06 | 0.77 | 0.04 | 1.35 | 1.67 |
| | A18D | | | | | | |
| | N29S | | | | | | |
| | E64D | | | | | | |
| | Q70L | | | | | | |
| | D71Q | | | | | | |
| | G80A | | | | | | |
| | D83K | | | | | | |
| t815900 | E17N | 1 | 0.04 | 1 | 0.11 | 1 | 0.31 |
| | A18D | | | | | | |
| | V31L | | | | | | |
| | D71S | | | | | | |
| | D83A | | | | | | |
| | V84F | | | | | | |
| t877444 | E17N | 1.12 | 0.09 | 0.82 | 0.02 | 1.37 | 3.86 |
| | A18D | | | | | | |
| | V31L | | | | | | |
| | I33N | | | | | | |
| | D71S | | | | | | |
| | G80A | | | | | | |
| | V84F | | | | | | |
| t877064 | E17N | 0.85 | 0.08 | 0.75 | 0.07 | 1.14 | 1.17 |
| | A18D | | | | | | |
| | V31L | | | | | | |
| | D71S | | | | | | |
| | D83K | | | | | | |
| | V84F | | | | | | |
| t877229 | V84K | 0.97 | 0.01 | 0.72 | 0.08 | 1.36 | 0.14 |
| | T26R | | | | | | |
| | D39G | | | | | | |
| | D83E | | | | | | |
| | D13R | | | | | | |
| | S65N | | | | | | |
| t876807 | E17N | 0.98 | 0.06 | 0.86 | 0.05 | 1.13 | 1.23 |
| | V31L | | | | | | |
| | I33N | | | | | | |
| | D39G | | | | | | |
| | D71S | | | | | | |
| | V84K | | | | | | |
| t877304 | E17N | 0.64 | 0.07 | 1.49 | 0.05 | 0.43 | 1.24 |
| | V31L | | | | | | |
| | I33N | | | | | | |
| | V66Y | | | | | | |
| | D71E | | | | | | |
| | V84F | | | | | | |
| t877320 | D13R | 1.04 | 0.1 | 0.77 | 0.12 | 1.35 | 0.87 |
| | A18D | | | | | | |
| | T26R | | | | | | |
| | D39G | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t877667 | E17N | 1.02 | 0.04 | 1.14 | 0.03 | 0.89 | 1.34 |
| | V31L | | | | | | |
| | I33N | | | | | | |
| | D71E | | | | | | |
| | G82R | | | | | | |
| | V84F | | | | | | |
| t877625 | E17N | 0.82 | 0.06 | 0.83 | 0.08 | 0.99 | 0.72 |
| | A18D | | | | | | |
| | T26R | | | | | | |
| | V31L | | | | | | |
| | D71S | | | | | | |
| | D83E | | | | | | |
| | V84F | | | | | | |
| t877354 | E17N | 0.61 | 0.08 | 1.44 | 0.28 | 0.42 | 0.27 |
| | V31L | | | | | | |
| | I33N | | | | | | |
| | D71S | | | | | | |
| | D83K | | | | | | |
| | V84F | | | | | | |
| | T98Y | | | | | | |
| t877544 | V84K | 0.87 | 0.28 | 0.93 | 0.11 | 0.94 | 2.5 |
| | D39G | | | | | | |
| | D83E | | | | | | |
| | D13R | | | | | | |
| | E64D | | | | | | |
| | T98K | | | | | | |
| t877616 | T26R | 0.92 | 0.04 | 0.89 | 0.03 | 1.03 | 1.4 |
| | I33H | | | | | | |
| | Y55F | | | | | | |
| | Q70A | | | | | | |
| | G80A | | | | | | |
| | D83K | | | | | | |
| | V84K | | | | | | |
| | F88N | | | | | | |
| t877185 | D13R | 1.06 | 0.04 | 0.8 | 0.19 | 1.32 | 0.2 |
| | E17N | | | | | | |
| | V31L | | | | | | |
| | D71S | | | | | | |
| | D83A | | | | | | |
| | V84K | | | | | | |
| t877620 | A18D | 0.91 | 0.1 | 0.88 | 0.04 | 1.03 | 2.26 |
| | E22K | | | | | | |
| | T26R | | | | | | |
| | E52H | | | | | | |
| | Y55F | | | | | | |
| | Q70A | | | | | | |
| | D71E | | | | | | |
| | G80A | | | | | | |
| | D83N | | | | | | |
| | V84K | | | | | | |
| t877709 | E17N | 0.89 | 0.1 | 0.86 | 0.29 | 1.02 | 0.34 |
| | T26R | | | | | | |
| | V31L | | | | | | |
| | I33N | | | | | | |
| | E64D | | | | | | |
| | D71S | | | | | | |
| | V84F | | | | | | |
| t877073 | V84K | 0.9 | 0.08 | 0.83 | 0.18 | 1.09 | 0.47 |
| | T26R | | | | | | |
| | D39G | | | | | | |
| | D13R | | | | | | |
| | E64D | | | | | | |
| | T98K | | | | | | |
| t861745 | D39Q | 0.99 | 0.08 | 0.9 | 0.07 | 1.09 | 1.11 |
| | F95M | | | | | | |
| t876837 | T26R | 0.97 | 0.04 | 0.89 | 0.12 | 1.09 | 0.34 |
| | I33H | | | | | | |
| | Y55F | | | | | | |
| | Q70A | | | | | | |
| | G80A | | | | | | |
| | D83E | | | | | | |
| | T98K | | | | | | |
| t876806 | D13R | 0.89 | 0.04 | 1.01 | 0.05 | 0.88 | 0.82 |
| | A18D | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| t876826 | A18D | 0.89 | 0 | 0.82 | 0.08 | 1.09 | 0.06 |
| | T26R | | | | | | |
| | D39G | | | | | | |
| | E52H | | | | | | |
| | Y55F | | | | | | |
| | Q70A | | | | | | |
| | I74G | | | | | | |
| | G80A | | | | | | |
| | D83N | | | | | | |
| | V84K | | | | | | |
| t877087 | V84K | 0.81 | 0.03 | 0.74 | 0.04 | 1.09 | 0.86 |
| | D39G | | | | | | |
| | D83E | | | | | | |
| | E64D | | | | | | |
| | T98K | | | | | | |
| | S65N | | | | | | |
| t877290 | V84K | 0.64 | 0.05 | 1.55 | 0.06 | 0.41 | 0.73 |
| | T26R | | | | | | |
| | D83E | | | | | | |
| | D13R | | | | | | |
| | T98K | | | | | | |
| | S65N | | | | | | |
| t877100 | T26R | 0.91 | 0.07 | 0.97 | 0.08 | 0.94 | 0.92 |
| | I33N | | | | | | |
| | Y55F | | | | | | |
| | Q70A | | | | | | |
| | D71S | | | | | | |
| | G80A | | | | | | |
| | D83K | | | | | | |
| t861743 | I33R | 0.96 | 0.09 | 0.89 | 0.1 | 1.08 | 0.83 |
| | E52H | | | | | | |
| | Y55F | | | | | | |
| | Q70A | | | | | | |
| | I74G | | | | | | |
| | D83K | | | | | | |
| t877269 | E17N | 1.02 | 0.06 | 0.79 | 0.11 | 1.29 | 0.49 |
| | V31L | | | | | | |
| | D71S | | | | | | |
| | D83A | | | | | | |
| | V84K | | | | | | |
| | T98K | | | | | | |
| t876751 | A18D | 0.88 | 0.06 | 1.01 | 0.08 | 0.87 | 0.77 |
| | T26R | | | | | | |
| | I33R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t876801 | V84K | 0.84 | 0.06 | 0.9 | 0.08 | 0.93 | 0.75 |
| | D83E | | | | | | |
| | D13R | | | | | | |
| | E64D | | | | | | |
| | T98K | | | | | | |
| | S65N | | | | | | |
| t877098 | A18D | 0.79 | 0.03 | 0.86 | 0.09 | 0.93 | 0.35 |
| | E22K | | | | | | |
| | T26R | | | | | | |
| | I33R | | | | | | |
| | E52H | | | | | | |
| | Y55F | | | | | | |
| | Q70A | | | | | | |
| | G80A | | | | | | |
| | G82R | | | | | | |
| | D83N | | | | | | |
| | V84K | | | | | | |
| t782661 | I33N | 0.89 | 0.11 | 0.96 | 0.2 | 0.93 | 0.55 |
| | D71S | | | | | | |
| | G80A | | | | | | |
| t877282 | E17N | 0.84 | 0.14 | 0.83 | 0.33 | 1.01 | 0.44 |
| | A18D | | | | | | |
| | V31L | | | | | | |
| | I33N | | | | | | |
| | E64D | | | | | | |
| | D71S | | | | | | |
| | V84K | | | | | | |
| t877612 | A18D | 0.88 | 0.04 | 0.91 | 0.05 | 0.97 | 0.65 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| t876957 | V84K | 0.85 | 0.05 | 0.94 | 0.09 | 0.9 | 0.64 |
| | T26R | | | | | | |
| | D13R | | | | | | |
| | E64D | | | | | | |
| | T98K | | | | | | |
| | S65N | | | | | | |
| t782504 | | 0.89 | 0.13 | 1 | 0.23 | 0.9 | 0.59 |
| t876956 | T26R | 0.95 | 0.03 | 0.93 | 0.08 | 1.02 | 0.35 |
| | I33H | | | | | | |
| | Y55F | | | | | | |
| | Q70A | | | | | | |
| | G80A | | | | | | |
| | D83E | | | | | | |
| | F88N | | | | | | |
| t877090 | D13R | 0.87 | 0.05 | 0.98 | 0.21 | 0.89 | 0.26 |
| | A18D | | | | | | |
| | N29S | | | | | | |
| | Q70L | | | | | | |
| | I74G | | | | | | |
| | G80A | | | | | | |
| | D83K | | | | | | |
| | Y85F | | | | | | |
| t876810 | E17N | 0.83 | 0.02 | 1.01 | 0.02 | 0.82 | 1.13 |
| | A18D | | | | | | |
| | V31L | | | | | | |
| | E64D | | | | | | |
| | D71S | | | | | | |
| | D83A | | | | | | |
| | V84F | | | | | | |
| | T98K | | | | | | |
| t877138 | D13R | 1.06 | 0.05 | 0.88 | 0.03 | 1.2 | 1.58 |
| | E17N | | | | | | |
| | V31L | | | | | | |
| | I33N | | | | | | |
| | D71S | | | | | | |
| | V84K | | | | | | |
| t876773 | A18D | 0.87 | 0.02 | 1.06 | 0.03 | 0.82 | 0.77 |
| | T26R | | | | | | |
| | I33R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t876968 | D13R | 0.95 | 0.04 | 1.16 | 0.05 | 0.82 | 0.92 |
| | A18D | | | | | | |
| | T26R | | | | | | |
| | N29S | | | | | | |
| | Q70L | | | | | | |
| | D71Q | | | | | | |
| | G80A | | | | | | |
| | D83E | | | | | | |
| t877255 | A18D | 0.47 | 0.54 | 0.39 | 0.45 | 1.21 | 1.21 |
| | T26R | | | | | | |
| | I33R | | | | | | |
| | D39G | | | | | | |
| | E52H | | | | | | |
| | Y55F | | | | | | |
| | Q70A | | | | | | |
| | G80A | | | | | | |
| | D83E | | | | | | |
| | V84K | | | | | | |
| t876755 | A18D | 0.87 | 0.03 | 1.06 | 0.04 | 0.82 | 0.74 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t876943 | E17N | 0.9 | 0.04 | 0.89 | 0.08 | 1.01 | 0.58 |
| | V31L | | | | | | |
| | D71S | | | | | | |
| | I74G | | | | | | |
| | D83K | | | | | | |
| | V84F | | | | | | |
| t877367 | E17N | 0.99 | 0.03 | 0.82 | 0.17 | 1.2 | 0.2 |
| | A18D | | | | | | |
| | V31L | | | | | | |
| | I33N | | | | | | |
| | D71S | | | | | | |
| | V84K | | | | | | |
| | T98Y | | | | | | |
| t877437 | T26R | 1.08 | 0.02 | 0.91 | 0.03 | 1.19 | 0.6 |
| | D39G | | | | | | |
| | D83E | | | | | | |
| | D13R | | | | | | |
| | E64D | | | | | | |
| | T98K | | | | | | |
| t876988 | D13R | 1.05 | 0.03 | 1.04 | 0.11 | 1.01 | 0.26 |
| | A18D | | | | | | |
| | N29S | | | | | | |
| | D39G | | | | | | |
| | Q70L | | | | | | |
| | D71Q | | | | | | |
| | G80A | | | | | | |
| | D83K | | | | | | |
| | V84K | | | | | | |
| t877071 | E17N | 0.94 | 0.11 | 0.94 | 0.15 | 1.01 | 0.76 |
| | A18D | | | | | | |
| | V31L | | | | | | |
| | E64D | | | | | | |
| | D71S | | | | | | |
| | I74G | | | | | | |
| | D83A | | | | | | |
| | V84F | | | | | | |
| t876753 | T26R | 0.89 | 0.04 | 1.1 | 0.04 | 0.81 | 0.98 |
| | I33H | | | | | | |
| | Y55F | | | | | | |
| | E64D | | | | | | |
| | Q70A | | | | | | |
| | G80A | | | | | | |
| | D83K | | | | | | |
| | V84K | | | | | | |
| t876878 | A18D | 0.87 | 0.04 | 1.07 | 0.02 | 0.81 | 1.71 |
| | T26R | | | | | | |
| | I33R | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| | T98Y | | | | | | |
| t877610 | D13R | 0.93 | 0.04 | 1 | 0.05 | 0.93 | 0.72 |
| | A18D | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t877467 | D13R | 1 | 0.04 | 0.85 | 0.05 | 1.17 | 0.76 |
| | E17N | | | | | | |
| | A18D | | | | | | |
| | V31L | | | | | | |
| | D71S | | | | | | |
| | D83E | | | | | | |
| | V84F | | | | | | |
| t877639 | D13R | 1.01 | 0.06 | 1.28 | 0.07 | 0.79 | 0.84 |
| | A18D | | | | | | |
| | N29S | | | | | | |
| | S65N | | | | | | |
| | Q70L | | | | | | |
| | D71Q | | | | | | |
| | I74G | | | | | | |
| | G80A | | | | | | |
| | D83K | | | | | | |
| t876936 | D71E | 0.84 | 0.06 | 1.05 | 0.38 | 0.8 | 0.14 |
| | V66Y | | | | | | |
| | E22K | | | | | | |
| | D83K | | | | | | |
| | T98K | | | | | | |
| | D13R | | | | | | |
| t876845 | V84K | 0.83 | 0.07 | 0.84 | 0.09 | 0.99 | 0.77 |
| | T26R | | | | | | |
| | D39G | | | | | | |
| | D83E | | | | | | |
| | D13R | | | | | | |
| | E64D | | | | | | |
| t877608 | E17N | 0.84 | 0.02 | 0.91 | 0.04 | 0.92 | 0.55 |
| | V31L | | | | | | |
| | V66Y | | | | | | |
| | D71S | | | | | | |
| | D83A | | | | | | |
| | V84F | | | | | | |
| | Y85F | | | | | | |
| t876882 | A18D | 0.85 | 0.03 | 1.07 | 0.09 | 0.79 | 0.35 |
| | T26R | | | | | | |
| | E52H | | | | | | |
| | Y55F | | | | | | |
| | Q70A | | | | | | |
| | D71E | | | | | | |
| | G80A | | | | | | |
| | D83N | | | | | | |
| | V84K | | | | | | |
| t877578 | D13R | 0.91 | 0.12 | 1.07 | 0.06 | 0.85 | 1.92 |
| | A18D | | | | | | |
| | N29S | | | | | | |
| | Q70L | | | | | | |
| | D71Q | | | | | | |
| | G80A | | | | | | |
| | D83K | | | | | | |
| t877577 | D13R | 0.84 | 0.1 | 0.98 | 0.06 | 0.85 | 1.75 |
| | A18D | | | | | | |
| | N29S | | | | | | |
| | Q70L | | | | | | |
| | D71Q | | | | | | |
| | G80A | | | | | | |
| | D83K | | | | | | |
| t876784 | D13R | 0.93 | 0.03 | 1.18 | 0.07 | 0.78 | 0.42 |
| | A18D | | | | | | |
| | E22K | | | | | | |
| | N29S | | | | | | |
| | Q70L | | | | | | |
| | D71E | | | | | | |
| | G80A | | | | | | |
| | D83K | | | | | | |
| t877744 | D13R | 0.89 | 0.03 | 1.05 | 0.03 | 0.85 | 0.95 |
| | A18D | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | T98Y | | | | | | |
| t876980 | D13R | 0.91 | 0.03 | 0.94 | 0.02 | 0.97 | 1.69 |
| | A18D | | | | | | |
| | T26R | | | | | | |
| | I33R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | Y85F | | | | | | |
| t877741 | E17N | 0.89 | 0.03 | 1.05 | 0.1 | 0.85 | 0.31 |
| | V31L | | | | | | |
| | S65N | | | | | | |
| | D71S | | | | | | |
| | D83A | | | | | | |
| | V84K | | | | | | |
| t876914 | E17N | 0.84 | 0.05 | 1.09 | 0.09 | 0.77 | 0.58 |
| | A18D | | | | | | |
| | V31L | | | | | | |
| | V66Y | | | | | | |
| | D71E | | | | | | |
| | D83A | | | | | | |
| | V84F | | | | | | |
| t876778 | D13R | 0.89 | 0.05 | 1.15 | 0.08 | 0.77 | 0.62 |
| | A18D | | | | | | |
| | N29S | | | | | | |
| | E64D | | | | | | |
| | Q70L | | | | | | |
| | D71Q | | | | | | |
| | G80A | | | | | | |
| | D83K | | | | | | |
| | V84K | | | | | | |
| t876917 | D13R | 0.95 | 0.13 | 1.14 | 0.06 | 0.83 | 2.04 |
| | A18D | | | | | | |
| | T26R | | | | | | |
| | N29S | | | | | | |
| | Q70L | | | | | | |
| | D71Q | | | | | | |
| | G80A | | | | | | |
| | D83K | | | | | | |
| t877507 | D13R | 0.93 | 0.08 | 1.11 | 0.09 | 0.84 | 0.89 |
| | E17N | | | | | | |
| | A18D | | | | | | |
| | V31L | | | | | | |
| | D71S | | | | | | |
| | D83K | | | | | | |
| | V84F | | | | | | |
| t877039 | E17N | 0.78 | 0.07 | 0.82 | 0.05 | 0.95 | 1.52 |
| | A18D | | | | | | |
| | V31L | | | | | | |
| | E64D | | | | | | |
| | D71S | | | | | | |
| | D83A | | | | | | |
| | V84K | | | | | | |
| t876904 | D13R | 0.9 | 0.08 | 1.17 | 0.08 | 0.77 | 1.04 |
| | A18D | | | | | | |
| | N29S | | | | | | |
| | Q70L | | | | | | |
| | D71E | | | | | | |
| | G80A | | | | | | |
| | D83K | | | | | | |
| t877615 | E17N | 1.06 | 0.08 | 1.4 | 0.54 | 0.75 | 0.14 |
| | T26R | | | | | | |
| | V31L | | | | | | |
| | D71S | | | | | | |
| | D83K | | | | | | |
| | V84F | | | | | | |
| t877061 | E17N | 0.96 | 0.06 | 1.01 | 0.1 | 0.95 | 0.6 |
| | A18D | | | | | | |
| | V31L | | | | | | |
| | D71S | | | | | | |
| | D83E | | | | | | |
| | V84F | | | | | | |
| | T98K | | | | | | |
| t876840 | D13R | 0.95 | 0.04 | 1.26 | 0.08 | 0.76 | 0.49 |
| | A18D | | | | | | |
| | N29S | | | | | | |
| | Q70L | | | | | | |
| | D71Q | | | | | | |
| | G80A | | | | | | |
| | D83K | | | | | | |
| | V84K | | | | | | |
| | Y85F | | | | | | |
| t877471 | E17N | 0.85 | 0.57 | 0.77 | 0.52 | 1.1 | 1.1 |
| | V31L | | | | | | |
| | D71S | | | | | | |
| | D83E | | | | | | |
| | V84F | | | | | | |
| | T98K | | | | | | |
| t876815 | V84K | 0.82 | 0.07 | 0.88 | 0.08 | 0.94 | 0.9 |
| | D39G | | | | | | |
| | D83E | | | | | | |
| | D13R | | | | | | |
| | E64D | | | | | | |
| | S65N | | | | | | |
| t877080 | E17N | 0.57 | 0.66 | 0.63 | 0.73 | 0.9 | 0.91 |
| | A18D | | | | | | |
| | V31L | | | | | | |
| | E64D | | | | | | |
| | D71S | | | | | | |
| | D83K | | | | | | |
| | V84F | | | | | | |
| t877383 | D13R | 0.62 | 0.03 | 1.76 | 0.13 | 0.35 | 0.24 |
| | A18D | | | | | | |
| | T26R | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| t876802 | A18D | 0.88 | 0.12 | 0.94 | 0.08 | 0.93 | 1.39 |
| | T26R | | | | | | |
| | E52H | | | | | | |
| | Y55F | | | | | | |
| | Q70A | | | | | | |
| | G80A | | | | | | |
| | D83E | | | | | | |
| | V84K | | | | | | |
| | T98Y | | | | | | |
| t877150 | A18D | 1.16 | 0.03 | 1.3 | 0.05 | 0.89 | 0.64 |
| | T26R | | | | | | |
| | I33R | | | | | | |
| | D39G | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| t877662 | A18D | 0.88 | 0.03 | 1.03 | 0.03 | 0.86 | 0.98 |
| | T26R | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| | T98K | | | | | | |
| t877593 | D13R | 1.06 | 0.05 | 1.45 | 0.06 | 0.73 | 0.82 |
| | A18D | | | | | | |
| | N29S | | | | | | |
| | Q70L | | | | | | |
| | D71Q | | | | | | |
| | G80A | | | | | | |
| | D83E | | | | | | |
| t877319 | E17N | 1.06 | 0.29 | 0.99 | 0.35 | 1.08 | 0.84 |
| | A18D | | | | | | |
| | V31L | | | | | | |
| | D71S | | | | | | |
| | D83A | | | | | | |
| | V84F | | | | | | |
| t877010 | D13R | 0.93 | 0.03 | 1.02 | 0.09 | 0.91 | 0.3 |
| | A18D | | | | | | |
| | N29S | | | | | | |
| | E64D | | | | | | |
| | S65N | | | | | | |
| | Q70L | | | | | | |
| | D71Q | | | | | | |
| | G80A | | | | | | |
| | D83K | | | | | | |
| t876911 | E17N | 0.81 | 0.05 | 0.89 | 0.07 | 0.91 | 0.67 |
| | A18D | | | | | | |
| | V31L | | | | | | |
| | D71S | | | | | | |
| | D83K | | | | | | |
| | V84K | | | | | | |
| t876790 | E17N | 0.79 | 0.03 | 0.87 | 0.08 | 0.91 | 0.35 |
| | V31L | | | | | | |
| | I33N | | | | | | |
| | V66Y | | | | | | |
| | D71S | | | | | | |
| | G82R | | | | | | |
| | V84F | | | | | | |
| t877352 | A18D | 0.6 | 0.03 | 1.74 | 0.2 | 0.34 | 0.17 |
| | T26R | | | | | | |
| | I33R | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| | T98K | | | | | | |
| t876781 | A18D | 0.88 | 0.05 | 0.99 | 0.06 | 0.89 | 0.84 |
| | E22K | | | | | | |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| t877581 | T26R | 0.82 | 0.01 | 1.05 | 0.06 | 0.79 | 0.21 |
| | I33H | | | | | | |
| | Y55F | | | | | | |
| | E64D | | | | | | |
| | Q70A | | | | | | |
| | G80A | | | | | | |
| | D83K | | | | | | |
| | T98K | | | | | | |
| t877387 | A18D | 0.6 | 0.03 | 1.77 | 0.22 | 0.34 | 0.14 |
| | T26R | | | | | | |
| | I33R | | | | | | |
| | E52H | | | | | | |
| | Y55F | | | | | | |
| | Q70A | | | | | | |
| | G80A | | | | | | |
| | D83E | | | | | | |
| | V84K | | | | | | |
| | T98Y | | | | | | |
| t877062 | D13R | 0.99 | 0.09 | 1.11 | 0.13 | 0.89 | 0.73 |
| | A18D | | | | | | |
| | T26R | | | | | | |
| | N29S | | | | | | |
| | Q70L | | | | | | |
| | D71Q | | | | | | |
| | G80A | | | | | | |
| | D83K | | | | | | |
| | T98K | | | | | | |
| t877397 | D13R | 0.63 | 0.11 | 1.9 | 0.15 | 0.33 | 0.76 |
| | A18D | | | | | | |
| | N29S | | | | | | |
| | Q70L | | | | | | |
| | D71Q | | | | | | |
| | G80A | | | | | | |
| | D83K | | | | | | |
| | V84K | | | | | | |
| t877031 | D13R | 0.97 | 0.04 | 1.11 | 0.1 | 0.88 | 0.44 |
| | A18D | | | | | | |
| | T26R | | | | | | |
| | N29S | | | | | | |
| | E64D | | | | | | |
| | Q70L | | | | | | |
| | D71Q | | | | | | |
| | G80A | | | | | | |
| | D83K | | | | | | |
| t877343 | T26R | 1.16 | 0.03 | 1.13 | 0.09 | 1.03 | 0.27 |
| | D83E | | | | | | |
| | D13R | | | | | | |
| | E64D | | | | | | |
| | T98K | | | | | | |
| | S65N | | | | | | |
| t877356 | A18D | 0.57 | 0.05 | 1.74 | 0.13 | 0.33 | 0.38 |
| | T26R | | | | | | |
| | I33H | | | | | | |
| | G80A | | | | | | |
| t877374 | D13R | 1 | 0.07 | 0.96 | 0.19 | 1.04 | 0.38 |
| | A18D | | | | | | |
| | Y55F | | | | | | |
| | N29S | | | | | | |
| | Q70L | | | | | | |
| | G80A | | | | | | |
| | D83E | | | | | | |
| | T98K | | | | | | |
| | Q70A | | | | | | |
| t877063 | E17N | 0.82 | 0.09 | 0.95 | 0.05 | 0.87 | 1.65 |
| | A18D | | | | | | |
| | T26R | | | | | | |
| | V31L | | | | | | |
| | D71S | | | | | | |
| | D83K | | | | | | |
| | V84F | | | | | | |
| t877414 | E17N | 0.93 | 0.05 | 0.9 | 0.1 | 1.04 | 0.52 |
| | V31L | | | | | | |
| | D71S | | | | | | |
| | D83K | | | | | | |
| | V84F | | | | | | |
| | T98K | | | | | | |
| | D83K | | | | | | |
| t876776 | E17N | 0.84 | 0.03 | 0.97 | 0.08 | 0.87 | 0.32 |
| | A18D | | | | | | |
| | V31L | | | | | | |
| | I33N | | | | | | |
| | D71S | | | | | | |
| | V84F | | | | | | |
| | T98K | | | | | | |
| t877180 | E17N | 0.91 | 0.06 | 1.09 | 0.04 | 0.83 | 1.61 |
| | V31L | | | | | | |
| | I33N | | | | | | |
| | E64D | | | | | | |
| | D71S | | | | | | |
| | V84K | | | | | | |
| t877503 | A18D | 0.88 | 0.01 | 1.17 | 0.06 | 0.75 | 0.24 |
| | T26R | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| | T98Y | | | | | | |
| t877566 | A18D | 1.02 | 0.07 | 1.5 | 0.05 | 0.68 | 1.33 |
| | T26R | | | | | | |
| | E52H | | | | | | |
| | Y55F | | | | | | |
| | Q70A | | | | | | |
| | G80A | | | | | | |
| | D83K | | | | | | |
| | V84K | | | | | | |
| | Y85F | | | | | | |
| t877349 | E17N | 1.08 | 0.06 | 1.07 | 0.01 | 1 | 5.11 |
| | A18D | | | | | | |
| | V31L | | | | | | |
| | D71S | | | | | | |
| | D83A | | | | | | |
| | V84K | | | | | | |
| t877246 | T26R | 0.92 | 0.1 | 1.12 | 0.1 | 0.83 | 0.93 |
| | I33H | | | | | | |
| | Y55F | | | | | | |
| | Q70A | | | | | | |
| | G80A | | | | | | |
| | D83E | | | | | | |
| | V84K | | | | | | |
| | Y85A | | | | | | |
| t877626 | A18D | 0.83 | 0.05 | 1.11 | 0. 1 | 0.75 | 0.5 |
| | T26R | | | | | | |
| | I33R | | | | | | |
| | D39G | | | | | | |
| | E52H | | | | | | |
| | Y55F | | | | | | |
| | Q70A | | | | | | |
| | G80A | | | | | | |
| | D83K | | | | | | |
| | V84K | | | | | | |
| t877614 | D13R | 0.92 | 0.09 | 1.17 | 0.09 | 0.78 | 0.95 |
| | A18D | | | | | | |
| | N29S | | | | | | |
| | S65N | | | | | | |
| | Q70L | | | | | | |
| | D71Q | | | | | | |
| | G80A | | | | | | |
| | D83K | | | | | | |
| | V84K | | | | | | |
| t877511 | D13R | 0.86 | 0.03 | 1.1 | 0.12 | 0.78 | 0.23 |
| | A18D | | | | | | |
| | N29S | | | | | | |
| | Q70L | | | | | | |
| | G80A | | | | | | |
| | D83K | | | | | | |
| t877705 | D13R | 0.91 | 0.02 | 1.18 | 0.07 | 0.78 | 0.23 |
| | A18D | | | | | | |
| | T26R | | | | | | |
| | E52H | | | | | | |
| | Y55F | | | | | | |
| | S65N | | | | | | |
| | Q70A | | | | | | |
| | G80A | | | | | | |
| | D83N | | | | | | |
| | V84K | | | | | | |
| t877681 | D13R | 1.11 | 0.06 | 1.68 | 0.1 | 0.66 | 0.64 |
| | A18D | | | | | | |
| | N29S | | | | | | |
| | Q70L | | | | | | |
| | D71Q | | | | | | |
| | G80A | | | | | | |
| | D83K | | | | | | |
| | T98Y | | | | | | |
| t876883 | T26R | 0.79 | 0.12 | 0.95 | 0.12 | 0.82 | 1 |
| | I33H | | | | | | |
| | Y55F | | | | | | |
| | Q70A | | | | | | |
| | I74G | | | | | | |
| | G80A | | | | | | |
| | D83K | | | | | | |
| | V84K | | | | | | |
| | Y85A | | | | | | |
| t876821 | T26R | 0.81 | 0.08 | 1.14 | 0.08 | 0.71 | 1.01 |
| | I33H | | | | | | |
| | Y55F | | | | | | |
| | Q70A | | | | | | |
| | G80A | | | | | | |
| | D83K | | | | | | |
| | V84K | | | | | | |
| | Y85A | | | | | | |
| | T98Y | | | | | | |
| t877125 | D13R | 0.95 | 0.37 | 0.99 | 0.31 | 0.96 | 1.19 |
| | A18D | | | | | | |
| | N29S | | | | | | |
| | Q70L | | | | | | |
| | D71Q | | | | | | |
| | G80A | | | | | | |
| | D83E | | | | | | |
| | T98Y | | | | | | |
| t876760 | D13R | 1.05 | 0.1 | 1.53 | 0.07 | 0.69 | 1.4 |
| | A18D | | | | | | |
| | N29S | | | | | | |
| | S65N | | | | | | |
| | Q70L | | | | | | |
| | D71Q | | | | | | |
| | G80A | | | | | | |
| | D83K | | | | | | |
| | T98K | | | | | | |
| t877110 | E17N | 0.94 | 0.09 | 1.26 | 0.11 | 0.75 | 0.76 |
| | V31L | | | | | | |
| | D71S | | | | | | |
| | D83K | | | | | | |
| | V84F | | | | | | |
| | T98Y | | | | | | |
| t877325 | A18D | 0.59 | 0.05 | 2.03 | 0.1 | 0.29 | 0.48 |
| | T26R | | | | | | |
| | E52H | | | | | | |
| | Y55F | | | | | | |
| | V61S | | | | | | |
| | Q70A | | | | | | |
| | G80A | | | | | | |
| | D83N | | | | | | |
| | V84K | | | | | | |
| | Y85F | | | | | | |
| t877026 | T26R | 0.77 | 0.03 | 1 | 0.06 | 0.77 | 0.44 |
| | I33H | | | | | | |
| | Y55F | | | | | | |
| | S65N | | | | | | |
| | Q70A | | | | | | |
| | I74G | | | | | | |
| | G80A | | | | | | |
| | D83K | | | | | | |
| | Y85A | | | | | | |
| t877654 | D13R | 0.86 | 0.04 | 1.28 | 0.04 | 0.67 | 0.89 |
| | A18D | | | | | | |
| | N29S | | | | | | |
| | Q70L | | | | | | |
| | D71Q | | | | | | |
| | G80A | | | | | | |
| | D83K | | | | | | |
| | V84K | | | | | | |
| | T98Y | | | | | | |
| t877683 | T26R | 0.91 | 0.05 | 1.5 | 0.16 | 0.6 | 0.31 |
| | I33H | | | | | | |
| | Y55F | | | | | | |
| | E64D | | | | | | |
| | Q70A | | | | | | |
| | G80A | | | | | | |
| | D83E | | | | | | |
| | Y85A | | | | | | |
| t877152 | E17N | 0.84 | 0.08 | 1.14 | 0.12 | 0.74 | 0.64 |
| | V31L | | | | | | |
| | I33N | | | | | | |
| | E64D | | | | | | |
| | D71S | | | | | | |
| | V84F | | | | | | |
| | T98Y | | | | | | |
| t877013 | T26R | 0.88 | 0.05 | 1.35 | 0.13 | 0.65 | 0.41 |
| | I33H | | | | | | |
| | Y55F | | | | | | |
| | E64D | | | | | | |
| | Q70A | | | | | | |
| | G80A | | | | | | |
| | D83K | | | | | | |
| | Y85A | | | | | | |
| t877459 | A18D | 0.93 | 0.33 | 1.05 | 0.3 | 0.89 | 1.11 |
| | T26R | | | | | | |
| | I33N | | | | | | |
| | E52H | | | | | | |
| | Y55F | | | | | | |
| | Q70A | | | | | | |
| | D71S | | | | | | |
| | G80A | | | | | | |
| | D83N | | | | | | |
| | V84K | | | | | | |
| t877360 | A18D | 0.61 | 0.06 | 2.18 | 0.16 | 0.28 | 0.36 |
| | T26R | | | | | | |
| | I33H | | | | | | |
| | Y55F | | | | | | |
| | E64D | | | | | | |
| | Q70A | | | | | | |
| | G80A | | | | | | |
| | D83K | | | | | | |
| | Y85A | | | | | | |
| t877323 | T26R | 0.58 | 0.03 | 2.1 | 0.05 | 0.28 | 0.58 |
| | I33H | | | | | | |
| | Y55F | | | | | | |
| | E64D | | | | | | |
| | S65N | | | | | | |
| | Q70A | | | | | | |
| | G80A | | | | | | |
| | D83K | | | | | | |
| t877700 | D13R | 0.87 | 0.02 | 1.36 | 0.06 | 0.64 | 0.27 |
| | A18D | | | | | | |
| | N29S | | | | | | |
| | V66Y | | | | | | |
| | Q70L | | | | | | |
| | G80A | | | | | | |
| | D83K | | | | | | |
| | Y85F | | | | | | |
| t877114 | E17N | 0.93 | 0.12 | 1.32 | 0.17 | 0.7 | 0.71 |
| | A18D | | | | | | |
| | V31L | | | | | | |
| | V66Y | | | | | | |
| | D71S | | | | | | |
| | D83A | | | | | | |
| | V84F | | | | | | |
| | Y85F | | | | | | |
| t877743 | D13R | 0.86 | 0.05 | 1.35 | 0.08 | 0.64 | 0.65 |
| | A18D | | | | | | |
| | E22K | | | | | | |
| | N29S | | | | | | |
| | V66Y | | | | | | |
| | Q70L | | | | | | |
| | G80A | | | | | | |
| | D83K | | | | | | |
| t876903 | D71E | 0.76 | 0.05 | 1.06 | 0.06 | 0.72 | 0.84 |
| | V66Y | | | | | | |
| | V61S | | | | | | |
| | E22K | | | | | | |
| | D83K | | | | | | |
| | T98K | | | | | | |
| t877509 | T26R | 0.93 | 0.09 | 1.4 | 0.1 | 0.66 | 0.93 |
| | I33H | | | | | | |
| | Y55F | | | | | | |
| | Q70A | | | | | | |
| | G80A | | | | | | |
| | D83K | | | | | | |
| | Y85A | | | | | | |
| t877072 | E17N | 0.91 | 0.29 | 1.33 | 0.52 | 0.68 | 0.56 |
| | A18D | | | | | | |
| | V31L | | | | | | |
| | D71S | | | | | | |
| | D83K | | | | | | |
| | V84F | | | | | | |
| | T98Y | | | | | | |
| t877078 | A18D | 0.97 | 0.07 | 1.42 | 0.06 | 0.68 | 1.13 |
| | T26R | | | | | | |
| | D.39G | | | | | | |
| | E52H | | | | | | |
| | Y55F | | | | | | |
| | E64D | | | | | | |
| | Q70A | | | | | | |
| | G80A | | | | | | |
| | D83N | | | | | | |
| | V84K | | | | | | |
| t877167 | E17N | 0.73 | 0.49 | 0.87 | 0.03 | 0.85 | 14.1 |
| | V31L | | | | | | |
| | D71S | | | | | | |
| | D83A | | | | | | |
| | V84K | | | | | | |
| | Y85F | | | | | | |
| t877275 | D13R | 0.99 | 0.05 | 1.17 | 0.7 | 0.84 | 0.08 |
| | A18D | | | | | | |
| | N29S | | | | | | |
| | S65N | | | | | | |
| | Q70L | | | | | | |
| | I74G | | | | | | |
| | G80A | | | | | | |
| | D83K | | | | | | |
| t877055 | A18D | 0.96 | 0.3 | 1.42 | 0.62 | 0.68 | 0.48 |
| | T26R | | | | | | |
| | E52H | | | | | | |
| | Y55F | | | | | | |
| | Q70A | | | | | | |
| | G80A | | | | | | |
| | D83N | | | | | | |
| | V84K | | | | | | |
| | F88N | | | | | | |
| | T98K | | | | | | |
| t877316 | T26R | 0.99 | 0.4 | 1.37 | 0.44 | 0.72 | 0.92 |
| | I33H | | | | | | |
| | Y55F | | | | | | |
| | Q70A | | | | | | |
| | G80A | | | | | | |
| | D83K | | | | | | |
| | Y85A | | | | | | |
| t876939 | D13R | 0.83 | 0.08 | 1.31 | 0.14 | 0.63 | 0.59 |
| | A18D | | | | | | |
| | N29S | | | | | | |
| | Q70L | | | | | | |
| | G80A | | | | | | |
| | D83K | | | | | | |
| | V84K | | | | | | |
| | T98K | | | | | | |
| t877447 | T26R | 0.58 | 0.03 | 2.42 | 0.18 | 0.24 | 0.16 |
| | I33H | | | | | | |
| | Y55F | | | | | | |
| | S65N | | | | | | |
| | Q70A | | | | | | |
| | G80A | | | | | | |
| | D83E | | | | | | |
| | Y85A | | | | | | |
| t877364 | A18D | 0.58 | 0.03 | 2.47 | 0.2 | 0.23 | 0.13 |
| | T26R | | | | | | |
| | I33H | | | | | | |
| | Y55F | | | | | | |
| | S65N | | | | | | |
| | Q70A | | | | | | |
| | G80A | | | | | | |
| | D83K | | | | | | |
| | Y85A | | | | | | |
| t877321 | D13R | 0.58 | 0.03 | 2.76 | 0.3 | 0.21 | 0.09 |
| | A18D | | | | | | |
| | N29S | | | | | | |
| | D39G | | | | | | |
| | Q70L | | | | | | |
| | G80A | | | | | | |
| | D83K | | | | | | |
| | Y85F | | | | | | |
| t877292 | D13R | 0.62 | 0.03 | 2.95 | 0.38 | 0.21 | 0.09 |
| | A18D | | | | | | |
| | N29S | | | | | | |
| | D39G | | | | | | |
| | S65N | | | | | | |
| | Q70L | | | | | | |
| | D71Q | | | | | | |
| | G80A | | | | | | |
| | D83K | | | | | | |
| t877294 | T26R | 0.83 | 0.47 | 1.43 | 0.6 | 0.58 | 0.78 |
| | I33H | | | | | | |
| | Y55F | | | | | | |
| | Q70A | | | | | | |
| | G80A | | | | | | |
| | D83K | | | | | | |
| t877230 | E17N | 0.69 | 0.36 | 1.39 | 0.63 | 0.5 | 0.56 |
| | E22K | | | | | | |
| | V31L | | | | | | |
| | D71E | | | | | | |
| | D83A | | | | | | |
| | V84F | | | | | | |
| t782404 | | 0.54 | 0.3 | 1.83 | 0.71 | 0.3 | 0.42 |
| t877481 | E17N | 0.01 | 0.01 | 0.57 | 1.15 | 0.01 | 0.01 |
| | V31L | | | | | | |
| | E64D | | | | | | |
| | D71S | | | | | | |
| | D83A | | | | | | |
| | V84K | | | | | | |
| t877455 | D71E | 0.01 | 0.01 | 0.55 | 1 1 | 0.01 | 0.01 |
| | V66Y | | | | | | |
| | E22K | | | | | | |
| | G82R | | | | | | |
| | T98K | | | | | | |
| | D13R | | | | | | |
| t877023 | D13R | 0 | 0 | 0.74 | 1.47 | 0 | 0 |
| | E17N | | | | | | |
| | A18D | | | | | | |
| | V31L | | | | | | |
| | I33N | | | | | | |
| | D71S | | | | | | |
| | V84K | | | | | | |
| t877057 | E17N | 0 | 0 | 0.68 | 1.36 | 0 | 0 |
| | V31L | | | | | | |
| | I33N | | | | | | |
| | D71S | | | | | | |
| | D83E | | | | | | |
| | V84K | | | | | | |

TABLE 10*
Non-limiting examples of OAC amino acid
substitutions relative to SEQ ID NO: 1

| Position | | | | | | | | |
|---|---|---|---|---|---|---|---|---|
| in SEQ | | | | | | | | |
| ID NO: | | | | | | | | |
| 1 | Amino acid substitutions | | | | | | | |
| 2 | A2G | | | | | | | |
| 3 | V3M | V3Y | | | | | | |
| 13 | D13R | D13K | | | | | | |
| 17 | E17N | | | | | | | |
| 18 | A18D | A18R | | | | | | |
| 19 | Q19E | Q19D | | | | | | |
| 20 | K20A | | | | | | | |
| 21 | E21K | | | | | | | |
| 22 | E22T | E22K | | | | | | |
| 24 | F24M | | | | | | | |
| 25 | K25A | K25N | | | | | | |
| 26 | T26H | T26R | T26A | T26E | | | | |
| 28 | V28A | V28S | V28E | V28H | | | | |
| 29 | N29D | N29S | N29E | N29T | | | | |
| 31 | V31L | V31A | | | | | | |
| 33 | I33H | I33R | I33D | I33E | I33K | I33Q | I33N | |
| 37 | M37L | | | | | | | |
| 38 | K38L | | | | | | | |
| 39 | D39G | D39S | D39Q | | | | | |
| 41 | Y41K | Y41R | | | | | | |
| 45 | D45I | | | | | | | |
| 46 | V46F | | | | | | | |
| 48 | Q48A | | | | | | | |
| 49 | K49Q | | | | | | | |
| 50 | N50K | N50F | N50W | | | | | |
| 52 | E52H | | | | | | | |
| 53 | E53H | | | | | | | |
| 55 | Y55F | | | | | | | |
| 56 | T56Y | | | | | | | |
| 57 | H57Y | | | | | | | |
| 59 | V59I | V59F | | | | | | |
| 60 | E601 | E60L | | | | | | |
| 61 | V61T | V61D | V61S | | | | | |
| 62 | T62M | | | | | | | |
| 64 | E64Q | E64D | | | | | | |
| 65 | S65N | | | | | | | |
| 66 | V66L | V66M | V66Y | | | | | |
| 67 | E67K | | | | | | | |
| 68 | T68E | T68G | T68H | | | | | |
| 69 | I69L | I69Y | | | | | | |
| 70 | Q70A | Q70L | | | | | | |
| 71 | D71Q | D71S | D71E | | | | | |
| 73 | I73L | | | | | | | |
| 74 | I74D | I74E | I74H | I74Q | I74S | I74G | I74L | |
| 80 | G80V | G80A | G80E | G80S | G80D | G80N | | |
| 81 | F81Y | | | | | | | |
| 82 | G82K | G82V | G82R | | | | | |
| 83 | D83A | D83E | D83G | D83K | D83N | D83Q | D83R | D83S |
| 84 | V84F | V84K | V84M | V84Q | | | | |
| 85 | Y85A | Y85F | | | | | | |
| 86 | R86N | R86G | R86S | | | | | |
| 87 | S87C | S87D | S87K | | | | | |
| 88 | F88Y | F88N | F88S | | | | | |
| 89 | W89V | W89M | | | | | | |
| 94 | I94V | | | | | | | |
| 95 | F95M | F95I | | | | | | |
| 97 | Y97F | | | | | | | |
| 98 | T98E | T98K | T98Y | | | | | |
| 100 | R100A | R100G | R100K | R100N | R100Q | R100S | | |
| 101 | K101A | K101E | | | | | | |

*Non-conservative amino acid substitutions are indicated in bold font and are underlined.

TABLE 11
Amino acid and nucleic acid sequences of OACs

| | Amino Acid | Nucleic acid |
|---|---|---|
| Strain | SEQ ID NO | SEQ ID NO |
| t618874 | 19 | 107 |
| t618875 | 20 | 108 |
| t618876 | 21 | 109 |
| t618877 | 22 | 110 |
| t618878 | 23 | 111 |
| t618879 | 24 | 112 |
| t618880 | 25 | 113 |
| t618881 | 26 | 114 |
| t618882 | 27 | 115 |
| t618883 | 28 | 116 |
| t618884 | 2 | 117 |
| t618885 | 30 | 118 |
| t618886 | 31 | 119 |
| t618887 | 32 | 120 |
| t618888 | 33 | 121 |
| t618889 | 34 | 122 |
| t618890 | 35 | 123 |
| t618891 | 36 | 124 |
| t618892 | 37 | 125 |
| t618893 | 38 | 126 |
| t618894 | 39 | 127 |
| t618895 | 40 | 128 |
| t618896 | 41 | 129 |
| t618897 | 42 | 130 |
| t618898 | 43 | 131 |
| t618899 | 44 | 132 |
| t618900 | 45 | 133 |
| t618901 | 46 | 134 |
| t618902 | 47 | 135 |
| t618903 | 18 | 136 |
| t618904 | 49 | 137 |
| t618905 | 50 | 138 |
| t618906 | 51 | 139 |
| t618907 | 52 | 140 |
| t618908 | 53 | 141 |
| t618909 | 54 | 142 |
| t618910 | 55 | 143 |
| t618911 | 56 | 144 |
| t618912 | 57 | 145 |
| t618913 | 58 | 146 |
| t618914 | 59 | 147 |
| t618915 | 60 | 148 |
| t621323 | 61 | 149 |
| t621324 | 62 | 150 |
| t621325 | 63 | 151 |
| t621326 | 64 | 152 |
| t703752 | 65 | 153 |
| t703753 | 66 | 154 |
| t703754 | 67 | 155 |
| t703755 | 68 | 156 |
| t703756 | 69 | 157 |
| t733419 | 70 | 158 |
| t733420 | 71 | 159 |
| t733421 | 72 | 160 |
| t733422 | 73 | 161 |
| t733423 | 74 | 162 |
| t733424 | 75 | 163 |
| t733426 | 76 | 164 |
| t733427 | 77 | 165 |
| t733428 | 78 | 166 |
| t733429 | 79 | 167 |
| t733430 | 80 | 168 |
| t733431 | 81 | 169 |
| t733432 | 82 | 170 |
| t733433 | 83 | 171 |
| t733434 | 84 | 172 |
| t733435 | 85 | 173 |
| t733436 | 86 | 174 |
| t733437 | 87 | 175 |
| t733438 | 88 | 176 |
| t733439 | 89 | 177 |
| t733440 | 90 | 178 |
| t733441 | 91 | 179 |
| t733442 | 92 | 180 |
| t733443 | 93 | 181 |
| t733444 | 94 | 182 |
| t733445 | 95 | 183 |
| t733446 | 96 | 184 |
| t733447 | 97 | 185 |
| t733448 | 98 | 186 |
| t733449 | 99 | 187 |
| t733450 | 100 | 188 |
| t733451 | 101 | 189 |
| t733452 | 102 | 190 |
| t733453 | 103 | 191 |
| t733454 | 104 | 192 |
| t733455 | 105 | 193 |
| t733456 | 106 | 194 |
| t815827 | 196 | 389 |
| t815828 | 197 | 390 |
| t815829 | 198 | 391 |
| t815830 | 199 | 392 |
| t815831 | 200 | 393 |
| t815832 | 201 | 394 |
| t815833 | 202 | 395 |
| t815834 | 203 | 396 |
| t815835 | 204 | 397 |
| t815836 | 205 | 398 |
| t815837 | 206 | 399 |
| t815838 | 207 | 400 |
| t815840 | 208 | 401 |
| t815841 | 209 | 402 |
| t815842 | 210 | 403 |
| t815843 | 211 | 404 |
| t815844 | 212 | 405 |
| t815846 | 213 | 406 |
| t815847 | 214 | 407 |
| t815849 | 215 | 408 |
| t815850 | 216 | 409 |
| t815851 | 217 | 410 |
| t815852 | 218 | 411 |
| t815853 | 219 | 412 |
| t815854 | 220 | 413 |
| t815855 | 221 | 414 |
| t815857 | 222 | 415 |
| t815858 | 223 | 416 |
| t815859 | 224 | 417 |
| t815860 | 225 | 418 |
| t815861 | 226 | 419 |
| t815863 | 227 | 420 |
| t815864 | 228 | 421 |
| t815865 | 229 | 422 |
| t815866 | 230 | 423 |
| t815867 | 231 | 424 |
| t815868 | 232 | 425 |
| t815869 | 233 | 426 |
| t815871 | 234 | 427 |
| t815872 | 235 | 428 |
| t815874 | 236 | 429 |
| t815875 | 237 | 430 |
| t815878 | 238 | 431 |
| t815879 | 239 | 432 |
| t815880 | 240 | 433 |
| t815882 | 241 | 434 |
| t815886 | 242 | 435 |
| t815888 | 243 | 436 |
| t815889 | 244 | 437 |
| t815890 | 245 | 438 |
| t815891 | 246 | 439 |
| t815892 | 247 | 440 |
| t815893 | 248 | 441 |
| t815894 | 249 | 442 |
| t815895 | 250 | 443 |
| t815896 | 251 | 444 |
| t815897 | 252 | 445 |
| t815898 | 253 | 446 |
| t815899 | 254 | 447 |
| t815900 | 255 | 448 |
| t815901 | 256 | 449 |
| t815902 | 257 | 450 |
| t815903 | 258 | 451 |
| t815904 | 259 | 452 |
| t815905 | 260 | 453 |
| t815906 | 261 | 454 |
| t815907 | 262 | 455 |
| t815909 | 263 | 456 |
| t815910 | 264 | 457 |
| t813741 | 265 | 458 |
| t814031 | 266 | 459 |
| t814033 | 267 | 460 |
| t814036 | 268 | 461 |
| t814044 | 269 | 462 |
| t814047 | 270 | 463 |
| t814049 | 271 | 464 |
| t814050 | 272 | 465 |
| t814054 | 273 | 466 |
| t814057 | 274 | 467 |
| t814062 | 275 | 468 |
| t814063 | 276 | 469 |
| t814064 | 277 | 470 |
| t814066 | 278 | 471 |
| t814069 | 279 | 472 |
| t814073 | 280 | 473 |
| t814075 | 281 | 474 |
| t814078 | 282 | 475 |
| t814086 | 283 | 476 |
| t814090 | 284 | 477 |
| t814099 | 285 | 478 |
| t814103 | 286 | 479 |
| t814109 | 287 | 480 |
| t814110 | 288 | 481 |
| t814112 | 289 | 482 |
| t814116 | 290 | 483 |
| t814118 | 291 | 484 |
| t814119 | 292 | 485 |
| t814120 | 293 | 486 |
| t814121 | 294 | 487 |
| t814122 | 295 | 488 |
| t814125 | 296 | 489 |
| t814127 | 297 | 490 |
| t814128 | 298 | 491 |
| t814129 | 299 | 492 |
| t814132 | 300 | 493 |
| t814133 | 301 | 494 |
| t814135 | 302 | 495 |
| t814136 | 303 | 496 |
| t814140 | 304 | 497 |
| t814141 | 305 | 498 |
| t814144 | 306 | 499 |
| t814148 | 307 | 500 |
| t814152 | 308 | 501 |
| t814158 | 309 | 502 |
| t814161 | 310 | 503 |
| t814166 | 311 | 504 |
| t814168 | 312 | 505 |
| t814177 | 313 | 506 |
| t814193 | 314 | 507 |
| t814194 | 315 | 508 |
| t814201 | 316 | 509 |
| t814203 | 317 | 510 |
| t814208 | 318 | 511 |
| t814209 | 319 | 512 |
| t814210 | 320 | 513 |
| t814213 | 321 | 514 |
| t814222 | 322 | 515 |
| t814225 | 323 | 516 |
| t814228 | 324 | 517 |
| t814229 | 325 | 518 |
| t814252 | 326 | 519 |
| t814254 | 327 | 520 |
| t814256 | 328 | 521 |
| t814262 | 329 | 522 |
| t814280 | 330 | 523 |
| t814296 | 331 | 524 |
| t814304 | 332 | 525 |
| t814313 | 333 | 526 |
| t814317 | 334 | 527 |
| t814323 | 335 | 528 |
| t814326 | 336 | 529 |
| t814334 | 337 | 530 |
| t814342 | 338 | 531 |
| t814347 | 339 | 532 |
| t814348 | 340 | 533 |
| t814355 | 341 | 534 |
| t814360 | 342 | 535 |
| t814363 | 343 | 536 |
| t814371 | 344 | 537 |
| t814384 | 345 | 538 |
| t814388 | 346 | 539 |
| t814390 | 347 | 540 |
| t814391 | 348 | 541 |
| t814411 | 349 | 542 |
| t814414 | 350 | 543 |
| t814416 | 351 | 544 |
| t814417 | 352 | 545 |
| t814420 | 353 | 546 |
| t814430 | 354 | 547 |
| t814431 | 355 | 548 |
| t814436 | 356 | 549 |
| t814440 | 357 | 550 |
| t814442 | 358 | 551 |
| t814450 | 359 | 552 |
| t814467 | 360 | 553 |
| t814477 | 361 | 554 |
| t814482 | 362 | 555 |
| t814483 | 363 | 556 |
| t814485 | 364 | 557 |
| t814486 | 365 | 558 |
| t814488 | 366 | 559 |
| t814490 | 367 | 560 |
| t814496 | 368 | 561 |
| t814497 | 369 | 562 |
| t814500 | 370 | 563 |
| t814504 | 371 | 564 |
| t814505 | 372 | 565 |
| t814506 | 373 | 566 |
| t814508 | 374 | 567 |
| t814510 | 375 | 568 |
| t814512 | 376 | 569 |
| t814516 | 377 | 570 |
| t814521 | 378 | 571 |
| t814526 | 379 | 572 |
| t814529 | 380 | 573 |
| t814532 | 381 | 574 |
| t814533 | 382 | 575 |
| t814537 | 383 | 576 |
| t814546 | 384 | 577 |
| t814547 | 385 | 578 |
| t814551 | 386 | 579 |
| t814573 | 387 | 580 |
| t814575 | 388 | 581 |
| t454529 | 582 | 949 |
| t442710 | 583 | 950 |
| t442707 | 584 | 951 |
| t500213 | 585 | 952 |
| t500274 | 586 | 953 |
| t500588 | 587 | 954 |
| t500192 | 588 | 955 |
| t500294 | 589 | 956 |
| t500440 | 590 | 957 |
| t500492 | 591 | 958 |
| t500544 | 592 | 959 |
| t500710 | 593 | 960 |
| t500726 | 594 | 961 |
| t500701 | 595 | 962 |
| t500269 | 596 | 963 |
| t500289 | 597 | 964 |
| t500267 | 598 | 965 |
| t500610 | 599 | 966 |
| t500693 | 600 | 967 |
| t500137 | 601 | 968 |
| t500431 | 602 | 969 |
| t500435 | 603 | 970 |
| t782395 | 604 | 971 |
| t782403 | 605 | 972 |
| t782407 | 606 | 973 |
| t782411 | 607 | 974 |
| t782415 | 608 | 975 |
| t782419 | 609 | 976 |
| t782423 | 610 | 977 |
| t782428 | 611 | 978 |
| t782405 | 612 | 979 |
| t782442 | 613 | 980 |
| t782446 | 614 | 981 |
| t782451 | 615 | 982 |
| t782459 | 616 | 983 |
| t782463 | 617 | 984 |
| t782467 | 618 | 985 |
| t782476 | 619 | 986 |
| t782480 | 620 | 987 |
| t782443 | 621 | 988 |
| t782447 | 622 | 989 |
| t782460 | 623 | 990 |
| t782464 | 624 | 991 |
| t782468 | 625 | 992 |
| t782472 | 626 | 993 |
| t782477 | 627 | 994 |
| t782481 | 628 | 995 |
| t782592 | 629 | 996 |
| t782604 | 630 | 997 |
| t782627 | 631 | 998 |
| t782589 | 632 | 999 |
| t782593 | 633 | 1000 |
| t782634 | 634 | 1001 |
| t782641 | 635 | 1002 |
| t782645 | 636 | 1003 |
| t782649 | 637 | 1004 |
| t782653 | 638 | 1005 |
| t782657 | 639 | 1006 |
| t782661 | 640 | 1007 |
| t782665 | 641 | 1008 |
| t782669 | 642 | 1009 |
| t782673 | 643 | 1010 |
| t782677 | 644 | 1011 |
| t782674 | 645 | 1012 |
| t782497 | 646 | 1013 |
| t782646 | 647 | 1014 |
| t782654 | 648 | 1015 |
| t782666 | 649 | 1016 |
| t782678 | 650 | 1017 |
| t782487 | 651 | 1018 |
| t782504 | 652 | 1019 |
| t782554 | 653 | 1020 |
| t782558 | 654 | 1021 |
| t782747 | 655 | 1022 |
| t782739 | 656 | 1023 |
| t782810 | 657 | 1024 |
| t782833 | 658 | 1025 |
| t782864 | 659 | 1026 |
| t782870 | 660 | 1027 |
| t782920 | 661 | 1028 |
| t783081 | 662 | 1029 |
| t783074 | 663 | 1030 |
| t783089 | 664 | 1031 |
| t783126 | 665 | 1032 |
| t783151 | 666 | 1033 |
| t783164 | 667 | 1034 |
| t783172 | 668 | 1035 |
| t783176 | 669 | 1036 |
| t783160 | 671 | 1038 |
| t783186 | 672 | 1039 |
| t783165 | 673 | 1040 |
| t783182 | 674 | 1041 |
| t783143 | 675 | 1042 |
| t783171 | 676 | 1043 |
| t783200 | 677 | 1044 |
| t783210 | 678 | 1045 |
| t783214 | 679 | 1046 |
| t783288 | 680 | 1047 |
| t783289 | 681 | 1048 |
| t783211 | 682 | 1049 |
| t783215 | 683 | 1050 |
| t783298 | 684 | 1051 |
| t783216 | 685 | 1052 |
| t783220 | 686 | 1053 |
| t783294 | 687 | 1054 |
| t783204 | 688 | 1055 |
| t783299 | 689 | 1056 |
| t783213 | 690 | 1057 |
| t783217 | 691 | 1058 |
| t783316 | 692 | 1059 |
| t783320 | 693 | 1060 |
| t783324 | 694 | 1061 |
| t783309 | 695 | 1062 |
| t783313 | 696 | 1063 |
| t783329 | 697 | 1064 |
| t783333 | 698 | 1065 |
| t783337 | 699 | 1066 |
| t783302 | 700 | 1067 |
| t783318 | 701 | 1068 |
| t783326 | 702 | 1069 |
| t783256 | 703 | 1070 |
| t783280 | 704 | 1071 |
| t815839 | 705 | 1072 |
| t815856 | 706 | 1073 |
| t815873 | 707 | 1074 |
| t815877 | 708 | 1075 |
| t815881 | 709 | 1076 |
| t815908 | 710 | 1077 |
| t814035 | 711 | 1078 |
| t814046 | 712 | 1079 |
| t814091 | 713 | 1080 |
| t814095 | 714 | 1081 |
| t814101 | 715 | 1082 |
| t814159 | 716 | 1083 |
| t814181 | 717 | 1084 |
| t814187 | 718 | 1085 |
| t814199 | 719 | 1086 |
| t814211 | 720 | 1087 |
| t814242 | 721 | 1088 |
| t814243 | 722 | 1089 |
| t814247 | 723 | 1090 |
| t814267 | 724 | 1091 |
| t814277 | 725 | 1092 |
| t814292 | 726 | 1093 |
| t814293 | 727 | 1094 |
| t814297 | 728 | 1095 |
| t814301 | 729 | 1096 |
| t814303 | 730 | 1097 |
| t814330 | 731 | 1098 |
| t814369 | 732 | 1099 |
| t814400 | 733 | 1100 |
| t814449 | 734 | 1101 |
| t814455 | 735 | 1102 |
| t814456 | 736 | 1103 |
| t814469 | 737 | 1104 |
| t814475 | 738 | 1105 |
| t814476 | 739 | 1106 |
| t814518 | 740 | 1107 |
| t814555 | 741 | 1108 |
| t814556 | 742 | 1109 |
| t877150 | 745 | 1112 |
| t877343 | 746 | 1113 |
| t877444 | 747 | 1114 |
| t877228 | 748 | 1115 |
| t877187 | 749 | 1116 |
| t877681 | 750 | 1117 |
| t877570 | 751 | 1118 |
| t877407 | 752 | 1119 |
| t877437 | 753 | 1120 |
| t877227 | 754 | 1121 |
| t877349 | 755 | 1122 |
| t877188 | 756 | 1123 |
| t877477 | 757 | 1124 |
| t877431 | 758 | 1125 |
| t877158 | 759 | 1126 |
| t877319 | 760 | 1127 |
| t877185 | 761 | 1128 |
| t877593 | 762 | 1129 |
| t877138 | 763 | 1130 |
| t877615 | 764 | 1131 |
| t877598 | 765 | 1132 |
| t877136 | 766 | 1133 |
| t876988 | 767 | 1134 |
| t877645 | 768 | 1135 |
| t876760 | 769 | 1136 |
| t877320 | 770 | 1137 |
| t877439 | 771 | 1138 |
| t877223 | 772 | 1139 |
| t877441 | 773 | 1140 |
| t877164 | 774 | 1141 |
| t877269 | 775 | 1142 |
| t877566 | 776 | 1143 |
| t877667 | 777 | 1144 |
| t877404 | 778 | 1145 |
| t877639 | 779 | 1146 |
| t877374 | 780 | 1147 |
| t877467 | 781 | 1148 |
| t877596 | 782 | 1149 |
| t861745 | 783 | 1150 |
| t877062 | 784 | 1151 |
| t877367 | 785 | 1152 |
| t877275 | 786 | 1153 |
| t877256 | 787 | 1154 |
| t877316 | 788 | 1155 |
| t876807 | 789 | 1156 |
| t877712 | 790 | 1157 |
| t877229 | 791 | 1158 |
| t877078 | 792 | 1159 |
| t877031 | 793 | 1160 |
| t876837 | 794 | 1161 |
| t877306 | 795 | 1162 |
| t861743 | 796 | 1163 |
| t877055 | 797 | 1164 |
| t877014 | 798 | 1165 |
| t877061 | 799 | 1166 |
| t876968 | 800 | 1167 |
| t876956 | 801 | 1168 |
| t877125 | 802 | 1169 |
| t876840 | 803 | 1170 |
| t876917 | 804 | 1171 |
| t876959 | 805 | 1172 |
| t877110 | 806 | 1173 |
| t877071 | 807 | 1174 |
| t877534 | 808 | 1175 |
| t877091 | 809 | 1176 |
| t877702 | 810 | 1177 |
| t877610 | 811 | 1178 |
| t877459 | 812 | 1179 |
| t877010 | 813 | 1180 |
| t877114 | 814 | 1181 |
| t877507 | 815 | 1182 |
| t877108 | 816 | 1183 |
| t877414 | 817 | 1184 |
| t876982 | 818 | 1185 |
| t876784 | 819 | 1186 |
| t877509 | 820 | 1187 |
| t877246 | 821 | 1188 |
| t877510 | 822 | 1189 |
| t877616 | 823 | 1190 |
| t876990 | 824 | 1191 |
| t877056 | 825 | 1192 |
| t877614 | 826 | 1193 |
| t876933 | 827 | 1194 |
| t876938 | 828 | 1195 |
| t876987 | 829 | 1196 |
| t877578 | 830 | 1197 |
| t877705 | 831 | 1198 |
| t877100 | 832 | 1199 |
| t877620 | 833 | 1200 |
| t877072 | 834 | 1201 |
| t877021 | 835 | 1202 |
| t877501 | 836 | 1203 |
| t876980 | 837 | 1204 |
| t877180 | 838 | 1205 |
| t877683 | 839 | 1206 |
| t877644 | 840 | 1207 |
| t877073 | 841 | 1208 |
| t876943 | 842 | 1209 |
| t877698 | 843 | 1210 |
| t876904 | 844 | 1211 |
| t877088 | 845 | 1212 |
| t877718 | 846 | 1213 |
| t877744 | 847 | 1214 |
| t876853 | 848 | 1215 |
| t876826 | 849 | 1216 |
| t876768 | 850 | 1217 |
| t876806 | 851 | 1218 |
| t876753 | 852 | 1219 |
| t876945 | 853 | 1220 |
| t876778 | 854 | 1221 |
| t877741 | 855 | 1222 |
| t877709 | 856 | 1223 |
| t876870 | 857 | 1224 |
| t876764 | 858 | 1225 |
| t877503 | 859 | 1226 |
| t876781 | 860 | 1227 |
| t877662 | 861 | 1228 |
| t876782 | 862 | 1229 |
| t877612 | 863 | 1230 |
| t876802 | 864 | 1231 |
| t876749 | 865 | 1232 |
| t876751 | 866 | 1233 |
| t877013 | 867 | 1234 |
| t877700 | 868 | 1235 |
| t877544 | 869 | 1236 |
| t876755 | 870 | 1237 |
| t876985 | 871 | 1238 |
| t877090 | 872 | 1239 |
| t876773 | 873 | 1240 |
| t876878 | 874 | 1241 |
| t877536 | 875 | 1242 |
| t877511 | 876 | 1243 |
| t877654 | 877 | 1244 |
| t877743 | 878 | 1245 |
| t877064 | 879 | 1246 |
| t877471 | 880 | 1247 |
| t877011 | 881 | 1248 |
| t876957 | 882 | 1249 |
| t876882 | 883 | 1250 |
| t876914 | 884 | 1251 |
| t877516 | 885 | 1252 |
| t876936 | 886 | 1253 |
| t877152 | 887 | 1254 |
| t877282 | 888 | 1255 |
| t876766 | 889 | 1256 |
| t876776 | 890 | 1257 |
| t876801 | 891 | 1258 |
| t877577 | 892 | 1259 |
| t877608 | 893 | 1260 |
| t877294 | 894 | 1261 |
| t876810 | 895 | 1262 |
| t876939 | 896 | 1263 |
| t877249 | 897 | 1264 |
| t877626 | 898 | 1265 |
| t876845 | 899 | 1266 |
| t877581 | 900 | 1267 |
| t876815 | 901 | 1268 |
| t877063 | 902 | 1269 |
| t877625 | 903 | 1270 |
| t877629 | 904 | 1271 |
| t876821 | 905 | 1272 |
| t876911 | 906 | 1273 |
| t877087 | 907 | 1274 |
| t877554 | 908 | 1275 |
| t877098 | 909 | 1276 |
| t876790 | 910 | 1277 |
| t876865 | 911 | 1278 |
| t876883 | 912 | 1279 |
| t877039 | 913 | 1280 |
| t876772 | 914 | 1281 |
| t877026 | 915 | 1282 |
| t876903 | 916 | 1283 |
| t877167 | 917 | 1284 |
| t877723 | 918 | 1285 |
| t877230 | 919 | 1286 |
| t877248 | 920 | 1287 |
| t877258 | 921 | 1288 |
| t877449 | 922 | 1289 |
| t877290 | 923 | 1290 |
| t877304 | 924 | 1291 |
| t877397 | 925 | 1292 |
| t877264 | 926 | 1293 |
| t877383 | 927 | 1294 |
| t877429 | 928 | 1295 |
| t877292 | 929 | 1296 |
| t877362 | 930 | 1297 |
| t877354 | 931 | 1298 |
| t877360 | 932 | 1299 |
| t876909 | 933 | 1300 |
| t877352 | 934 | 1301 |
| t877387 | 935 | 1302 |
| t877109 | 936 | 1303 |
| t877325 | 937 | 1304 |
| t877323 | 938 | 1305 |
| t877321 | 939 | 1306 |
| t877364 | 940 | 1307 |
| t877447 | 941 | 1308 |
| t877080 | 942 | 1309 |
| t877356 | 943 | 1310 |
| t877255 | 944 | 1311 |
| t877481 | 945 | 1312 |
| t877455 | 946 | 1313 |
| t877023 | 947 | 1314 |
| t877057 | 948 | 1315 |

### EQUIVALENTS

Those skilled in the art will recognize, or be able to ascertain using no more than routine experimentation, many equivalents to the specific embodiments of the invention described here. Such equivalents are intended to be encompassed by the following claims.

All references, including patent documents, are incorporated by reference in their entirety.

1. A host cell that comprises a heterologous polynucleotide encoding a polyketide cyclase (PKC), wherein the PKC comprises an amino acid deletion or substitution at one or more residues corresponding to position 2, 3, 13, 17, 18, 19, 20, 21, 22, 24, 25, 26, 28, 29, 31, 33, 37, 38, 39, 41, 45, 46, 48, 49, 50, 52, 53, 55, 56, 57, 59, 60, 61, 62, 64, 65, 66, 67, 68, 69, 70, 71, 73, 74, 80, 81, 82, 83, 84, 85, 86, 87, 88, 89, 94, 95, 97, 98, 100, and/or 101 in SEQ ID NO: 1.

2. The host cell of claim 1, wherein the PKC comprises:
a) the amino acid G at a residue corresponding to position 2 in SEQ ID NO: 1 or a deletion of a residue corresponding to position 2 in SEQ ID NO: 1;
b) the amino acid M or Y at a residue corresponding to position 3 in SEQ ID NO: 1;
c) the amino acid R or K at a residue corresponding to position 13 in SEQ ID NO: 1;
d) the amino acid N at a residue corresponding to position 17 in SEQ ID NO: 1;
e) the amino acid D or R at a residue corresponding to position 18 in SEQ ID NO: 1;
f) the amino acid E or D at a residue corresponding to position 19 in SEQ ID NO: 1;
g) the amino acid A at a residue corresponding to position 20 in SEQ ID NO: 1;
h) the amino acid K at a residue corresponding to position 21 in SEQ ID NO: 1;
i) the amino acid T or K at a residue corresponding to position 22 in SEQ ID NO: 1;
j) the amino acid M at a residue corresponding to position 24 in SEQ ID NO: 1;
k) the amino acid A or N at a residue corresponding to position 25 in SEQ ID NO: 1;
l) the amino acid H, R, A, or E at a residue corresponding to position 26 in SEQ ID NO: 1;
m) the amino acid A, S, E, or H at a residue corresponding to position 28 in SEQ ID NO: 1;
n) the amino acid D, S, E, or T at a residue corresponding to position 29 in SEQ ID NO: 1;
o) the amino acid L or A at a residue corresponding to position 31 in SEQ ID NO: 1;
p) the amino acid H, R, D, E, K, Q, or N at a residue corresponding to position 33 in SEQ ID NO: 1;
q) the amino acid L at a residue corresponding to position 37 in SEQ ID NO: 1;
r) the amino acid L at a residue corresponding to position 38 in SEQ ID NO: 1;
s) the amino acid G, S, or Q at a residue corresponding to position 39 in SEQ ID NO: 1;
t) the amino acid K or R at a residue corresponding to position 41 in SEQ ID NO: 1;
u) the amino acid I at a residue corresponding to position 45 in SEQ ID NO: 1;
v) the amino acid F at a residue corresponding to position 46 in SEQ ID NO: 1;
w) the amino acid A at a residue corresponding to position 48 in SEQ ID NO: 1;
x) the amino acid Q at a residue corresponding to position 49 in SEQ ID NO: 1;
y) the amino acid K, F, or W at a residue corresponding to position 50 in SEQ ID NO: 1;
z) the amino acid H at a residue corresponding to position 52 in SEQ ID NO: 1;
aa) the amino acid H at a residue corresponding to position 53 in SEQ ID NO: 1;
bb) the amino acid F at a residue corresponding to position 55 in SEQ ID NO: 1;
cc) the amino acid Y at a residue corresponding to position 56 in SEQ ID NO: 1;
dd) the amino acid Y at a residue corresponding to position 57 in SEQ ID NO: 1;
ee) the amino acid I or F at a residue corresponding to position 59 in SEQ ID NO: 1;
ff) the amino acid I or L at a residue corresponding to position 60 in SEQ ID NO: 1;
gg) the amino acid T, D, or S at a residue corresponding to position 61 in SEQ ID NO: 1;
hh) the amino acid M at a residue corresponding to position 62 in SEQ ID NO: 1;
ii) the amino acid Q or D at a residue corresponding to position 64 in SEQ ID NO: 1;
jj) the amino acid N at a residue corresponding to position 65 in SEQ ID NO: 1;
kk) the amino acid L, M, or Y at a residue corresponding to position 66 in SEQ ID NO: 1;
ll) the amino acid K at a residue corresponding to position 67 in SEQ ID NO: 1;
mm) the amino acid E, G, or H at a residue corresponding to position 68 in SEQ ID NO: 1;
nn) the amino acid L or Y at a residue corresponding to position 69 in SEQ ID NO: 1;
oo) the amino acid A or L at a residue corresponding to position 70 in SEQ ID NO: 1;
pp) the amino acid Q, S, or E at a residue corresponding to position 71 in SEQ ID NO: 1;
qq) the amino acid L at a residue corresponding to position 73 in SEQ ID NO: 1;
rr) the amino acid D, E, H, Q, S, G, or L at a residue corresponding to position 74 in SEQ ID NO: 1;
ss) the amino acid V, A, E, S, D, or N at a residue corresponding to position 80 in SEQ ID NO: 1;
tt) the amino acid Y at a residue corresponding to position 81 in SEQ ID NO: 1;
uu) the amino acid K, V, or R at a residue corresponding to position 82 in SEQ ID NO: 1;
vv) the amino acid A, E, G, K, N, Q, R, or S at a residue corresponding to position 83 in SEQ ID NO: 1;
ww) the amino acid F, K, M, or Q at a residue corresponding to position 84 in SEQ ID NO: 1;
xx) the amino acid A or F at a residue corresponding to position 85 in SEQ ID NO: 1;
yy) the amino acid N, G, or S at a residue corresponding to position 86 in SEQ ID NO: 1;
zz) the amino acid C, D, or K at a residue corresponding to position 87 in SEQ ID NO: 1;
aaa) the amino acid Y, N, or S at a residue corresponding to position 88 in SEQ ID NO: 1;
bbb) the amino acid V or M at a residue corresponding to position 89 in SEQ ID NO: 1;
ccc) the amino acid V at a residue corresponding to position 94 in SEQ ID NO: 1;
ddd) the amino acid M or I at a residue corresponding to position 95 in SEQ ID NO: 1;
eee) the amino acid F at a residue corresponding to position 97 in SEQ ID NO: 1;
fff) the amino acid E, K, or Y at a residue corresponding to position 98 in SEQ ID NO: 1;
ggg) the amino acid A, G, K, N, Q, or S at a residue corresponding to position 100 in SEQ ID NO: 1; and/or
hhh) the amino acid A or E at a residue corresponding to position 101 in SEQ ID NO: 1.

3. The host cell of claim 1 or 2, wherein the PKC comprises an amino acid deletion or substitution at one or more residues corresponding to position 2, 3, 13, 17, 18, 19, 20, 21, 22, 24, 25, 26, 28, 29, 31, 33, 37, 38, 39, 45, 46, 48, 49, 50, 52, 53, 55, 56, 57, 59, 60, 61, 62, 64, 65, 66, 67, 68, 70, 71, 74, 80, 82, 83, 84, 85, 86, 87, 88, 89, 94, 95, 97, 98, 100, and 101 in SEQ ID NO: 1.

4. The host cell of claim 3, wherein the PKC comprises:
a) the amino acid G at a residue corresponding to position 2 in SEQ ID NO: 1 or a deletion of a residue corresponding to position 2 in SEQ ID NO: 1;
b) the amino acid Y at a residue corresponding to position 3 in SEQ ID NO: 1;
c) the amino acid R or K at a residue corresponding to position 13 in SEQ ID NO: 1;
d) the amino acid N at a residue corresponding to position 17 in SEQ ID NO: 1;
e) the amino acid D or R at a residue corresponding to position 18 in SEQ ID NO: 1;
f) the amino acid D at a residue corresponding to position 19 in SEQ ID NO: 1;
g) the amino acid A at a residue corresponding to position 20 in SEQ ID NO: 1;
h) the amino acid K at a residue corresponding to position 21 in SEQ ID NO: 1;
i) the amino acid K at a residue corresponding to position 22 in SEQ ID NO: 1;
j) the amino acid M at a residue corresponding to position 24 in SEQ ID NO: 1;
k) the amino acid A or N at a residue corresponding to position 25 in SEQ ID NO: 1;
l) the amino acid H, R, or A at a residue corresponding to position 26 in SEQ ID NO: 1;
m) the amino acid E or H at a residue corresponding to position 28 in SEQ ID NO: 1;
n) the amino acid S, E, or T at a residue corresponding to position 29 in SEQ ID NO: 1;
o) the amino acid L at a residue corresponding to position 31 in SEQ ID NO: 1;
p) the amino acid H, R, N at a residue corresponding to position 33 in SEQ ID NO: 1;
q) the amino acid L at a residue corresponding to position 37 in SEQ ID NO: 1;
r) the amino acid L at a residue corresponding to position 38 in SEQ ID NO: 1;
s) the amino acid G or Q at a residue corresponding to position 39 in SEQ ID NO: 1;
t) the amino acid I at a residue corresponding to position 45 in SEQ ID NO: 1;
u) the amino acid F at a residue corresponding to position 46 in SEQ ID NO: 1;
v) the amino acid A at a residue corresponding to position 48 in SEQ ID NO: 1;
w) the amino acid Q at a residue corresponding to position 49 in SEQ ID NO: 1;
x) the amino acid F, W, or K at a residue corresponding to position 50 in SEQ ID NO: 1;
y) the amino acid H at a residue corresponding to position 52 in SEQ ID NO: 1;
z) the amino acid H at a residue corresponding to position 53 in SEQ ID NO: 1;
aa) the amino acid F at a residue corresponding to position 55 in SEQ ID NO: 1;
bb) the amino acid Y at a residue corresponding to position 56 in SEQ ID NO: 1;
cc) the amino acid Y at a residue corresponding to position 57 in SEQ ID NO: 1;
dd) the amino acid F at a residue corresponding to position 59 in SEQ ID NO: 1;
ee) the amino acid I or L at a residue corresponding to position 60 in SEQ ID NO: 1;
ff) the amino acid D, S, or T at a residue corresponding to position 61 in SEQ ID NO: 1;
gg) the amino acid M at a residue corresponding to position 62 in SEQ ID NO: 1;
hh) the amino acid D at a residue corresponding to position 64 in SEQ ID NO: 1;
ii) the amino acid N at a residue corresponding to position 65 in SEQ ID NO: 1;
jj) the amino acid Y at a residue corresponding to position 66 in SEQ ID NO: 1;
kk) the amino acid K at a residue corresponding to position 67 in SEQ ID NO: 1;
ll) the amino acid G or H at a residue corresponding to position 68 in SEQ ID NO: 1;
mm) the amino acid A or L at a residue corresponding to position 70 in SEQ ID NO: 1;
nn) the amino acid Q, S, or E at a residue corresponding to position 71 in SEQ ID NO: 1;
oo) the amino acid G at a residue corresponding to position 74 in SEQ ID NO: 1;
pp) the amino acid V, A, D, or N at a residue corresponding to position 80 in SEQ ID NO: 1;
qq) the amino acid K or R at a residue corresponding to position 82 in SEQ ID NO: 1;
rr) the amino acid A, E, G, K, N, Q, R, or S at a residue corresponding to position 83 in SEQ ID NO: 1;
ss) the amino acid F or K at a residue corresponding to position 84 in SEQ ID NO: 1;
tt) the amino acid A or F at a residue corresponding to position 85 in SEQ ID NO: 1;
uu) the amino acid S at a residue corresponding to position 86 in SEQ ID NO: 1;
vv) the amino acid C or K at a residue corresponding to position 87 in SEQ ID NO: 1;
ww) the amino acid Y, N, or S at a residue corresponding to position 88 in SEQ ID NO: 1;
xx) the amino acid M at a residue corresponding to position 89 in SEQ ID NO: 1;
yy) the amino acid V at a residue corresponding to position 94 in SEQ ID NO: 1;
zz) the amino acid M or I at a residue corresponding to position 95 in SEQ ID NO: 1;
aaa) the amino acid F at a residue corresponding to position 97 in SEQ ID NO: 1;
bbb) the amino acid E, K, or Y at a residue corresponding to position 98 in SEQ ID NO: 1;
ccc) the amino acid A, G, K, N, Q, or S at a residue corresponding to position 100 in SEQ ID NO: 1; and/or
ddd) the amino acid A or E at a residue corresponding to position 101 in SEQ ID NO: 1.

5. The host cell of any one of claims 1-2, wherein the PKC comprises an amino acid substitution at a residue corresponding to position 2, 3, 13, 17, 18, 19, 20, 21, 22, 25, 26, 28, 29, 31, 33, 37, 38, 39, 41, 45, 50, 52, 53, 55, 56, 57, 60, 62, 64, 65, 67, 68, 70, 71, 74, 83, 84, 85, 86, 87, 88, 95, 97, 98, 100, and/or 101 in SEQ ID NO: 1 wherein the PKC is capable of producing olivetolic acid from 3,5,7-trioxododecanoyl-CoA, and wherein, if present:
a) the amino acid substitution at the residue corresponding to position 13 is D13R or D13K;
b) the amino acid substitution at the residue corresponding to position 21 is E21K;
c) the amino acid substitution at the residue corresponding to position 39 is D39G, D39S, or D39Q;
d) the amino acid substitution at the residue corresponding to position 50 is N50K;
e) the amino acid substitution at the residue corresponding to position 52 is E52H;
f) the amino acid substitution at the residue corresponding to position 67 is E67K;
g) the amino acid substitution at the residue corresponding to position 70 is Q70A or Q70L;
h) the amino acid substitution at the residue corresponding to position 71 is D71Q or D71S;
i) the amino acid substitution at the residue corresponding to position 74 is I74D, 174E, 174H, or 174Q;
j) the amino acid substitution at the residue corresponding to position 83 is D83A or D83G;
k) the amino acid substitution at the residue corresponding to position 86 is R86G;
l) the amino acid substitution at the residue corresponding to position 87 is S87D or S87K; and
m) the amino acid substitution at the residue corresponding to position 100 is R100A, R100G, R100K, R100N, R100Q, or R100S.

6. The host cell of any one of claims 1-2, wherein the PKC comprises an amino acid substitution at a residue corresponding to position 2, 3, 13, 17, 18, 19, 20, 21, 22, 25, 26, 28, 29, 31, 33, 37, 38, 39, 45, 50, 52, 53, 55, 56, 57, 60, 62, 64, 65, 67, 68, 70, 71, 83, 84, 85, 87, 88, 95, 97, 98, 100, and/or 101 in SEQ ID NO: 1, and wherein, if present:
a) the amino acid substitution at the residue corresponding to position 13 is D13R or D13K;
b) the amino acid substitution at the residue corresponding to position 21 is E21K;
c) the amino acid substitution at the residue corresponding to position 39 is D39G or D39Q;
d) the amino acid substitution at the residue corresponding to position 50 is N50K;
e) the amino acid substitution at the residue corresponding to position 52 is E52H;
f) the amino acid substitution at the residue corresponding to position 67 is E67K;
g) the amino acid substitution at the residue corresponding to position 70 is Q70A or Q70L;
h) the amino acid substitution at the residue corresponding to position 71 is D71Q or D71S;
i) the amino acid substitution at the residue corresponding to position 83 is D83A or D83G;
j) the amino acid substitution at the residue corresponding to position 87 is S87K; and
k) the amino acid substitution at the residue corresponding to position 100 is R100S.

7. The host cell of any one of claims 1-2, wherein the PKC comprises one or more amino acid substitutions or deletions located in a domain corresponding to:
a) beta sheet 1 (positions 1-11 in SEQ ID NO: 1);
b) loop 1 (positions 12-16 in SEQ ID NO: 1);
c) helix 1 (positions 17-29 in SEQ ID NO: 1);
d) 310 helix 1 (positions 30-32 in SEQ ID NO: 1);
e) loop 2 (positions 33-38 in SEQ ID NO: 1);
f) beta sheet 2 (positions 39-44 in SEQ ID NO: 1);
g) loop 3 (positions 45-56 in SEQ ID NO: 1);
h) beta sheet 3 (positions 57-62 in SEQ ID NO: 1);
i) loop 4 (positions 63-65 in SEQ ID NO: 1);
j) helix 2 (positions 66-73 in SEQ ID NO: 1);
k) turn 1 (positions 74-75 in SEQ ID NO: 1);
l) helix 3 (positions 76-85 in SEQ ID NO: 1);
m) 310 helix 2 (positions 86-88 in SEQ ID NO: 1);
n) beta sheet 4 (positions 89-97 in SEQ ID NO: 1); and/or
o) loop 5 (positions 98-101 in SEQ ID NO: 1).

8. The host cell of any one of claims 1-2 or claim 7, wherein one or more amino acid substitutions are located in a domain corresponding to:
a) helix 1 (positions 17-29 in SEQ ID NO: 1);
b) loop 3 (positions 45-56 in SEQ ID NO: 1);
c) helix 2 (positions 66-73 in SEQ ID NO: 1); and/or
d) helix 3 (positions 76-85 in SEQ ID NO: 1).

9. The host cell of claim 8, wherein the one or more amino acid substitutions increase the stability of a kinked helix defined by the domains corresponding to helix 2 (positions 66-73 in SEQ ID NO: 1), turn 1 (positions 74-75 in SEQ ID NO: 1), and helix 3 (positions 76-85 in SEQ ID NO: 1) and/or improve conformation of a binding pocket of the PKC to 3,5,7-trioxododecanoyl-CoA and/or olivetolic acid.

10. The host cell of claim 9, wherein one or more amino acid substitutions are located in a domain corresponding to: loop 3 (positions 45-56 in SEQ ID NO: 1); helix 2 (positions 66-73 in SEQ ID NO: 1); and helix 3 (positions 76-85 in SEQ ID NO: 1).

11. The host cell of any one of claims 1-2 or 7-10, wherein the PKC comprises a deletion or amino acid substitution at a residue corresponding to position 2, 18, 22, 24, 25, 26, 28, 29, 33, 52, 53, 57, 60, 62, 70, 82, 83, 84, 87, and/or 88 in SEQ ID NO: 1.

12. The host cell of claim 11, wherein the PKC comprises an amino acid substitution at a residue corresponding to position 18, 29, 53, 70, 82 and/or 83 in SEQ ID NO:1.

13. The host cell of claim 12, wherein the PKC comprises:
a) a deletion at a residue corresponding to position 2 in SEQ ID NO: 1;
b) the amino acid D or R at a residue corresponding to position 18 in SEQ ID NO: 1;
c) the amino acid K at a residue corresponding to position 22 in SEQ ID NO: 1;
d) the amino acid M at a residue corresponding to position 24 in SEQ ID NO: 1;
e) the amino acid N at a residue corresponding to position 25 in SEQ ID NO: 1;
f) the amino acid A or H at a residue corresponding to position 26 in SEQ ID NO: 1;
g) the amino acid E at a residue corresponding to position 28 in SEQ ID NO: 1;
h) the amino acid E or S at a residue corresponding to position 29 in SEQ ID NO: 1;
i) the amino acid H or R at a residue corresponding to position 33 in SEQ ID NO: 1;
j) the amino acid H at a residue corresponding to position 52 in SEQ ID NO: 1;
k) the amino acid H at a residue corresponding to position 53 in SEQ ID NO: 1;
l) the amino acid Y at a residue corresponding to position 57 in SEQ ID NO: 1;
m) the amino acid I at a residue corresponding to position 60 in SEQ ID NO: 1;
n) the amino acid M at a residue corresponding to position 62 in SEQ ID NO: 1;
o) the amino acid L at a residue corresponding to position 70 in SEQ ID NO: 1;
p) the amino acid K at a residue corresponding to position 82 in SEQ ID NO: 1;
q) the amino acid A, K, or R at a residue corresponding to position 83 in SEQ ID NO: 1;
r) the amino acid F or K at a residue corresponding to position 84 in SEQ ID NO: 1;
s) the amino acid C or K at a residue corresponding to position 87 in SEQ ID NO: 1; and/or
t) the amino acid Y at a residue corresponding to position 88 in SEQ ID NO: 1.

14. The host cell of claim 12 or 13, wherein relative to SEQ ID NO: 1, the PKC comprises: E53H, G82K, and Q70L.

15. The host cell of claim 1 or 2, wherein relative to SEQ ID NO: 1, the PKC comprises:
a) A18D, T26A, V28E, N29S, E53H, E60I, Q70L, G82K, D83A, and S87K;
b) A18D, E22K, N29S, E53H, Q70L, G82K, D83R, S87K, and F₈₈Y;
c) A18D, E22K, N29S, E53H, Q70L, G82K, D83K, S87K, and F₈₈Y;
d) A18D, E22K, N29S, I33R, E53H, Q70L, G82K, D83A, S87K, and F₈₈Y;
e) A18D, E22K, K25N, T26A, N29S, E53H, Q70L, G82K, D83A, and S87K;
f) A18D, E22K, N29S, E53H, Q70L, G82K, D83A, V84K, S87K, and F₈₈Y;
g) A18D, E22K, T26A, N29S, E53H, Q70L, G82K, D83A, S87K, and F₈₈Y;
h) A18D, E22K, T26H, N29S, E53H, Q70L, G82K, D83A, S87K, and F₈₈Y;
i) A18D, E22K, N29S, I33H, E53H, Q70L, G82K, D83A, S87K, and F₈₈Y;
j) A18D, E22K, N29S, E53H, Q70L, G82K, D83A, V84F, S87K, and F₈₈Y;
k) A18D, T26A, N29S, E53H, Q70L, G82K, D83A, and F₈₈Y;
l) A18D, E22K, T26A, N29S, E53H, Q70L, G82K, D83A, and S87K;
m) A18D, T26A, N29S, E53H, Q70L, G82K, D83A, S87K, and F₈₈Y;
n) A18D, T26A, N29S, E53H, Q70L, G82K, and D83A;
o) A18D, E22K, T26A, N29S, E53H, Q70L, G82K, D83A, and F₈₈Y;
p) A18D, E22K, N29S, E53H, Q70L, G82K, D83A, S87K, and F₈₈Y;
q) A18D, T26A, V28E, N29S, E53H, Q70L, G82K, D83A, and S87K;
r) A18R, E22K, N29S, E53H, Q70L, G82K, D83A, S87K, and F₈₈Y;
s) deletion of a residue corresponding to position 2 in SEQ ID NO: 1, A18D, E22K, N29S, E53H, Q70L, G82K, D83A, S87K, and F₈₈Y;
t) A18D, E22K, N29S, E53H, T62M, Q70L, G82K, D83A, S87K, and F₈₈Y;
u) A18D, E22K, T26A, N29E, E53H, Q70L, G82K, D83A, and S87K;
v) A18D, E22K, N29S, E52H, E53H, Q70L, G82K, D83A, S87K, and F₈₈Y;
w) A18D, E22K, N29S, E53H, Q70L, G82K, D83A, S87C, and F₈₈Y;
x) A18D, E22K, F₂₄M, N29S, E53H, Q70L, G82K, D83A, S87K, and F₈₈Y;
y) A18D, N29S, E53H, Q70L, G82K, D83A, and F₈₈Y;
z) A18D, N29S, E53H, Q70L, G82K, D83A, S87K, and F₈₈Y;
aa) A18D, E22K, T26A, N29S, E53H, H₅₇Y, Q70L, G82K, D83A, and S87K;
bb) I33R, E52H, Y55F, Q70A, G80A, D83K, and V84K;
cc) D13R, A18D, N29S, Q70L, D71Q, G80A, and D83K;
dd) I33R, E52H, Y55F, Q70A, 174G, and D83K;
ee) T26R, I33H, E52H, Y55F, Q70A, and D83K;
ff) A18D, T26R, E52H, Y55F, Q70A, G80A, D83N, V84K, and Y85A;
gg) T26R, I33H, Y55F, Q70A, G80A, D83K, and Y85A;
hh) E22K, T26H, N29S, D39G, K49Q, E52H, D83N, and V84K;
ii) E17N, A18D, V31L, D71S, D83A, and V84F; or
jj) A18D, V31L, D39Q, D71S, D83A, and V84F.

16. The host cell of claim 1 or 2, wherein the PKC comprises an amino acid substitution at a residue corresponding to position 17, 18, 26, 29, 31, 33, 37, 39, 71, 80, 83, 84, and/or 95 in SEQ ID NO: 1.

17. The host cell of claim 16, wherein the PKC comprises:
a) the amino acid N at a residue corresponding to position 17 in SEQ ID NO: 1;
b) the amino acid D at a residue corresponding to position 18 in SEQ ID NO: 1;
c) the amino acid A or H at a residue corresponding to position 26 in SEQ ID NO: 1;
d) the amino acid T at a residue corresponding to position 29 in SEQ ID NO: 1;
e) the amino acid L at a residue corresponding to position 31 in SEQ ID NO: 1;
f) the amino acid R, H, or N at a residue corresponding to position 33 in SEQ ID NO: 1;
g) the amino acid L at a residue corresponding to position 37 in SEQ ID NO: 1;
h) the amino acid Q or G at a residue corresponding to position 39 in SEQ ID NO: 1;
i) the amino acid S at a residue corresponding to position 71 in SEQ ID NO: 1;
j) the amino acid A at a residue corresponding to position 80 in SEQ ID NO: 1;
k) the amino acid Q, N, or A at a residue corresponding to position 83 in SEQ ID NO: 1;
l) the amino acid F at a residue corresponding to position 84 in SEQ ID NO: 1; and/or
m) the amino acid M at a residue corresponding to position 95 in SEQ ID NO: 1.

18. The host cell of claim 16 or 17, wherein relative to SEQ ID NO: 1, the PKC comprises:
a) E17N, A18D, and D39G;
b) D39Q, and F₉₅M;
c) I33N and F₉₅M;
d) E17N, I33R, and F₉₅M;
e) T26H, D83Q, and F₉₅M;
f) I33H, D39Q, and D71S;
g) N29T, I33N, and D71S;
h) I33N, D39Q, and F₉₅M;
i) D71S, D83N, and F₉₅M;
j) D39Q and D83Q;
k) A18D and M37L;
l) E17N, A18D, and I33N;
m) D39G, D71S, and V84F;
n) N29T and D39G;
o) A18D, D71S, and G80A;
p) N29T, V31L, and D39Q;
q) A18D, V31L, and D39G;
r) T26H, N29T, and D39Q;
s) T26A, N29T, and I33H;
t) D39Q, D71S, and G80A;
u) A18D, D39Q, and V84F;
v) E17N, I33R, and D39Q;
w) E17N, D71S, and F₉₅M;
x) E17N, I33N, and F₉₅M;
y) T26A, N29T, and V31L;
z) E17N, T26A, and D39G;
aa) A18D, D39Q, and D71S;
bb) V31L, D39G, and V84F;
cc) N29T, 133R, and D39Q;
dd) D39G, D71S, and D83N;
ee) V31L, D71S, and D83Q;
ff) I33R, D39Q, and V84F;
gg) D39G and D71S;
hh) E17N, 133H, and D71S;
ii) T26H, D39G, and V84F;
jj) A18D, 133R, and D39G;
kk) T26A, D83N, and F₉₅M;
ll) V31L, I33H, and D71S;
mm) V31L, D39G, and D71S;
nn) A18D and D39Q;
oo) 133R, D39G, and D71S;
pp) E17N, G80A, and D83N;
qq) A18D, N29T, and V31L;
rr) D39Q, D71S, and D83A;
ss) A18D, T26H, and D39Q;
tt) T26A and F₉₅M;
uu) I33H, D39G, and D83Q;
vv) T26H, 133N, and G80A;
ww) I33R, D39G, and F₉₅M;
xx) V31L, 133R, and D83N;
yy) N29T, D39G, and V84F;
zz) D71S and D83Q;
aaa) I33H, D39Q, and G80A;
bbb) E17N, D39G, and D83A;
ccc) E17N, 133N, and V84F;
ddd) N29T, 133N, and D83Q;
eee) I33N, D71S, and G80A;
fff) I33R, D39G, and D83N;
ggg) T26A and N29T;
hhh) I33H, D83Q, and F₉₅M;
iii) T26A, V31L, and D39Q;
jjj) E17N, V31L, and I33N;
kkk) E17N, T26A, and M37L; or
111) N29T and 133N.

19. The host cell of claim 1 or 2, wherein the PKC comprises an amino acid substitution at a residue corresponding to position 13, 18, 22, 26, 29, 33, 39, 49, 52, 55, 61, 64, 65, 66, 68, 70, 71, 74, 80, 83, 84, 85, and/or 98 in SEQ ID NO: 1.

20. The host cell of claim 19, wherein the PKC comprises:
a) the amino acid R at a residue corresponding to position 13 in SEQ ID NO: 1;
b) the amino acid D at a residue corresponding to position 18 in SEQ ID NO: 1;
c) the amino acid K at a residue corresponding to position 22 in SEQ ID NO: 1;
d) the amino acid R or H at a residue corresponding to position 26 in SEQ ID NO: 1;
e) the amino acid S at a residue corresponding to position 29 in SEQ ID NO: 1;
f) the amino acid R or H at a residue corresponding to position 33 in SEQ ID NO: 1;
g) the amino acid G at a residue corresponding to position 39 in SEQ ID NO: 1;
h) the amino acid Q at a residue corresponding to position 49 in SEQ ID NO: 1;
i) the amino acid H at a residue corresponding to position 52 in SEQ ID NO: 1;
j) the amino acid F at a residue corresponding to position 55 in SEQ ID NO: 1;
k) the amino acid D at a residue corresponding to position 61 in SEQ ID NO: 1;
l) the amino acid D at a residue corresponding to position 64 in SEQ ID NO: 1;
m) the amino acid N at a residue corresponding to position 65 in SEQ ID NO: 1;
n) the amino acid Y at a residue corresponding to position 66 in SEQ ID NO: 1;
o) the amino acid G at a residue corresponding to position 68 in SEQ ID NO: 1;
p) the amino acid A or L at a residue corresponding to position 70 in SEQ ID NO: 1;
q) the amino acid E or Q at a residue corresponding to position 71 in SEQ ID NO: 1;
r) the amino acid G at a residue corresponding to position 74 in SEQ ID NO: 1;
s) the amino acid N or A at a residue corresponding to position 80 in SEQ ID NO: 1;
t) the amino acid K or N at a residue corresponding to position 83 in SEQ ID NO: 1;
u) the amino acid K at a residue corresponding to position 84 in SEQ ID NO: 1;
v) the amino acid F or A at a residue corresponding to position 85 in SEQ ID NO: 1; and/or
w) the amino acid E or K at a residue corresponding to position 98 in SEQ ID NO: 1.

21. The host cell of claim 20, wherein relative to SEQ ID NO: 1, the PKC comprises:
a) Y85F;
b) E64D, D71E, and T98K;
c) S65N;
d) V66Y;
e) G80N;
f) T68G;
g) D71E;
h) E64D and D71E;
i) D71E and Y85F;
j) I33R, E52H, Y55F, Q70A, G80A, D83K, and V84K;
k) D13R, A18D, N29S, Q70L, D71Q, G80A, and D83K;
l) I33R, E52H, Y55F, Q70A, I74G, and D83K;
m) Y55F, G80A, and D83K;
n) T26R, I33H, E52H, Y55F, Q70A, and D83K;
o) A18D, T26R, E52H, Y55F, Q70A, G80A, D83N, V84K, and Y85A;
p) T98E;
q) T26R, I33H, Y55F, Q70A, G80A, D83K, and Y85A;
r) V61D; or
s) E22K, T26H, N29S, D39G, K49Q, E52H, D83N, and V84K.

22. The host cell of any one of claims 1-2, wherein the PKC comprises an amino acid substitution at a residue corresponding to position 13, 17, 18, 22, 26, 29, 31, 33, 39, 52, 55, 61, 64, 65, 66, 70, 71, 74, 80, 82, 83, 84, 85, 88, 95, and/or 98 in SEQ ID NO: 1.

23. The host cell of claim 22, wherein the PKC comprises:
a) the amino acid R at a residue corresponding to position 13 in SEQ ID NO: 1;
b) the amino acid N at a residue corresponding to position 17 in SEQ ID NO: 1;
c) the amino acid D at a residue corresponding to position 18 in SEQ ID NO: 1;
d) the amino acid K at a residue corresponding to position 22 in SEQ ID NO: 1;
e) the amino acid R at a residue corresponding to position 26 in SEQ ID NO: 1;
f) the amino acid S at a residue corresponding to position 29 in SEQ ID NO: 1;
g) the amino acid L at a residue corresponding to position 31 in SEQ ID NO: 1;
h) the amino acid H, N, or R at a residue corresponding to position 33 in SEQ ID NO: 1;
i) the amino acid G or Q at a residue corresponding to position 39 in SEQ ID NO: 1;
j) the amino acid H at a residue corresponding to position 52 in SEQ ID NO: 1;
k) the amino acid F at a residue corresponding to position 55 in SEQ ID NO: 1;
l) the amino acid S at a residue corresponding to position 61 in SEQ ID NO: 1;
m) the amino acid D at a residue corresponding to position 64 in SEQ ID NO: 1;
n) the amino acid N at a residue corresponding to position 65 in SEQ ID NO: 1;
o) the amino acid Y at a residue corresponding to position 66 in SEQ ID NO: 1;
p) the amino acid A or L at a residue corresponding to position 70 in SEQ ID NO: 1;
q) the amino acid E, Q, or S at a residue corresponding to position 71 in SEQ ID NO: 1;
r) the amino acid G at a residue corresponding to position 74 in SEQ ID NO: 1;
s) the amino acid A at a residue corresponding to position 80 in SEQ ID NO: 1;
t) the amino acid R at a residue corresponding to position 82 in SEQ ID NO: 1;
u) the amino acid A, E, K, or N at a residue corresponding to position 83 in SEQ ID NO: 1;
v) the amino acid F or K at a residue corresponding to position 84 in SEQ ID NO: 1;
w) the amino acid A or F at a residue corresponding to position 85 in SEQ ID NO: 1;
x) the amino acid N at a residue corresponding to position 88 in SEQ ID NO: 1;
y) the amino acid M at a residue corresponding to position 95 in SEQ ID NO: 1; and/or
z) the amino acid K or Y at a residue corresponding to position 98 in SEQ ID NO: 1.

24. The host cell of claim 22, wherein the PKC comprises an amino acid substitution at a residue corresponding to position 13, 17, 18, 22, 26, 31, 33, 39, 52, 55, 64, 65, 66, 70, 71, 74, 80, 82, 83, 84, 85, 88, and/or 98 in SEQ ID NO: 1.

25. The host cell of claim 24, wherein the PKC comprises:
a) the amino acid R at a residue corresponding to position 13 in SEQ ID NO:1;
b) the amino acid N at a residue corresponding to position 17 in SEQ ID NO:1;
c) the amino acid D at a residue corresponding to position 18 in SEQ ID NO:1;
d) the amino acid K at a residue corresponding to position 22 in SEQ ID NO:1;
e) the amino acid R at a residue corresponding to position 26 in SEQ ID NO:1;
f) the amino acid L at a residue corresponding to position 31 in SEQ ID NO:1;
g) the amino acid R, H or N at a residue corresponding to position 33 in SEQ ID NO:1;
h) the amino acid G at a residue corresponding to position 39 in SEQ ID NO:1;
i) the amino acid H at a residue corresponding to position 52 in SEQ ID NO:1;
j) the amino acid F at a residue corresponding to position 55 in SEQ ID NO:1;
k) the amino acid D at a residue corresponding to position 64 in SEQ ID NO:1;
l) the amino acid N at a residue corresponding to position 65 in SEQ ID NO:1;
m) the amino acid Y at a residue corresponding to position 66 in SEQ ID NO:1;
n) the amino acid A at a residue corresponding to position 70 in SEQ ID NO:1;
o) the amino acid S or E at a residue corresponding to position 71 in SEQ ID NO:1;
p) the amino acid R at a residue corresponding to position 82 in SEQ ID NO: 1;
q) the amino acid G at a residue corresponding to position 74 in SEQ ID NO:1;
r) the amino acid A at a residue corresponding to position 80 in SEQ ID NO:1;
s) the amino acid E, A, K, or N at a residue corresponding to position 83 in SEQ ID NO:1;
t) the amino acid K or F at a residue corresponding to position 84 in SEQ ID NO:1;
u) the amino acid F at a residue corresponding to position 85 in SEQ ID NO:1;
v) the amino acid N at a residue corresponding to position 88 in SEQ ID NO: 1; and/or
w) the amino acid K or Y at a residue corresponding to position 98 in SEQ ID NO:1.

26. The host cell of claim 24 or 25, wherein relative to SEQ ID NO: 1, the PKC comprises:
a) A18D, T26R, I33R, D39G, E52H, Y55F, E64D, Q70A, G80A, D83N, and V84K;
b) E17N, T26R, V31L, D71S, D83E, and V84F;
c) E17N, V31L, I33N, E64D, D71S, and V84F;
d) E17N, A18D, V31L, D71S, D83A, and V84F;
e) E17N, E22K, V31L, 133N, D71E, and V84F;
f) E17N, A18D, V31L, D71E, D83A, and V84F;
g) E17N, V31L, D71S, D83E, V84F, and T98Y;
h) V84K, T26R, D39G, D83E, T98K, and S65N;
i) E17N, A18D, V31L, I33N, D71S, D83E, and V84K;
j) E17N, A18D, V31L, D71S, I74G, D83E, and V84F;
k) E17N, V31L, I33N, D71S, V84K, and Y85F;
1) A18D, T26R, I33R, E52H, Y55F, V66Y, Q70A, G80A, D83N, V84K, and Y85F;
m) E17N, A18D, V31L, I33N, D71S, D83K, and V84K;
n) E17N, A18D, T26R, V31L, I33N, D71S, and V84K;
o) V84K, T26R, D39G, D13R, T98K, and S65N;
p) E17N, V31L, I33N, S65N, D71S, and V84K;
q) E17N, A18D, E22K, V31L, I33N, D71E, and V84F;
r) D13R, E17N, A18D, V31L, D71S, D83A, and V84K;
s) E17N, V31L, D39G, D71S, D83A, and V84K;
t) D13R, A18D, T26R, E52H, Y55F, Q70A, G80A, D83N, V84K, and Y85F;
u) E17N, A18D, T26R, V31L, D71S, D83A, and V84K;
v) E17N, V31L, I33N, D71S, G82R, and V84F;
w) E17N, V31L, I33N, D71S, V84K, and T98K;
x) V84K, T26R, D39G, D83E, D13R, and T98K;
y) V84K, T26R, D83E, D13R, T98K, and S65N;
z) E17N, V31L, I33N, V66Y, D71E, and V84F;
aa) E17N, A18D, V31L, I33N, D71E, and V84F;
bb) E17N, A18D, V31L, I33N, D71S, G80A, and V84F;
cc) A18D, T26R, E52H, Y55F, Q70A, G80A, D83N, V84K, Y85F, and F₈₈N;
dd) E17N, A18D, V31L, I33N, D71S, V84K, and T98K;
ee) E17N, A18D, V31L, I33N, S65N, D71S, and V84K;
ff) T26R, I33H, Y55F, Q70A, G80A, D83K, V84K, and F₈₈N;
gg) A18D, E22K, T26R, E52H, Y55F, Q70A, D71E, G80A, D83N, and V84K; or
hh) E17N, V31L, I33N, D71E, G82R, and V84F.

27. The host cell of any one of claims 1-26, wherein the PKC comprises a sequence that is at least 90% identical to any one of SEQ ID NOs: 19-106, 196-388, 582-669, 671-742, and 745-948.

28. The host cell of claim 27, wherein the PKC comprises a sequence that is at least 90% identical to any one of SEQ ID NOs: 19, 21, 23, 24, 26, 27, 29, 32, 33, 34, 35, 38, 40, 42, 43, 45, 48, 49, 50, 51, 53, 58, 60, 62, 65, 66, 67, 68, 78, 87, 91, 92, 94, 99, 103, 105, 219, 220, 231, 234, 240, 254, 255, 258 283, 289, 294, 344, 345, 349, 365, 582-603, 605, 606, 607, 608, 610, 611, 612, 614, 616, 617, 618, 622, 625, 627, 629, 631, 632, 633, 634, 637, 638, 639, 640, 641, 642, 643, 644, 646, 648, 651, 653, 654, 655, 656, 657, 658, 659, 660, 661, 662, 663, 666, 667, 668, 672, 673, 674, 675, 676, 679, 680, 683, 684, 685, 686, 691, 693, 694, 695, 696, 697, 699, 700, 703, 707, 711, 713, 714, 715, 724, 732, 734, 735, 736, 737, 738, 739, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

29. The host cell of claim 27, wherein the PKC comprises a sequence selected from any one of SEQ ID NOs: 19-106, 196-388, 582-669, 671-742, and 745-948.

30. The host cell of claim 28, wherein the PKC comprises a sequence selected from any one of SEQ ID NOs: 19, 21, 23, 24, 26, 27, 29, 32, 33, 34, 35, 38, 40, 42, 43, 45, 48, 49, 50, 51, 53, 58, 60, 62, 65, 66, 67, 68, 78, 87, 91, 92, 94, 99, 103, 105, 219, 220, 231, 234, 240, 254, 255, 258 283, 289, 294, 344, 345, 349, 365, 582-603, 605, 606, 607, 608, 610, 611, 612, 614, 616, 617, 618, 622, 625, 627, 629, 631, 632, 633, 634, 637, 638, 639, 640, 641, 642, 643, 644, 646, 648, 651, 653, 654, 655, 656, 657, 658, 659, 660, 661, 662, 663, 666, 667, 668, 672, 673, 674, 675, 676, 679, 680, 683, 684, 685, 686, 691, 693, 694, 695, 696, 697, 699, 700, 703, 707, 711, 713, 714, 715, 724, 732, 734, 735, 736, 737, 738, 739, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777,

31. The host cell of any one of claims 1-30, wherein the heterologous polynucleotide comprises a sequence that is at least 90% identical to any one of SEQ ID NOs: 107-194, 389-581, 949-1036, 1038-1109 and 1112-1315.

32. The host cell of claim 31, wherein the heterologous polynucleotide comprises a sequence selected from any one of SEQ ID NOs: 107-194, 389-581, 949-1036, 1038-1109 and 1112-1315.

33. The host cell of claim 31 or 32, wherein the heterologous polynucleotide comprises a sequence that is at least 90% identical to any one of SEQ ID NOs: 107, 109, 111, 112, 114, 115, 117, 120, 121, 122, 123, 126, 128, 130, 131, 133, 136, 137, 138, 139, 141, 146, 148, 150, 153, 154, 155, 156, 166, 175, 179, 180, 182, 187, 191, 193, 412, 413, 424, 427, 433, 447, 448, 451, 476, 482, 487, 537, 538, 542, 558, 949-970, 1074, 972, 973, 974, 975, 977, 978, 979, 981, 983, 984, 985, 989, 992, 994, 996, 998, 999, 1000, 1001, 1004, 1005, 1006, 1007, 1008, 1009, 1010, 1011, 1013, 1015, 1018, 1020, 1021, 1022, 1023, 1024, 1025, 1026, 1027, 1028, 1029, 1030, 1033, 1034, 1035, 1039, 1040, 1041, 1042, 1043, 1046, 1047, 1050, 1051, 1052, 1053, 1058, 1060, 1061, 1062, 1063, 1064, 1066, 1067, 1070, 1078, 1080, 1081, 1082, 1091, 1099, 1101, 1102, 1103, 1104, 1105, 1106, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

34. The host cell of claim 33, wherein the heterologous polynucleotide comprises a sequence selected from any one of SEQ ID NOs: 107, 109, 111, 112, 114, 115, 117, 120, 121, 122, 123, 126, 128, 130, 131, 133, 136, 137, 138, 139, 141, 146, 148, 150, 153, 154, 155, 156, 166, 175, 179, 180, 182, 187, 191, 193, 412, 413, 424, 427, 433, 447, 448, 451, 476, 482, 487, 537, 538, 542, 558, 949-970, 1074, 972, 973, 974, 975, 977, 978, 979, 981, 983, 984, 985, 989, 992, 994, 996, 998, 999, 1000, 1001, 1004, 1005, 1006, 1007, 1008, 1009, 1010, 1011, 1013, 1015, 1018, 1020, 1021, 1022, 1023, 1024, 1025, 1026, 1027, 1028, 1029, 1030, 1033, 1034, 1035, 1039, 1040, 1041, 1042, 1043, 1046, 1047, 1050, 1051, 1052, 1053, 1058, 1060, 1061, 1062, 1063, 1064, 1066, 1067, 1070, 1078, 1080, 1081, 1082, 1091, 1099, 1101, 1102, 1103, 1104, 1105, 1106, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

35. The host cell of any one of claims 1-34, wherein the PKC comprises at least 6, 7, 8, 9, or 10 amino acid substitutions or deletions relative to SEQ ID NO: 1.

36. The host cell of any one of claims 1-35, wherein the PKC is capable of producing at least 10%, 20%, 30%, 40%, 50%, 60%, 70%, 80%, 90%, 100%, or more olivetolic acid relative to a control.

37. The host cell of claim 36, wherein the control is a PKC that does not comprise one or more of the amino acid substitutions or deletions.

38. A host cell that comprises a heterologous polynucleotide encoding a polyketide cyclase (PKC), wherein the PKC comprises an amino acid substitution at a residue corresponding to position 13, 17, 18, 22, 26, 31, 33, 39, 52, 55, 64, 65, 66, 70, 71, 74, 80, 82, 83, 84, 85, 88, and/or 98 in SEQ ID NO: 1.

39. The host cell of claim 38, wherein the PKC comprises:
a) the amino acid R at a residue corresponding to position 13 in SEQ ID NO:1;
b) the amino acid N at a residue corresponding to position 17 in SEQ ID NO:1;
c) the amino acid D at a residue corresponding to position 18 in SEQ ID NO:1;
d) the amino acid K at a residue corresponding to position 22 in SEQ ID NO:1;
e) the amino acid R at a residue corresponding to position 26 in SEQ ID NO:1;
f) the amino acid L at a residue corresponding to position 31 in SEQ ID NO:1;
g) the amino acid R, H or N at a residue corresponding to position 33 in SEQ ID NO:1;
h) the amino acid G at a residue corresponding to position 39 in SEQ ID NO:1;
i) the amino acid H at a residue corresponding to position 52 in SEQ ID NO:1;
j) the amino acid F at a residue corresponding to position 55 in SEQ ID NO:1;
k) the amino acid D at a residue corresponding to position 64 in SEQ ID NO:1;
l) the amino acid N at a residue corresponding to position 65 in SEQ ID NO:1;
m) the amino acid Y at a residue corresponding to position 66 in SEQ ID NO:1;
n) the amino acid A at a residue corresponding to position 70 in SEQ ID NO:1;
o) the amino acid S or E at a residue corresponding to position 71 in SEQ ID NO:1;
p) the amino acid R at a residue corresponding to position 82 in SEQ ID NO: 1;
q) the amino acid G at a residue corresponding to position 74 in SEQ ID NO:1;
r) the amino acid A at a residue corresponding to position 80 in SEQ ID NO:1;
s) the amino acid E, A, K, or N at a residue corresponding to position 83 in SEQ ID NO:1;
t) the amino acid K or F at a residue corresponding to position 84 in SEQ ID NO:1;
u) the amino acid F at a residue corresponding to position 85 in SEQ ID NO:1;
v) the amino acid N at a residue corresponding to position 88 in SEQ ID NO: 1; and/or
w) the amino acid K or Y at a residue corresponding to position 98 in SEQ ID NO:1.

40. The host cell of claim 38 or 39, wherein relative to SEQ ID NO: 1, the PKC comprises:
a) A18D, T26R, I33R, D39G, E52H, Y55F, E64D, Q70A, G80A, D83N, and V84K;
b) E17N, T26R, V31L, D71S, D83E, and V84F;
c) E17N, V31L, I33N, E64D, D71S, and V84F;
d) E17N, A18D, V31L, D71S, D83A, and V84F;
e) E17N, E22K, V31L, I33N, D71E, and V84F;
f) E17N, A18D, V31L, D71E, D83A, and V84F;
g) E17N, V31L, D71S, D83E, V84F, and T98Y;
h) V84K, T26R, D39G, D83E, T98K, and S65N;
i) E17N, A18D, V31L, I33N, D71S, D83E, and V84K;
j) E17N, A18D, V31L, D71S, I74G, D83E, and V84F;
k) E17N, V31L, I33N, D71S, V84K, and Y85F;
1) A18D, T26R, I33R, E52H, Y55F, V66Y, Q70A, G80A, D83N, V84K, and Y85F;
m) E17N, A18D, V31L, I33N, D71S, D83K, and V84K;
n) E17N, A18D, T26R, V31L, I33N, D71S, and V84K;
o) V84K, T26R, D39G, D13R, T98K, and S65N;
p) E17N, V31L, I33N, S65N, D71S, and V84K;
q) E17N, A18D, E22K, V31L, I33N, D71E, and V84F;
r) D13R, E17N, A18D, V31L, D71S, D83A, and V84K;
s) E17N, V31L, D39G, D71S, D83A, and V84K;
t) D13R, A18D, T26R, E52H, Y55F, Q70A, G80A, D83N, V84K, and Y85F;
u) E17N, A18D, T26R, V31L, D71S, D83A, and V84K;
v) E17N, V31L, I33N, D71S, G82R, and V84F;
w) E17N, V31L, I33N, D71S, V84K, and T98K;
x) V84K, T26R, D39G, D83E, D13R, and T98K;
y) V84K, T26R, D83E, D13R, T98K, and S65N;
z) E17N, V31L, I33N, V66Y, D71E, and V84F;
aa) E17N, A18D, V31L, I33N, D71E, and V84F;
bb) E17N, A18D, V31L, I33N, D71S, G80A, and V84F;
cc) A18D, T26R, E52H, Y55F, Q70A, G80A, D83N, V84K, Y85F, and F₈₈N;
dd) E17N, A18D, V31L, I33N, D71S, V84K, and T98K;
ee) E17N, A18D, V31L, I33N, S65N, D71S, and V84K;
ff) T26R, I33H, Y55F, Q70A, G80A, D83K, V84K, and F₈₈N;
gg) A18D, E22K, T26R, E52H, Y55F, Q70A, D71E, G80A, D83N, and V84K; or
hh) E17N, V31L, I33N, D71E, G82R, and V84F.

41. The host cell of any one of claims 38-40, wherein the PKC comprises a sequence that is at least 90% identical to any one of SEQ ID NOs: 255, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

42. The host cell of claim 41, wherein the PKC comprises any one of SEQ ID NOs: 255, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, and 777.

43. The host cell of any one of claims 38-42, wherein the heterologous polynucleotide comprises a sequence that is at least 90% identical to any one of SEQ ID NOs: 448, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

44. The host cell of claim 43, wherein the heterologous polynucleotide comprises any one of SEQ ID NOs: 448, 1116, 1118, 1132, 1135, 1157, 1175, 1177, 1189, 1195, 1207, 1213, 1224, 1229, 1232, 1288, 1289, 1225, 1217, 1218, 1300, 1238, 1176, 1293, 1290, 1291, 1125, 1114, 1124, 1242, 1275, 1190, 1200, and 1144.

45. The host cell of claim 40, wherein relative to SEQ ID NO: 1, the PKC comprises A18D, T26R, I33R, D39G, E52H, Y55F, E64D, Q70A, G80A, D83N, and V84K.

46. The host cell of claim 40, wherein relative to SEQ ID NO: 1, the PKC comprises E17N, T26R, V31L, D71S, D83E, and V84F.

47. The host cell of claim 40, wherein relative to SEQ ID NO: 1, the PKC comprises E17N, V31L, I33N, E64D, D71S, and V84F.

48. The host cell of claim 40, wherein relative to SEQ ID NO: 1, the PKC comprises E17N, A18D, V31L, D71S, D83A, and V84F.

49. The host cell of claim 40, wherein relative to SEQ ID NO: 1, the PKC comprises E17N, E22K, V31L, I33N, D71E, and V84F.

50. The host cell of claim 40, wherein relative to SEQ ID NO: 1, the PKC comprises E17N, A18D, V31L, D71E, D83A, and V84F.

51. The host cell of claim 40, wherein relative to SEQ ID NO: 1, the PKC comprises E17N, V31L, D71S, D83E, V84F, and T98Y.

52. The host cell of claim 40, wherein relative to SEQ ID NO: 1, the PKC comprises V84K, T26R, D39G, D83E, T98K, and S65N.

53. The host cell of claim 40, wherein relative to SEQ ID NO: 1, the PKC comprises E17N, A18D, V31L, I33N, D71S, D83E, and V84K.

54. The host cell of claim 40, wherein relative to SEQ ID NO: 1, the PKC comprises E17N, A18D, V31L, D71S, I74G, D83E, and V84F.

55. The host cell of claim 40, wherein relative to SEQ ID NO: 1, the PKC comprises E17N, V31L, I33N, D71S, V84K, and Y85F.

56. The host cell of claim 40, wherein relative to SEQ ID NO: 1, the PKC comprises A18D, T26R, I33R, E52H, Y55F, V66Y, Q70A, G80A, D83N, V84K, and Y85F.

57. The host cell of claim 40, wherein relative to SEQ ID NO: 1, the PKC comprises E17N, A18D, V31L, I33N, D71S, D83K, and V84K.

58. The host cell of claim 40, wherein relative to SEQ ID NO: 1, the PKC comprises E17N, A18D, T26R, V31L, I33N, D71S, and V84K.

59. The host cell of claim 40, wherein relative to SEQ ID NO: 1, the PKC comprises V84K, T26R, D39G, D13R, T98K, and S65N.

60. The host cell of claim 40, wherein relative to SEQ ID NO: 1, the PKC comprises E17N, V31L, I33N, S65N, D71S, and V84K.

61. The host cell of claim 40, wherein relative to SEQ ID NO: 1, the PKC comprises E17N, A18D, E22K, V31L, I33N, D71E, and V84F.

62. The host cell of claim 40, wherein relative to SEQ ID NO: 1, the PKC comprises D13R, E17N, A18D, V31L, D71S, D83A, and V84K.

63. The host cell of claim 40, wherein relative to SEQ ID NO: 1, the PKC comprises E17N, V31L, D39G, D71S, D83A, and V84K.

64. The host cell of claim 40, wherein relative to SEQ ID NO: 1, the PKC comprises D13R, A18D, T26R, E52H, Y55F, Q70A, G80A, D83N, V84K, and Y85F.

65. The host cell of claim 40, wherein relative to SEQ ID NO: 1, the PKC comprises E17N, A18D, T26R, V31L, D71S, D83A, and V84K.

66. The host cell of claim 40, wherein relative to SEQ ID NO: 1, the PKC comprises E17N, V31L, I33N, D71S, G82R, and V84F.

67. The host cell of claim 40, wherein relative to SEQ ID NO: 1, the PKC comprises E17N, V31L, I33N, D71S, V84K, and T98K.

68. The host cell of claim 40, wherein relative to SEQ ID NO: 1, the PKC comprises V84K, T26R, D39G, D83E, D13R, and T98K.

69. The host cell of claim 40, wherein relative to SEQ ID NO: 1, the PKC comprises V84K, T26R, D83E, D13R, T98K, and S65N.

70. The host cell of claim 40, wherein relative to SEQ ID NO: 1, the PKC comprises E17N, V31L, I33N, V66Y, D71E, and V84F.

71. The host cell of claim 40, wherein relative to SEQ ID NO: 1, the PKC comprises E17N, A18D, V31L, I33N, D71E, and V84F.

72. The host cell of claim 40, wherein relative to SEQ ID NO: 1, the PKC comprises E17N, A18D, V31L, I33N, D71S, G80A, and V84F.

73. The host cell of claim 40, wherein relative to SEQ ID NO: 1, the PKC comprises A18D, T26R, E52H, Y55F, Q70A, G80A, D83N, V84K, Y85F, and F₈₈N.

74. The host cell of claim 40, wherein relative to SEQ ID NO: 1, the PKC comprises E17N, A18D, V31L, I33N, D71S, V84K, and T98K.

75. The host cell of claim 40, wherein relative to SEQ ID NO: 1, the PKC comprises E17N, A18D, V31L, I33N, S65N, D71S, and V84K.

76. The host cell of claim 40, wherein relative to SEQ ID NO: 1, the PKC comprises T26R, I33H, Y55F, Q70A, G80A, D83K, V84K, and F₈₈N.

77. The host cell of claim 40, wherein relative to SEQ ID NO: 1, the PKC comprises A18D, E22K, T26R, E52H, Y55F, Q70A, D71E, G80A, D83N, and V84K.

78. The host cell of claim 40, wherein relative to SEQ ID NO: 1, the PKC comprises E17N, V31L, 133N, D71E, G82R, and V84F.

79. A host cell that comprises a heterologous polynucleotide encoding a polyketide cyclase (PKC), wherein the PKC comprises at least 6 amino acid substitutions or deletions relative to SEQ ID NO: 1, wherein the PKC comprises at least two amino acid substitutions in a domain corresponding to helix 3 (positions 76-85 in SEQ ID NO: 1), and wherein the PKC is capable of producing olivetolic acid from 3,5,7-trioxododecanoyl-CoA.

80. The host cell of claim 79, wherein the at least two amino acid includes an amino acid substitution at position 84 relative to SEQ ID NO: 1.

81. The host cell of claim 79-80, wherein at least one of the at least two amino acid substitutions increases the stability of a kinked helix defined by the domains corresponding to (positions 66-73 in SEQ ID NO: 1), turn 1 (positions 74-75 in SEQ ID NO: 1), and helix 3 (positions 76-85 in SEQ ID NO: 1) and/or improves conformation of a binding pocket of the PKC to 3,5,7-trioxododecanoyl-CoA and/or olivetolic acid.

82. The host cell of any one of claims 79-81, wherein the at least two amino acid substitutions in the domain corresponding to helix 3 (positions 76-85 in SEQ ID NO: 1) are at residues corresponding to positions 80 and 82 in SEQ ID NO: 1.

83. The host cell of any one of claims 79-82, wherein the PKC comprises:
a) the amino acid E, A, or S at the residue corresponding to position 80 in SEQ ID NO: 1; and/or
b) the amino acid V or K at the residue corresponding to position 82 in SEQ ID NO: 1.

84. The host cell of any one of claims 79-83 wherein the PKC further comprises an amino acid substitution at a residue corresponding to position 73 or 74 in SEQ ID NO: 1.

85. The host cell of claim 84, wherein:
a) the PKC comprises the amino acid L at the residue corresponding to position 73 in SEQ ID NO: 1; and/or
b) the PKC comprises the amino acid S or L at the residue corresponding to position 74 in SEQ ID NO: 1.

86. The host cell of any one of claims 79-85, wherein the PKC comprises an amino acid substitution at a residue corresponding to any of positions position 84 and/or 86-89 in SEQ ID NO: 1.

87. The host cell of claim 86, wherein the PKC comprises:
a) the amino acid M at the residue corresponding to position 84 in SEQ ID NO: 1;
b) the amino acid G at the residue corresponding to position 86 in SEQ ID NO: 1;
c) the amino acid D at the residue corresponding to position 87 in SEQ ID NO: 1;
d) the amino acid Y at the residue corresponding to position 88 in SEQ ID NO: 1; and/or
e) the amino acid V at the residue corresponding to position 89 in SEQ ID NO: 1.

88. The host cell of any one of claims 79-87, wherein the host cell produces at least 10%, 20%, 30%, 40%, 50%, 60%, 70%, 80%, 90%, 100%, or more of the PKC relative to a control host cell.

89. The host cell of claim 88, wherein the control host cell does not comprise the PKC.

90. The host cell of any one of claims 79-89, wherein the PKC is capable of producing at least 10%, 20%, 30%, 40%, 50%, 60%, 70%, 80%, 90%, 100%, or more of olivetolic acid relative to a control.

91. The host cell of claim 90, wherein the control is a PKC that does not comprise one or more of the at least two amino acid substitutions in the domain corresponding to helix 3 (positions 76-85 in SEQ ID NO: 1).

92. The host cell of claim 5, wherein the PKC comprises one or more of the following:
a) the amino acid G at a residue corresponding to position 2 in SEQ ID NO: 1;
b) the amino acid M or Y at a residue corresponding to position 3 in SEQ ID NO: 1;
c) the amino acid R or K at a residue corresponding to position 13 in SEQ ID NO: 1;
d) the amino acid N at a residue corresponding to position 17 in SEQ ID NO: 1;
e) the amino acid D or R at a residue corresponding to position 18 in SEQ ID NO: 1;
f) the amino acid E or D at a residue corresponding to position 19 in SEQ ID NO: 1;
g) the amino acid A at a residue corresponding to position 20 in SEQ ID NO: 1;
h) the amino acid K at a residue corresponding to position 21 in SEQ ID NO: 1;
i) the amino acid T or K at a residue corresponding to position 22 in SEQ ID NO: 1;
j) the amino acid A or N at a residue corresponding to position 25 in SEQ ID NO: 1;
k) the amino acid H, R, A, or E at a residue corresponding to position 26 in SEQ ID NO: 1;
l) the amino acid A, S, E, or H at a residue corresponding to position 28 in SEQ ID NO: 1;
m) the amino acid D, S, E, or T at a residue corresponding to position 29 in SEQ ID NO: 1;
n) the amino acid L or A at a residue corresponding to position 31 in SEQ ID NO: 1;
o) the amino acid H, R, D, E, K, Q, or N at a residue corresponding to position 33 in SEQ ID NO: 1;
p) the amino acid L at a residue corresponding to position 37 in SEQ ID NO: 1;
q) the amino acid L at a residue corresponding to position 38 in SEQ ID NO: 1;
r) the amino acid G, S, or Q at a residue corresponding to position 39 in SEQ ID NO: 1;
s) the amino acid K or R at a residue corresponding to position 41 in SEQ ID NO: 1;
t) the amino acid I at a residue corresponding to position 45 in SEQ ID NO: 1;
u) the amino acid K at a residue corresponding to position 50 in SEQ ID NO: 1;
v) the amino acid H at a residue corresponding to position 52 in SEQ ID NO: 1;
w) the amino acid H at a residue corresponding to position 53 in SEQ ID NO: 1;
x) the amino acid F at a residue corresponding to position 55 in SEQ ID NO: 1;
y) the amino acid Y at a residue corresponding to position 56 in SEQ ID NO: 1;
z) the amino acid Y at a residue corresponding to position 57 in SEQ ID NO: 1;
aa) the amino acid I or L at a residue corresponding to position 60 in SEQ ID NO: 1;
bb) the amino acid M at a residue corresponding to position 62 in SEQ ID NO: 1;
cc) the amino acid Q or D at a residue corresponding to position 64 in SEQ ID NO: 1;
dd) the amino acid N at a residue corresponding to position 65 in SEQ ID NO: 1;
ee) the amino acid K at a residue corresponding to position 67 in SEQ ID NO: 1;
ff) the amino acid E, G, or H at a residue corresponding to position 68 in SEQ ID NO: 1;
gg) the amino acid A or L at a residue corresponding to position 70 in SEQ ID NO: 1;
hh) the amino acid Q or S, at a residue corresponding to position 71 in SEQ ID NO: 1;
ii) the amino acid D, E, H, or Q at a residue corresponding to position 74 in SEQ ID NO: 1;
jj) the amino acid A or G at a residue corresponding to position 83 in SEQ ID NO: 1;
kk) the amino acid F, K, M, or Q at a residue corresponding to position 84 in SEQ ID NO: 1;
ll) the amino acid A or F at a residue corresponding to position 85 in SEQ ID NO: 1;
mm) the amino acid G at a residue corresponding to position 86 in SEQ ID NO: 1;
nn) the amino acid D or K at a residue corresponding to position 87 in SEQ ID NO: 1;
oo) the amino acid Y, N, or S at a residue corresponding to position 88 in SEQ ID NO: 1;
pp) the amino acid M or I at a residue corresponding to position 95 in SEQ ID NO: 1;
qq) the amino acid F at a residue corresponding to position 97 in SEQ ID NO: 1;
rr) the amino acid E, K, or Y at a residue corresponding to position 98 in SEQ ID NO: 1;
the amino acid A, G, K, N, Q, or S at a residue corresponding to position 100 in SEQ ID NO: 1; and/or
ss) the amino acid A or E at a residue corresponding to position 101 in SEQ ID NO: 1.

93. The host cell of claim 92, wherein the PKC further comprises one or more of the following:
a) the amino acid M at a residue corresponding to position 24 in SEQ ID NO: 1;
b) the amino acid F at a residue corresponding to position 46 in SEQ ID NO: 1;
c) the amino acid A at a residue corresponding to position 48 in SEQ ID NO: 1;
d) the amino acid Q at a residue corresponding to position 49 in SEQ ID NO: 1;
e) the amino acid I or F at a residue corresponding to position 59 in SEQ ID NO: 1;
f) the amino acid T, D, or S at a residue corresponding to position 61 in SEQ ID NO: 1;
g) the amino acid L, M, or Y at a residue corresponding to position 66 in SEQ ID NO: 1;
h) the amino acid L or Y at a residue corresponding to position 69 in SEQ ID NO: 1;
i) the amino acid L at a residue corresponding to position 73 in SEQ ID NO: 1;
j) the amino acid S at a residue corresponding to position 74 in SEQ ID NO: 1;
k) the amino acid V, A, E, S, D, or N at a residue corresponding to position 80 in SEQ ID NO: 1;
l) the amino acid Y at a residue corresponding to position 81 in SEQ ID NO: 1;
m) the amino acid K, V, or R at a residue corresponding to position 82 in SEQ ID NO: 1;
n) the amino acid V or M at a residue corresponding to position 89 in SEQ ID NO: 1; and/or
o) the amino acid V at a residue corresponding to position 94 in SEQ ID NO: 1.

94. The host cell of claim 92 or 93, wherein the PKC comprises at least 6, 7, 8, 9, or 10 amino acid substitutions relative to SEQ ID NO: 1.

95. The host cell of any one of claims 90-94, wherein the PKC is capable of producing at least 10%, 20%, 30%, 40%, 50%, 60%, 70%, 80%, 90%, 100%, or more of olivetolic acid relative to a control.

96. The host cell of claim 95, wherein the control is a PKC that does not comprise an amino acid substitution at a residue corresponding to position 2, 3, 13, 17, 18, 19, 20, 21, 22, 25, 26, 28, 29, 31, 33, 37, 38, 39, 41, 45, 50, 52, 53, 55, 56, 57, 60, 62, 64, 65, 67, 68, 70, 71, 74, 83, 84, 85, 86, 87, 88, 95, 97, 98, 100, and/or 101 in SEQ ID NO: 1.

97. A host cell that comprises a heterologous polynucleotide encoding a polyketide cyclase (PKC), wherein the PKC comprises one or more of the following amino acid substitutions 17, 18, 29, 31, 33, 39, 71, 83, and/or 84 relative to SEQ ID NO: 1, and wherein the PKC is capable of producing olivetolic acid from 3,5,7-trioxododecanoyl-CoA.

98. The host cell of claim 97, wherein the PKC comprises one or more of the following amino acid substitutions E17N, A18D, N29T, V31L, I33N, D39Q, D71S, D83A, and/or V84F relative to SEQ ID NO: 1.

99. The host cell of claim 97 or 98, wherein the PKC comprises at least 6, 7, 8, 9, or 10 amino acid substitutions or deletions relative to SEQ ID NO: 1.

100. The host cell of any one of claims 97-99, wherein the PKC comprises:
a) E17N, A18D, V31L, D39Q, D71S, and D83A relative to SEQ ID NO: 1;
b) E17N, A18D, V31L, D71S, D83A, and V84F relative to SEQ ID NO: 1; or
c) A18D, V31L, D39Q, D71S, D83A, and V84F relative to SEQ ID NO: 1.

101. The host cell of claim 100, wherein the PKC comprises a sequence that is at least 90% identical to SEQ ID NO: 255, 220, or 707.

102. The host cell of claim 101, wherein the PKC comprises SEQ ID NO: 255, 220, 707.

103. The host cell of any one of claims 97-102, wherein the PKC produces more olivetolic acid as compared to a control PKC comprising SEQ ID NO: 1.

104. The host cell of any one of claims 97-103, wherein the PKC produces more than 41,000 μg/L olivetolic acid.

105. The host cell of any one of claims 97-104, wherein relative to a control PKC comprising SEQ ID NO: 1, the PKC produces a greater ratio of olivetolic acid to olivetol.

106. The host cell of any one of claims 97-105, wherein the PKC produces a ratio of olivetolic acid to olivetol that is higher than 3.1.

107. A host cell that comprises a heterologous polynucleotide encoding a polyketide cyclase (PKC), wherein the PKC comprises one or more amino acid substitutions relative to SEQ ID NO: 1 selected from the group consisting of:
a) D13R;
b) A18D;
c) E22K;
d) T26R or T26H;
e) N29S;
f) I33R or I33H;
g) D39G;
h) K49Q;
i) E52H;
j) Y55F;
k) Q70A or Q70L;
l) D71Q;
m) I74G;
n) G80A;
o) D83K or D83N;
p) V84K; and
q) Y85A,
wherein the PKC is capable of producing olivetolic acid from 3,5,7-trioxododecanoyl-CoA (4a).

108. The host cell of claim 107, wherein the PKC comprises at least 6, at least 7, at least 8, at least 9, or at least 10 amino acid substitutions or deletions relative to SEQ ID NO: 1.

109. The host cell of claim 107 or 108, wherein relative to SEQ ID NO: 1, the PKC comprises:
a) I33R, E52H, Y55F, Q70A, G80A, D83K, and V84K;
b) D13R, A18D, N29S, Q70L, D71Q, G80A, and D83K;
c) I33R, E52H, Y55F, Q70A, 174G, and D83K;
d) T26R, I33H, E52H, Y55F, Q70A, and D83K;
e) A18D, T26R, E52H, Y55F, Q70A, G80A, D83N, V84K, and Y85A;
f) T26R, I33H, Y55F, Q70A, G80A, D83K, and Y85A; or
g) E22K, T26H, N29S, D39G, K49Q, E52H, D83N, and V84K.

110. The host cell of claim 109, wherein the PKC comprises a sequence that is at least 90% identical to SEQ ID NO: 283, 289, 294, 344, 345, 349, or 365.

111. The host cell of claim 110, wherein the PKC comprises SEQ ID NO: 283, 289, 294, 344, 345, 349, or 365.

112. The host cell of any one of claims 107-111, wherein the PKC produces more olivetolic acid as compared to a control PKC comprising SEQ ID NO: 1.

113. The host cell of any one of claims 107-112, wherein the PKC produces more than 94,000 ug/L olivetolic acid.

114. The host cell of any one of claims 1-113, wherein the host cell is a plant cell, an algal cell, a yeast cell, a bacterial cell, or an animal cell.

115. The host cell of claim 114, wherein the host cell is a yeast cell.

116. The host cell of claim 115, wherein the yeast cell is a Saccharomyces cell, a Yarrowia cell, or a Komagataella cell.

117. The host cell of claim 116, wherein the Saccharomyces cell is a Saccharomyces cerevisiae cell.

118. The host cell of claim 116, wherein the yeast cell is a Yarrowia cell.

119. The host cell of claim 114, wherein the host cell is a bacterial cell.

120. The host cell of claim 119, wherein the bacterial cell is an E. coli cell.

121. The host cell of any one of claims 1-120, wherein the host cell further comprises one or more heterologous polynucleotides comprising one or more of: an acyl activating enzyme (AAE), a polyketide synthase (PKS), a prenyltransferase (PT), and/or a terminal synthase (TS).

122. The host cell of claim 121, wherein the polyketide synthase is an olivetol synthase (OLS).

123. The host cell of claim 121 or 122, wherein the terminal synthase is a cannabidiolic acid synthase (CBDAS).

124. The host cell of claim 121 or 122, wherein the terminal synthase is a tetrahydrocannabinolic acid synthase (THCAS).

125. The host cell of claim 121 or 122, wherein the terminal synthase is a cannabichromenic acid synthase (CBCAS).

126. A method comprising culturing the host cell of any one of claims 1-125.

127. A method for producing 2,4-dihydroxybenzoic acid optionally substituted at position 6 comprising contacting: a tetraketide with a polyketide cyclase (PKC), wherein the PKC comprises an amino acid substitution at one or more residues corresponding to one more or more positions selected from the group consisting of position 17, 18, 29, 31, 33, 39, 71, 83 and 84 in SEQ ID NO: 1.

128. The method of claim 127, wherein the PKC comprises at least 6, 7, 8, 9, or 10 amino acid substitutions or deletions relative to SEQ ID NO: 1.

129. The method of claim 127 or 128, wherein the tetraketide is 3,5,7-trioxododecanoyl-CoA.

130. The method of any one of claims 127-129, wherein the 2,4-dihydroxybenzoic acid optionally substituted at position 6 is olivetolic acid.

131. The method of any one of claims 127-130, wherein contacting the tetraketide with the PKC occurs in vitro.

132. The method of any one of claims 127-130, wherein contacting the tetraketide with the PKC occurs in vivo.

133. The method of any one of claims 127-132, wherein the PKC comprises at least six amino acid substitutions selected from the group consisting of E17N, A18D, N29T, V31L, I33N, D39Q, D71S, D83A, and V84F relative to SEQ ID NO: 1.

134. A non-naturally occurring nucleic acid encoding a polyketide cyclase (PKC), wherein the non-naturally occurring nucleic acid encodes a PKC comprising a sequence with at least one amino acid substitution relative to SEQ ID NO: 1 and with at least 90% identity to SEQ ID NO: 62, 87, 220, 234, 219, 258, 231, 240, 255, 707, 749, 751, 765, 768, 790, 808, 810, 822, 828, 840, 846, 857, 862, 865, 921, 922, 858, 850, 851, 933, 871, 809, 926, 923, 924, 758, 747, 757, 875, 908, 823, 833, or 777.

135. A non-naturally occurring nucleic acid encoding a polyketide cyclase (PKC) wherein relative to the sequence of SEQ ID NO: 1 the wherein the PKC comprises an amino acid substitution at one or more residues corresponding to one or more positions selected from the group consisting of position 17, 18, 29, 31, 33, 39, 71, 83 and 84 in SEQ ID NO: 1 and wherein the PKC is capable of producing olivetolic acid from 3,5,7-trioxododecanoyl-CoA.

136. The non-naturally occurring nucleic acid of claim 135, wherein the PKC comprises at least 6 amino acid substitutions or deletions relative to SEQ ID NO: 1.

137. A non-naturally occurring nucleic acid encoding a polyketide cyclase (PKC) wherein relative to the sequence of SEQ ID NO: 1, the PKC comprises an amino acid substitution at one or more residues corresponding to one or more positions selected from the group consisting of position 13, 17, 18, 22, 26, 31, 33, 39, 52, 55, 64, 65, 66, 70, 71, 74, 80, 82, 83, 84, 85, 88, and/or 98 in SEQ ID NO: 1 and wherein the PKC is capable of producing olivetolic acid from 3,5,7-trioxododecanoyl-CoA.

138. A vector comprising the non-naturally occurring nucleic acids of any one of claims 134-137.

139. An expression cassette comprising the non-naturally occurring nucleic acids of any one of claims 134-137.

140. A non-naturally occurring polyketide cyclase (PKC) wherein the PKC comprises an amino acid substitution at one or more residues corresponding to one or more positions selected from the group consisting of position 17, 18, 29, 31, 33, 39, 71, 83 and 84 in SEQ ID NO: 1, and wherein the PKC is capable of producing olivetolic acid from 3,5,7-trioxododecanoyl-CoA.

141. A non-naturally occurring polyketide cyclase (PKC) wherein the PKC comprises an amino acid substitution at one or more residues corresponding to one or more positions selected from the group consisting of position 13, 17, 18, 22, 26, 31, 33, 39, 52, 55, 64, 65, 66, 70, 71, 74, 80, 82, 83, 84, 85, 88, and/or 98 in SEQ ID NO: 1, and wherein the PKC is capable of producing olivetolic acid from 3,5,7-trioxododecanoyl-CoA.

142. The non-naturally occurring PKC of claim 140, wherein the PKC comprises at least 6, 7, 8, 9, or 10 amino acid substitutions or deletions relative to SEQ ID NO: 1.

143. A host cell comprising the non-naturally occurring nucleic acid of any one of claims 134-137, the vector of claim 138, the expression cassette of claim 139 or the non-naturally occurring PKC of any one of claims 140-142.

144. A host cell that comprises a heterologous polynucleotide encoding a polyketide cyclase (PKC), wherein the PKC comprises one or more amino acid substitutions relative to SEQ ID NO: 1 selected from the group consisting of:
a) A2G;
b) V3M or V3Y;
c) D13R or D13K;
d) E17N;
e) A18D or A18R;
f) Q19E or Q19D;
g) K20A;
h) E21K;
i) E22T or E22K;
j) K25A or K25N;
k) T26H, T26R, T26A, or T26E;
l) V28A, V28S, V28E, or V28H;
m) N29D, N29S, N29E, or N29T;
n) V31L or V31A;
o) 133H, 133R, 133D, 133E, 133K, 133Q, or 133N;
p) M37L;
q) K38L;
r) D39G, D39S, or D39Q;
s) Y41K or Y41R;
t) D45I;
u) N50K;
v) E52H;
w) E53H;
x) Y55F;
y) T56Y;
z) H₅₇Y;
aa) E601 or E60L;
bb) T62M;
cc) E64Q or E64D;
dd) S65N;
ee) E67K;
ff) T68E, T68G, or T68H;
gg) Q70A or Q70L;
hh) D71Q or D71S;
ii) 174D, 174E, 174H, or 174Q;
jj) D83A or D83G;
kk) V84F, V84K, V84M, or V84Q;
ll) Y85A or Y85F;
mm) R86G;
nn) S87C, S87D, or S87K;
oo) F₈₈Y, F₈₈N, or F₈₈S;
pp) F₉₅M or F₉₅I;
qq) Y97F;
rr) T98E, T98K, or T98Y;
ss) R100A, R100G, R100K, R100N, R100Q, or R100S; and
tt) K101A or K101E.

145. The host cell of claim 144, wherein the PKC further comprises one or more of the following amino acid substitutions relative to SEQ ID NO: 1:
a) F₂₄M;
b) V46F;
c) Q48A;
d) K49Q;
e) N50F or N50W;
f) V59I or V59F;
g) V61T, V61D, or V61S;
h) V66L, V66M, or V66Y;
i) 169L or 169Y;
j) D71E;
k) I73L;
l) 174S, 174G, or 174L;
m) G80V, G80A, G80E, G80S, G80D, or G80N;
n) F₈₁Y;
o) G82K, G82V, or G82R;
p) D83E, D83K, D83N, D83Q, D83R, or D83S;
q) R86N or R86S;
r) W89V or W89M; and/or
s) I94V.

146. The host cell of claim 144 or 145, wherein the PKC comprises at least 6, 7, 8, 9, or 10 amino acid substitutions or deletions relative to SEQ ID NO: 1.
